(19) United States
(12) Patent Application Publication
(10) Pub. No.: US 2022/0340849 A1
(43) Pub. Date: Oct. 27, 2022

(54) BRAIN-CHIP MODELING NEURODEGENERATION AND NEUROINFLAMMATION IN PARKINSON'S DISEASE

(71) Applicant: EMULATE, INC., Boston, MA (US)

(72) Inventors: Iosif Pediaditakis, Boston, MA (US); William R. Tien-Street, Burtonsville, MD (US); S. Jordan Kerns, Reading, MA (US); Geraldine Hamilton, Boston, MA (US); Daniel Levner, Brookline, MA (US)

(21) Appl. No.: 17/719,627

(22) Filed: Apr. 13, 2022

Related U.S. Application Data
(63) Continuation of application No. PCT/US2020/056245, filed on Oct. 19, 2020.
(60) Provisional application No. 63/045,608, filed on Jun. 29, 2020.
(60) Provisional application No. 62/923,256, filed on Oct. 18, 2019.

(51) Int. Cl. C12M 3/06; C12N 5/079; C12N 5/00; C07K 14/47; C12M 1/12; G01N 33/50

(52) U.S. Cl. CPC C12M 23/16; C12N 5/0618; C12N 5/0075; C07K 14/47; C12M 25/02; G01N 33/5058; C12N 2502/086

(57) ABSTRACT

The invention relates to modeling brain neuronal disease in a microfluidic device, comprising a co-culture of iPS-derived brain endothelial cells; iPS-derived dopaminergic neurons; primary microglia; and primary astrocytes, a Blood-Brain-Barrier (BBB)-Chip and a Brain-Chip. In particular, cross-talk between glial cells (e.g. microglia and astrocytes) with neuronal cells, in further contact with endothelial cells is contemplated for use for identifying drug targets under conditions for inducing in vivo relevant neuronal inflammation, neurodegeneration and neuronal death. Thus, in one embodiment, a microfluidic Brain-Chip comprising a co-culture of brain cells is exposed to α-synuclein preformed fibrils (PFF), a type of pathogenic form of α-synuclein. Such α-synuclein PFF exposure demonstrates an in vivo relevant disease pathogenesis on a microfluidic device as a concentration- and time-controlled manner that may be used for preclinical drug evaluation for diseases related to neuronal inflammation, e.g. Parkinson's disease (PD). In some embodiments, modulation of complement in the presence of neuronal inflammation is contemplated. In some embodiments, drug delivery to brain cells across the BBB is contemplated for preclinical testing of drug efficacy for slowing or stopping neuronal inflammation and degeneration.

This application is a Continuation of, and claims priority to, co-pending PCT Patent Application Serial No. PCT/US2020/056245, filed Oct. 19, 2020, which claims priority to Provisional Application Serial Nos. 62/923,256 filed on Oct. 18, 2019 and 63/045,608 filed on Jun. 29, 2020 now expired, the contents of which are incorporated herein in their entirety.

### STATEMENT OF GOVERNMENT SUPPORT

This invention was made with government support under grant numbers UG3TR002188 awarded by the National Institute of Health, National Center for Advancing Translational Sciences. The government has certain rights in the invention.

### SEQUENCE LISTING

A Sequence Listing has been submitted in an ASCII text file named “200445T25,” created on Jul. 1, 2022, consisting of 1,617 bytes, the entire content of which is herein incorporated by reference.

### FIELD OF THE INVENTION

The invention relates to modeling brain neuronal disease in a microfluidic device, comprising a co-culture of a variety of cells type such as iPS-derived brain endothelial cells; iPS-derived dopaminergic neurons; primary microglia; and primary astrocytes, a Blood-Brain-Barrier (BBB)-Chip and a Brain-Chip. In particular, cross-talk between glial cells (e.g. microglia and astrocytes) with neuronal cells, in further contact with endothelial cells is contemplated for use for identifying drug targets under conditions for inducing in vivo relevant neuronal inflammation, neurodegeneration and neuronal death. Thus, in one embodiment, a microfluidic Brain-Chip comprising a co-culture of brain cells is exposed to α-synuclein preformed fibrils (PFF), a type of pathogenic form of α-synuclein. Such α-synuclein PFF exposure demonstrates an in vivo relevant disease pathogenesis on a microfluidic device as a concentration- and time-controlled manner that may be used for preclinical drug evaluation for diseases related to neuronal inflammation, e.g. Parkinson's Disease (PD). In some embodiments, modulation of complement in the presence of neuronal inflammation is contemplated. In some embodiments, drug delivery to brain cells across the BBB is contemplated for preclinical testing of drug efficacy for slowing or stopping neuronal inflammation and degeneration.

### BACKGROUND

Animal models are used for preclinical drug testing for treating Parkinson's Disease (PD). Genetic mouse models (e.g. α-synuclein, LRRK2, PINK1/Parkin, and DJ-1 engineered mice strains) are able to recapitulate specific aspects of PD, although none has reproduced so far the neuronal degeneration associated with PD.

Neurotoxic models (e.g. 6-OHDA, MPTP engineered mouse strains) mimic many aspects of the disease, including having the ability to induce oxidative stress and to cause cell death in dopaminergic (DA) neuronal populations. This type of DA neuronal cell death reflects what is seen in PD, however this is merely one aspect related to DA death in PD.

There are many drawbacks to the use of these models, including lacking a parallel for mimicking the time factor of symptoms/neuronal degeneration in these models versus the time factor in the human condition; many clinical features in humans with PD are not present. The main problem with animal-identified drug targets is they do not correlate well with human targets. Thus, there is a lack of development of associated efficacious drugs using mouse models.

Therefore, there is a need for a clinically relevant in vitro model for identifying clinically relevant drug targets and testing drugs for those targets for treating neuronal inflammation.

### SUMMARY OF THE INVENTION

The invention relates to methods, devices and systems for modeling brain neuronal disease in a microfluidic device, comprising a co-culture of a variety of cell types such as iPS-derived brain endothelial cells; iPS-derived dopaminergic neurons; primary microglia; and primary astrocytes, a Blood-Brain-Barrier (BBB)-Chip and a Brain-Chip. In particular, cross-talk between glial cells (e.g. microglia and astrocytes) with neuronal cells, in further contact with endothelial cells is contemplated for use for identifying drug targets under conditions for inducing in vivo relevant neuronal inflammation, neurodegeneration and neuronal death. Thus, in one embodiment, a microfluidic Brain-Chip comprising a co-culture of brain cells is exposed to α-synuclein preformed fibrils (PFF), a type of pathogenic form of α-synuclein. Such α-synuclein PFF exposure demonstrates an in vivo relevant disease pathogenesis on a microfluidic device as a concentration- and time-controlled manner that may be used for preclinical drug evaluation for diseases related to neuronal inflammation, e.g. Parkinson's Disease (PD). In some embodiments, modulation of complement in the presence of neuronal inflammation is contemplated. In some embodiments, drug delivery to brain cells across the BBB is contemplated for preclinical testing of drug efficacy for slowing or stopping neuronal inflammation and degeneration.

Moreover, the invention relates to the use of a sensory neuron ECM consisting of collagen IV, laminin and fibronectin. In particular, the use of sensory neuron ECM provided longer term benefits when used with iPSC progenitor neurons for seeding microfluidic chips. Sensory neuron ECM is contemplated for use in microfluidic innervated chips, such as those described herein. Thus, use of Sensory neuron ECM encompasses other types of innervated microfluidic chips, including but not limited to brain chips and intestine (gut) chips.

In one embodiment, a brain chip is provided by seeding induced pluripotent stem cells (iPSC)-derived cortical neurons, primary astrocytes and primary pericytes in the neuronal channel (top), and iPSC-derived brain microvascular endothelial cells in the vascular channel (bottom). It is not meant to limit a Brain chip to a particular mammal, indeed, a Brain chip may be seeded with cells including but not limited to humans, monkeys, rats and mice. In one embodiment, a human brain chip was provided by seeding human induced pluripotent stem cells (iPSC)-derived cortical neurons, human primary astrocytes and human primary pericytes in the neuronal channel (top), and iPSC-derived human brain microvascular endothelial cells in the vascular channel (bottom). Neuronal cells may also include iPSC-derived neurons, glutamatergic neurons, cortical neurons, and cortical glutamatergic neurons.

An exemplary method, comprising, a) providing, i) a plurality of altered α-synuclein proteins, wherein said molecules are capable of crossing an intact blood brain barrier comprising brain endothelial cells having a permeability level; and ii) a microfluidic device comprising a membrane, said membrane separating two microfluidic channels, wherein one channel is seeded with brain endothelial cells forming an intact cell barrier, and b) contacting said cells with said plurality of altered α-synuclein protein molecules, wherein said contacting comprises flowing said α-synuclein protein molecules into said channel for reducing said permeability level of said blood brain barrier. In one embodiment, said method further providing a test compound, wherein said compound does not cross an intact blood brain barrier, and comprising a step c) treating said cells with said test compound for determining the amount of said compound crossing said barrier (e.g. because the barrier is not intact, i.e. has become permeable or simply more permeable to the compound), wherein treating comprises flowing said test compound into said channel. In one embodiment, said other channel is seeded with cell types selected from the group consisting of pericytes, astrocytes, microglial, sensory neurons, sensory neuronal progenitors, cortex neurons and cortex neuronal progenitors, wherein at least one cell type expresses an inflammatory biomarker after said contacting with said altered α-synuclein proteins for identifying changes in said inflammatory biomarker before and after said treatment with said test compound for identifying a test compound as an anti-inflammatory treatment. In one embodiment, said other channel is seeded with cell types selected from the group consisting of pericytes, astrocytes, microglial, sensory neurons, sensory neuronal progenitors, cortex neurons and cortex neuronal progenitors, wherein at least one cell type expresses an inflammatory biomarker after said contacting with said altered α-synuclein proteins for identifying changes in said biomarkers for identifying a drug target. In one embodiment, said method further providing a test compound for said identified drug target, and comprising a step c) adding said test compound for identifying changes in said biomarker expression for increasing said permeability level of said barrier. In one embodiment, said altered α-synuclein are α-synuclein preformed fibrils (PFF). In one embodiment, said method is for identifying cellular changes induced by altered α-synuclein. In one embodiment, said cellular changes are selected from the group consisting of identifying changes in cellular interactions, changes in biomarker expression, changes in Ca++ signaling, changes in cytokine expression, changes in cytokine secretion and changes in cell viability.

In one embodiment, the invention provides a method of identifying a drug target for a neural disease, comprising, a) providing, i) one or more inflammatory inducing molecules (including but not limited to TNF); and ii) a microfluidic device comprising a membrane, said membrane separating two microfluidic channels, wherein one channel is seeded with brain endothelial cells forming an intact cell barrier, and the other channel is seeded with cell types selected from the group consisting of pericytes, astrocytes, microglial, sensory neurons, sensory neuronal progenitors, cortex neurons and cortex neuronal progenitors, wherein each cell type is capable of expression a biomarker associated with inflammation, and iii) a test compound, and b) contacting said cells with said inflammatory inducing molecules, wherein said contacting comprises flowing said inflammatory inducing molecules into said channel for inducing expression of a biomarker associated with inflammation for identifying a drug target; and c) treating said inflamed cells with said test compound for reducing expression of said inflammatory biomarker. In one embodiment, said biomarker is selected from the group consisting of a biomarker for barrier function (permeability), a biomarker for cellular interactions, a biomarker for changes in Ca++ signaling, a biomarker for changes in cytokine expression, biomarker for changes in cytokine secretion and a biomarker for changes in cell viability. In one embodiment, said inflammatory inducing molecules are selected from the group consisting of TNF-alpha, a blocking antibody for a complement protein and altered α-synuclein.

In one embodiment, the invention provides a method of modulating complement proteins in a microfluidic chip, comprising, a) providing, i) a blocking antibody for a complement protein (anti-complement antibody), ii) an inflammatory inducing molecule selected from the group consisting of TNF-alpha and altered α-synuclein (PFF), and iii) a microfluidic device comprising a membrane, said membrane separating two microfluidic channels, wherein one channel is seeded with endothelial cells forming an intact cell barrier, and the other channel seeded with parenchyma cells, wherein cells in at least one said channel are capable of expressing an inflammatory associated biomarker, and b) contacting said cells with said anti-complement antibody, wherein said contacting comprises flowing said antibody into said channel for reducing said inflammatory associated level of biomarker expression. In one embodiment, said complement protein is C1q (and said antibody is an Anti-C1q antibody). In one embodiment, said endothelial cells are primary brain endothelial cells and said other channel is seeded with cells selected from the group consisting of pericytes, astrocytes, microglial, sensory neurons, sensory neuronal progenitors, cortex neuron, for identifying changes in said inflammatory biomarker for identifying a drug target. In one embodiment, said cells are from the group consisting of primary cells, iPSC derived cells, biopsy derived cells and cell lines. In one embodiment, said biomarker is selected from the group consisting of a biomarker for: barrier function (permeability), cellular interactions, Ca++ signaling, cytokine expression, cytokine secretion and cell viability.

In yet another embodiment, the present invention contemplates a method, comprising a) providing i) α-synuclein (αSyn) fibrils or aggregates (as distinct from monomers, which can be used as a control); ii) a microfluidic device comprising at least one channel and, more preferably, a membrane, said membrane separating first and second microfluidic channels; and iii) a plurality of dopaminergic neurons in said first channel; b) contacting said dopaminergic neurons said α-synuclein fibrils or aggregates. In one embodiment, the cells are cultured under flow conditions (e.g. culture media is flowed through the channels at a flow rate, e.g. 30 ul/hr) prior to step b). It is not intended that the present invention be limited by the method by which the fibrils or aggregates are introduced into the device. In one embodiment, said contacting comprises flowing said α-synuclein fibrils into said first channel. In another embodiment, they are flowed into the second channel. In yet another embodiment, they are flowed into both channels. In one embodiment, said contacting with said fibrils causes accumulation of phosphorylated αSyn in said neurons. The present invention contemplates, in one embodiment, wherein the method further comprises detecting accumulation of phosphorylated αSyn in said neurons. In one embodiment, said accumulation is in a time dependent manner. In one embodiment, said contacting with said fibrils results in mitochondrial damage. In one embodiment, the present method further comprises detecting said mitochondrial damage in said neurons (or other cells in the microfluidic device). In one embodiment, the contacting with fibrils results in an increase in reactive oxygen species. In one embodiment, the present method further comprises detecting an increase in reactive oxygen species over time. In yet another embodiment, the method further comprises detecting an increase in caspase 3-positive neurons over time. In one embodiment, said contacting with said fibrils results in neuroinflammation. In one embodiment, the present method further comprises detecting neuroinflammation. In one embodiment, said contacting with said fibrils results in apoptosis. In one embodiment, the present method further comprises detecting said apoptosis. In one embodiment, said contacting with said fibrils results in some neuronal death. In one embodiment, the present method further comprises detecting neuronal death.

It is not intended that the present invention be limited to the situation where only neurons are in the device. In one embodiment, the present invention contemplates that said first channel further comprises a plurality of cells selected from the group consisting of pericytes, astrocytes, and microglia, and combinations thereof. It is not intended to be limited to specific combinations. Nonetheless, in one embodiment, said first channel further comprises astrocytes and microglial cells. In one embodiment, the present method further comprises detecting microglia activation. In one embodiment, the present method further comprises detecting astrocyte activation. In one embodiment, further comprises detecting astrogliosis.

It is not intended that the present invention be limited to just cells in the first channel. In one embodiment, said second channel comprises a population of endothelial cells. In one embodiment, the present invention contemplates detecting accumulation of αSyn in said endothelial cells. In one embodiment, the present invention contemplates detecting an inflammatory response of said endothelial cells. In one embodiment, said αSyn fibrils are introduced into said second channel.

A variety of read-outs for inflammation are contemplated, including but not limited to biomarker detection, whether at the protein level or RNA level. In one embodiment, the method further comprises detecting induced cytokine secretion. In one embodiment, said cytokine is a proinflammatory cytokine. In one embodiment, said cytokine is selected from the group consisting of IL-6, Interferon-gamma, IL-1B and TNF-alpha.

Endothelial cells allow one to measure and assess barrier function. In this regard, it is contemplated that said endothelial cells form tight junctions prior to step b). In one embodiment, said tight junctions define a barrier having a level of permeability. One embodiment, the present method further comprises detecting a change in said level of permeability after step b).

The present invention contemplates methods for testing compounds, including compounds that can reduce the negative impact of contacting said cells with said fibrils. In one embodiment, the method further comprises c) contacting said endothelial cells with a test compound. In one embodiment, the method contemplates detecting the impact of said test compound on said permeability.

It is believed that the introduction of fibrils into the microfluidic device can cause inflammation in one cell type, said inflammation propagating to other cells types in the device over time. In this regard, the present invention contemplates that the method, in one embodiment, further comprising detecting the propagation of inflammation to said microglia, astrocytes or pericytes (e.g. from said neurons or from said endothelial cells).

It is contemplated that the present invention offers a platform for testing cells from healthy humans (or other animals) along with humans with disease. In this regard, the present invention contemplates in one embodiment that said neurons are from a human patient. In one embodiment, said human patient has a neurological disease (e.g. Parkinson's, ALS, etc.).

The present invention contemplates, in one embodiment, exposing a more complete BBB to the fibrils. In this regard, the present invention contemplates, in one embodiment, a method, comprising a) providing, i) α-synuclein (αSyn) fibrils or aggregates (as distinct from monomers); ii) a microfluidic device comprising a membrane, said membrane separating first and second microfluidic channels; iii) a population of neurons in said first channel along with a plurality of cells selected from the group consisting of pericytes, astrocytes, microglia and combinations thereof; and v) a population of endothelial cells in said second channel; and b) culturing said plurality of cells in said first channel and culturing said population of endothelial cells in said second channel, under conditions such that a cell barrier forms having a level of permeability (e.g. under flow conditions, i.e. where culture media is introduced into the channels at a flow rates as discussed above); and c) contacting at least a portion of said cultured cells (or all of the cells) with said α-synuclein (αSyn) fibrils. For example, just the endothelial cells can be contacted in one embodiment, or just the neurons in another embodiment. In any event, the present invention contemplates, in one embodiment, a method further comprises d) detecting a change in said permeability (or in another feature of the BBB or the cells of the BBB). Again, it is not intended that the present invention be limited to how or where the fibrils are introduced. In one embodiment, said contacting comprises flowing said α-synuclein fibrils into said first channel.

Again, it is contemplated that the introduction of the fibrils will cause a number of phenotypes. In certain embodiments, these phenotypes can be detected. For example, in one embodiment, the present method further comprises detecting accumulation of phosphorylated αSyn in said neurons. In one embodiment, said accumulation is in a time dependent manner. In yet another embodiment, the present method further comprises detecting mitochondrial damage in said neurons. In still another embodiment, the present method further comprises detecting an increase in reactive oxygen species (e.g. over time). In an additional embodiment, the present method further comprises detecting an increase in caspase 3-positive neurons (e.g. over time). It yet another embodiment, the present method further comprises detecting neuroinflammation. In still another embodiment, the present method further comprises detecting apoptosis. In another embodiment, the present method contemplates, the method further comprises detecting some neuronal death (e.g. over time).

The presence of other cell types allows one to assess their state of activation. Thus, in one embodiment, the present invention contemplates that the method further comprises detecting microglia activation and or astrocyte activation. In one embodiment, the method further comprises detecting astrogliosis.

It was found, using the above described method that said contacting of the endothelial cells with fibrils results in accumulation of αSyn in said neurons. In one embodiment, the method further comprises detecting accumulation of αSyn in said endothelial cells. In one embodiment, the method further comprises detecting an inflammatory response of said endothelial cells. In one embodiment, said αSyn fibrils are introduced into said second channel.

As noted above, a variety of read outs of inflammation are contemplated including biomarkers, whether at the protein level or RNA level. In one embodiment, the method further comprises detecting induced cytokine secretion. In one embodiment, said cytokine is a proinflammatory cytokine. In one embodiment, said cytokine is IL-6.

It is contemplated that the introduction of fibrils can change in the level of permeability of the BBB. In one embodiment, the fibrils cause an increase in permeability. In one embodiment, the method further comprises detecting this increase.

As noted earlier, it is believed that the present invention offers a platform for testing drugs and test compounds that may reduce the negative impact of the fibrils. Thus, in one embodiment, the method further comprises d) exposing at least a portion of said cells to a test compound. In one embodiment, the method further comprising e) detecting an impact of said test compound. In one embodiment, the method further comprises detecting a reduction in permeability after exposure to said test compound.

Again, it is not intended that the present invention be limited to how or where the test compound is introduced in the device. In one embodiment, said treating comprises flowing said test compound into said first channel, second channel or both.

A variety of compounds can be tested. In one embodiment, said test compound is trehalose.

In a preferred embodiment, said neurons are dopaminergic neurons. In some embodiment, the functionality of said neurons is detected. For example, in one embodiment, the method further comprises detecting transient Ca++ signaling of said dopaminergic neurons prior to step c). In one embodiment, the method further comprises detecting the loss of said transient Ca++ signaling after step c).

As noted above, the exposure of one cell type to said fibrils can result in inflammation that is propagated to other cells in the microfluidic device. In one embodiment, the method further comprises detecting the propagation of inflammation to said microglia, astrocytes or pericytes (e.g. from said neurons and/or endothelial cells).

As noted above, it is believed that the present invention provides a platform for testing human cells (and the cells of other animals), whether healthy or diseased. In one embodiment, said neurons are from a human patient. In one embodiment, said human patient has a neurological disease (Parkinson's, ALS, etc.).

While there was extensive discussion above in terms of the use of the fibrils, the present invention is not limited to using only fibrils. Thus, in one embodiment, the present invention contemplates a method, comprising a) providing i) an inflammation inducing compound (e.g. a compound that can induce cells into an inflammatory response); ii) a microfluidic device comprising a membrane, said membrane separating first and second microfluidic channels; and iii) a plurality of dopaminergic neurons in said first channel; b) contacting said dopaminergic neurons said inflammation inducing compound. Again, it is not intended that the present method be limited to how or where the compound is introduced. In one embodiment, said contacting comprises flowing said inflammation inducing compound into said first channel, second channel or both.

It is contemplated that said compound will have a number of results and these results can be detected in a variety of ways. For example, in one embodiment, the method further comprising detecting mitochondrial damage in said neurons. In one embodiment, the method further comprises detecting an increase in reactive oxygen species over time. In one embodiment, the method further comprises detecting an increase in caspase 3-positive neurons over time. In one embodiment, the method further comprises detecting neuroinflammation. In one embodiment, the method further comprising detecting apoptosis. In one embodiment, the method further comprises detecting neuronal death.

It is not intended that the present invention be limited to having just neurons in the microfluidic device. Indeed, in a preferred embodiment, other cells of the BBB are included. Thus, in one embodiment, said first channel further comprises a plurality of cells selected from the group consisting of pericytes, astrocytes, and microglia, and combinations thereof. It is not intended that the invention be limited to particular combinations of cells. Nonetheless, in one embodiment, said first channel further comprises astrocytes and microglial cells.

The presence of other cells allows one to assess their responses. Thus, in one embodiment, the method further comprises detecting microglia activation and/or astrocyte activation. In one embodiment, the method further comprises detecting astrogliosis.

Still additional cells are contemplated. In one embodiment, said second channel comprises a population of endothelial cells. In one embodiment, the method further comprises detecting an inflammatory response of said endothelial cells.

As noted earlier, a variety of read outs can be used. In one embodiment, the present method further comprises detecting induced cytokine secretion. In one embodiment, said cytokine is a proinflammatory cytokine. In one embodiment, said cytokine is selected from the group consisting of IL-6, Interferon-gamma, IL-1B and TNF-alpha.

The presence of endothelial cells allows for the formation of tight junctions, e.g. prior to step c). These can be detected and assessed. In one embodiment, said tight junctions define a barrier having a level of permeability. In one embodiment, the present method further comprises detecting a change in said level of permeability after step c).

Again, it is believed that the present invention offers a platform for drug testing. In one embodiment, the method further comprises d) contacting said endothelial cells with a test compound. In one embodiment, the method further comprises, detecting the impact of said test compound on said permeability.

As noted above, the introduction of an inflammatory inducing compound may cause changes in one cell, with the inflammation thereafter propagated to other cells in the device. Thus, in one embodiment, the method further comprises detecting the propagation of inflammation to said microglia, astrocytes or pericytes (e.g. from the neurons, endothelial cells, or both).

As noted above, cells from humans and other animals can be assessed using the present invention. In one embodiment, said neurons are from a human patient. In one embodiment, said human patient has a neurological disease.

In a preferred embodiment, the microfluidic device is populated with more of the cells that make up the BBB in vivo. Thus, in one embodiment, the present invention contemplates a method, comprising a) providing i) an inflammation inducing compound; ii) a microfluidic device comprising a membrane, said membrane separating first and second microfluidic channels; iii) a population of neurons in said first channel along with a plurality of cells selected from the group consisting of pericytes, astrocytes, microglia and combinations thereof; and iv) a population of endothelial cells in said second channel; and b) culturing said plurality of cells in said first channel and culturing said population of endothelial cells in said second channel, under conditions such that a cell barrier forms having a level of permeability (e.g. under flow conditions, i.e. where culture media is introduced into the channels at a flow rate); and c) contacting at least a portion of said cultured cells with said inflammation inducing compound.

The inflammation inducing compound may cause changes which can be detected and assessed. Thus, in one embodiment, the method further comprises d) detecting a change in said permeability. However, it is not intended that the method be limited to just assessing permeability. In one embodiment, the method further comprises detecting mitochondrial damage in said neurons. In one embodiment, the method further comprises detecting an increase in reactive oxygen species (e.g. over time). In one embodiment, the method further comprises detecting an increase in caspase 3-positive neurons (e.g. over time). In one embodiment, the method further comprising detecting neuroinflammation. In one embodiment, the method further comprises detecting apoptosis. In one embodiment, the method further comprises detecting neuronal death.

With additional cells in the device, the state of activation of cells (other than neurons) can also be assessed. Thus, in one embodiment, the method further comprises detecting microglia activation and/or astrocyte activation. In one embodiment, the method further comprises detecting astrogliosis. In one embodiment, the method further Comprises detecting an inflammatory response of said endothelial cells.

Again, it is not intended that the present invention be limited to how or where the compound is introduced. In one embodiment, said inflammation inducing compound is introduced into said second channel.

A variety of read outs are contemplated. In one embodiment, the method further comprises detecting induced cytokine secretion. In one embodiment, said cytokine is a proinflammatory cytokine. In one embodiment, said cytokine is IL-6.

In one embodiment, the change in the level of permeability of said cell barrier is an increase in permeability. In one embodiment, the present method further comprises detecting this increase.

Again, test compounds can be assessed for their efficacy in reducing the negative impact of inflammation. Thus, in one embodiment, the method further comprises d) exposing at least a portion of said cells to a test compound. In one embodiment, the method further comprises e) detecting an impact of said test compound. In one embodiment, the method further comprises detecting a reduction in permeability after exposure to said test compound.

Again, the test compound can be introduced in a number of ways. In one embodiment, said treating comprises flowing said test compound into said first channel, second channel or both.

A variety of test compounds can be used. In one embodiment, said test compound is trehalose.

In a preferred embodiment, said neurons are dopaminergic neurons. The functionality of such neurons can be assessed. Thus, in one embodiment, the method further comprises detecting transient Ca++ signaling of said dopaminergic neurons prior to step c). In one embodiment, the method further comprises detecting the loss of said transient Ca++ signaling after step c).

As noted earlier, inflammation may be propagated. Thus, in one embodiment, the method further comprises detecting the propagation of inflammation to said microglia, astrocytes or pericytes.

A variety of cell types may be used. Both healthy and diseased cells can be tested. In one embodiment, said neurons are from a human patient. In one embodiment, said human patient has a neurological disease.

It was found empirically that the presence of microglia increases the inflammatory response on the microfluidic device. Therefore, in one embodiment, the present invention contemplates a method, comprising a) providing i) an inflammation inducing compound; ii) a microfluidic device comprising a membrane, said membrane separating first and second microfluidic channels; iii) a plurality of cells comprising microglial cells mixed with cells, said cells selected from the group consisting of pericytes, astrocytes, and neurons and combinations thereof; and iv) a population of endothelial cells; b) culturing said plurality of cells in said first channel and culturing said population of endothelial cells in said second channel; and c) contacting said cultured cells with said inflammation inducing compound under conditions such that inflammation is induced. It is not intended that the invention be limited to the nature of the inflammation inducing compound. However, in eon embodiment, said inflammation inducing compound is TNF-alpha. In another embodiment, said inflammation inducing compound comprises α-synuclein (αSyn) fibrils.

A variety of read outs of inflammation are contemplated. In one embodiment, one or more cytokines are induced by said inflammation inducing compound. In one embodiment, the amount of cytokine produced is larger than the amount produced in the absence of microglial cells. In one embodiment, said cytokine is IL-6.

Again, it is not intended that the present invention be limited to how or where the compound is introduced into the device. In one embodiment, said contacting comprises flowing said inflammation inducing compound into said first channel, second channel or both.

In a preferred embodiment, said neurons are dopaminergic neurons. The functionality of such neurons can be assessed. Thus, in one embodiment, the method further comprises detecting transient Ca++ signaling of said dopaminergic neurons prior to step c). In one embodiment, the method further comprises detecting the loss of said transient Ca++ signaling after step c).

It is contemplated that inflammation can be propagated from one cell type to the next. Thus, in one embodiment, the method further comprises detecting the propagation of inflammation to said microglia, astrocytes or pericytes.

In one embodiment, said neurons are from a human patient. In one embodiment, said human patient has a neurological disease.

It was found empirically that the presence of the additional cells improves the barrier function of the endothelial cells. Thus, in one embodiment, the present invention contemplates a method comprising a) providing i) a microfluidic device comprising a membrane, said membrane separating first and second microfluidic channels; ii) a plurality of cells selected from the group consisting of pericytes, astrocytes, microglial, neurons and combinations thereof; and iii) a population of endothelial cells; and b) culturing said plurality of cells in the first channel and said population of endothelial cells in said second channel, under conditions such that a cell barrier forms having a permeability, wherein the permeability of said barrier is less than the permeability of a barrier where endothelial cells are cultured alone.

A particular combination of cells may be used. Thus, in one embodiment, said plurality of cells comprises a combination of pericytes, astrocytes, microglial, and neurons. In a preferred embodiment, said neurons are dopaminergic neurons. The functionality of these neurons can be assessed. Thus, in one embodiment, the method further comprises detecting transient Ca++ signaling of said dopaminergic neurons during or after step b).

In one embodiment, said neurons are from a human patient. In one embodiment, said human patient has a neurological disease. In one embodiment, the present invention contemplates a PD model using mutant (A53T) α-synuclein for inducing neuroinflammation in a microfluidic brain-chip. In some embodiments, iPS cells were generated from a PD patient known to have α-synuclein comprising an A53T mutation to provide disease (mutant) derived dopaminergic neurons in an A53T Brain-chip.

Described herein is the unexpected discovery that the endothelial cells behave differently in response to neuroinflammation in the brain compartment (e.g. upper channel) of the chip as compared to when endothelial cells are inflamed by circulating inflammatory compounds (i.e. a systemic response) in the vascular channel. Thus, results shown herein provide a clear demonstration that neuroinflammation responses are different than systemic responses, albeit some overlapping characteristics. Moreover, microglia cells were discovered to contribute to neuroinflammation responses in microfluidic Brain Chips.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Exemplary embodiments are illustrated in referenced Figures. It is intended that the embodiments and Figures disclosed herein are to be considered illustrative rather than restrictive.

FIGS. 1A-F shows exemplary reconstruction of one embodiment of a neurovascular unit in a microfluidic device.

FIG. 1A shows one embodiment of a tall two-channel microfluidic BBB chip in vitro: 1. Upper neuronal channel, comprising human iPS-derived neuronal cells co-cultured with 2. Pericytes and 3. Astrocytes; 4. Optional vacuum chambers for providing membrane stretch. 5. Porous Membrane. 6. Endothelial cells. 7. Vascular channel. In one embodiment, microfluidic devices (chips) are seeded with induced pluripotent stem cells (iPSC)-derived cortical neurons, (Glutamatergic and GABAergic neurons), human primary astrocytes and pericytes in the neuronal channel (top), and iPSC-derived human brain microvascular endothelial cells in the vascular channel (bottom).

FIG. 1B upper florescent micrographs show exemplary PDGR-beta (red) stained pericytes and GFAP expressing astrocytes cultured on chip.

FIG. 1C florescent micrographs show exemplary immunocytochemical analysis of hiPSC-derived neuronal cultures in direct contact with astrocytes and pericytes after seven days. Specific markers were used to discriminate neurons (MAP2) and astrocytes (GFAP) from pericytes (NG2). Blue represents Hoechst-stained nuclei.

FIG. 1D right, shows exemplary quantitative barrier function analysis via permeability to 3 KDa fluorescent dextran, crossing through the vascular to the neuronal channel. Results are mean±s.e.m. *P<0.05. n=3. Scale bar: 100 μm.

FIG. 1E shows exemplary representative merged confocal image of the vascular channel stained for tight junction protein marker (ZO-1, green) and Glucose transporter (GLUT1, red) on day 7 in culture (bar, 100 μm).

FIG. 1F shows exemplary quantitative barrier function analysis via permeability of 3 kDa fluorescent dextran, for two independent iPSC donors crossing through the vascular into the neuronal channel on day 5, 6 and 7 in culture (n=6-9 independent chips, NS, not significant). Data are mean±S.E.M. Statistical analysis was by Student's t-test.

FIGS. 2A and 2B show exemplary confocal images of brain and vascular channels.

FIG. 2A upper sets of panels show images of the entire length of an upper channel showing the organization of cell types and coverage across the entire channel on day 7 in culture. Immunofluorescence staining of the brain channel includes MAP2 (green), GFAP (magenta), NG2 (red) and DAPI (blue). Representative merged confocal image of the brain channel stained for iPS-derived cortical neurons (MAP2, green), astrocytes (GFAP, magenta) and pericytes (NG2, red) on day 7 in culture (bar, 50 μm).

FIG. 2B lower sets of panels show images of brain endothelial and tight junction marker staining for morphological characterization from the vascular channel at 7 days in culture. Lower image shows immunofluorescence staining of a vascular channel stained for tight junction protein marker ZO-1 on day 7 in culture (bar, 50 μm).

FIG. 3 shows exemplary schematics, florescent micrographs and charts demonstrating embodiments of a Human BBB chip as a mono-culture of HBMECs (left) and one embodiment of a Human Brain chip comprising neurons, astrocytes, microglia, pericytes and BMECs (right) for immunohistochemical (IHC) analysis. A lower left chart shows permeability assay results demonstrating that a Brain chip maintains a tighter barrier function by day 7 of co-culture over a monoculture of BMECs. A lower right chart shows a comparative assessment of the permeability of three different models (embodiments) on day 7 in culture, including a mono-culture of iPS-derived microvascular endothelial cells (iBMECs), a Brain-Chip cultured using the hCMEC/D3 endothelial cell line (Brain-Chip hCMEC/D3), and a Brain-Chip using iPS-derived microvascular endothelial cell (Brain-Chip iBMECs).

FIG. 4 shows exemplary florescent micrographs demonstrating spontaneous calcium transients identified using fluorescence indicators (Fluo-4 AM) where neurons consistently exhibited spontaneous neuronal activity. Shades of green upper panels-heat mapped lower panels, while the charts show exemplary daily secreted neurotransmitter e.g. glutamate, levels throughout the experiment confirming synaptic activity of cells in a microfluidic chip.

Middle panels show representative time course images of Ca 2+ transients (pseudocolored red represents high levels of Ca 2+ fluorescence while blue represents low levels of Ca 2+ fluorescence). Scale bar: 50 μm.

Lower left chart: Daily secreted levels of a neurotransmitter, e.g. glutamate, confirm the proper synaptic activity in the neuronal channel over time from 4-7 days in culture. Lower right chart: ELISA for glutamate secreted levels into the medium of the brain channel on day 5, 6, and 7 in culture (n=3 independent chips with duplicate technical replicates assayed per condition. Data are mean±S.E.M.

FIG. 5 shows an exemplary tanscytosis receptor as a transferrin receptor, as depicted by schematic diagrams and by flow cytometry analysis of cells tagged with an antibody recognizing a C-terminal region of transferrin receptor compared to cells tagged with a control isotype IgG.

FIG. 6 shows exemplary florescent micrographs demonstrating Immunocytochemistry of Phalloidin (green) staining of actin filaments (also known as F-actin), pHrodoRed Transferrin (red), along with Hoechst staining of nuclei (blue) in one embodiment of a Brain chip comprising iPS-derived HBMECs. Scale bar: 50 μm. Receptor mediated transcytosis of mAb IgG across the BBB is demonstrated in an exemplary chart. Data are means±SEM (n=6 chips), t-test with Tukey's post-hoc test, **P<0.01, ***P<0.001.

FIG. 7 shows exemplary florescent micrographs of fluorescently labeled (green) extracellular matrix proteins demonstrating ECM staining on an ECM coated membrane of a microfluidic chip under flow. It appears that SureBond-XF detaches and flows away while sensory neuron ECM1, shows a greater number of extracellular matrix proteins attached to the chip membrane than laminin alone in ECM2.

FIG. 8 shows an exemplary bright field image showing the results of direct seeding of iPSC-derived Sensory Neuron Progenitors on-Chip: comparing two types of ECM. SureBond-XF; Control upper panel). A combination of Collagen IV (400 μg/mL); Fibronectin (100 μg/mL); Laminin (20 μg/mL); lower panel).

FIG. 9 shows an exemplary bright field image showing the results of direct Seeding on-Chip of iPSC-derived Sensory Neuron Progenitors comparing results of using sensory neuron ECM over time, the Day after seeding upper image and Day 7, lower image. A combination of Collagen IV (400 μg/mL); Fibronectin (100 μg/mL); Laminin (20 μg/mL) was used to coat the membrane prior to seeding cells. iPSC-derived sensory neuron progenitors are treated Day 5 with Mitomycin C to eliminate proliferating cells among the progenitor pool and maintain the population of terminally differentiated non-proliferating neurons.

FIG. 10 shows an exemplary sensory neuron ECM coating of Collagen IV, Fibronectin, Laminin overnight at 4° C. that supported the differentiation and maturation of sensory neuron progenitors (Axol Bioscience) on a tall channel (S-1) chip confirmed by mature sensory neuron and nociception specific markers MAP2 (green), TRPV1 (red-left panel), and Nav1.7 (red-right panel). Merge shows superimposed images from the column of panels above the merged image. Co-merged staining is orange and yellow. Day 10, 5 days of flow.

FIG. 11 shows exemplary florescent micrographs of ECM proteins colored as magenta and green fluorescently labeled macrophages and T-cells on chips as one embodiment of a contemplated Live-Image Tile of Innervated Intestine-Chip. Merged image shows both ECM, macrophages (MO) and T-cells.

FIG. 12 shows exemplary results using one embodiment of an Intestine-Chip. Upper left panel is a representative image of labeled immune cells on and within the epithelial layer of a Caco-2 Intestine-Chip. Middle panel is a representative FACs forward vs side scatter plot of differentiated lymphocytes. Right panel is a FACs histogram showing the expression of macrophage marker CD86 after differentiation of lymphocytes. These were immune cells that were incorporated on the chip. Lower panes show immune cell counts from chips co-cultured for 7 and 14 days. Identical rounds of macrophage differentiation from different PBMC donors. Co-stimulation with anti-human CD3 & anti-human CD28.

FIG. 13 shows exemplary florescent images of one embodiment of a Brain-Chip exposed to TNF-alpha (100 ng/ml) (right panel where arrows point to cell membranes lacking ZO-1 attachments) via the neuronal channel. TNF-alpha treatment also significantly increases GFAP expression as well as neuronal death up to 24 hours after stimulation. Scale bar: 50 μm.

Chart demonstrates TNF-alpha induced increase in permeability by an increase in 3 kDa Dextran diffusion from the lower to upper channel. Data are means±SEM (n=6 chips), t-test with Tukey's post-hoc test, **P<0.01.

FIG. 14 shows exemplary florescent images of one embodiment of a Brain-Chip exposed to TNF-alpha showing Neurons (MAP2-green), Astrocytes (GFAP-pink) and staining for Nuclei: Hoechst (blue). Scale bars: 50 micom. Chart demonstrates % of specific cell subtypes over total brain cells. Data are means±SEM, n=6 Chips, *P<0.05.

FIG. 15 shows exemplary florescent images of one embodiment of a healthy Brain-Chip exposed to TNF-alpha (100 ng/ml) (left panel Microglia (Iba1)-red and Neurons (MAP-2)-green. Live Imaging (right panel) of an Inflamed Brain-Chip (right panel Microglia (Cell Tracker Red CMPTX dye) and green Fluorescent latex beads. Phagocytosis of beads are an indication of activation of glial cells.

FIG. 16 shows an exemplary comparison of IL-6 secretion in pg/ml from Brain-Chip (−Microglia) and Brain-Chip (+Microglia) at 24 and 48 hours. Data are means±SEM (n=6 chips), Anova with Tukey's post-hoc test, **P<0.01, ***P<0.001.

FIG. 17 shows an exemplary schematic diagram of examples of C1q with neurodegenerative diseases. Cho, “Emerging Roles of Complement Protein C1q in Neurodegeneration.” Aging and Disease, 10(3): 652-663. June, 2019.

FIG. 18 shows an exemplary chart demonstrating that Treatment with C1q neutralizing antibody attenuates TNF-mediated inflammation, as indicated by IL-6 levels. Data are means±SEM (n=6 chips), Anova with Tukey's post-hoc test, **P<0.01, ***P<0.001.

FIG. 19 shows an exemplary immunostained Brain-Chip on Day 10 demonstrating iPS-derived Dopaminergic Neurons double positive (yellow) for a MAP2: Neuronal Marker (green) and TH: Selective Marker for Dopaminergic Neurons-tyrosine hydroxylase (red). Scale bar: 50 μm. Shows an exemplary chart demonstrating Neurotransmitter Secretion, e.g. Dopamine in the range of pg/mL, at Day 7 and Day 10 (n=6 chips).

FIG. 20 shows an exemplary schematic diagram of a human brain cortex containing GABAergic and glutamatergic neurons representing two neuronal classes, which establish inhibitory and excitatory synapses, respectively. Human Dopaminergic neurons are localized in the substantia nigra (SN). In one embodiment, for comparing some pathological similarities and differences between Parkinson's disease and Alzheimer's disease. For Parkinson's disease, red shade indicates sites of major cell loss and α-synuclein pathology, e.g. near the brain stem. For Alzheimer's disease, green shade throughout the cortex indicates major regions of cell loss and β-amyloid plaques and tau pathology.

FIG. 21 shows exemplary schematic diagrams depicting the progression of Parkinson's Disease in one embodiment of a Brain-Chip. Healthy alpha-synuclein (alpha-Syn) (monomeric) becomes phosphorylated at P Ser-129 (amino acid 129) forming alpha-Syn oligomers which aggregate into fibril aggregates with pathologic alpha-Syn (PFFs). Dopaminergic neurons and other brain cells take up extracellular PFFs inducing on a Brain-chip one or more of neuronal dysfunction, e.g. Impaired Calcium activity; impaired Mitochondrial Function e.g. Expression measured by JC-1; Neuroinflammation, e.g. Increased IL-6 secretion, Microglia activation, Astrocyte proliferation; and Neuronal Loss e.g. reduced number of cells after staining with MAP2, symptoms and pathology also observed in clinical/pathology of a PD brain.

FIG. 22 shows exemplary fluorescently stained micrographs and a chart demonstrating a dose response of pathogenic alpha-Syn PFFs contacting neurons in one embodiment of a microfluidic brain-chip over time for inducing an increasing amount of pSer129 within neurons simulating α-Syn deposition in Lewy bodies of a PD brain. Dose response is 400 ng/ml vs. 4000 ng/ml of alpha-Syn, e.g. alpha-Syn PFFs at Day 3 and Day 6 of exposure. Panels show results of cellular exposure to monomers (normal alpha-Syn) in a brain chip in contrast to panels showing exposure of cells in a Brain-Chip to PFFs (Pathogenic alpha-Syn). pSer129-αSyn (green) and DAPI stained nuclei (blue). Scale bar: 50 μm. An exemplary chart shows increasing amounts of a toxic form of Ser129-αSyn activity (Fold change vs monomers) where at Day 3 there is a similar amount with 400 ng/ml vs. 4000 ng/ml (NS—not significant).

FIG. 23 shows exemplary florescent micrographs of fluorescently stained embodiments of Brain Chips and a chart demonstrating a dose response of pathogenic alpha-Syn PFFs contacting neurons in one embodiment of a microfluidic brain-chip over time for inducing an increasing amount of JC-1 within neurons simulating JC-1 staining of a PD brain. Dose response is 400 ng/ml vs. 4000 ng/ml of JC-1, e.g. alpha-Syn PFFs at Day 3 and Day 6 of exposure. Red fluorescence indicated normal mitochondrial potential, whereas green fluorescence indicated damage to mitochondrial potential. Panels show results of cellular exposure to monomers (normal alpha-Syn) in a brain chip in contrast to panels showing exposure of cells in a Brain-Chip to PFFs (Pathogenic alpha-Syn). JC-1 (green) and DAPI stained nuclei (blue). Scale bar: 50 μm.

FIG. 24 shows exemplary loss of transient Ca++ signaling (no change in Ca++ levels) over time after Alpha-Syn PFFS treatment compared to Alpha-Syn monomer treatment (signaling off and on, see insets). FUOR-4AM fluorescent staining of Brain chips after 6 Days of Exposure to Monomer and PFFS, 4000 ng/ml. Column of panels, left to right, 0 sec, 10 sec, 20 sec, 30 sec. Scale bar: 50 μm. Electrical read-outs show an almost complete loss of transient signaling after Alpha-Syn PFFS treatment, lower charts.

FIG. 25 shows exemplary florescent micrographs and charts comparing fluorescently stained Neurons (MAP2-green), Astrocytes (GFAP-red), Activated Microglia (CD11b-red), Nuclei (DAPI-blue) 6 Days of Exposure after Alpha-Syn PFFS treatment compared to Alpha-Syn monomer treatment, 400 vs. 4000 ng/ml. Left chart demonstrates % of specific cell subtypes over total brain cells (normalized to DAPI stained nuclei). n=6 chips means±SEM.*P<0.05, **P<0.01, ***P<0.001. Right chart demonstrates IL-6 levels pg/mL in neuronal IL-6 channels. ** indicates a significant difference.

FIG. 26 shows exemplary results of LIVE-DEAD assay comparisons indicating neuronal death after 3 days of exposure to Monomer and PFFS, 4000 ng/ml along with charts showing LIVE/DEAD Ratios after 3 and 10 days of exposure. n=6 chips means±SEM. **P<0.01, ***P<0.001.

FIG. 27 shows exemplary results of a loss of barrier function by a Alpha-Syn PFFS treated a Brain chip compared to alpha-Syn monomer treatment. n=8 chips. means±SEM. ****P<0.0001.

FIG. 28A-I shows exemplary embodiments of schematics and images of Intestine-chips. Villi-like formations in the Intestine-Chip, FIG. 28F. Morphology was characterized with immunofluorescence cross-sectional view FIG. 28A of intestinal epithelial cells in the Caco-2 Intestine-Chip and Scanning Electron Micrograph (SEM) of Caco-2 FIG. 28B. Epithelial thickness is reduced after an inflammatory treatment FIG. 28D compared to control FIG. 28C. A representative whole-chip tile was taken showing expression of tight junction protein ZO-1 with immunofluorescence in the bottom image, FIG. 28E. Epithelial Layer Morphology and Barrier Function, FIG. 28F. Epithelial cells and iPSC-derived sensory neuron progenitors co-cultured within the chip in the presence of continuous flow for 14 days maintain barrier function, FIG. 28H. Maturation and differentiation of the epithelial morphology and villi-like structures were monitored via bright field imaging over 14 days. The overall viability of the epithelium was assessed by measurement of effluent LDH and found less than 5% leakage for each co-culture condition, FIG. 28G.

FIG. 29 shows exemplary Neuronal Immunofluorescence Staining on one embodiment of an Innervated Intestine-Chip by day 7-Chip, demonstrating interactions (i.e. merged image) between sensory neurons, e.g. nociception specific marker, TRPV1 (red), MAP-2 (green), nuclear stain (blue).

FIGS. 30A-F shows exemplary schematic depiction of one embodiment of a microfluidic human Substantia Nigra (SN) Brain-Chip and immunohistochemistry of iPS-derived brain endothelial cells cultured on 4 surfaces of the lower vascular channel, and 4 additional cell types of iPS-derived dopaminergic neurons, primary human brain astrocytes, microglia and pericytes on the upper surface of the central horizontal membrane in the apical brain channel.

FIG. 30A shows a schematic depiction of one embodiment of a SN Brain-Chip of a 2-channel microfluidic Organ-Chip having 5 cell types. In one channel (brain channel) is a co-culture of microglia, astrocytes, dopaminergic neurons and pericytes. In an opposing channel, separated by a porous membrane, are endothelial cells (vascular channel).

FIG. 30B shows a 3D reconstruction of a confocal z-stack of fluorescent images showing the organization of five cell types in one embodiment of a SN Brain-Chip. Nuclei (blue); GFAP+(pink); pericytes (light blue); and endothelial cells stained for a tight junction protein (ZO-1: red) as shown in cross section.

FIG. 30C shows a representative image of iPS-derived dopaminergic neurons that are stained with DAPI (colored blue), Microtubule-associated protein 2 (MAP2—green), TH (red), and a merged image on day 8. Scale bars: 100 μm.

FIG. 30D shows an immunofluorescence micrographs of the human brain endothelium cultured on the vascular channel of Brain-Chip for 7 days post-seeding (D8) labeled with Claudin-1 (red), Claudin-5 (cyan), Occludin (yellow), and CD31 (white). Scale bars: 100 μm.

BBB integrity was observed for up to 8 days in one embodiment of a Brain-Chip.

FIG. 30E shows immunofluorescence micrographs demonstrate high levels of expression of ZO-1 (red) across the entire endothelial monolayer. Scale bars: 100 μm.

FIG. 30F shows a quantitative barrier function analysis via permeability to 3 kDa fluorescent dextran and 0.5 kDa lucifer yellow (left) crossing through the vascular to the neuronal channel on day 5 and 8 (n=6-9 independent chips). Error bars present mean±SEM. Quantitative barrier function analysis via permeability of 3 kDa fluorescent dextran (right), for two independent iPSC donors crossing through the vascular into the neuronal channel on day 5, 6 and 7 in culture (n=6-9 independent chips, NS, not significant). Data are mean±S.E.M. Statistical analysis was by Student's t-test.

FIGS. 31A-F shows exemplary characterization of neurons and endothelial cells in one embodiment of a Human Substantia Nigra Brain-Chip.

FIG. 31A Graph shows neurotransmitter release over time between 5 and 8 days of co-culture. Neurotransmitter release is shown as ELISA results for dopamine secreted into the medium of the brain channel on days 5 and 8. (n=3 independent chips with duplicate technical replicates assayed per condition). n=6 chips. Error bars present mean±SEM.

FIG. 31B shows exemplary immunofluorescent microphotographs (left) validate the dopaminergic neurons with MAP2+(green), astrocytes with GFAP (magenta) and pericytes (red), and the DAPI (blue) for cell nuclei. Immunofluorescent microphotograph (right) validates the glia culture: astrocytes (magenta, GFAP staining), and resting microglia (yellow, TMEM119). Scale bars: 50 μm.

FIG. 31C shows exemplary immunofluorescent images of MAP2+(green); TH (red); Hoechst stained nuclei (blue) of iPS-derived dopaminergic neurons. Scale bar=10 μm.

FIG. 31D shows exemplary Iimmunocytochemical analysis of iPS-derived neuronal cultures in direct contact with astrocytes and pericytes. Specific markers were used to identify neurons (MAP-2), astrocytes (GFAP), and pericytes (NG2). Blue represents Hoechst-stained nuclei.

FIG. 31E shows exemplary immunocytochemical analysis that demonstrated endothelial monolayer tightness and brain specificity using ZO-1, GLUT-1, CD31, and Occludin markers at day 7 in culture.

FIG. 31F shows exemplary representative merged confocal image of the brain channel co-stained for iPS-derived cortical neurons (MAP2, green) and vesicular Glutamate transporter 1 (VGLUT1, red) (bar, 100 μm).

FIGS. 32A-E shows exemplary Differentially Expressed (DE) genes and enriched gene ontology (GO) categories in SN Brain-Chip and conventional cell culture (CCC) system, as compared to the adult in vivo substantia nigra; day 8.

FIG. 32A shows schematic drawings of devices, Transwell and microfluidic brain-chips, along with a volcano plot resulting from DGE analysis between SN Brain-Chip and CCC. For the selection of the DE genes, the following thresholds were used: adjusted p-value<0.05 and |Log 2(foldchange)|>1. The identified up- (down-) regulated genes are highlighted in cyan (magenta) color respectively. Sample sizes were as follows: SN Brain-Chip, n=4, conventional cell culture system, n=4.

FIG. 32B and FIG. 32C shows exemplary list of biological processes identified by Gene Ontology (GO) enrichment analysis using the up- and down-regulated genes respectively resulted by the differentially gene expression analysis between SN Brain-Chip and CCC.

FIG. 32B shows exemplary GO Term Enrichment Biological Processes Upregulated Genes in Brain Chip.

FIG. 32C shows exemplary GO Term Enrichment Biological Processes Upregulated Genes in Conventional Cell Culture.

FIG. 32D shows exemplary DGE analysis identified up- and down-regulated genes in SN Brain-Chip compared to CCC (cyan circle), and human adult substantia nigra compared to CCC (yellow circle). Sample sizes were as follows: SN Brain-Chip, n=4, Conventional cell culture system, n=4, and adult substantia nigra, n=8 (independent biological specimens). Culture in Brain-Chips and CCC were done in parallel. Samples were collected and processed for analyses 8 days post-seeding (D8).

FIG. 32E shows an exemplary SN Brain-Chip exhibits higher transcriptomic similarity to adult substantia nigra than conventional cell culture. The results of the GO terms analysis using the 209 DE genes showed 6 significantly enriched (FDR adjusted p-value<0.05) biological processes related to tissue development, response to a stimulus, biological adhesion, and cell surface receptor signaling pathway. The size of the bars indicates the fold-enrichment of the corresponding pathways.

FIGS. 33A-D shows exemplary embodiments of a microfluidic Brain-Chip that exhibits higher transcriptomic similarity to adult cortex tissue than Transwell cultures (not under flow); days 5 and 7 of culture.

FIG. 33A shows an exemplary Principal component analysis (PCA) generated using RNA-seq data generated by the samples collected from the brain channel of the Brain-Chips and transwells on days 5 and 7 in culture (n=4 per condition), as well as human brain cortex. A 2D-principal component plot is shown with the first component along the X-axis and the second along the Y-axis. The proportion of explained variance is shown for each component.

FIG. 33B shows an exemplary Quantitative analysis on the distances of the Brain-Chip or Transwell culture from Human Brain Cortex on days 5 and 7 of culture.

FIG. 33C shows an exemplary Differential Gene Expression (DEG) analysis identified up- and down-regulated genes in the Brain-Chip compared to conventional cell cultures (blue circle), and human adult cortex brain tissue compared to conventional cell cultures (yellow circle). Gene lists summarized in the Venn diagram are provided in Extended Data. Sample sizes were as follows: Brain-Chip, n=4, transwells n=4, and adult cortex tissue, n=8 (independent biological specimens). Culture in Brain-Chips and conventional cell cultures were done in parallel.

FIG. 33D shows an exemplary Curated heatmap generated to examine particular genes that belong to the enriched KEGG pathways and to show the expression levels log 2(FPKM) of these genes across different samples. Genes belonging to four different pathways, including: intermediate filament cytoskeleton organization, neuronal action potential, axon guidance, and extracellular matrix organization, are shown. Each heatmap has its own color scale, which corresponds to a different range of log 2 (FPKM) values, as indicated on the color bars located to the left.

FIGS. 34A-B shows exemplary schematic diagrams demonstrating contemplated embodiments of drug targets and biomarkers for normal (noninflammatory) and disease associated inflammatory conditions. Smyth et al., Journal of Neuroinflammation 2018.

FIG. 34A shows exemplary systemic inflammation that causes a breach in the blood brain-barrier (BBB) thereby allowing for the entry of immune/inflammatory cells and proinflammatory cytokines into the brain.

FIG. 34B shows exemplary neuroinflammation that induces and accelerates pathogenesis of Parkinson's disease (PD), Alzheimer's disease (AD) and Multiple sclerosis (MS).

FIG. 35 shows an exemplary schematic experimental timeline of one embodiment of a neuroinflammation culture model. On days 0 and 1 cells were seeded on the top and bottom channel of one embodiment of a Brain-Chip, respectively. The Brain-Chip is then allowed to mature under flow conditions for 4 more days and on day 5, cells on the brain side or the vascular side are treated with TNFα, for 48 hrs. During exposure, effluent is collected, and on the last day of the experiment, cells in chips may be imaged, stained for immunohistochemistry and imaged, effluent may be collected from the brain channel and the vascular channel. In some embodiments, cells are lysed within each channel for channel specific transcriptomic analysis.

FIG. 36A shows one embodiment of neuroinflammation as a schematic illustration of the Brain-Chip showing the TNF-α perfusion within the brain channel and shows an exemplary chart of comparative apparent permeability showing time-depended BBB disruption measured after 24 hrs and 48 hrs of exposure to TNFα (100 ng/mL) vs. vehicle control, introduced on the brain side. Transport through the BBB was significantly increased by three times over the control. Data are means±SEM, *p<0.5 (n>4 chips), vehicle compared to TNF-α treated group after 48 hrs of treatment). Statistical analysis is two-way ANOVA with Tukey's multiple comparisons test.

FIG. 36B shows exemplary results of TNF-alpha induced neuroinflammation by immunofluorescent double staining of neurons (MAP2, green), astrocytes (GFAP, magenta), microglia (CD68, red), and nuclei (DAPI, blue), following two days of exposure with TNF-α compared to the untreated group (bar, 100 nm).

FIG. 36C, FIG. 36D, and FIG. 36E shows exemplary results of TNF-alpha induced neuroinflammation by secreted levels of proinflammatory cytokines (IFN-γ, IL-1β and IL-6) in the healthy or 48 hr TNF-α treated Brain-Chips in the presence or absence of microglia (n=4-7 independent chips, **p<0.01, ****p<0.0001, NS, not significant). Data are mean±S.E.M. Statistical analysis was by Student's t-test.

FIG. 36F shows an exemplary quantification of the number of GFAP-positive and MAP2 events per field of view. Statistical analysis is Student's t-test (n=3 independent chips with 3-4 randomly selected different areas per chip, *p<0.05, **p<0.01 compared to the untreated group).

FIG. 36G shows an exemplary quantification of the number of CD68-positive events per field of view. (n=3 Brain-Chips with 3˜5 randomly selected different areas per chip, *P<0.05 compared to the untreated group). Data are mean±S.E.M. Statistical analysis was by Student's t-test.

FIG. 36H shows an exemplary ELISA for glutamate secreted levels into the medium of the brain channel on day 7. (n=3 independent chips with duplicate technical replicates assayed per condition, **p<0.01, compared to the untreated group). Data are mean±S.E.M. Statistical analysis was by Student's t-test.

FIG. 37A shows an exemplary schematic illustration of the Brain-Chip showing the TNF-α perfusion within the vascular channel as a systemic inflammation culture model. On days 0 and 1 cells were seeded on the top and bottom channel, respectively. The Brain-Chip is then allowed to mature under flow conditions for 4 more days and on day 5, cells on the vascular side are treated with TNFα, for 48 hrs. During exposure, effluent is collected, and on the last day of the experiment, cells in chips may be imaged, stained for immunohistochemistry and imaged. In some embodiments, cells are lysed for transcriptomic analysis.

FIG. 37B shows an exemplary Quantitative barrier function analysis via permeability to 3 kDa fluorescent dextran, over 48 hs of treatment with TNF-α perfused at the vascular channel, showing time-depended BBB disruption (n=3-4 independent chips, *P<0.05, vehicle compared to TNF-α treated group after 48 hs of treatment). Data are mean±S.E.M. Statistical analysis is two-way ANOVA with Tukey's multiple comparisons test.

FIG. 38A Brain Channel: Neuroinflammation vs Healthy Brain-Chip on day 7, Table 1, shows an exemplary volcano plot illustrating the number of the differentially expressed (DE) genes (up-magenta dots) and down-cyan dots) regulated) and how they stratify based on their expression changes. DE genes significantly up- or down-regulated (adj.p-value<0.01 and |log 2FoldChange|>1); black dots: non-DE expressed genes. In total, 1174 genes were found significantly DE in cells of brain parenchyma, 801 up-regulated (in the inflamed Brain-Chips) and 373 down-regulated (in the inflamed Brain-Chips).

FIG. 38B shows an exemplary list of biological processes identified by Gene Ontology (GO) enrichment analysis based on the 801 up-regulated DE genes between the TNFαBrain-Chips and Healthy Brain Chips. Bar plot presents a subset of the significantly enriched biological processes identified by the enrichment analysis.

FIG. 39A Brain Channel: Systemic Inflammation vs Healthy Brain-Chip, Table 2, shows an exemplary volcano plot illustrating the number of the differentially expressed (DE) genes (up- and down-regulated) and how they stratify based on their expression changes. Red dots: DE genes significantly up- or down-regulated (adj.p-value<0.01 and |log 2FoldChange|>1); black dots: non-DE expressed genes. In total, 528 genes were found significantly DE in cells of brain parenchyma, 473 up-regulated (in the inflamed Brain-Chips) and 55 down-regulated (in the inflamed Brain-Chips).

FIG. 39B shows an exemplary list of biological processes identified within the brain channel by Gene Ontology (GO) enrichment analysis based on the 473 up-regulated DE genes between the TNFα Brain-Chips and Healthy Brain Chips. Bar plot presents a subset of the significantly enriched biological processes identified by the enrichment analysis.

FIG. 40 shows an exemplary chart of comparative apparent permeability measured after 24 hrs and 48 hrs of exposure to TNFα (100 ng/mL) vs. vehicle control, introduced on the brain side. Transport through the BBB was significantly increased by three times over the control. Data are means±SEM, *p<0.5 (n>4 chips).

FIGS. 41A-B Vascular Channel: DGE Analysis (Neuroinflammation). See, Table 3.

FIG. 41A shows an exemplary volcano plot illustrating the number of the differentially expressed (DE) genes (up- and down-regulated) and how they stratify based on their expression changes. Red dots: DE genes significantly up- or down-regulated (adj.p-value<0.01 and |log 2FoldChange|>1); black dots: non-DE expressed genes. In total, 387 genes were found significantly DE in endothelial cells, 371 up-regulated (in the inflamed Brain-Chips) and 16 down-regulated (in the inflamed Brain-Chips).

Significantly Enriched GO terms from the list of the 473 DE up-regulated genes upon TNF-α exposure.

FIG. 41B shows an exemplary Gene Ontology (GO) enrichment analysis based on the 371 up-regulated DE genes between the TNFα treated Brain-Chips and Healthy Brain Chips.

Bar plot presents a subset of the significantly enriched biological processes identified by the enrichment analysis. Vascular Channel: Significantly Enriched Biological Processes. Significantly Enriched GO terms from the list of the 371 DE up-regulated genes upon TNF-α exposure

FIGS. 42A-B shows exemplary differential Gene Expression analysis between TNFα exposed Brain-Chips and Healthy Brain-Chips on day 7. See, Table 4.

FIG. 42A shows an exemplary volcano plot illustrating the number of the differentially expressed (DE) genes (up- and down-regulated) and how they stratify based on their expression changes. Red dots: DE genes significantly up- or down-regulated (adj.p-value<0.01 and |log 2FoldChange|>1); black dots: non-DE expressed genes. In total, 1174 genes were found significantly DE in endothelial cells, 801 up-regulated (in the inflamed Brain-Chips) and 373 down-regulated (in the inflamed Brain-Chips).

FIG. 42B shows an exemplary Gene Ontology (GO) enrichment analysis based on the 801 up-regulated DE genes between the TNFαBrain-Chips and Healthy Brain Chips. Bar plot presents a subset of the significantly enriched biological processes identified by the enrichment analysis.

FIG. 42C Brain Channel: Systemic Inflammation vs. Healthy Chip shows an exemplary Gene Ontology (GO) enrichment analysis based on the 371 up-regulated DE genes between the TNFα Brain-Chips and Healthy Brain Chips. Bar plot presents a subset of the significantly enriched biological processes identified by the enrichment analysis.

FIG. 43A Brain channel: neuroinflammation shows exemplary secretion of pro-inflammatory cytokines. One embodiment of a brain channel of a Brain-Chip was exposed to continuous flow of media containing 100 ng/mL TNFα for 48 hrs. The effluent was collected at the 48 h timepoint; cytokines were measured and analyzed using MSD human proinflammatory panel. Data are means±SEM, ***p<0.001, ****p<0.0001 (n>4 chips).

FIG. 43B Brain channel: systemic inflammation

shows exemplary secretion of pro-inflammatory cytokines. One embodiment of a brain channel of a Brain-Chip was exposed to continuous flow of media containing 100 ng/mL TNFα for 48 hrs. The effluent was collected at the 48 h timepoint; cytokines were measured and analyzed using MSD human proinflammatory panel. Data are means±SEM, ***p<0.001, ****p<0.0001 (n>4 chips).

FIG. 44A vascular channel: neuroinflammation shows exemplary representative immunofluorescent staining of endothelial and tight junction markers after 48 hrs of exposure to TNFα on the brain-side showing a decreased expression of tight junction protein, ZO-1, and increased expression of intercellular adhesion molecule-1 (ICAM-1).

FIG. 44B vascular channel: Systemic inflammation shows exemplary representative immunofluorescent staining of endothelial and tight junction markers after 48 hrs of exposure to TNFα on the brain-side showing a decreased expression of tight junction protein, ZO-1, and increased expression of intercellular adhesion molecule-1 (ICAM-1).

FIG. 45 brain channel: Neuroinflammation vs. Systemic inflammation shows an exemplary curated heatmap generated to examine particular genes resulted by the differentially gene expression analysis of the brain channel, between the systemic inflammation and neuroinflammation condition.

FIG. 46A vascular channel: Neuroinflammation vs. Systemic inflammation

FIG. 46B curated heatmaps were generated to examine particular genes resulted by the differentially gene expression analysis of the vascular channel, between the systemic inflammation and neuroinflammation condition.

FIG. 47A brain channel: Neuroinflammation vs. Systemic inflammation shows an exemplary Venn diagram demonstrating an overlap of 508 DE genes associated with inflammation between 1174 DE genes in an embodiment of neuroinflammation vs. 528 healthy on Day 6 vs. Brain-Channel.

FIG. 47B vascular channel: Neuroinflammation vs. Systemic inflammation shows an exemplary Venn diagram of DE genes identified in the vascular channel (endothelium) between both types of neuroinflammation vs healthy conditions. Inflamed Brain-Chips (Neuroinflammation) Vs. Healthy on Day 6 (blue). Inflamed Brain-Chips (Systemic Inflammation) Vs. Healthy on Day 6 (blue).

FIG. 48A shows exemplary schematic diagrams demonstrating native and toxic conformations of α-syn. Alpha-synuclein transforms into multiple different conformations, including monomers (predominant in a α-helical confirmation), tetramers, higher-level oligomers (soluble conformations), and fibrils (highly ordered insoluble conformations characterized by β-sheet conformation). Alpha-synuclein exists in a native conformation as monomers as well in a dynamic equilibrium with tetramers. The tetramer, less likely to form aggregate, may form an aggregate after disrupted into monomers in order to misfold. Many factors, such as the posttranscriptional modification and SNCA mutations in A53T and E46K promote formation of pathological oligomers, presently considered to be the most toxic structure of α-syn, which is further folded to form amyloid fibril (rich in β-sheet structure), the accumulation of which leads to the formation of intracellular inclusions called Lewy Body.

FIG. 48B shows exemplary interactions between α-syn and cellular components contemplated as drug targets for use in drug screening methods as described herein. Misfolded α-syn is degraded through, the autophagy-lysosomal pathway (ALP) and the ubiquitin-proteasome system (UPS). Certain oligomeric species present toxicity via interactions with cellular components by mechanisms that include: (1) alteration of cytoskeletal integrity; (2) membrane disruption and pore formation; (3) nuclear dysfunction; (4) inhibition of vesicle docking; (5) UPS dysfunction; (6) ALP impairment; (7) reduction of mitochondrial activity; and (8) chronic ER stress. UPS, ubiquitin-proteasomal system; ALP, autophagy-lysosomal pathway; ER, endoplasmic reticulum.

FIG. 48C shows exemplary schematic summary of interactions between α-synuclein and cellular components, such interactions are contemplated for use as drug targets in methods of use for microfluidic Brain-Chips as described herein. At least six different exemplary intracellular pathways are affected by α-synuclein (α-syn). The protein α-syn is enriched at the pre-synaptic terminals of the majority of types of neurons in the brain, where it participates in the vesicle recycling, thereby modulating synaptic function. α-syn can be degraded by the ubiquitin-proteasome system (UPS) and inside the lysosomes. α-syn interacts strongly with membranes, such as plasma membrane and mitochondrion. When misfolded, α-syn forms distinct structures that are prone to aggregation, into oligomers, then into larger structures. α-syn oligomers in a toxic form may impair basic neuronal processes, such as ER-Golgi trafficking, lysosome and UPS functions, reduced mitochondrial activity and alter the plasma membrane through the pore/perforations that can dysregulate calcium and cation homeostasis. In fact, many of these pathways were identified as GO categories of Genes that were upregulated genes in Brain-Chips.

FIG. 48D shows exemplary autophagy-lysosomal pathway (ALP) and ubiquitin-proteasome system (UPS) pathways under normal and pathological conditions. Proteins are tagged with ubiquitin conjugates through a sequential enzymatic mechanism involving three classes of enzymes, E1, E2 and E3. Under normal conditions, ubiquitylated substrates are recognized by ubiquitin receptors present in ALP and UPS pathways and efficiently eliminated. In the UPS, substrates are subsequently deubiquitylated by RPN11, a step for substrate degradation and amino acid recycling. Free-Ub chains formed by RPN11 activity promote ALP function. Ubiquitin receptors in the ALP, in contrast to the UPS, form oligomers to facilitate substrate recognition and autophagosomal recruitment. Under aging and Alzheimer's disease conditions there is a decrease in the function of the ALP and the UPS that reduces substrate degradation and amino acid recycling. Downregulation of RPN11 in Alzheimer's disease (AD) decreases free-Ub chains disrupting substrate recognition, their recruitment into autophagosomes and their final degradation by the ALP. Altogether, leading to the accumulation of deleterious protein aggregates. Transcriptional regulation (Nrf1/2) and phosphorylation (kinases/phosphatases) play a crucial role in ALP and UPS function whereas their dysregulation is the focus of intense studies in aging and Alzheimer's disease

FIG. 49A shows exemplary schematic diagrams depicting steps towards accumulation of Alpha-synuclein protein (SNCA). Natural SNCA becomes misfolded under stress and becomes oligomers, oligomers, profibril oligomers that form fibril aggregates that form Lewy bodies in affected neurons of a patient's PD brain leading to dopamine (DA) neuronal loss.

FIG. 49B shows an exemplary schematic depiction of α-synuclein fibril contributions to Alpha/Beta plaques, Tau tangles and α-synuclein Lewy bodies found in degenerating neurons.

FIG. 50A shows exemplary schematics for providing embodiments of a Brain Chip: comprising Neurons, Astrocytes, microglia, pericytes, and endothelial cells.

FIG. 50B shows an exemplary Principal Components Analysis (PCA) of healthy vs. PD disease associated brain channel cells.

FIG. 51A Brain Channel: Volcano plot: Brain-chip A53T vs. healthy.

FIG. 51B Brain Channel: GO-terms Enrichment Analysis Results. Go-term enrichment analysis results using the 320 up-regulated genes in A53T brain-Chips.

FIG. 52 Brain Channel: Principal Components Analysis (PCA) comparing the same brain cells cultured in either Transwell cultures or Brain Chips, as healthy cultures without exposure to a monomer or fibril, or exposed to monomers, fibrils or fibrils comprising an A53T mutation.

FIG. 53 shows exemplary schematic depictions of dopaminergic neurons with synaptic terminals. While increasing dopamine (blue dots) in the wild-type (WT) setting is benign (left), similar increases in the setting of human mutant (A53T) α-synuclein (α-syn) lead to progressive neurodegeneration (middle and right) in mice. Synaptic loss (red X marks) in presynaptic striatal terminals precedes somatic degeneration, and toxicity is thought to be mediated by α-synuclein (red) oligomers in the presence of dopamine.

FIG. 54A-E shows images from a pathological examination of a healthy patient (FIG. 54A) reveals typical pigmented DA neurons in the SN (arrows); in contrast, loss of SN neurons leads to pigment disappearance in the PD brain (FIG. 54B, arrows). Magnification of the SN area reveals a dense network of melanin-pigmented SN neurons in the healthy brain (FIG. 54C) while most of SN neurons are lost in PD (FIG. 54D). Some of the remaining neurons in PD contain insoluble cytoplasmic protein aggregates (Lewy Bodies, FIG. 54E) that are made of aggregated alpha-synuclein and other proteins. The melanin-containing granules have a red-brown hue and are distributed in the cytosol of all SN neurons (FIG. 54C-E). The image in FIG. 54E is the higher magnification of the dark-boxed area in FIG. 54D. Adapted from Agamanolis, 2006.

FIGS. 55A-B shows exemplary schematic depictions of a comparison of cellular interactions in the upper channel of one embodiment of a SN Brain-Chip contemplated to provide an intact BBB (healthy) in one embodiment of a Brain-chip FIG. 55A vs. FIG. 55B contemplated cellular interactions between three types of brain cells as activated microglia and reactive astrocytes both cause damage inflammatory response of dopaminergic neurons, including dystrophic neurites, shrinkage of the soma, and neuronal loss, resulting in breakdown of BBB as a SN Brain-Chip model for Parkinson's Disease.

FIG. 56 shows exemplary schematic depictions of a model of aSyn actively secreted or released by dying neurons, e.g. neuron 1, into the extracellular space. Extracellular aSyn can then activate surrounding astrocytes and microglia, eliciting glial pro-inflammatory activity. Upon activation microglia produce pro-inflammatory cytokines, nitric oxide, and reactive oxygen species, which may be toxic to neurons. aSyn can be directly transferred between neurons, e.g. neuron 1 to neuron 2, and so on, leading to propagation of an aberrant aSyn aggregation process.

FIG. 57A-D shows exemplary pathological αSyn accumulation in the brain channel was observed following exposure to human αSyn fibrils.

FIG. 57A shows exemplary schematic depiction of one embodiment of an Experimental design for assessing the effects of αSyn toxic aggregates (fibrils) in the SN Brain-Chip, including the seeding in the Brain-Chip, the timeline for medium changes, as well as sampling times.

FIG. 57B Immunofluorescence micrographs show the accumulation of phosphorylated αSyn (green, phospho-αSyn129 staining; blue, DAPI) at day six post-exposure (D8). Pathology is absent in the brain channel following exposure to monomer or PBS. Scale bars: 100 μm.

FIG. 57C Quantitative analysis of fluorescence intensity in each group at day three and six post-exposure (D5 and D8, respectively).

FIG. 57D Immunofluorescence staining shows phospho-αSyn129 (green) accumulation within the TH (red) positive neurons in the SN Brain-Chip. yellow indicates co-localization of phospho-αSyn129 and TH. Statistical analysis is two-way ANOVA with Tukey's multiple comparisons test (n=3-4 independent chips with 3˜5 randomly selected different areas per chip, *P<0.05, ****P<0.0001 compared to monomeric or PBS group). Error bars present mean±SEM.

FIG. 58A-B shows exemplary accumulation of phosphorylated αSyn and mitochondrial impairment in the αSyn fibril model at day 5.

FIG. 58A shows exemplary assessment of phosphorylated αSyn resulting from a three day post-exposure to αSyn fibrils shown in lower row of panels. Immunofluorescence micrographs show the accumulation of phosphorylated αSyn (green, phospho-αSyn129 staining-vertical middle panels; blue, DAPI stained nuclei-vertical left panels) and merged images-vertical right panels. PBS treated controls upper row of panels, αSyn monomer treated middle row of panels. Scale bars: 100 μm.

FIG. 58B shows exemplary effects of αSyn fibrils on mitochondrial membrane potential at three days after exposure. Mitochondrial membrane potential assessed by JC-1 staining on the brain side. Dual emission images (527 and 590 nm) represent the signals from monomeric (green) and J-aggregate (red) JC-1 fluorescence. Scale bars: 100 μm.

FIG. 59A-D shows exemplary reduction of mitochondrial activity and increase in ROS production in the αSyn fibril model.

FIG. 59A shows exemplary mitochondrial membrane potential assessed by JC-1 staining in the brain side at day six post-exposure. Dual emission images (527 and 590 nm) represent the signals from monomeric (green) and J-aggregate (red) JC-1 fluorescence. Scale bars: 100 μm.

FIG. 59B Quantitative analysis of the ratio of Red/Green fluorescence intensity in each group at day three and six post-exposure (D5 and D8, respectively). Statistical analysis is two-way ANOVA with Tukey's multiple comparisons test (n=3 independent chips with 3-4 randomly selected different areas per chip, *P<0.05, ****P<0.0001 compared to monomeric group).

FIG. 59C shows exemplary representative images of ROS levels (green, CellROX) show higher levels of intercellular ROS in the cells of the brain channel exposed to αSyn fibrils than those exposed to αSyn monomer at day six post-exposure. Scale bars: 100 μm.

FIG. 59D shows exemplary quantification of the number of CellROX-positive events per field of view in each group. Statistical analysis is Student's t test (n=3 independent chips with 3-4 randomly selected different areas per chip, ****p<0.0001 compared to monomeric group). Error bars present mean±SEM.

FIG. 60A-F shows exemplary αSyn-induced apoptotic cell dopaminergic neuronal death indicated by caspase-3 activation (red) and neuroinflammation in the presence of activated astrocytes and activated microglia/macrophages. Increasing expression of glial fibrillary acidic protein (GFAP) represents astroglial activation and gliosis during neurodegeneration. Increasing expression of CD68 represents

FIG. 60A shows exemplary representative merged images showing double immunostaining for dopaminergic neurons by MAP2 (grey) and Cleaved Caspase-3 (red, CC3) in the brain channel at six-days post-exposure. Scale bars: 50 μm.

FIG. 60B shows exemplary quantitative data on the number of CC3 positive neurons. Statistical analysis is Student's t test (n=3 independent chips with 3-4 randomly selected different areas per chip, ***p<0.001 compared to monomeric group).

FIG. 60C shows exemplary immunostaining of the astrocyte marker GFAP (magenta) demonstrating activation of astrocytes at day 8 following exposure to αSyn fibrils compared to monomeric αSyn. Scale bar, 100 μm.

FIG. 60D shows exemplary immunostaining of the microglial CD68 (red) demonstrated activation of astrocytes and microglia at day 8 following exposure to αSyn fibrils compared to monomeric αSyn. Scale bar, 100 μm.

FIG. 60E shows exemplary secreted levels of TNF-α in the αSyn fibril model. Statistical analysis was by Student's t-test (n=6-7 independent chips, **p<0.01).

FIG. 60F shows exemplary secreted levels of proinflammatory cytokine IL-6 in the αSyn fibril model. Statistical analysis was by Student's t-test (n=4˜7 independent chips, ****p<0.0001). Error bars present mean±SEM.

FIG. 61A-D shows exemplary αSyn-induced cell death and neuroinflammation.

FIG. 61A shows exemplary cell viability (live/dead) assay following exposure to human αSyn fibrils. Live/Dead cell staining assay was designed to test the potential cytotoxicity of αSyn fibrils at days 5 and 8 of culture. Scale bars: 100 μm.

FIG. 61B shows exemplary data are expressed as the average live cells/total number of cells (sum of calcein AM positive and ethidium homodimer positive cells). Statistical analysis is two-way ANOVA with Tukey's multiple comparisons test (n=3 independent chips with 3˜5 randomly selected different areas per chip, ****P<0.0001 compared to monomeric or PBS group, NS: Not Significant).

FIG. 61C shows exemplary quantification of the number of GFAP-positive events per field of view. Statistical analysis is Student's t test (n=3 independent chips with 3-4 randomly selected different areas per chip, ***p<0.001 compared to monomeric group).

FIG. 61D shows exemplary quantification of the number of CD68-positive events per field of view. (n=3 Brain-Chips with 3˜5 randomly selected different areas per chip, ****P<0.0001 compared to the monomeric group). Error bars present mean±SEM.

FIG. 62 shows an exemplary schematic model for measuring BBB breakdown. In one embodiment modeling neuroinflammation when a-Syn is added to the brain channel. In one embodiment modeling systemic inflammation when a-Syn is added to the vascular channel.

FIG. 63A-D shows exemplary Blood-Brain Barrier dysfunction in the αSyn fibril model.

FIGS. 63A and 56 FIG. 63B shows exemplary quantitative barrier function analysis via permeability to 0.5 kDa lucifer yellow and 3 kDa fluorescent dextran at day 5 and 8 following exposure to αSyn fibrils or αSyn monomers. Statistical analysis is two-way ANOVA with Tukey's multiple comparisons test (n=6˜9 independent chips, ****P<0.0001 compared to monomeric group, NS: Not Significant).

FIG. 63C shows exemplary principal component analysis generated using the RNA-seq data generated by the samples collected from the vascular channel of the SN Brain-Chip upon exposure to αSyn monomers or αSyn fibrils (n=4 per condition). A 2D-principal component plot is shown with the first component along the X-axis and the second along the Y-axis. The proportion of explained variance is shown for each component.

FIG. 63D shows exemplary volcano plot indicating DE genes between αSyn fibrils and αSyn monomers, as identified by the RNA-sequencing analysis. For the selection of the DE genes the following thresholds were applied: adjusted p-value<0.05 and |Log 2(foldchange)|>0.5. The identified up- (down-) regulated genes are highlighted in cyan (magenta) color. Sample sizes were as follows: Brain-Chip (αSyn monomers), n=4, Brain-Chip (αSyn fibrils), n=4.

FIG. 64A-B shows exemplary Blood-Brain Barrier dysfunction in the αSyn fibril model. IgG Penetration through BBB. See, Table 6.

FIG. 64A shows exemplary quantitative barrier function analysis via permeability to immunoglobulin G (IgG1) at day 5 and 8 following exposure to αSyn fibrils, αSyn monomers or PBS. Statistical analysis is two-way ANOVA with Tukey's multiple comparisons test (n=5˜8 independent chips, ****P<0.0001 compared to monomeric group, NS: Not Significant). Error bars present mean±SEM.

FIG. 64 B shows exemplary selection of the 739 up-regulated and 541 down-regulated genes identified after performing DGE analysis between αSyn fibrils and αSyn monomers. The size of the bars indicates the log₂(Fold-Change) of the corresponding gene expressions and the colors the statistical significance (FDR adjusted p-values) of the corresponding changes.

FIG. 65 shows exemplary schematic depictions toxicity of α-syn as a therapeutic target. Toxicity of alpha-synuclein to neurodegeneration is associated tightly with the dynamic equilibrium of the protein synthesis, aggregation, and clearance. Levels of specific conformations (oligomers and protofibrils) vary in different stages of PD. Disease-modifying therapeutic strategies are mainly focused on these processes as well as inhibiting cell-to-cell propagation: (i) reducing a-syn synthesis with small interfering RNA (siRNA), microRNA (miRNA), small hairpin RNA (shRNA), and transcription inhibitors; (ii) increasing degradation of a-syn via UPS and ALP; (iii) reducing aggregation of a-syn via heat-shock proteins (hsp40/70/104), aggregation inhibitors, antioxidant, and posttranslational modification approaches (oxidation, nitration, phosphorylation, and C-terminal cleavage); (iv) blocking the propagation of a-syn with immunotherapies by targeting extracellular a-syn or exosome and by blocking putative receptors in recipient cells; and (v) seeking neuroprotective strategies including anti-inflammation and antioxidant.

FIG. 66A shows exemplary potential mechanisms involved in propagation of α-syn. Spreading mechanisms of α-syn in neighboring cells are multiple and can occur via (1) passive transmission through membrane fusion; (2) classical exocytosis and endocytosis; (3) packaged-exosomes; (4) tunneling nanotubes (a direct connection between two cells); (5) axonal transport and transsynaptic junction; and (6) receptor-mediated internalization.

FIG. 66B shows exemplary molecules and signaling pathways involved in α-syn-mediated microglial activation. Excessive microglial activation can increase the production of pro-inflammatory cytokines (TNF-α, IL-1β, IL-6, and TNF-γ), and induce an oxidative stress response, including the release of reactive oxygen species (ROS) and nitric oxide (NO) as well as the production of NADPH oxidase. Toll-like receptors (TLRs) play a vital role in recognizing pathogen-associated molecular patterns (PAMPs) and initiating innate immune responses via distinct signaling pathways, including NF-κB and MAPK activation. Activation of TLR2 resulted in the accumulation of α-syn as a result of the inhibition of autophagic activity through regulation of the AKT/mTOR pathway. Other receptors that are involved in the α-syn-induced microglial response include FcγRs/CD36/P2×7R/EP2/Mac-1/Ion channels. Also, α-syn induced the expression of matrix metalloproteinases (MMPs) and stimulated the activities of MAPK, NF-κB, and AP-1. In addition, MMPs may activate microglial protease-activated receptor-1 (PAR-1) in an autocrine or paracrine manner and increase microglial inflammatory signals (not shown in the diagram). Furthermore, major histocompatibility complex II (MHC-II) and Th1 cells were targeted recently for the activation of microglia. Exosomes are specifically and efficiently taken up by microglia via a macropinocytotic mechanism and are released via activation of 5-hydroxytryptamine (5-HT2a, 2b, and 5-HT4) receptors. Activated exosomes expressed a high level of MHC-II, which may be a potentially pathway for the activation of microglia. In contrast, regulator of G-protein signaling 10 (RGS10), RING finger protein 11 (RNF11), and NF-κB essential modulator (NEMO) inhibitors exert negative regulation on NF-κB signaling, producing a dampened immune response. Finally, microglial cells are also able to phagocytose different forms of extracellular α-syn, via ubiquitin-proteasomal system (UPS) and autophagy-lysosomal pathway (ALP), presenting a mechanism of clearance that might be even beneficial for neuronal survival. The CD36 (a scavenger receptor), FcγRs (Fc gamma receptors), Mac-1 (macrophage antigen-1 receptor), EP2 (prostaglandin E2 receptor subtype 2), P2×7R (purinergic receptor P2×, ligand-gated ion channel 7), and plasma membrane ion channels.

FIG. 66C shows exemplary internalization of α-synuclein fibrils and aggregation of endogenous α-syn protein. Recombinant α-syn fibrils are transported into the cell through endocytosis. This process is facilitated by the binding of α-syn PFFs to the cell membrane through interactions with cell surface molecules. In particular, the cell surface receptor LAG3 (lymphocyte activation gene 3) can bind and mediate the endocytosis of fibrillary α-syn. Additionally, α-syn fibrils can bind and cluster a number of other surface receptors at the plasma membrane. It is currently unknown whether any of these cell surface proteins can regulate the uptake of α-syn as well. Heparan sulfate proteoglycans (HSPG), abundant extracellular glycoproteins that are able to interact with a large number of extracellular proteins and ligands, are able to bind α-syn fibrils and promote their uptake. Internalized PFFs travel through the early and late endosomal compartment to the lysosome, where they are destined for degradation. Through some unknown process, α-syn PFFs can escape the lumen of the endosomal compartment and template the misfolding of soluble endogenously expressed α-syn in the cytoplasm. (??) indicates additional mechanisms and molecular players.

FIG. 67 shows an exemplary schematic diagram contemplating a-Synuclein fibrils (PFF) (red circles) recruiting endogenous a-Synuclein (aSyn) (yellow circles) to form aggregates and induce neuron death. At least some aggregates are released and propagate to neighboring cells and further pathological damage to the brain. Enhanced lysosomal efficiency/hydrolytic capacity through increased Cathepsin D or enhanced autophagosome production through trehalose treatment may promote the sequestering and degradation of toxic aSyn species. Redmann et al., Aging and Disease, 2016.

FIG. 68A shows an exemplary timeline for methods of use in testing therapeutic test compounds.

FIG. 68B shows exemplary morphological analysis of tight junctions in endothelial cells in the αSyn fibril model with or without trehalose treatment. The junction protein expression of ZO-1 was visualized by immunofluorescence staining with a ZO-1 antibody. Scale bars: 50 μm.

FIGS. 69A-B shows an exemplary effect of a test compound as an autophagic inducer, trehalose on BBB integrity.

FIG. 69A shows an exemplary quantitative barrier function analysis via permeability to 3 kDa fluorescent dextran at day 8 in the αSyn fibril model with or without trehalose treatment. Statistical analysis is Student's t test (n=5˜8 independent chips, ****P<0.0001 compared to monomeric group, ***P<0.001 compared to αSyn fibrils). Error bars present mean±SEM.

FIG. 69B shows an exemplary morphological analysis of tight junctions in endothelial cells in the αSyn fibril model with or without trehalose treatment. The junction protein expression of Claudin-5 was visualized by immunofluorescence staining with a Claudin-5 antibody and DAPI for cell nuclei. Scale bars: 50 μm.

### DEFINITIONS

An Organ-Chip refers to a living, micro-engineered 3-D environment that recreates the natural physiology and mechanical forces that cells experience within the human body. An organ-on-α-chip is contemplated for use as described in the U.S. Pat. No. 8,647,861, and the International Patent App. No. PCT/US2014/071611, the contents of each of which are incorporated herein by reference in their entireties.

As used herein, the phrases “linked,” “connected to,” “coupled to,” “in contact with” and “in communication with” refer to any form of interaction between two or more entities, including mechanical, electrical, magnetic, electromagnetic, fluidic, and thermal interaction. For example, in one embodiment, channels in a microfluidic device are in fluidic communication with cells and (optionally) a fluid reservoir. Two components may be coupled to each other even though they are not in direct contact with each other. For example, two components may be coupled to each other through an intermediate component (e.g. tubing or other conduit).

“Channels” are pathways (whether straight, curved, single, multiple, in a network, etc.) through a medium (e.g., silicon, plastic, etc.) that allow for movement of liquids and gasses. Channels thus can connect other components, i.e., keep components “in communication” and more particularly, “in fluidic communication” and still more particularly, “in liquid communication.” Such components include, but are not limited to, liquid-intake ports and gas vents.

“Microchannels” are channels with dimensions less than 1 millimeter and greater than 1 micron. Additionally, the term “microfluidic” as used herein relates to components where moving fluid is constrained in or directed through one or more channels wherein one or more dimensions are 1 mm or smaller (microscale). Microfluidic channels may be larger than microscale in one or more directions, though the channel(s) will be on the microscale in at least one direction. In some instances the geometry of a microfluidic channel may be configured to control the fluid flow rate through the channel (e.g. increase channel height to reduce shear). Microfluidic channels can be formed of various geometries to facilitate a wide range of flow rates through the channels.

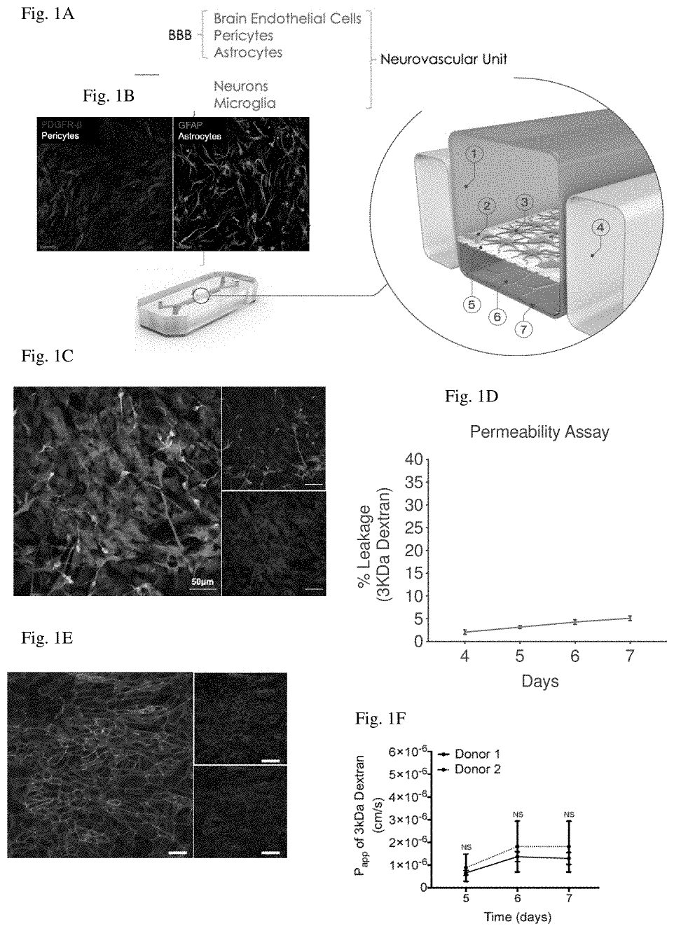

The present invention contemplates a variety of “microfluidic devices,” including but not limited to microfluidic chips (such as that shown in FIG. 1A), perfusion manifold assemblies (without chips), and perfusion manifold assemblies (10) with a cover or lid (11) engaged with microfluidic chips (16) carrier (17) such as that shown in FIG. 28I). However, the methods described herein for engaging microfluidic devices (e.g. by drop-to-drop connections), and for perfusing microfluidic devices are not limited to the particular embodiments of microfluidic devices described herein, and may be applied generally to microfluidic devices, e.g. devices having one or more microchannels and ports.

“Parenchyma” refers in general to functional cells or parts of an organ that may also be referred to descriptively as “parenchymal”. As one example, in brain tissue, “parenchyma” refers to the functional tissue comprising at least two types of “parenchyma cells”, i.e. brain cells, e.g. neurons and glia (glial) cells. As one example, in intestinal tissue, “parenchyma” refers to epithelial cells, goblet cells, L-cells, etc.

Glia cells include but are not limited to oligodendrocytes (including myelin producing cells), ependymal cells, astrocytes and microglia (resident specialized brain macrophages). Such cells comprise a ventricular system (examples include oligodendrocytes and ependymal cells) and a neurovascular unit comprising neurons, astrocytes, pericytes and endothelial cells.

“Gliosis” refers to a reactivate change of glial cells, including astrocytes, microglia, and oligodendrocytes. Examples of change include but are not limited to activation, proliferation (increased number), or hypertrophy (larger cells). In some embodiments, gliosis results from any one or more of traumatic brain injury (TBI), cerebral infarction (stroke), disease, etc.

“Non-parenchymal” refers to cells including but not limited to endothelial cells, macrophages, ependymal cells, etc.

“Brain cells” in general refer to any cell type found in vivo in any part of the brain. Examples of brain cells include but are not limited to parenchymal cells, non-parenchymal cells, neuroepithelial cells, pericytes, astrocytes, etc.

“Immune cells” refer to white blood cells.

### GENERAL DESCRIPTION OF THE INVENTION

The invention relates to methods, devices and systems for modeling brain neuronal disease in a microfluidic device, comprising a co-culture of a variety of cell types such as iPS-derived brain endothelial cells; iPS-derived dopaminergic neurons; primary neurons, primary microglia; and primary astrocytes, a Blood-Brain-Barrier (BBB)-Chip and a Brain-Chip. In particular, cross-talk between glial cells (e.g. microglia and astrocytes) with neuronal cells, in further contact with endothelial cells is contemplated for use for identifying drug targets under conditions for inducing in vivo relevant neuronal inflammation, neurodegeneration and neuronal death. Thus, in one embodiment, a microfluidic Brain-Chip comprising a co-culture of brain cells is exposed to α-synuclein preformed fibrils (PFF), a type of pathogenic form of α-synuclein. Such α-synuclein PFF exposure demonstrates an in vivo relevant disease pathogenesis on a microfluidic device as a concentration- and time-controlled manner that may be used for preclinical drug evaluation for diseases related to neuronal inflammation, e.g. Parkinson's Disease (PD). In some embodiments, modulation of complement in the presence of neuronal inflammation is contemplated. In some embodiments, drug delivery to brain cells across the BBB is contemplated for preclinical testing of drug efficacy for slowing or stopping neuronal inflammation and degeneration.

The invention related to study of the effect of compounds altering neuronal health on the Blood-Brain Barrier (BBB) and the forebrain, including but not limited to cellular cytoskeleton, barrier function, neuronal activity, and RNA levels. In one embodiment, seeded cells induced pluripotent stem cells (iPSC)-derived cortical neurons, human primary astrocytes and pericytes in the neuronal channel (top), and iPSC-derived human brain microvascular endothelial cells in the vascular channel (bottom). Health of neuronal populations are shown by readouts including but not limited to Calcium imaging and stable release of neurotransmitter, e.g. Glutamate. Blood-Brain-Barrier integrity was measured using diffusion and biomarker expression. Presynaptic vesicles are shown to be colocalized in the neuronal axons which validate the proper synaptic activity of neurons. TNF-α treatment on the neuronal channel shows neuroinflammatory response by neuronal death and astrogliosis, while in some embodiments, treatment of the vascular channel with TNF-α resulted in the breakdown of the BBB.

Parkinson's disease (PD) and related synucleinopathies are characterized by the abnormal accumulation of alpha-synuclein (αSyn) aggregates, loss of dopaminergic neurons, and gliosis in the substantia nigra (SN). Although clinical evidence and in vitro studies indicate disruption of the Blood-Brain Barrier (BBB) in PD, the mechanisms mediating the endothelial dysfunction remain elusive. Lack of relevant models able to recapitulate the order of events driving the development of the disease in humans was a significant bottleneck in the identification of specific druggable factors, such as drug targets. Organ-on-Chip type technology to design a human SN Brain-Chip containing dopaminergic neurons, astrocytes, microglia, pericytes, and brain endothelial cells, cultured under fluid flow. This embodiment of a Brain-Chip, having dopaminergic neurons, astrocytes, microglia, pericytes in the brain channel with brain endothelial cells in a vascular channel, was exposed to αSyn fibrils. αSyn fibrils surprising disrupted the BBB in addition to showing inflammatory and neurodegerative conditions in the Brain channel. This αSyn fibril-induced neuroinflammation model was capable of reproducing several aspects of Parkinson's disease, including accumulation of phosphorylated αSyn (pSer129-αSyn), mitochondrial impairment, neuroinflammation, and compromised barrier function. These findings open areas of research that could help to elucidate the dynamics of cell-cell interactions in human synucleinopathies and screening of novel factors for specific therapeutic interventions.

### I. Examples of Main Advantages Using Embodiments of Microflidic Brain-Chips.

Embodiments of a Brain-Chip described herein, recapitulates the complexity of human brain to study disease pathology and for use in testing therapeutic treatments. Brain chips may also be used for identifying side effects of therapeutics for use in determining treatments on an individual basis. Embodiments of brain chips include but are not limited to: a neurovascular unit, a cellular interface between circulation and central nervous system (FIG. 1A). One embodiment of a Brain-Chip has two microfluidic channels, separated by a thin porous PDMS membrane that combines a brain endothelial monolayer with brain parenchymal cells, enabling highly sensitive quantification of molecular distribution in each space independently. The brain channel of the Brain-Chip accommodates excitatory neurons, inhibitory interneurons, pericytes astrocytes, and microglia, essential cellular elements of the NVU (McConnell et al., 2017), interacting with brain endothelial cells underneath the ECM-coated membrane creating a capillary-like structure at the vascular channel.

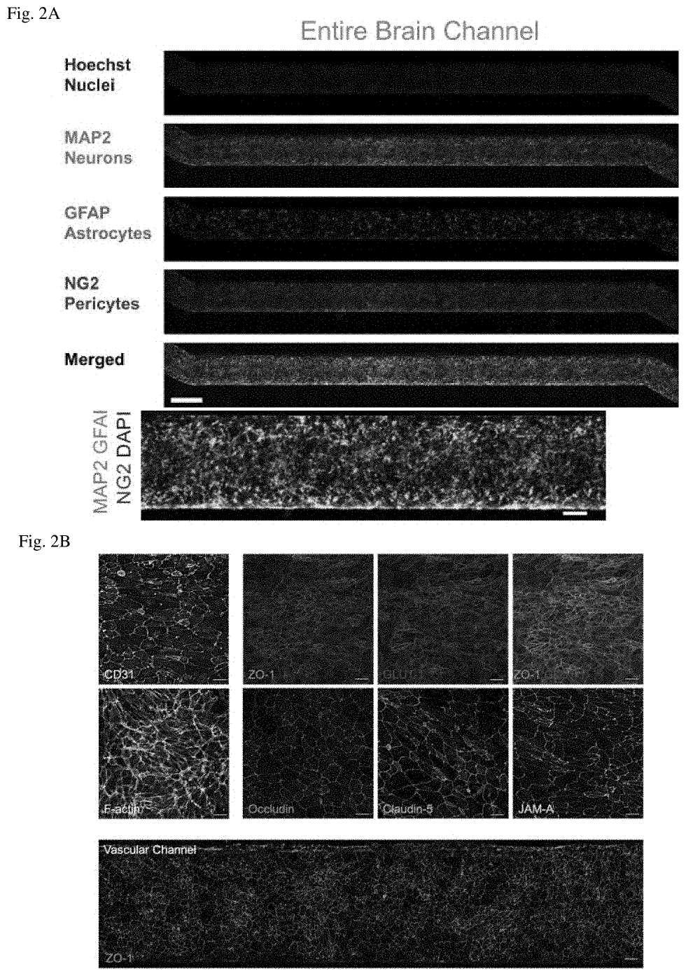

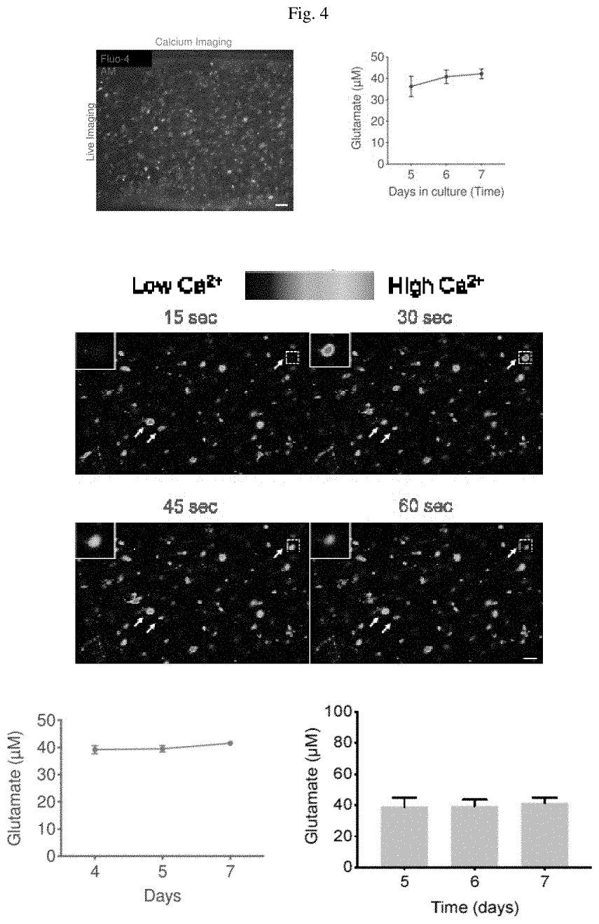

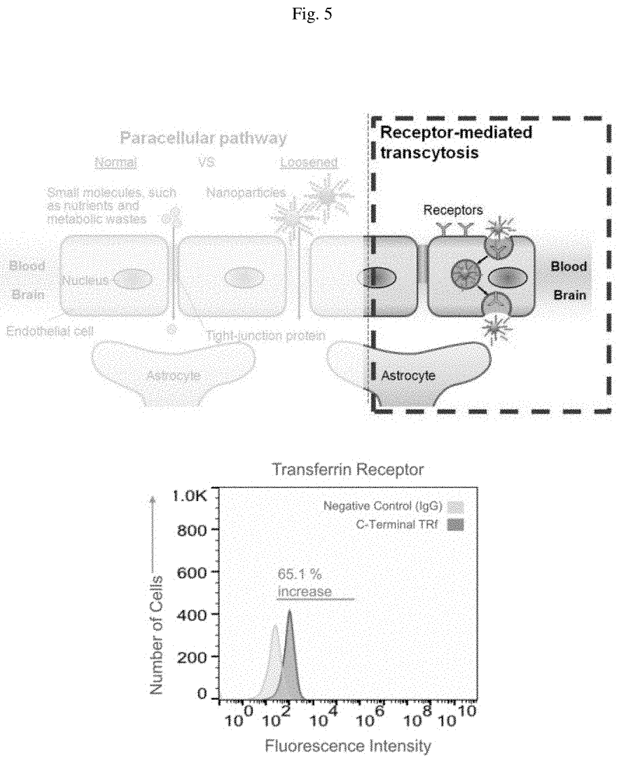

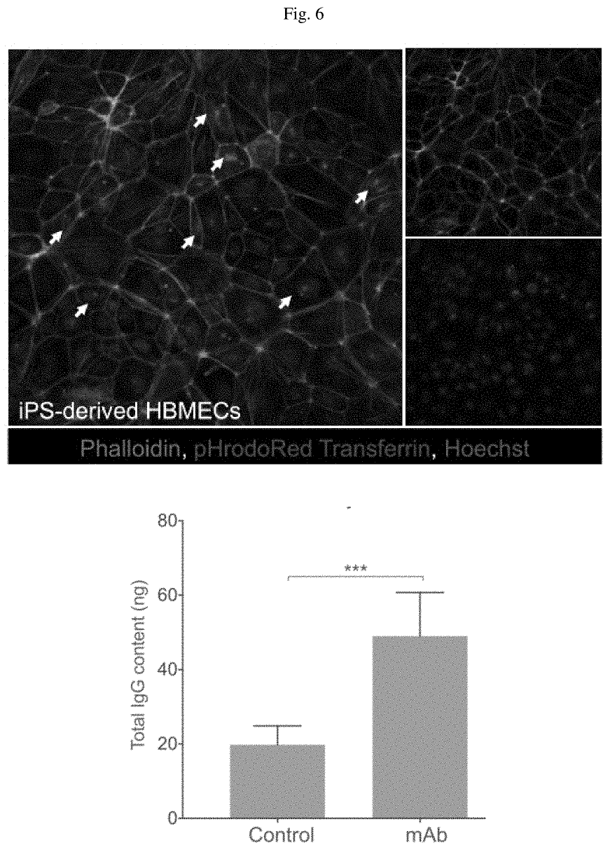

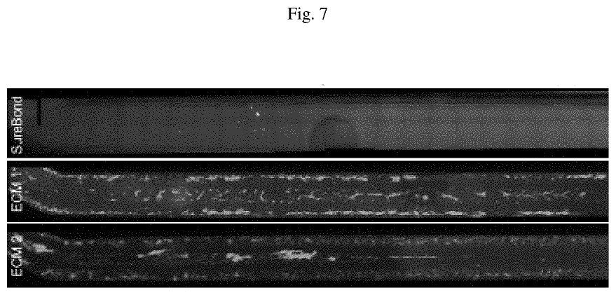

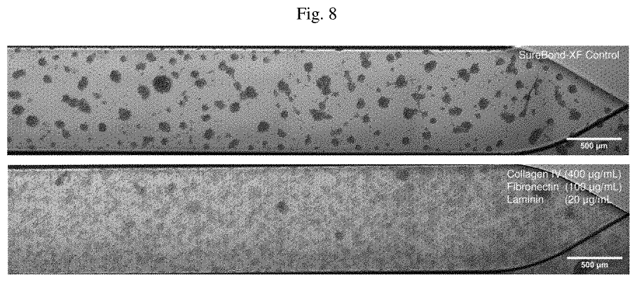

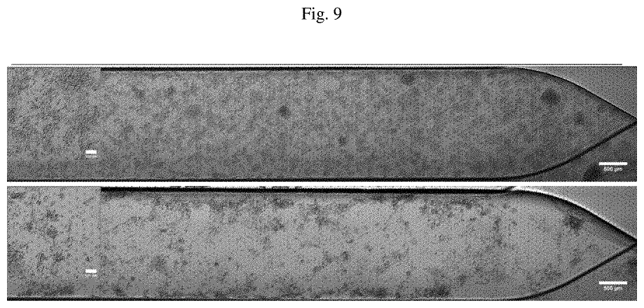

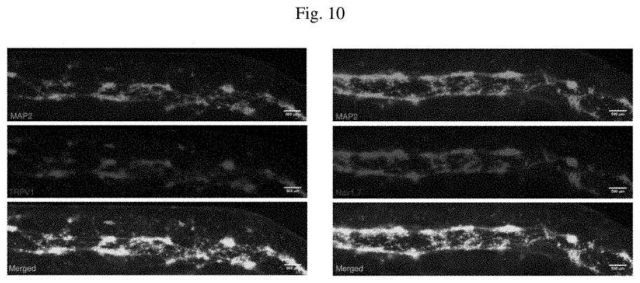

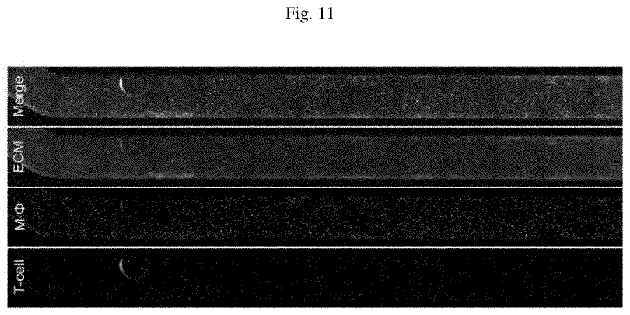

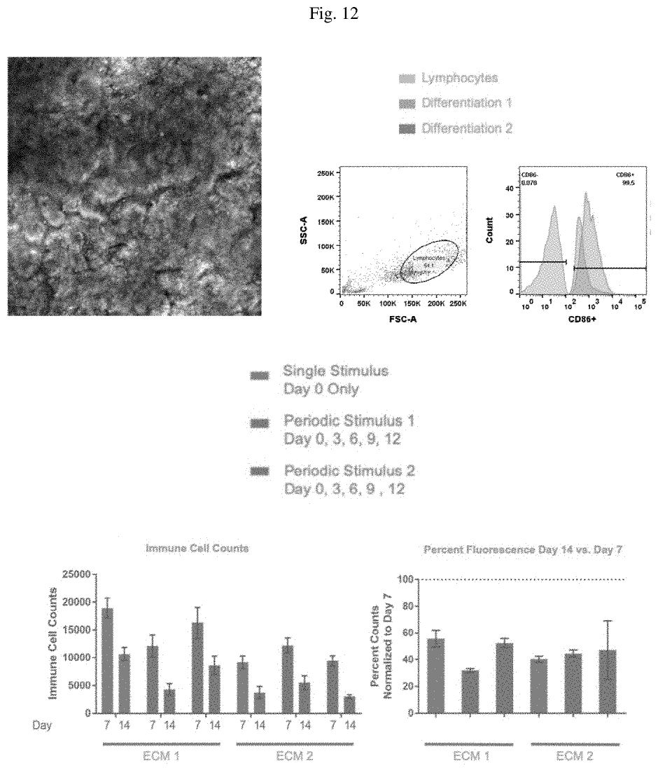

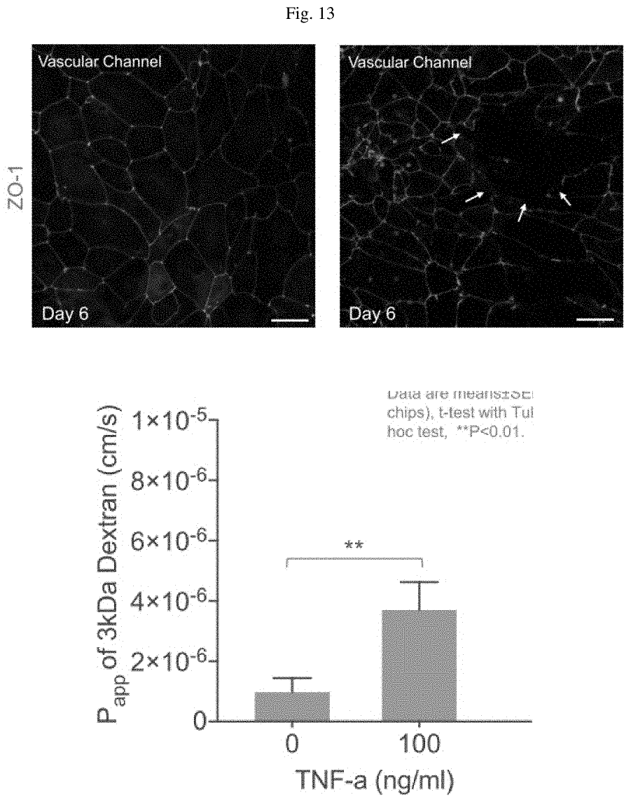

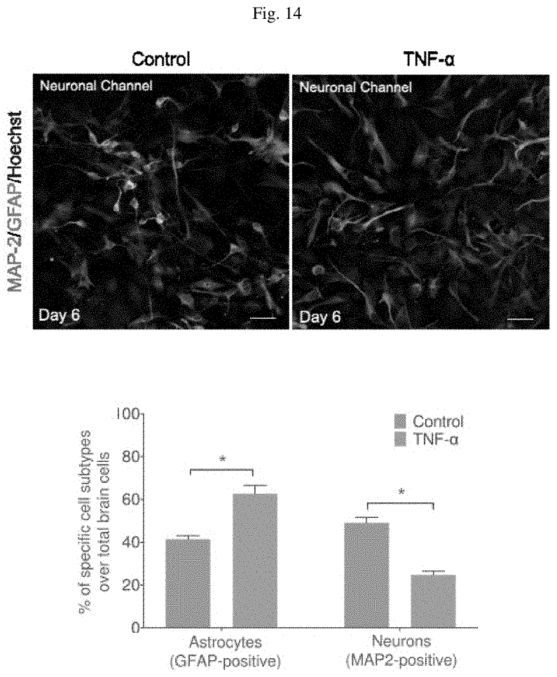

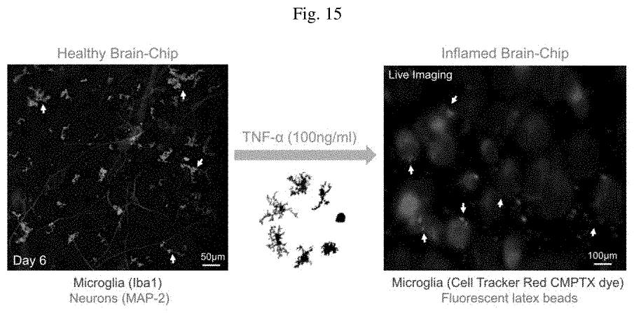

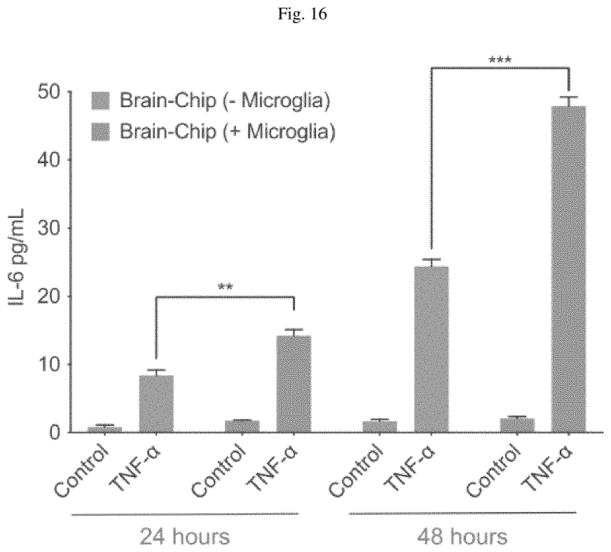

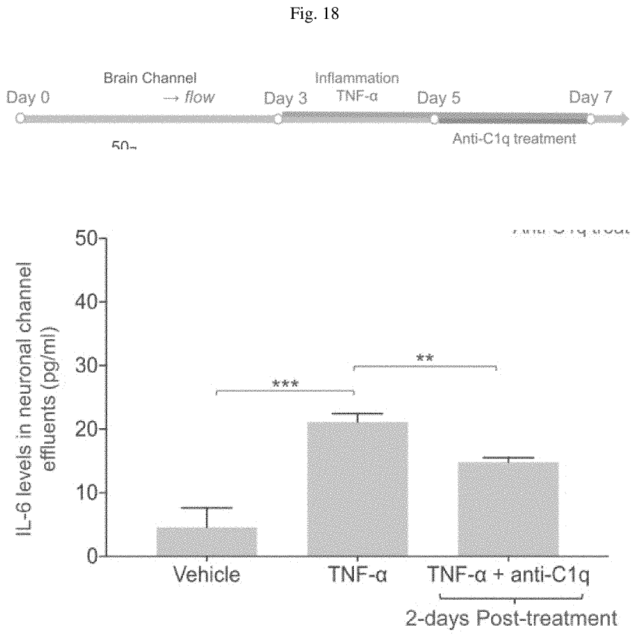

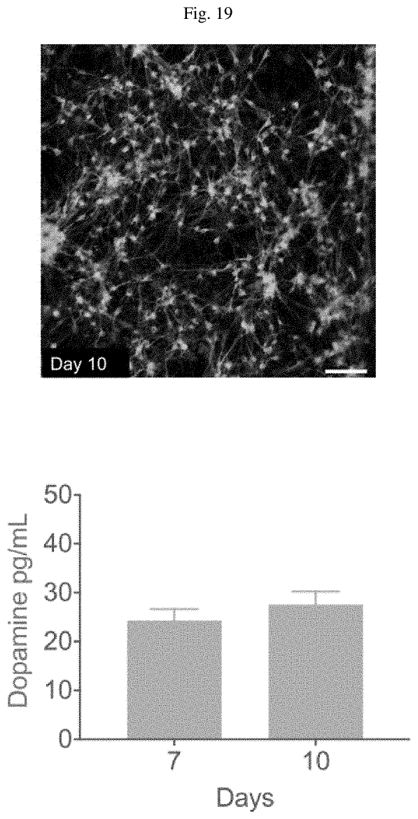

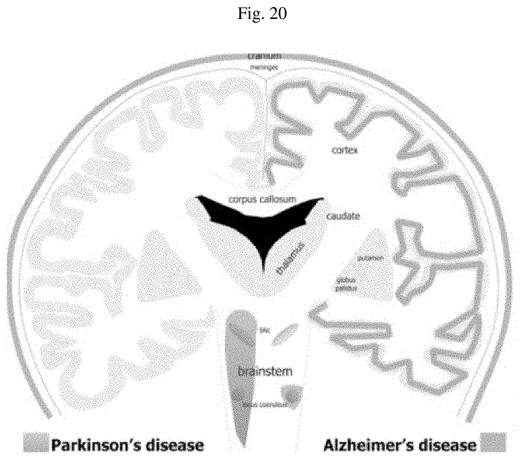

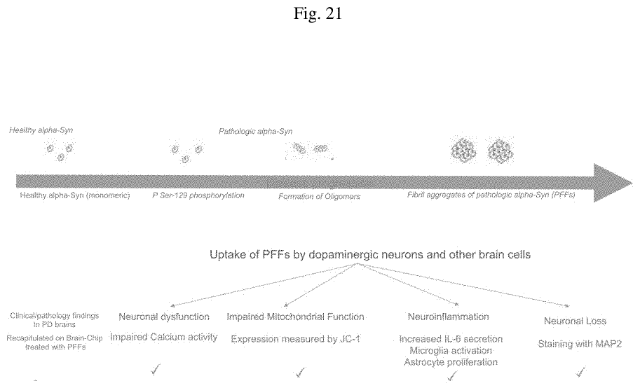

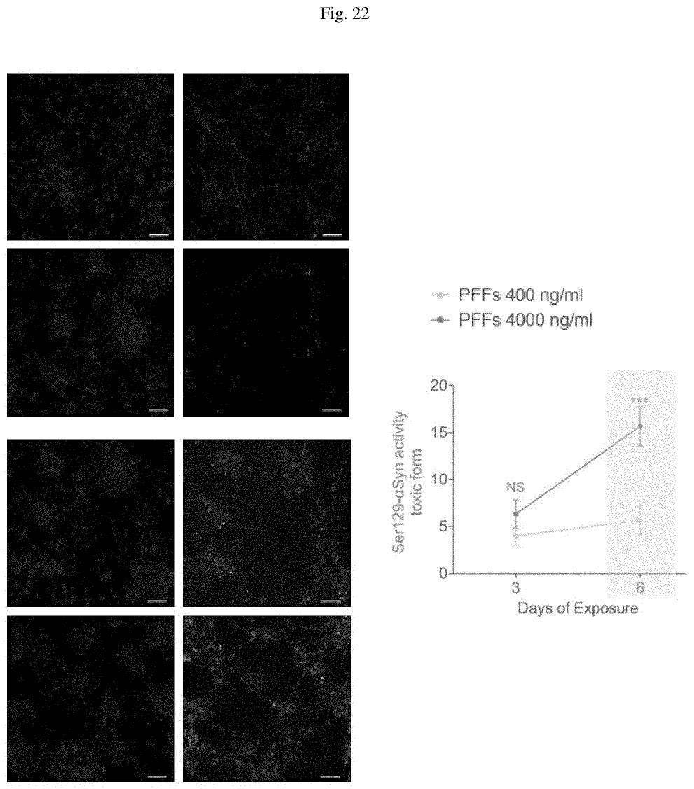

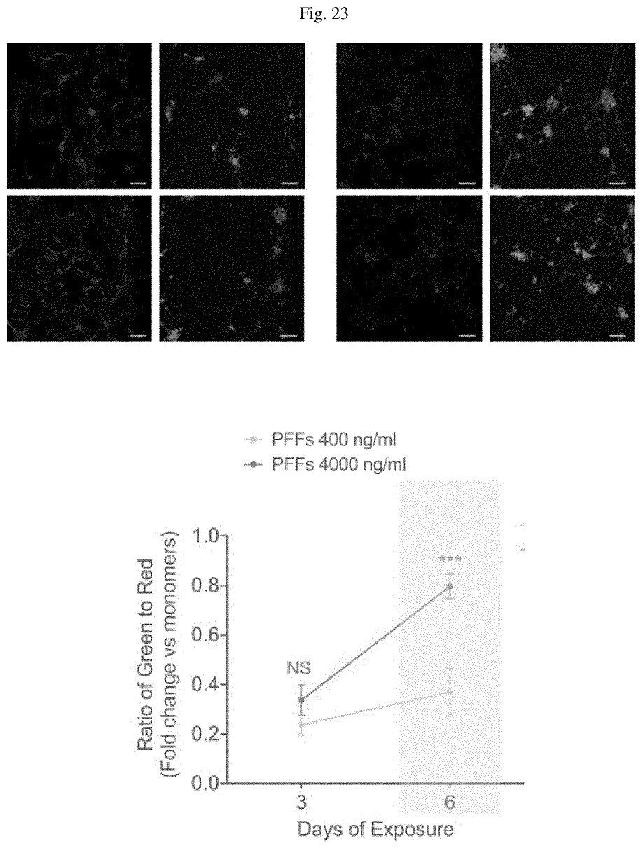

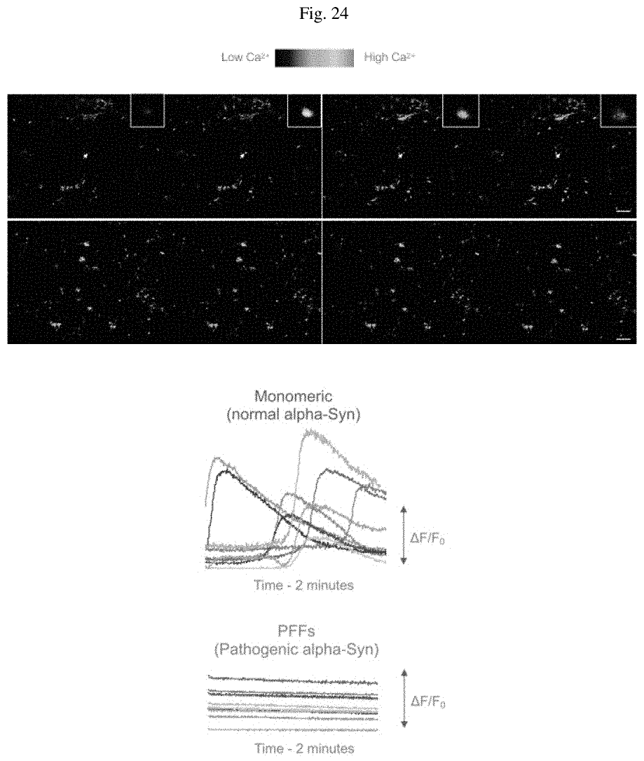

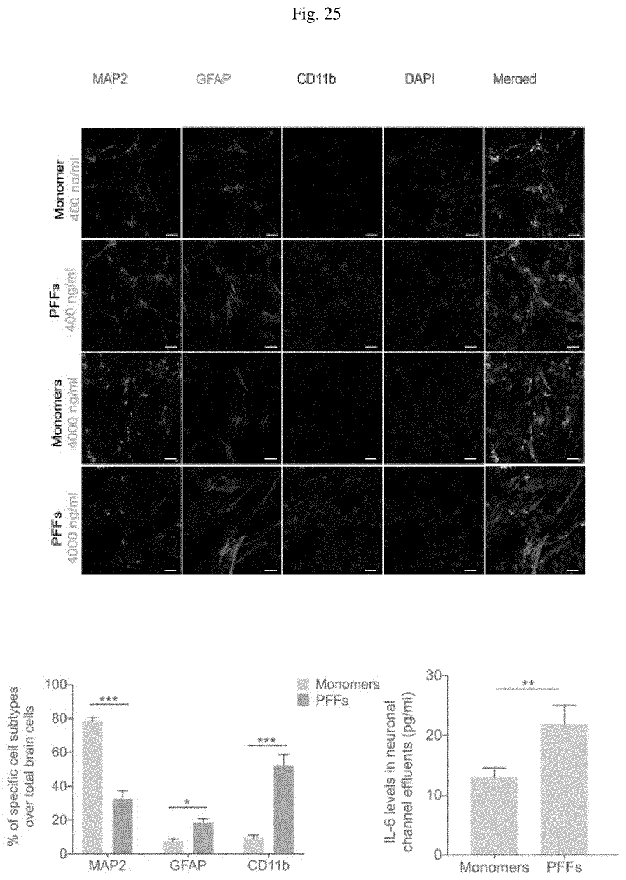

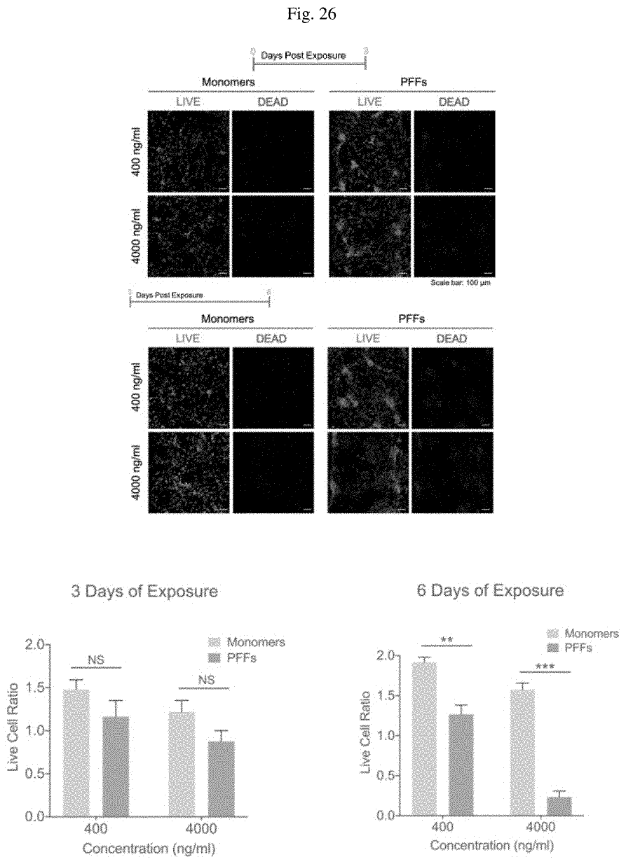

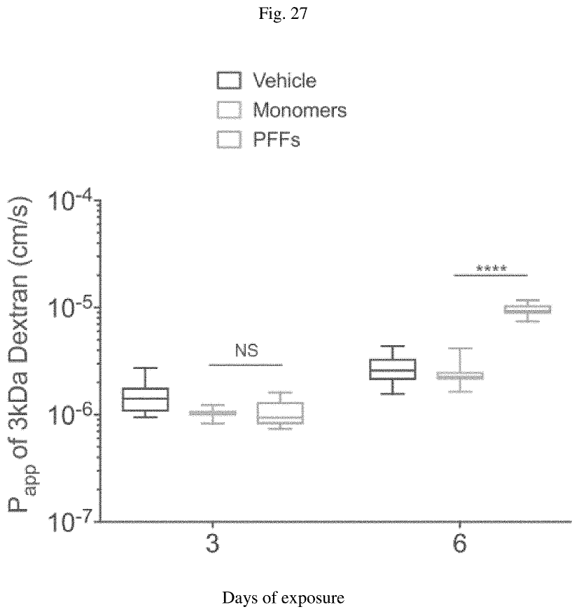

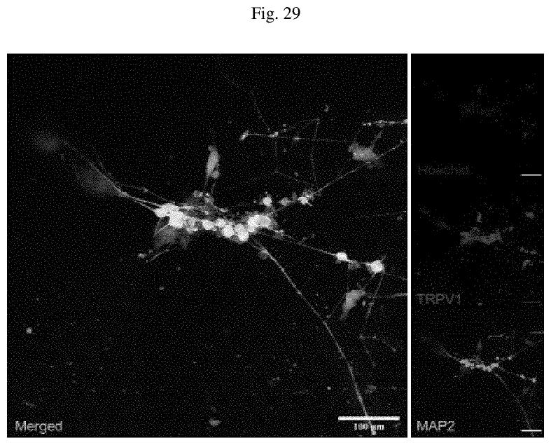

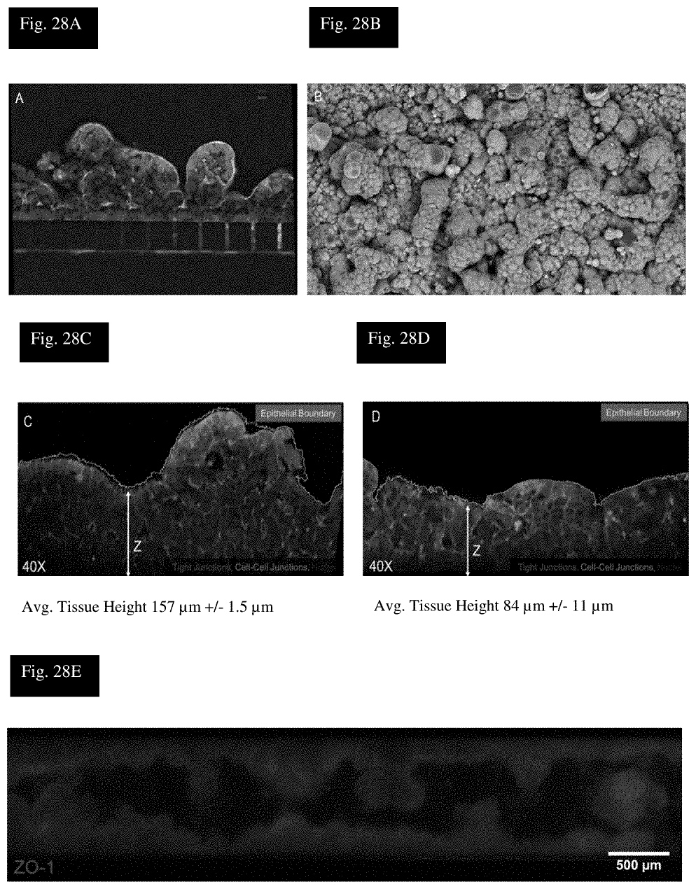

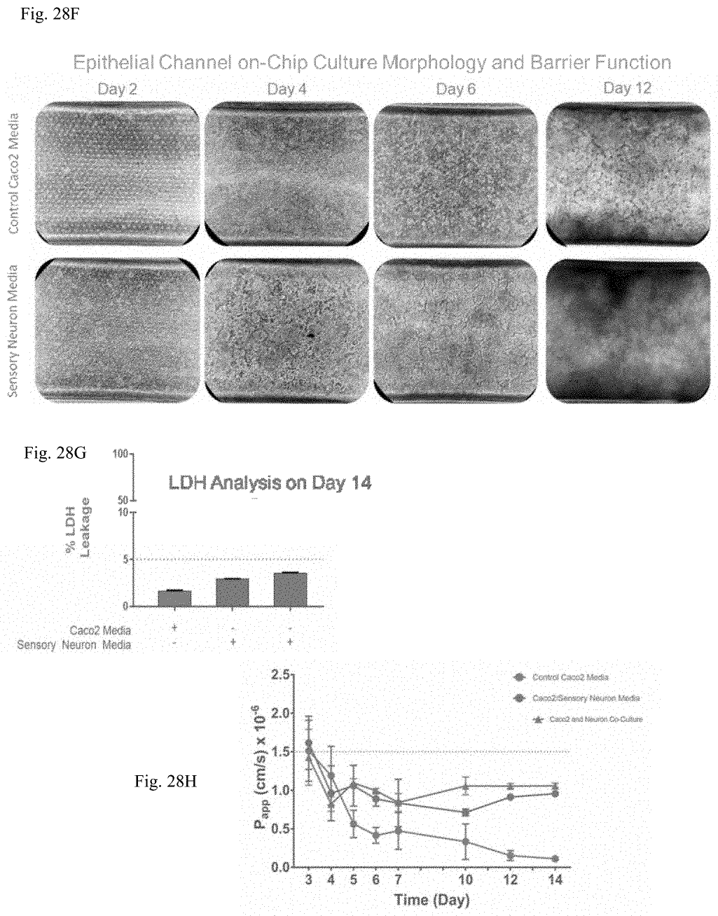

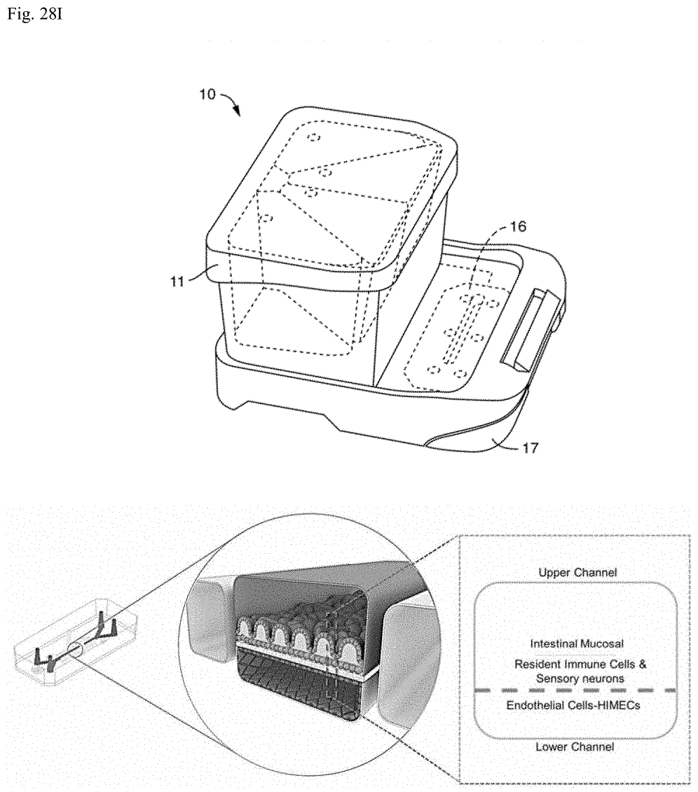

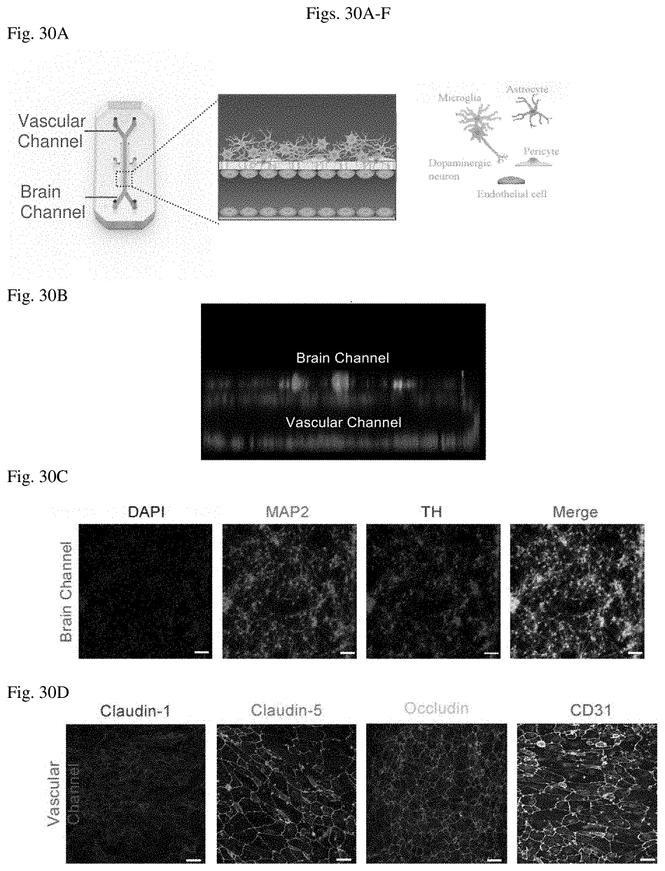

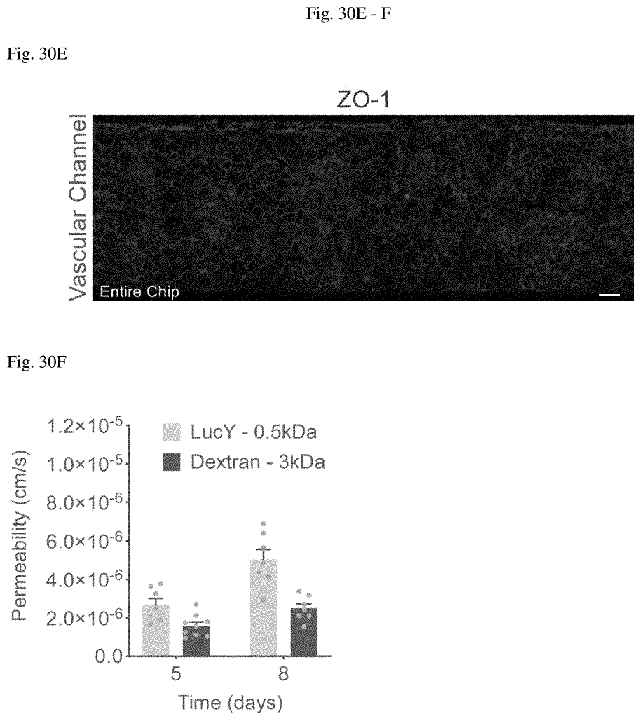

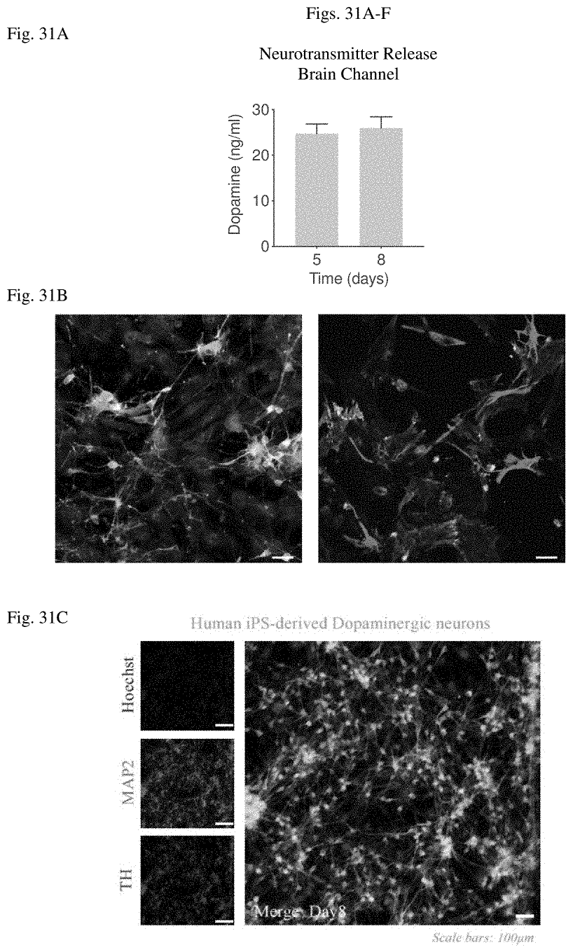

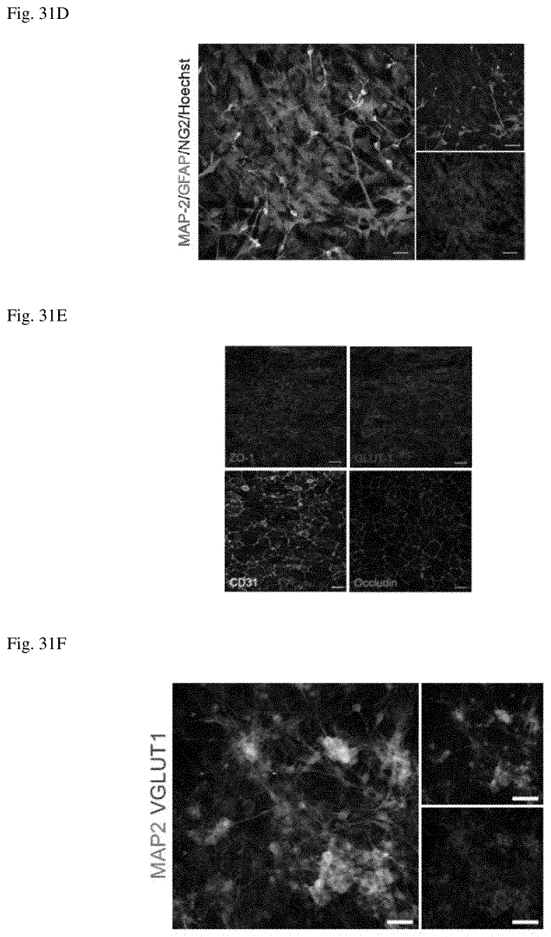

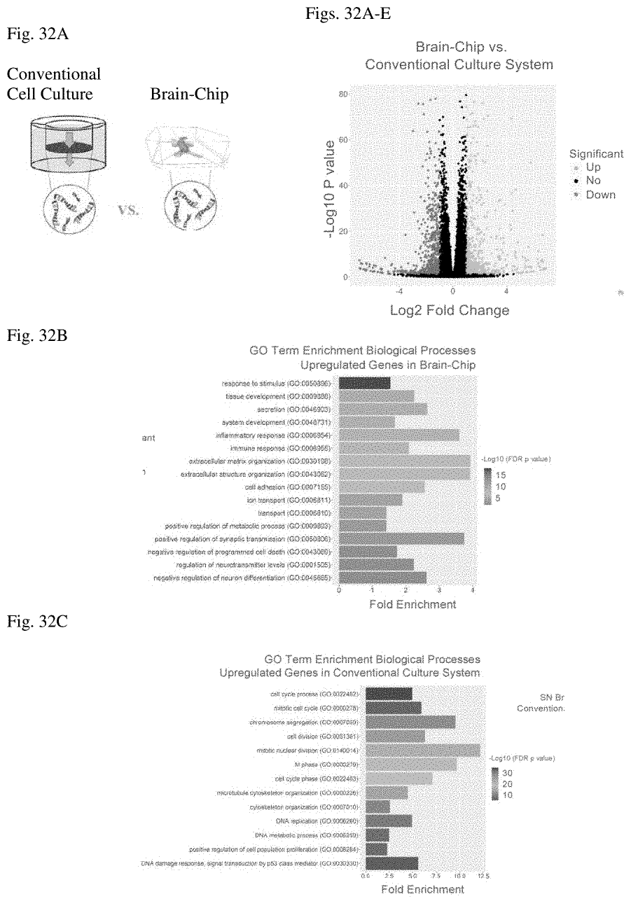

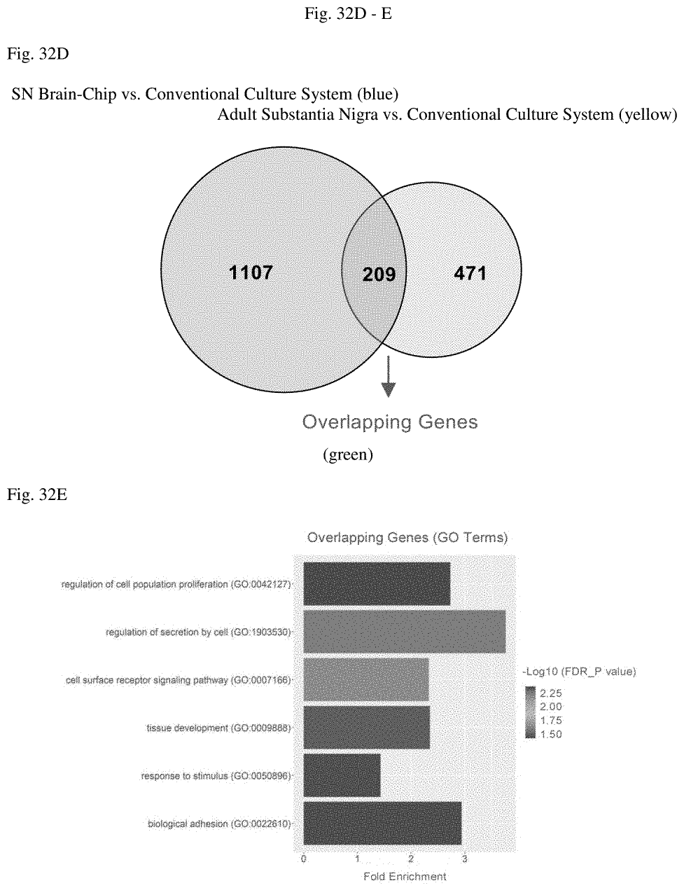

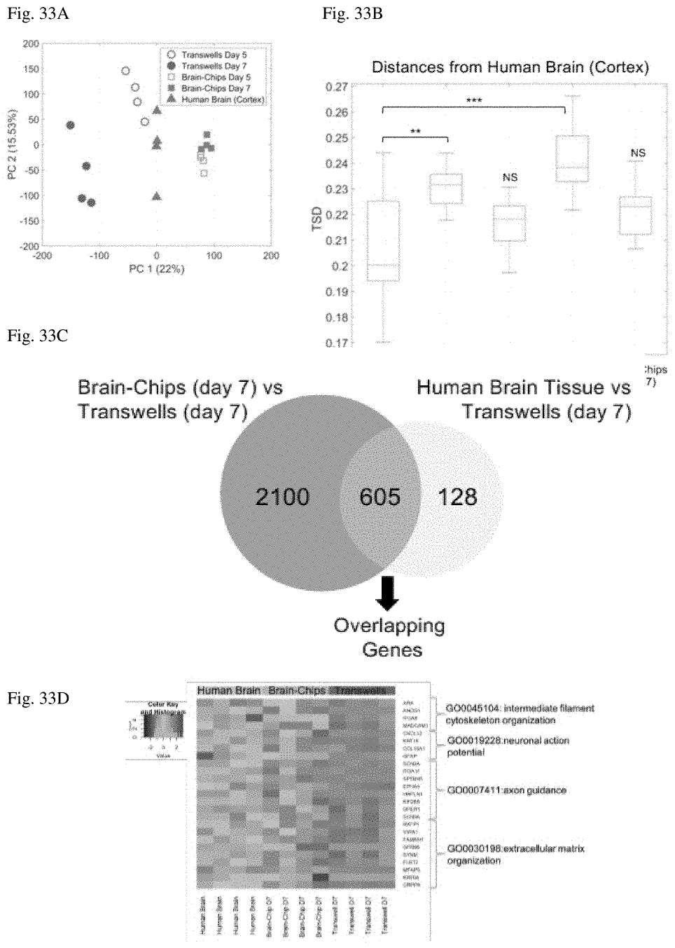

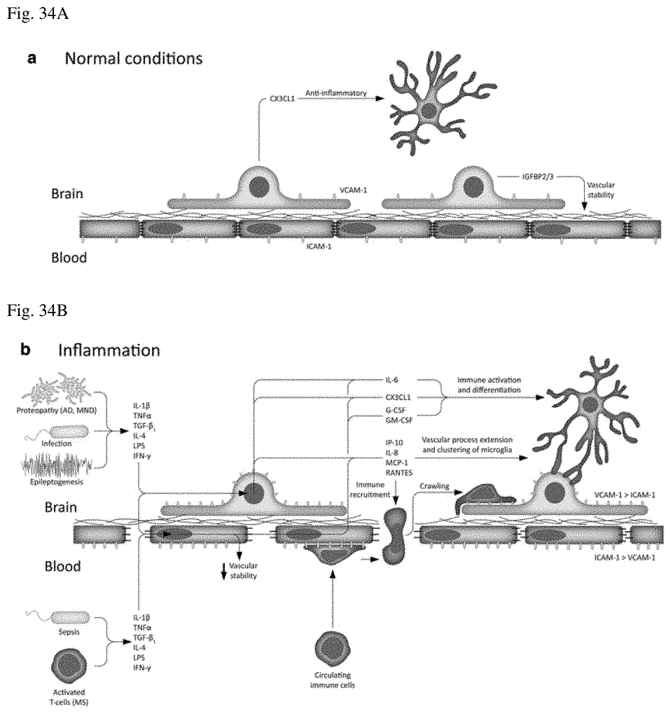

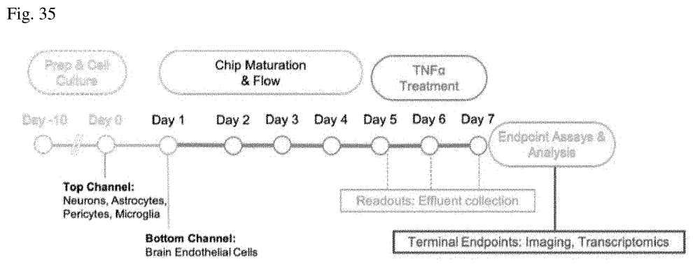

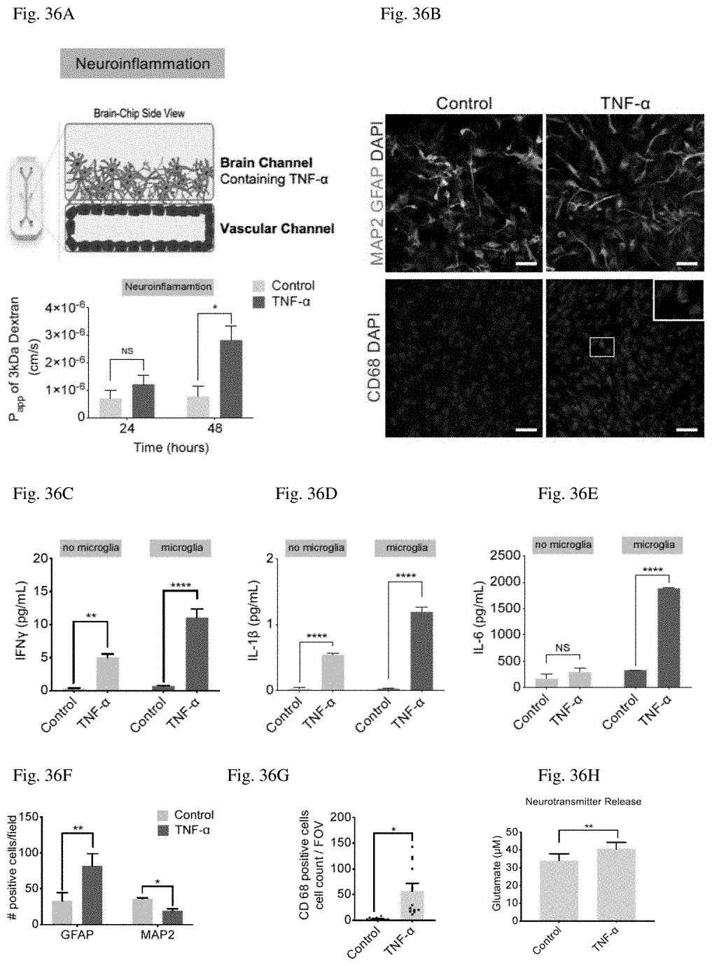

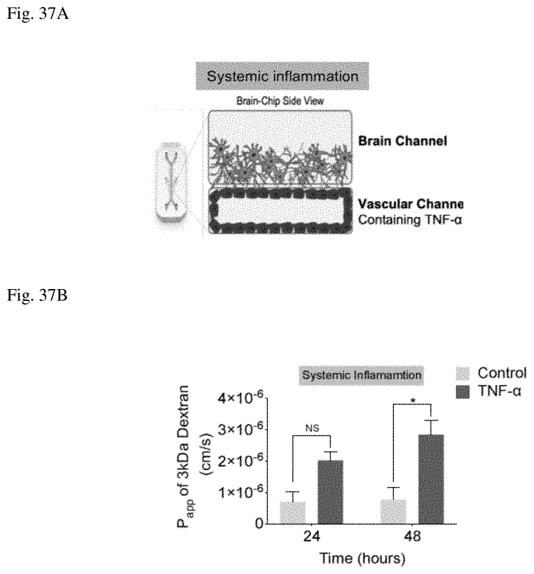

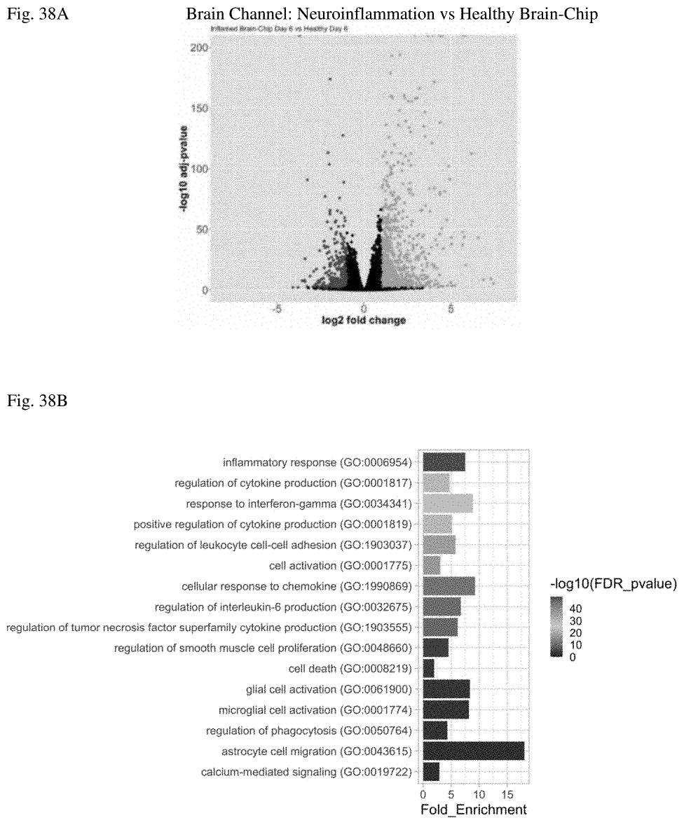

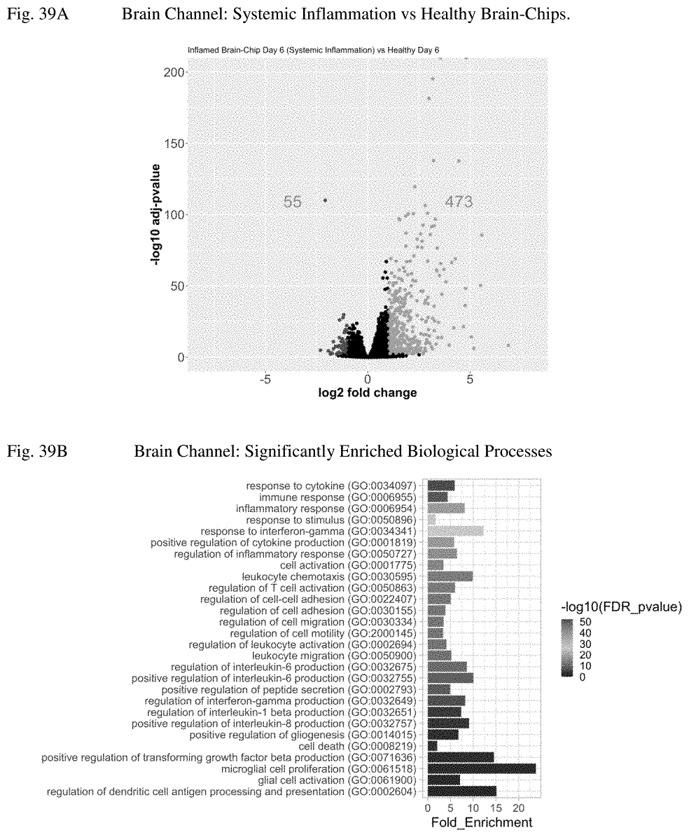

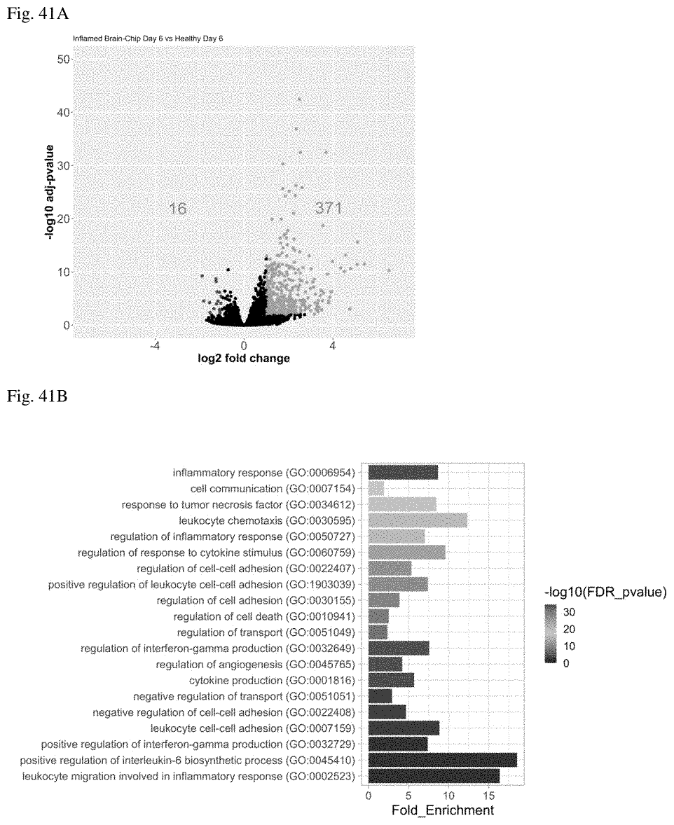

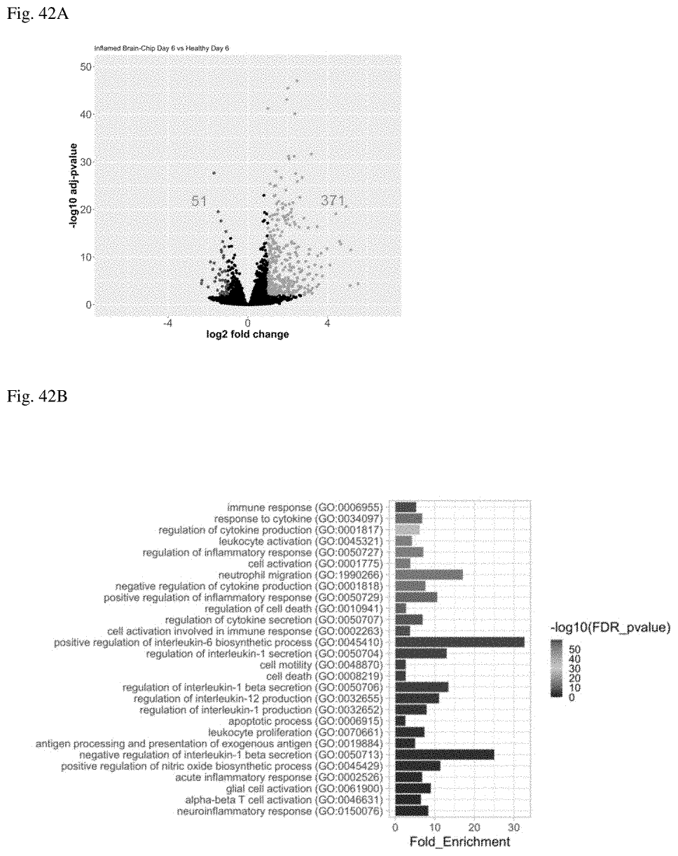

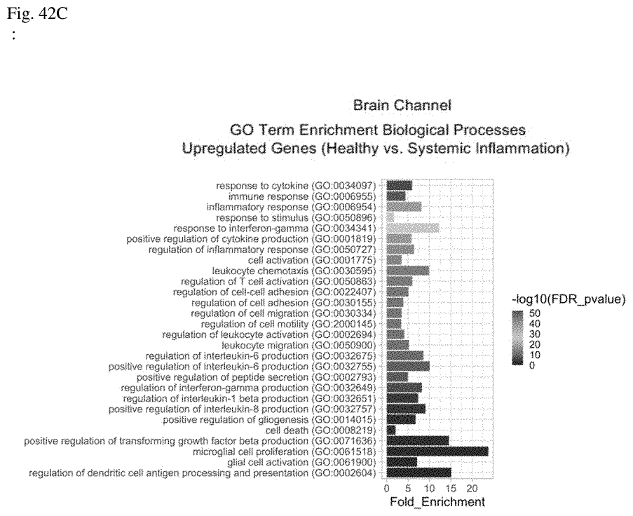

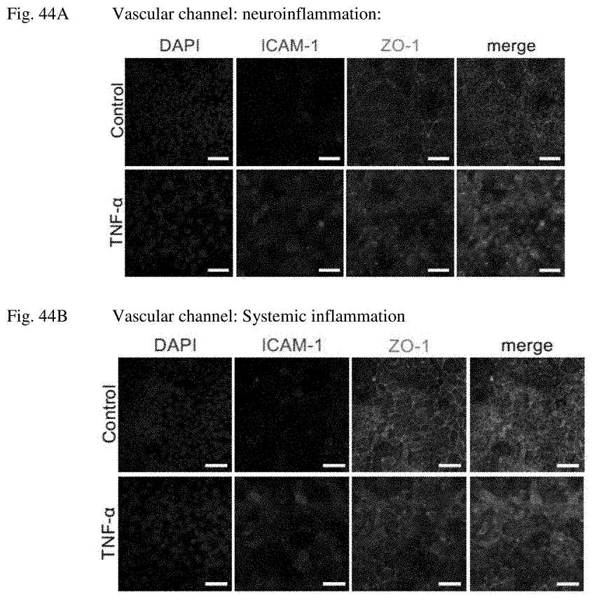

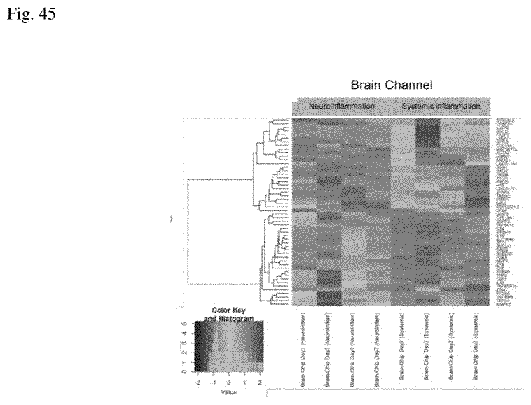

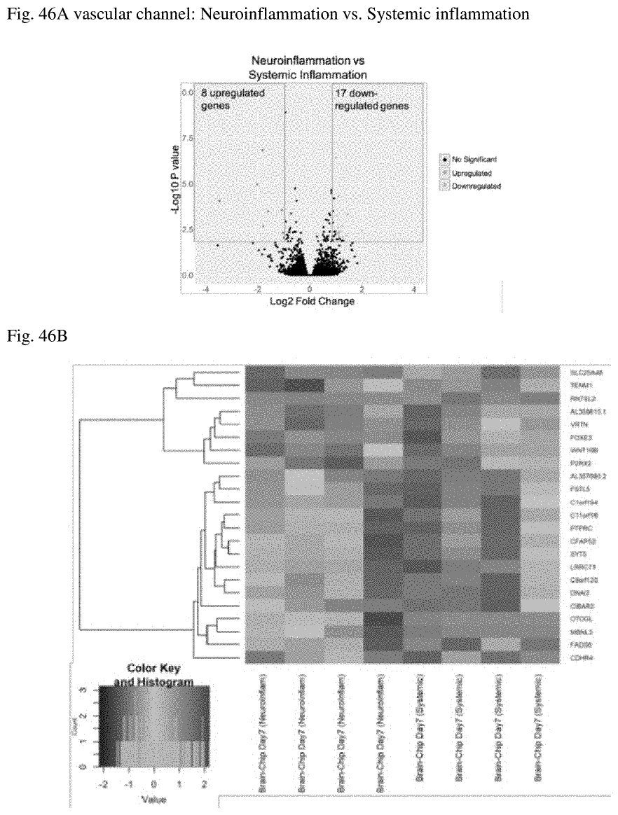

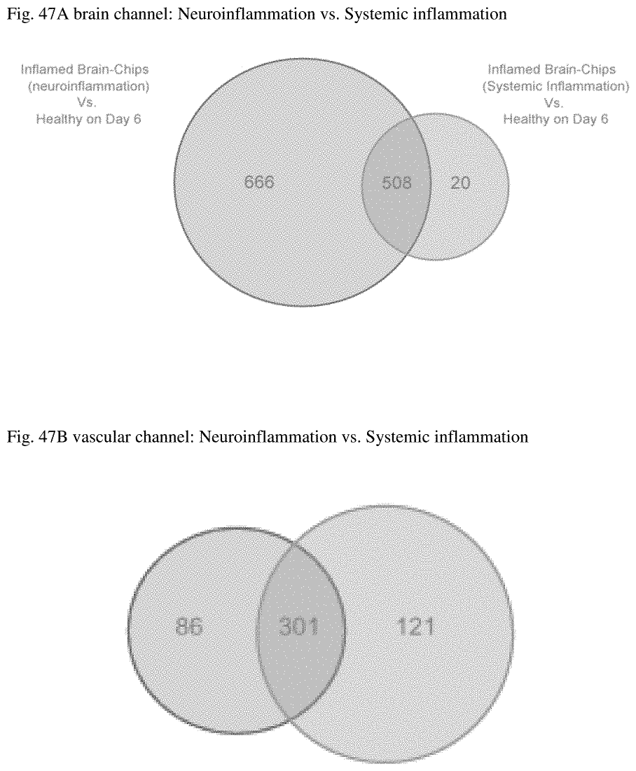

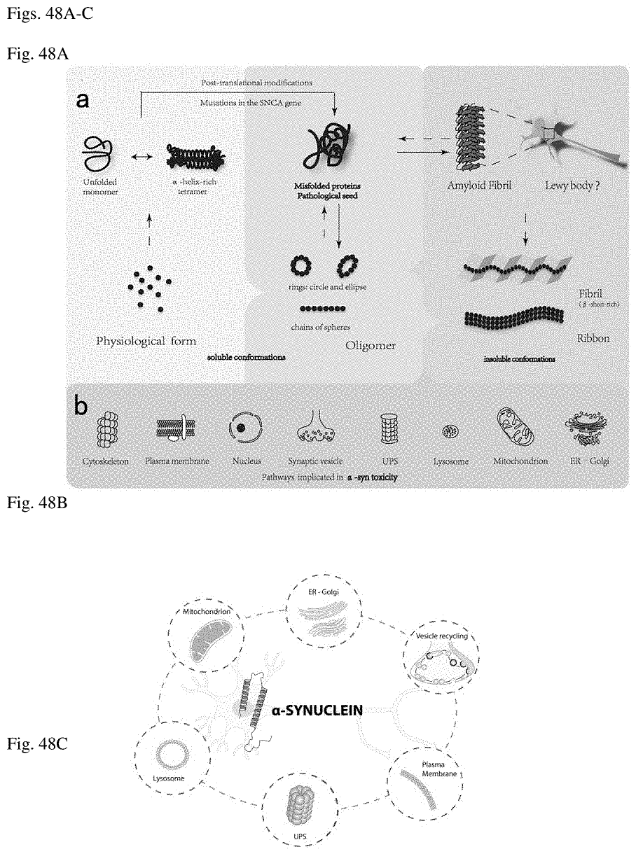

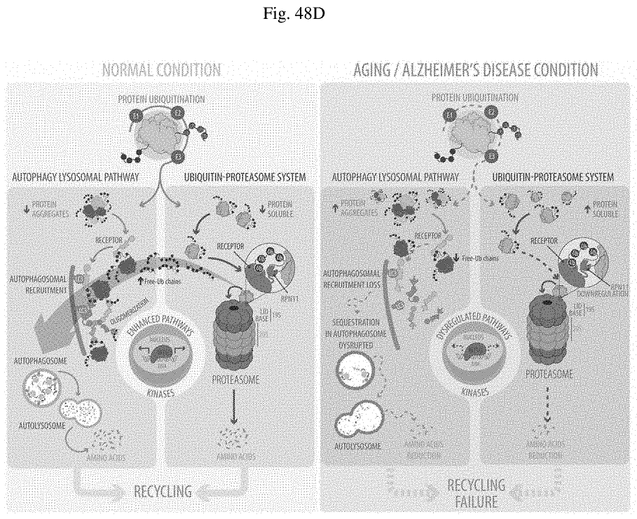

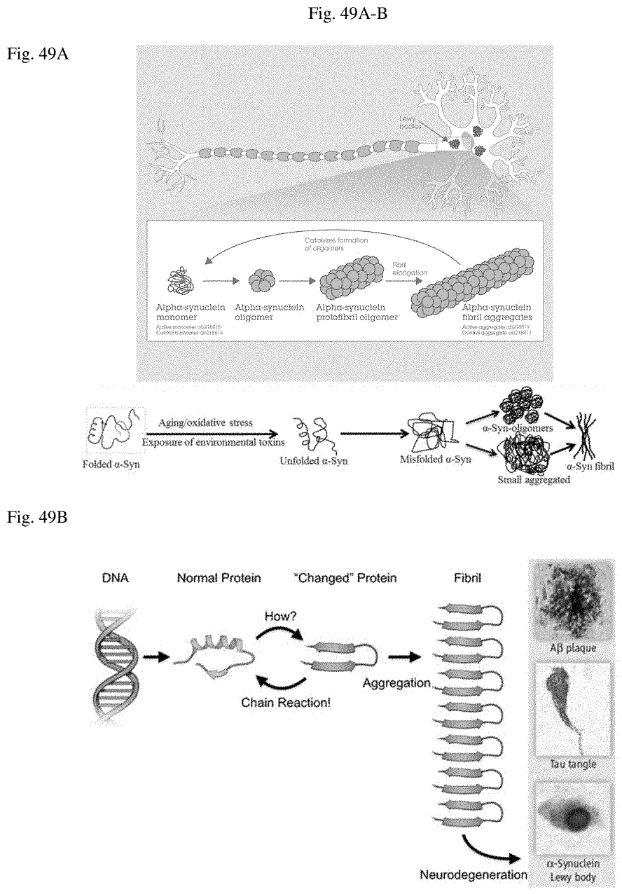

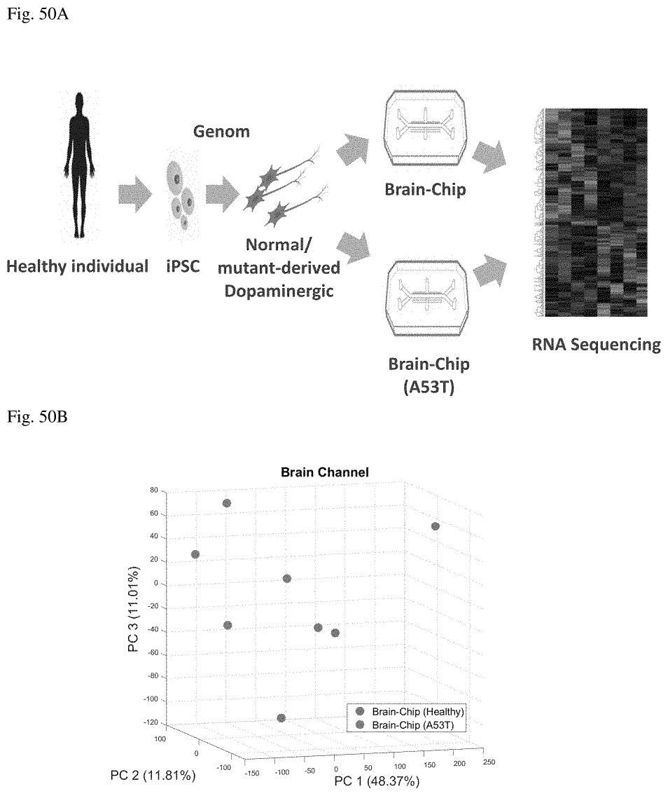

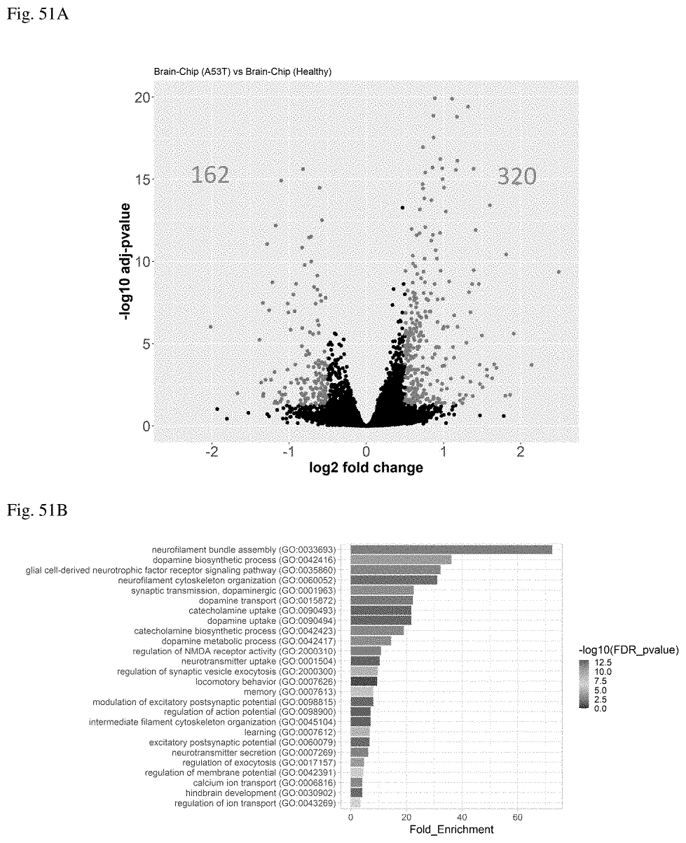

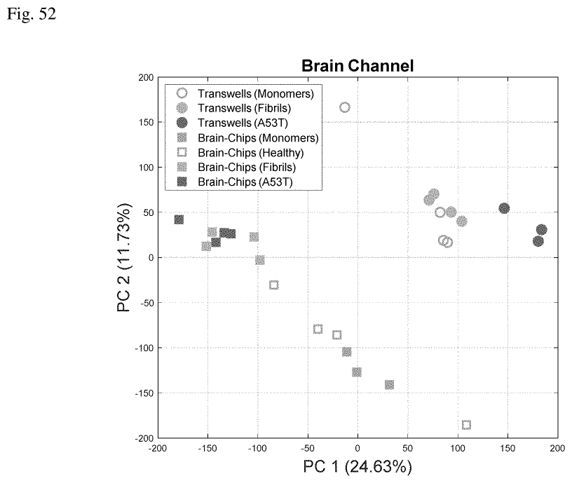

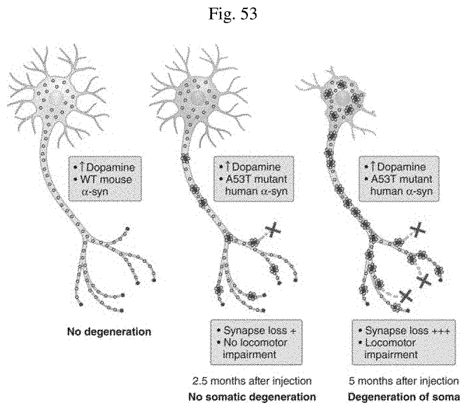

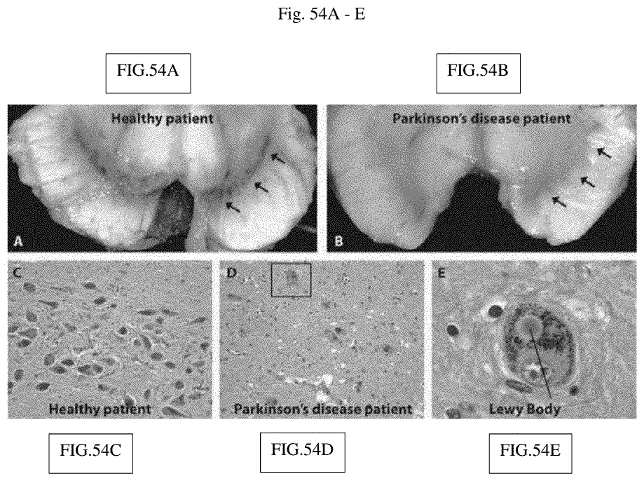

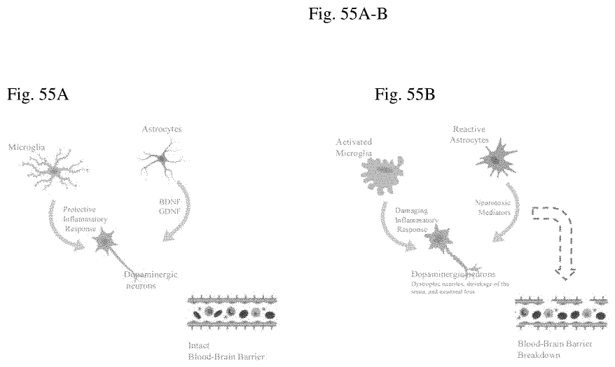

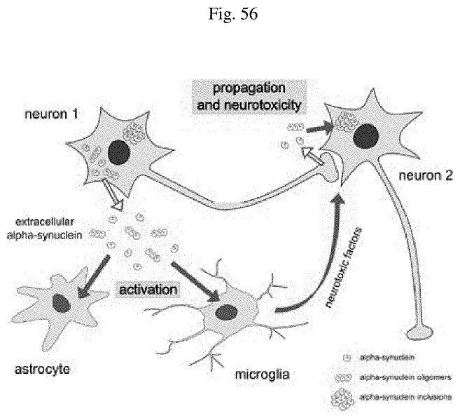

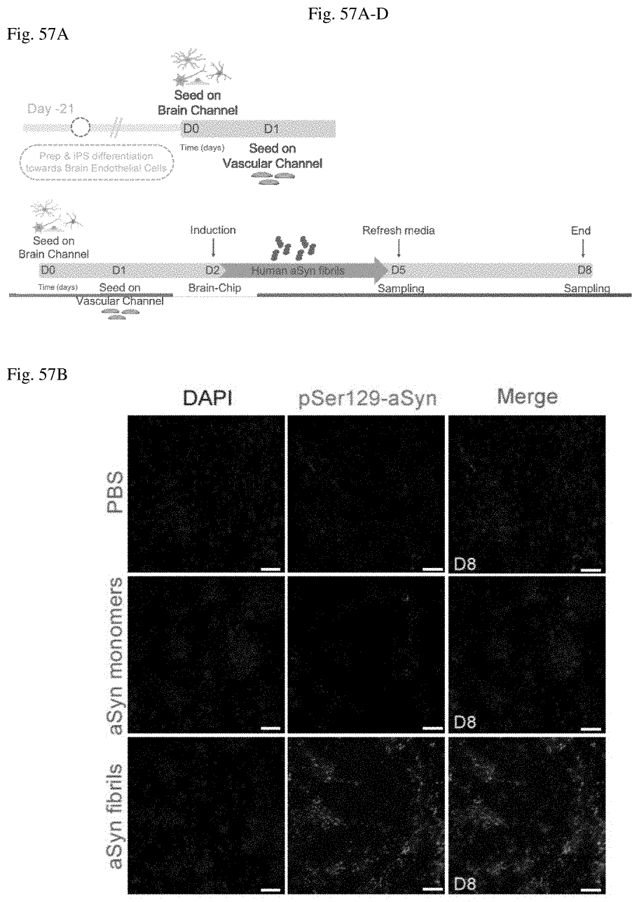

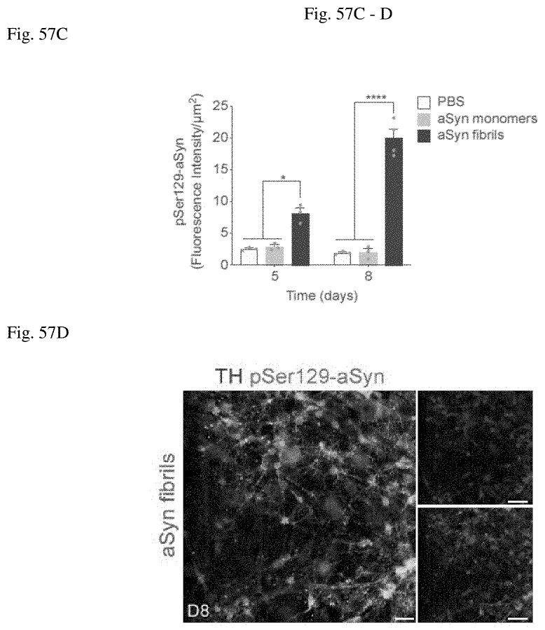

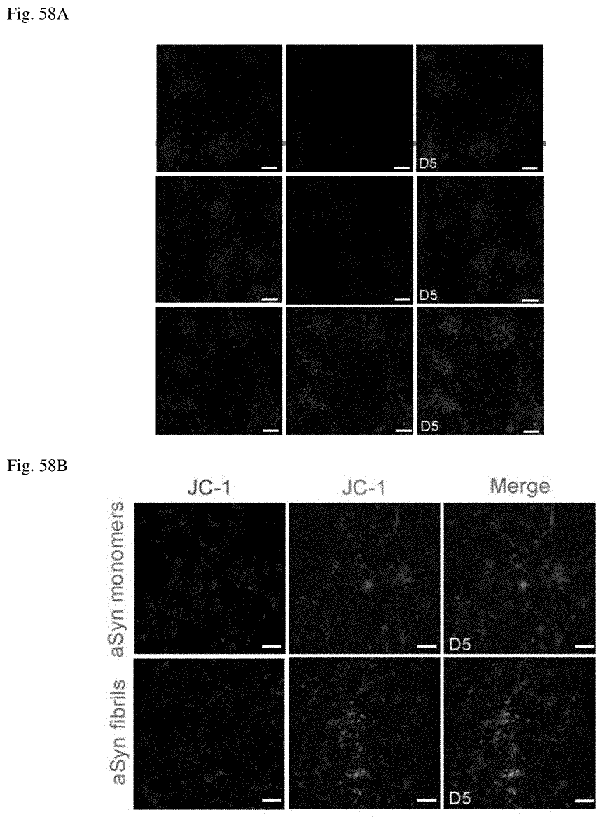

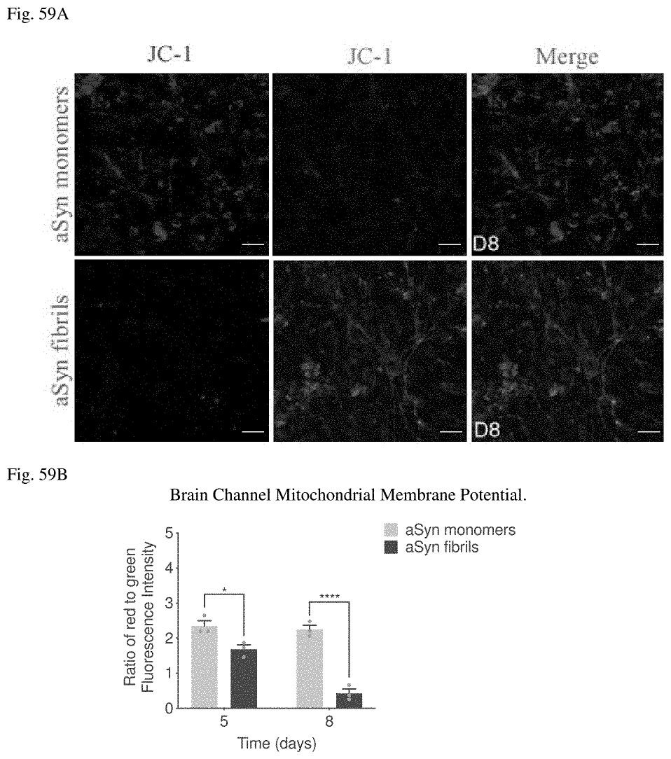

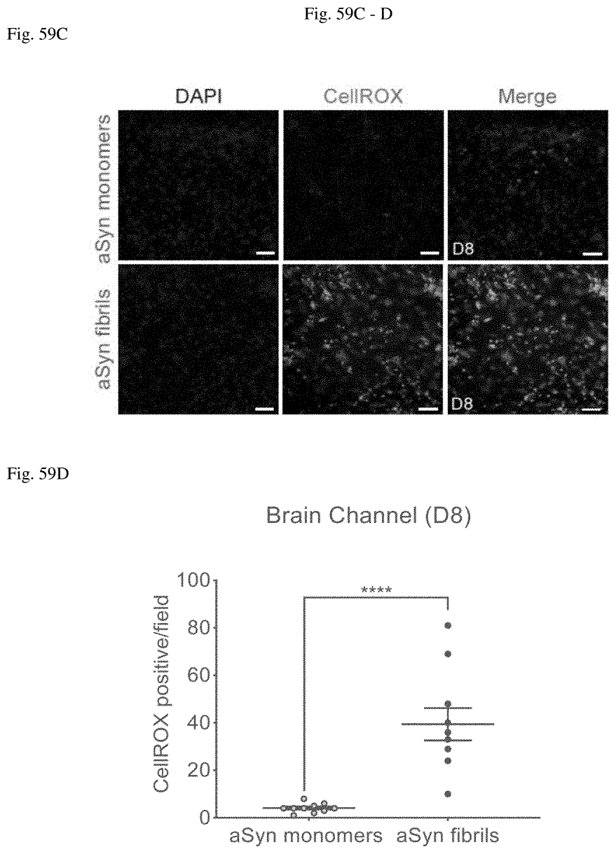

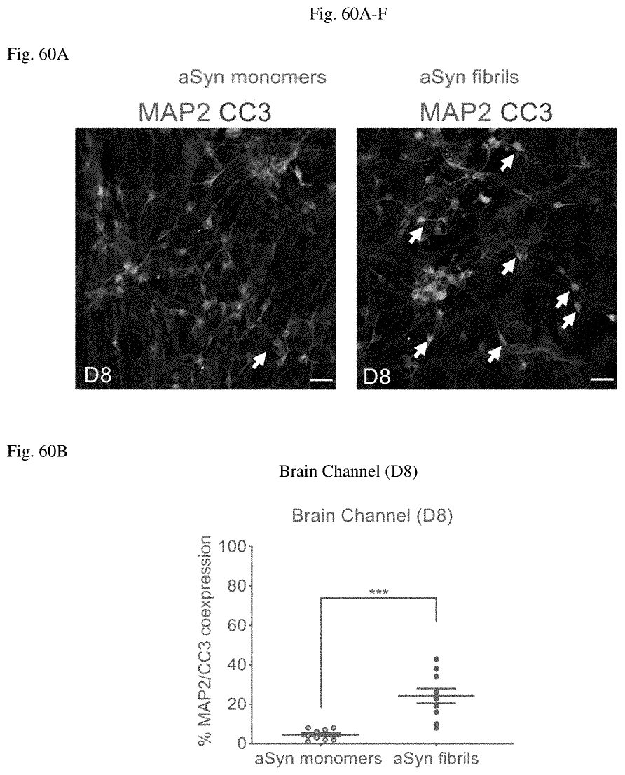

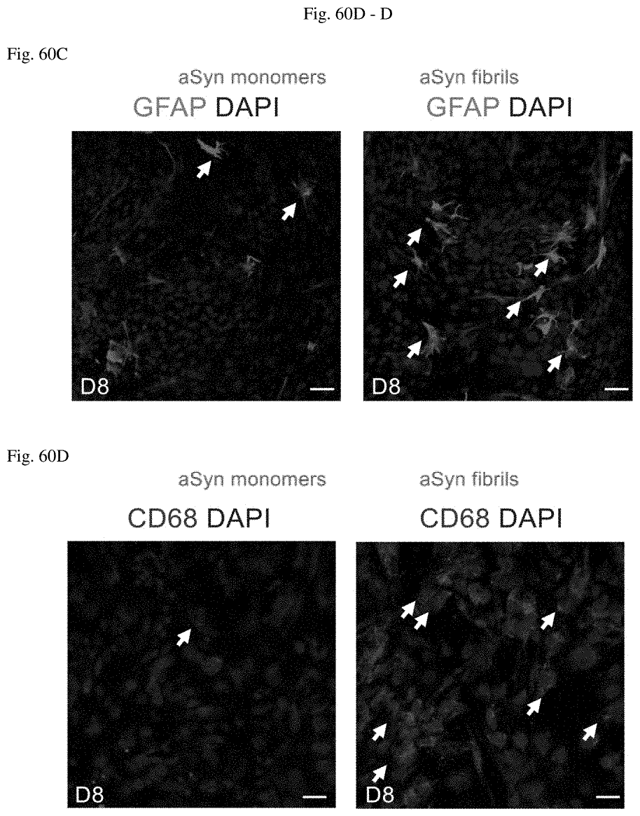

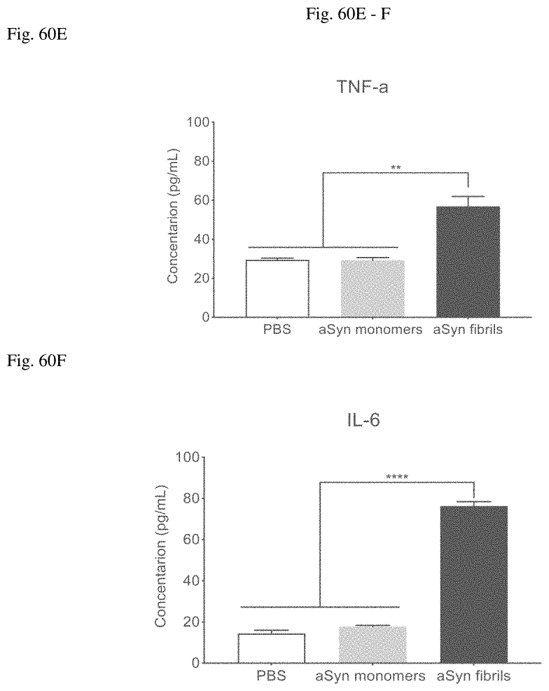

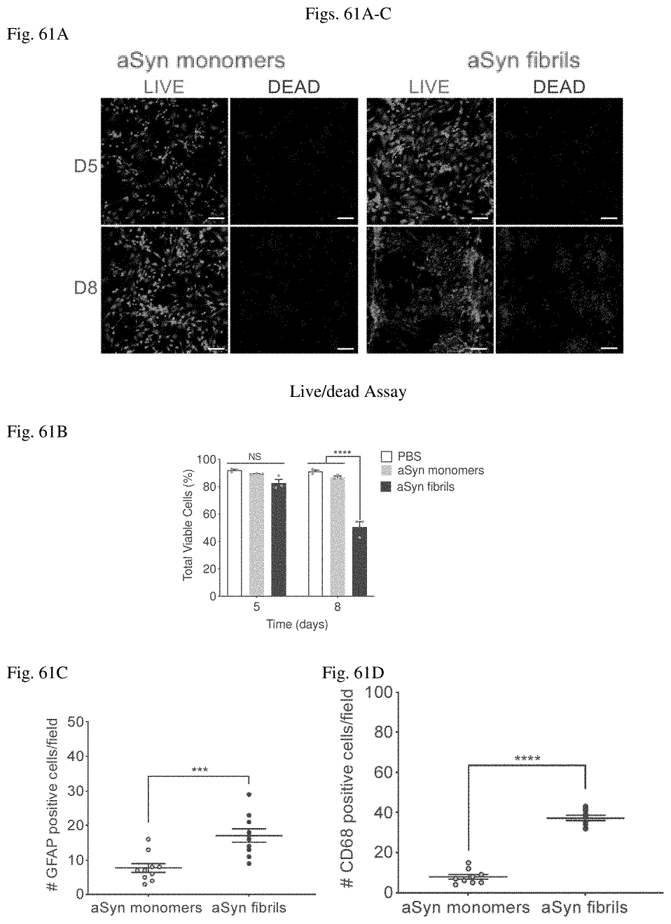

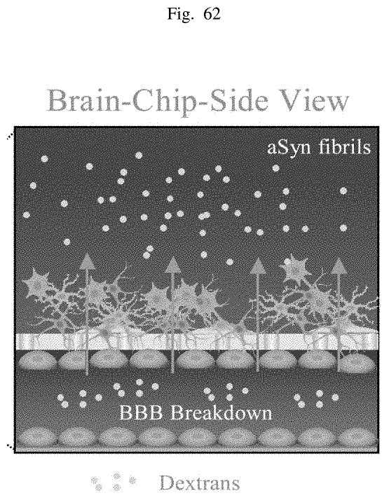

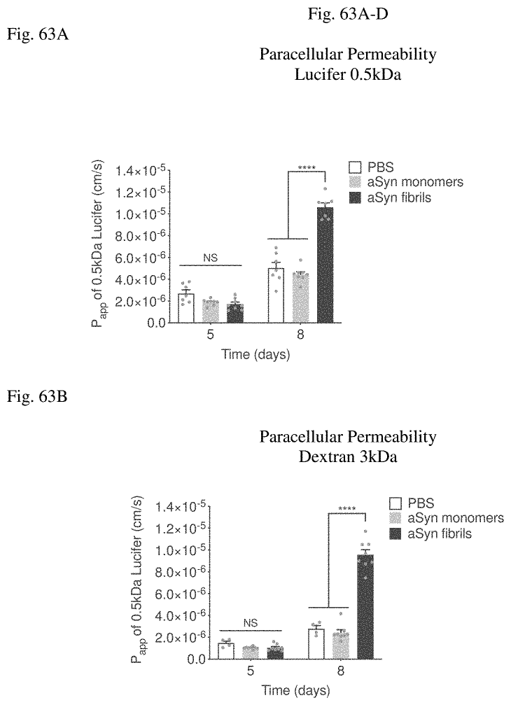

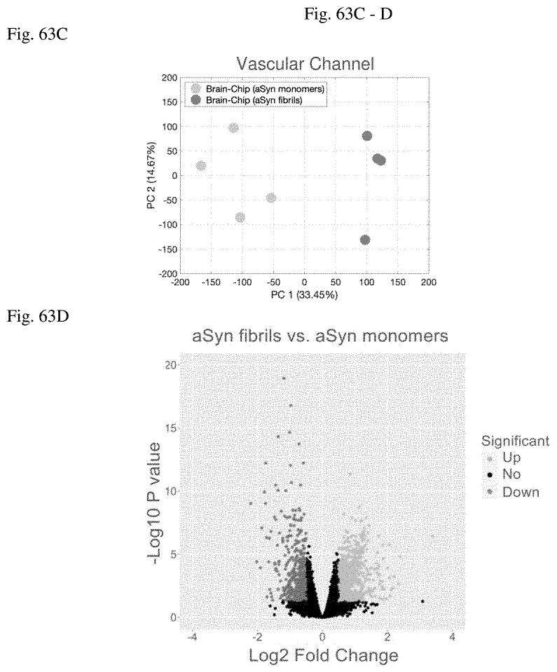

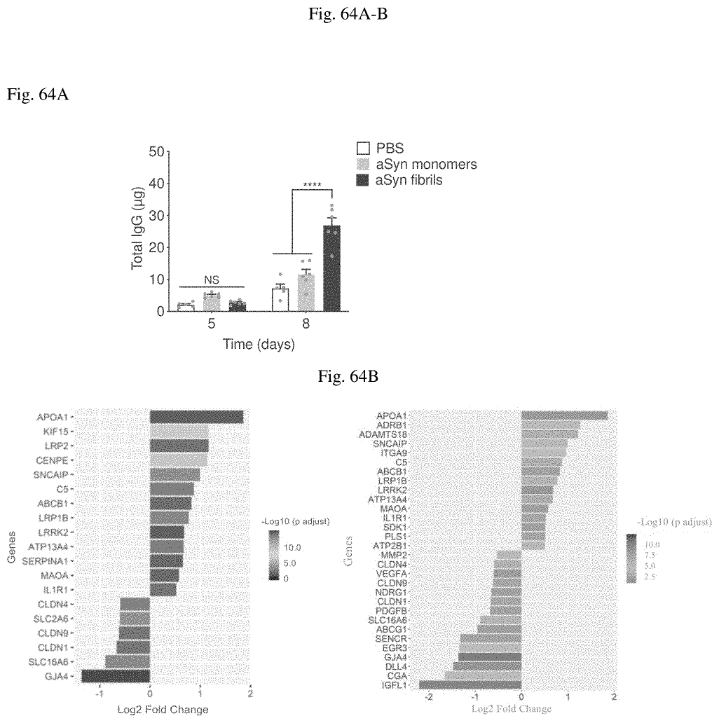

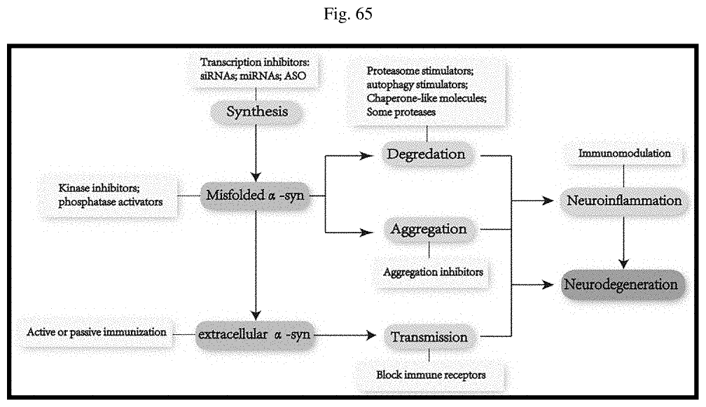

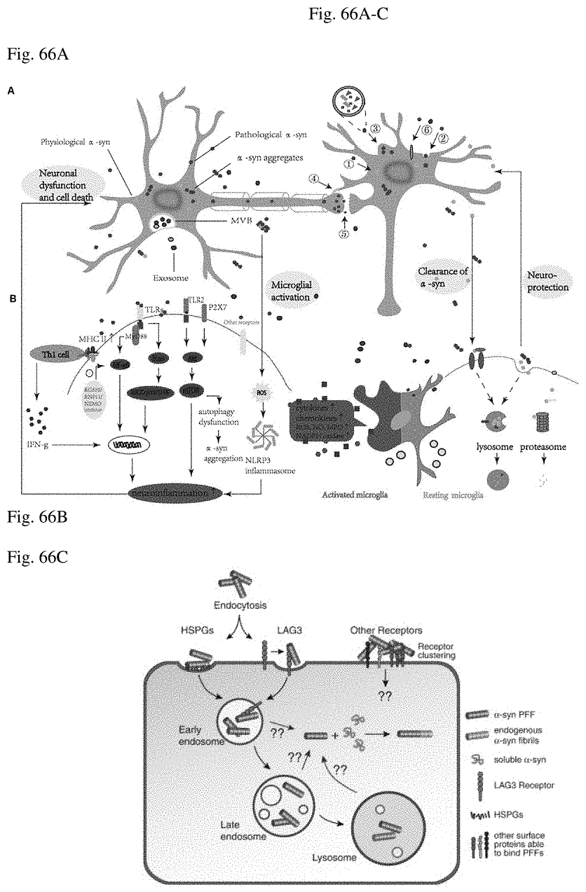

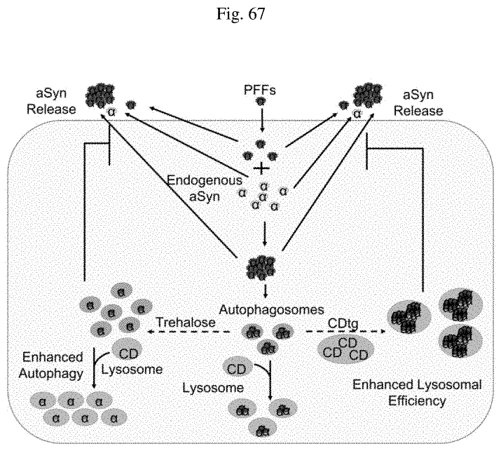

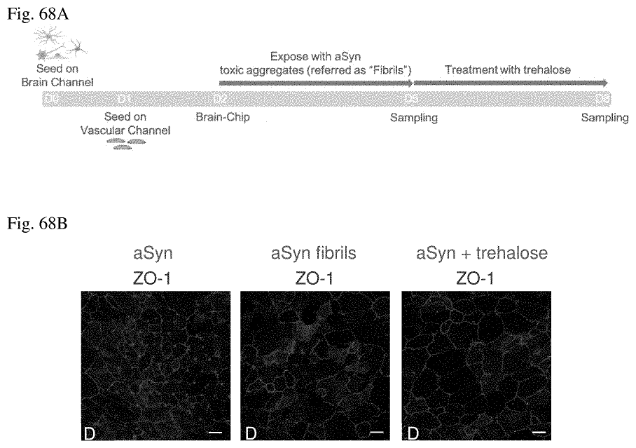

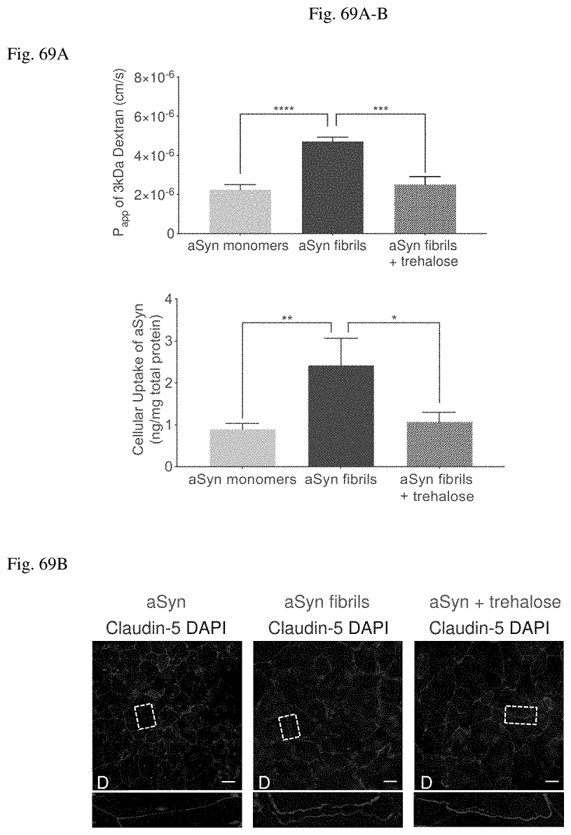

Confocal immunofluorescence microscopic analysis after 7 days of microfluidic culture showed specific expression of microtubule-associated protein 2 (MAP2) for neurons, glial fibrillary acidic protein (GFAP) for astrocytes, and neuron-glial antigen 2 (NG2) for pericytes (FIGS. 1C, 2A and 2B). In addition, expression of the vesicular glutamate transport (VGLUT1) in neurons was verified (FIG. 31F), a component of glutamatergic neurons, and the sustained levels of glutamate release (FIG. 4), confirming the proper synaptic transmission during the culture period.

Once stable neuronal function within a Brain-Chip was achieved, the next challenge was to evaluate how effectively it generated tight junctions contributing to the Blood-Brain Barrier's selective permeability. The expression of tight junctions of the brain endothelial monolayer was verified with immunofluorescent staining for tight junction marker zona occludens-1 (ZO-1) (FIG. 31F). Notably, the human iPS-derived brain endothelial cells formed a continuous band of ZO-1-containing tight junctions lining the entire vascular channel (FIG. 2B). We also confirmed the expression of GLUT-1, the BBB glucose transporter (FIG. 31F), and the internalization of transferrin within the cytoplasm of iPS-derived brain endothelial cells (FIG. 5), both mechanisms of transport across the blood-brain barrier.

To further evaluate the BBB integrity, we measured the permeability of cascade-blue-labeled 3-kDa dextran. The apparent permeability (Papp) of the Brain-Chip lined by human iPS-derived brain endothelial cells generated from two healthy donors (Donor 1; RUCDR, Donor 2; iXcell) was as low as the values measured in vivo, and comparable to previous BBB studies. FIG. 1F.

FIGS. 1A-F shows exemplary reconstruction of one embodiment of a neurovascular unit in a microfluidic device.

FIG. 1A shows one embodiment of a tall two-channel microfluidic BBB chip in vitro: 1. Upper neuronal channel, comprising human iPS-derived neuronal cells co-cultured with 2. Pericytes and 3. Astrocytes; 4. Optional vacuum chambers for providing membrane stretch. 5. Porous Membrane. 6. Endothelial cells. 7. Vascular channel. In one embodiment, microfluidic devices (chips) are seeded with induced pluripotent stem cells (iPSC)-derived cortical neurons, (Glutamatergic and GABAergic neurons), human primary astrocytes and pericytes in the neuronal channel (top), and iPSC-derived human brain microvascular endothelial cells in the vascular channel (bottom).

FIG. 1B upper florescent micrographs show exemplary PDGR-beta (red) stained pericytes and GFAP expressing astrocytes cultured on chip.

FIG. 1C florescent micrographs show exemplary immunocytochemical analysis of hiPSC-derived neuronal cultures in direct contact with astrocytes and pericytes after seven days. Specific markers were used to discriminate neurons (MAP2) and astrocytes (GFAP) from pericytes (NG2). Blue represents Hoechst-stained nuclei.

FIG. 1D right, shows exemplary quantitative barrier function analysis via permeability to 3 KDa fluorescent dextran, crossing through the vascular to the neuronal channel. Results are mean±s.e.m. *P<0.05. n=3. Scale bar: 100 μm.

FIG. 1E shows exemplary representative merged confocal image of the vascular channel stained for tight junction protein marker (ZO-1, green) and Glucose transporter (GLUT1, red) on day 7 in culture (bar, 100 μm).

FIG. 1F shows exemplary quantitative barrier function analysis via permeability of 3 kDa fluorescent dextran, for two independent iPSC donors crossing through the vascular into the neuronal channel on day 5, 6 and 7 in culture (n=6-9 independent chips, NS, not significant). Data are mean±S.E.M. Statistical analysis was by Student's t-test.

In addition, applications of specific human cell sources have enhanced the physiological relevance of in vitro models to the unique properties of the BBB endothelium.

FIGS. 2A and 2B show exemplary confocal images of the brain and vascular channels. FIG. 2A upper sets of panels show images of the entire length of an upper channel showing the organization of cell types and coverage across the entire channel on day 7 in culture. Immunofluorescence staining of the brain channel includes MAP2 (green), GFAP (magenta), NG2 (red) and DAPI (blue). Representative merged confocal image of the brain channel stained for iPS-derived cortical neurons (MAP2, green), astrocytes (GFAP, magenta) and pericytes (NG2, red) on day 7 in culture (bar, 50 μm).

FIG. 2B lower sets of panels show images of Brain Endothelial and Tight junction marker staining for morphological characterization from the vascular channel at 7 days in culture. Lower image shows immunofluorescence staining of a vascular channel stained for tight junction protein marker ZO-1 on day 7 in culture (bar, 50 μm).

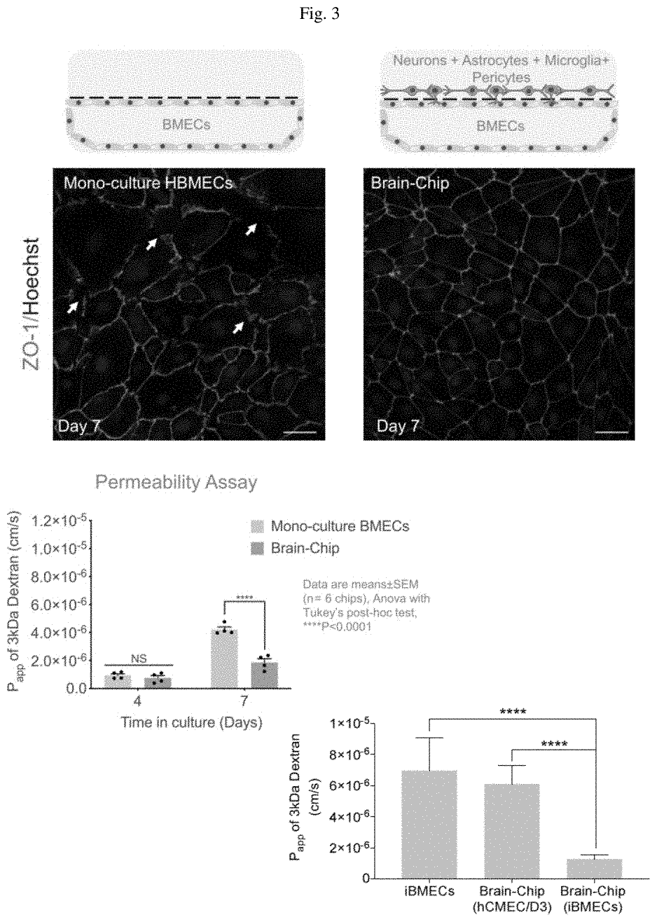

Thus, brain-specific endothelial cells as described herein, when cultured with the other neurovascular unit cells (i.e., astrocytes, microglia, neurons, and pericytes), exhibited barrier function with lower permeability than chips that include either monoculture of brain endothelial cells or co-culture with other sources of endothelial cells (hDMEC/D3, HBMEC) (FIG. 3). Taken together, these findings demonstrate that embodiments of microfluidic Brain-Chip recreates a microenvironment capable of supporting multiple cell types of the neurovascular unit, maintaining BBB functional integrity.

FIG. 3 shows exemplary schematics, florescent micrographs and charts demonstrating embodiments of a Human BBB chip as a mono-culture of HBMECs (left) and one embodiment of a Human Brain chip comprising neurons, astrocytes, microglia, pericytes and BMECs (right) for immunohistochemical (IHC) analysis. A lower left chart shows permeability assay results demonstrating that a Brain chip maintains a tighter barrier function by day 7 of co-culture over a monoculture of BMECs. A lower right chart shows a comparative assessment of the permeability of three different models (embodiments) on day 7 in culture, including a mono-culture of iPS-derived microvascular endothelial cells (iBMECs), a Brain-Chip cultured using the hCMEC/D3 endothelial cell line (Brain-Chip hCMEC/D3), and a Brain-Chip using iPS-derived microvascular endothelial cell (Brain-Chip iBMECs).

In one embodiment, an exemplary tall channel microfluidic BBB chip further comprises microglia cells. In one embodiment, an exemplary tall channel microfluidic Brain chip further comprises brain neurons. In one embodiment, an exemplary tall channel microfluidic Brain chip further comprises brain cortex neurons. In one embodiment, an exemplary tall channel microfluidic Brain chip further comprises dopaminergic neurons. Thus in some embodiments, an exemplary tall channel microfluidic Brain chip comprises a co-culture of pericytes, astrocytes, neurons, microglia and endothelial cells. In some embodiments, membrane stretch may be used to mimic movement of blood vessels within a living organism. In some embodiments, cells are cultured under flow for particular applications.

In some embodiments, one embodiment of a brain chip may be used to study the pathogenesis of Parkinson's Disease as it contains interacting: Human iPS-derived dopaminergic neurons; Human Primary Microglia; Human Primary Astrocytes and brain endothelial cells. Thus allowing the study of cross-talk between glial cells and neuronal cells, and thus address current hypotheses the field by investigating and dissecting roles for each specific cell type in the pathogenesis of PD. The system described herein, can provide new targets for developing cell and gene therapy approaches as it can provide a model for cellular interactions in a microfluidic device. Embodiments of Brain-Chips also includes brain endothelial cells recapitulating the neurovascular unit and enabling systemic delivery of potential therapeutic factors for in vivo relevant delivery, efficacy and safety, e.g. using Human iPS-derived Brain Endothelial cells.

This system will allow the study of: Blood-Brain Barrier pathology in PD. Alterations in tight junction, transport and endothelial cell surface proteins, and permeability; Spreading of alpha-synuclein (systemic exposure)-Blood to brain/brain to blood (addressing the “hot idea” in the field of peripheral involvement in neurodegenerative diseases pathogenesis and progress); Mechanism of transport of alpha-synuclein from other organs (e.g. intestine); and identify routes of delivery of therapeutic agents in neuronal diseases, e.g. PD.

### II. Brain Channel: Morphological Characterization: Cortical Neurons.

Cortical neurons, astrocytes and pericytes maintain their typical morphological characteristics over 7 days in culture. Human iPS-derived brain endothelial cells express tight junction proteins and brain endothelium-specific markers in the vascular channel of the chip. Brain endothelial cells successfully maintained at the vascular channel in the presence of fluid shear exhibited hallmark features of the human BBB, such as development of complex tight junctions and minimal barrier permeability.

FIG. 2A shows exemplary morphological characterization as florescent micrographs demonstrating immunohistochemical analysis of hPSC-derived neuronal cultures in direct contact with astrocytes, and pericytes in the brain channel. FIG. 2B Lower sets of panels show images of brain endothelial and tight junction marker staining for morphological characterization of iPSC derived endothelial cells in the vascular channel at 7 days in culture.

A. Impact of Supporting Cells on Barrier Formation.

iPSC-derived brain endothelium exhibits stable, long-term barrier function in the Brain-Chip alone or in the presence of brain cell types in the opposite channel having 4 cell types: neurons, astrocytes, microglia and pericytes. Surprisingly, the presence of the brain cells interacting with BMECs provided a less permeable, i.e., more normal BBB, than with BMECs alone.

FIG. 3 shows exemplary schematics, florescent micrographs and a chart demonstrating one embodiment of a Human BBB chip as a mono-culture of HBMECs (left) and one embodiment of a Human Brain chip comprising neurons, astrocytes, microgila, pericytes and BMECs (right) for immunocytochemical analysis. A chart shows permeability assay results demonstrating that a Brain chip maintains a tighter barrier function by day 7 of co-culture over a monoculture of BMECs.

B. Functionality of Cortical Neurons in the Brain-Chip.

Calcium imaging demonstrated that neurons consistently exhibited spontaneous neuronal activity, while daily secreted glutamate levels throughout the experiment confirmed proper synaptic activity.

FIG. 4 shows exemplary florescent micrographs demonstrating spontaneous calcium transients identified using fluorescence indicators (Fluo-4 AM) where neurons consistently exhibited spontaneous neuronal activity. Shades of green upper panels-heat mapped lower panels, while the charts show exemplary daily secreted neurotransmitter e.g. glutamate, levels throughout the experiment confirming synaptic activity of cells in a microfluidic chip.

Middle panels show representative time course images of Ca 2+ transients (pseudocolored red represents high levels of Ca 2+ fluorescence while blue represents low levels of Ca 2+ fluorescence). Scale bar: 50 μm. Lower left chart: Daily secreted levels of a neurotransmitter, e.g. glutamate, confirm the proper synaptic activity in the neuronal channel over time from 4-7 days in culture. Lower right chart: ELISA for glutamate secreted levels into the medium of the brain channel on day 5, 6, and 7 in culture (n=3 independent chips with duplicate technical replicates assayed per condition. Data are mean±S.E.M.

C. Transport Across the BBB.

iPSC-derived brain endothelial cells transport molecules across the blood brain barrier by receptor-mediated transcytosis. Representative merged confocal image of the vascular channel showing the positive staining of transferrin (transferrin conjugate, pHrodo, red) within the cell cytoplasm of iPS-derived brain endothelial cells (F-actin, green, DAPI, blue) (bar, 100 μm).

FIG. 5 shows an exemplary tanscytosis receptor as a transferrin receptor, as depicted by schematic diagrams and by flow cytometry analysis of cells tagged with an antibody recognizing a C-terminal region of transferrin receptor compared to cells tagged with a control isotype IgG.

D. Transferrin Receptor: Major Mechanism of Drug Delivery.

Intracellular localization of Transferrin confirms the functionality of the transferrin system on-chip. Proof-of-concept for transferrin receptor function in the Brain-Chip, by demonstrating active transport of a monoclonal antibody, utilizing the Transferrin Receptor-mediated mechanism.

FIG. 6 shows exemplary florescent micrographs demonstrating Immunocytochemistry of Phalloidin, pHrodoRed Transferrin, along with Hoechst staining of one embodiment of a Brain chip comprising iPS-derived HBMECs. Scale bar: 50 μm. Transcytosis of mAb IgG is demonstrated in an exemplary chart. Data are means±SEM (n=6 chips), t-test with Tukey's post-hoc test, **P<0.01, ***P<0.001.

E. ECM Analysis.

A comparison of commercial ECM to a sensory neuron ECM demonstrated that an ECM comprising a combination of Collagen IV, Fibronectin, Laminin enhanced development of and differentiation of iPSC progenitor sensory neuronal cells on chip. Thus, in one embodiment, a sensory neuron ECM consist of Collagen IV, Fibronectin, and Laminin. In one embodiment, a sensory neuron ECM consist of Collagen IV (400 μg/mL), Fibronectin (100 μg/mL), Laminin (20 μg/mL). One example of a commercially available ECM used for comparison (control) was SUREBond-XF, (ax0060) Axol. In one embodiment, sensory neurons are seeded onto ECM coated plates. In one embodiment, sensory neurons are seeded in ECM solutions onto plates. In one embodiment, sensory neurons are seeded into ECM coated channels of microfluidic devices. In one embodiment, sensory neurons are seeded in ECM solutions into channels of microfluidic devices. In one embodiment, sensory neurons are seeded in ECM solutions into ECM coated channels of microfluidic devices. In one embodiment, ECM solution is sensory neuron ECM. In one embodiment, ECM solution is laminin. In one embodiment, ECM solution is SUREBond. After seeding iPSC progenitor sensory neuronal cells undergo maturation and differentiation. After seeding iPSC progenitor sensory neuronal cells, cells are induced for undergoing maturation and differentiation. In one embodiment, maturation and differentiation is on plate. In one embodiment, maturation and differentiation is on chip. In one embodiment, microfluidic devices comprising iPSC sensory neuronal cells further comprise parenchymal cells for providing an innervated microfluidic device for use as described herein.

In one embodiment, immune cells are seeded into a microfluidic device. In one embodiment, parenchymal cells are seeded for overlaying immune cells. In one embodiment, immune cells are seeded into a microfluidic device for overlaying parenchymal cells. In one embodiment, iPSC sensory neuronal cells are seeded into a microfluidic device for overlaying immune cells. In one embodiment, iPSC sensory neuronal cells are overlaid with parenchymal cells.

Chip ECM coating of combined collagen IV, fibronectin, laminin overnight at 4° C. supported seeding of iPSC-derived sensory neuron progenitors (Axol BioScience, ax0053) on the S-1 Chip. compared to the commercially supplied SUREBond-XF (Axol BioScience, ax0060) typically used for neuronal culture on plates.

FIG. 7 shows exemplary florescent micrographs of fluorescently labeled extracellular matrix proteins demonstrating ECM staining on an ECM coated membrane of a microfluidic chip under flow. It appears that SUREBond-XF detaches and flows away while sensory neuron, ECM1, shows a greater number of extracellular matrix proteins attached to the chip membrane than laminin alone, ECM2.

FIG. 8 shows an exemplary bright field image showing the results of direct Seeding on-Chip iPSC-derived Sensory Neuron Progenitors comparing two types of ECM. SUREBond-XF Control. Collagen IV (400 μg/mL); Fibronectin (100 μg/mL); Laminin (20 μg/mL).

F. Time Course of iPSC-Derived Sensory Neuron Differentiation and Maturation.

Chip sensory neuron ECM coating of Collagen IV, Fibronectin, Laminin overnight at 4° C. supported the differentiation and maturation of sensory neuron progenitors on the S-1 chip (Axol) both the day after seeding (upper panel) and on Day 7 where neuronal cells have spread out and extended dendrites. Axol Human iPSC-Sensory Neuron Progenitors refer to cells derived from integration-free iPSCs and have been differentiated to neurons using small molecules by Axol BioScience. Headquarters: Axol Bioscience Ltd., Science Village, Chesterford Research Park, Little Chesterford, Cambridge, United Kingdom, CB10 1XL. US Office: Ground Floor, Jean Mayer Administration Building, 201 Westboro Road, North Grafton, Mass. 01536. United States.

FIG. 9 shows an exemplary bright field image showing the results of direct Seeding on-Chip of iPSC-derived Sensory Neuron Progenitors comparing results of using sensory neuron ECM over time, the Day after seeding upper image and Day 7, lower image. A combination of Collagen IV (400 μg/mL); Fibronectin (100 μg/mL); Laminin (20 μg/mL) was used to coat the membrane prior to seeding cells. iPSC-derived sensory neuron progenitors are treated Day 5 with Mitomycin C to eliminate proliferating cells among the progenitor pool and maintain the population of terminally differentiated non-proliferating neurons.

G. Immunofluorescent Analysis of Sensory Neuron Progenitors (Axol).

Chip ECM coating of Collagen IV, Fibronectin, Laminin overnight at 4° C. supported the differentiation and maturation of Axol (Axol Bioscience, commercial source of integration-free iPSCs differentiated to neurons using small molecules) sensory neuron progenitors on the S-1 chip confirmed by mature sensory neuron and nociception specific markers MAP2, TRPV1, and Nav1.7.

FIG. 10 shows an exemplary sensory neuron ECM coating of Collagen IV, Fibronectin, Laminin overnight at 4° C. that supported the differentiation and maturation of sensory neuron progenitors (Axol Bioscience) on a tall channel (S-1) chip confirmed by mature sensory neuron and nociception specific markers MAP2 (green), TRPV1 (red-left panel), and Nav1.7 (red-right panel). Merge shows superimposed images from the column of panels above the merged image. Co-merged staining is orange and yellow. Day 10, 5 days of flow.

H. ECM Characterization and Immune Cells Embedded within the Chip.

Optimization of the extracellular matrix (ECM) layer established within the chip was assessed by immunofluorescent staining. Optimized ECM conditions were then used for embedding immune cell types within the chip for further experiments. Optimized real-time, imaging based methods was used to assess immune cell proliferation and viability.

I. Microfluidic Channels were Coated with Sensory Neuron ECM Prior to Seeding.

As one embodiment of an Innervated Intestine-Chip as described herein, resident immune cells and sensory neurons are incorporated in a gel in between the intestinal epithelial cells and the chip membrane, to recapitulate intestinal lamina propria.

FIG. 11 shows exemplary florescent micrographs of ECM proteins colored as magenta and green fluorescently labeled macrophages and T-cells on chips as one embodiment of a contemplated Live-Image Tile of Innervated Intestine-Chip. Merged image shows both ECM, macrophages (MO) and T-cells.

Macrophage subtypes are involved in different immune responses. Merely as example, M1 macrophages produce characteristic cytokines when stimulated while M2 subtypes produce other subtypes for helping coordinate the immune response to parasites and fungal infections.

FIG. 12 shows exemplary results using one embodiment of an Intestine-Chip. Upper left panel is a representative image of labeled immune cells on and within the epithelial layer of a Caco-2 Intestine-Chip. Middle panel is a representative FACs forward vs side scatter plot of differentiated lymphocytes. Right panel is a FACs histogram showing the expression of macrophage marker CD86 after differentiation of lymphocytes. These were immune cells that were incorporated on the chip. Lower panes show immune cell counts from chips co-cultured for 7 and 14 days. Identical rounds of macrophage differentiation from PBMC donors 1&2 Co-stimulation with anti-human CD3 & anti-human CD28.

As demonstrated herein, embodiments of a human Brain-Chip were used to model inflammation. In part, human brain chips were used because species differences in brain function and blood-brain barrier (BBB) often preclude accurate extrapolation from animal models to human patients. Thus, there is an unmet need for human relevant systems that can recreate aspects of brain physiology and pathophysiology of common diseases, in particular for neurodegenerative diseases and changes associated with aging. Data shown herein, demonstrates that embodiments of a human Brain-Chip exhibits physiologically relevant levels of human BBB function for at least seven days in vitro, including low barrier permeability and expression of tight junction proteins. Furthermore, RNA sequencing expression profiles showed closer relevance to the human cortex tissue than in conventional cell cultures. The subsequent addition of TNF-α significantly increased the number of GFAP-positive astrocytes (GFAP positive) and microglial activation (CD68 positive) while inducing the release of proinflammatory cytokines (IFNγ, IL-β, and IL-6) after two days of exposure. The paracellular permeability of the barrier was increased in the TNF-α induced model, and it was accompanied by decreased expression of tight junction protein, ZO-1, as assessed by immunofluorescence. Experiments with transcriptomics were used for comparison for a more comprehensive characterization of the molecular mechanisms and pathways activated by this model. In summary, current findings demonstrate the development of a multicellular Brain-Chip support the development of models for the study of neuroinflammation and other altered BBB permeability disorders. The use of microfluidic brain chips enables studies on mechanistic aspects of neural pathology and disease progression which may be used for drug discovery and therapeutic tests.

Neurodegenerative diseases represent a significant proportion of diseases burden and affect up to one billion people globally. Inflammatory responses in the brain have been found to induce the pathogenesis of multiple diseases such as Alzheimer's disease (AD), Parkinson's disease (PD), and Multiple sclerosis (MS). Thus, pathways of inflammation have been the aim of novel therapeutic targets in such diseases. Although there is evidence that inflammation is associated with structural and functional alterations of the neurovascular unit (NVU), often early in the disease course, several questions remain to be addressed, and it is unclear if the NVU pathology is a cause or consequence of these disorders. Even after significant advancements on the study of such pathologies, there is still no treatment that can cure degenerative diseases, one reason being a lack of knowledge about the cerebral development, function and disease states is still very poor due to the lack of access to the human brain. Animal studies have contributed to current knowledge on brain function; however, they fail to recapitulate human brain cellular interactions due to species differences.

The introduction of more realistic multicellular in vitro models of the NVU, such as the organ-on-chip technology for the Blood-Brain Barrier (BBB) (Adriani et al., 2017) and cerebrovascular organoids derived from human induced pluripotent stem cells (iPSCs) (Appelt-Menzel et al., 2017), provides the opportunity to explore the molecular interactions among NVU cells beyond what can be achieved with conventional co-culture systems.

Recently, complex Brain/BBB-Chips have been constructed; however, they are not brain region-specific. In addition, most models lack a microglia component, the resident immune cells in the brain. To address the limitations of the classic BBB models (transwells) and extend the capabilities of the microfluidic ones, we have developed a human Brain-Chip that supports growth and development of human iPS-derived cortical neurons, human primary astrocytes, microglia, pericytes, and human iPS-derived microvascular endothelial cells, all of which are involved in the proper formation of the BBB. The Brain-Chip sustained low barrier permeability levels similar to those observed in the human brain for seven days in vitro. By leveraging next-generation sequencing data and information retrieved from well-curated databases providing signature gene sets characteristic for the human cortex, we were able to show that the Brain-Chip exhibits higher transcriptomic similarity to the adult cortical tissue than the conventional culture systems with cellular constituents of the neurovascular unit.

To aid further understanding of how the Brain-Chip responds to inflammation, a typical NVU pathophysiology feature, we used tumor necrosis factor-alpha (TNF-α). Elevated levels of TNF-α have been linked to a wide variety of diseases, including traumatic brain injury, ischemia, AD, PD, MS, and amyotrophic lateral sclerosis (ALS). TNF-α has also been reported to cause increased blood-brain barrier permeability, participating in cerebral edema. Moreover, systemic inflammation can result in neuroinflammation, mainly exhibited as microglial activation, production of inflammatory molecules, and recruitment of peripheral immune cells in the brain, thus shaping a cerebral inflammatory milieu that may seriously impact neuronal function. However, the communication between peripheral inflammatory factors, brain inflammatory factors, and NVU cells is not yet clearly understood. Thus, targeting systemic and neuroinflammatory pathways for treating brain disorders is a high priority for neuropharmacological drug development.

A compartmentalized culture platform using fluid flow allows spatiotemporally controlled microenvironments for monitoring several types of inflammatory responses of endothelial cells and brain parenchymal cells. As shown herein, microfluidic Brain chips successfully reproduced several neurovascular unit responses to TNF-α, such as astrogliosis, microglial activation, elevated pro-inflammatory cytokine release, neuronal death, and disruption of the BBB. Further, experiments with transcriptomics provided a more comprehensive characterization of model inflammatory diseases associated molecular mechanisms for several types of inflammatory pathways.

Overall, the use of a microfluidic Brain-Chip as described herein, will enable bridging the gap between animal and human models. In part, this will be achieved by assisting with understanding how various assaults and manipulations can perturb the function of the NVU, e.g. for testing therapeutics.

### III. TNF-Alpha Induced Inflammation for Use in Modeling Neuroinflammation and Systemic Inflammation.

TNF-alpha is a potent pro-inflammatory cytokine implicated in neurodegenerative diseases that is used for modeling inflammatory diseases. However, because conventional neuronal cultures spontaneously degenerate over time, actual inflammatory specific damage is not accurately modeled. However, in one embodiment, as described herein, TNFa treated microfluidic Brain Chips successfully demonstrated a human relevant inflammatory response of co-cultures in a Brain-Chip, including astrogliosis and neuronal death. Moreover, TNFa treated Brain chips, in addition to inducing permeability, altered ZO-1, GFAP expression as well as inducing neuronal death up to 24 hours after stimulation. Such markers can be used as a baseline inflammatory state which will aid in the study and comparison between types of anti-inflammatory or other types of drug treatments.

FIG. 13 shows exemplary florescent images of one embodiment of a Brain-Chip exposed to TNF-alpha (100 ng/ml) (right panel where arrows point to cell membranes lacking ZO-1 attachments) via the neuronal channel. TNF-alpha treatment also significantly increases GFAP expression as well as neuronal death up to 24 hours after stimulation. Scale bar: 50 μm. The chart demonstrates TNF-alpha induced increase in permeability by an increase in 3 kDa Dextran diffusion from the lower to upper channel. Data are means±SEM (n=6 chips), t-test with Tukey's post-hoc test, **P<0.01.

This study uses one embodiment of a Brain chip to identify cell secretions (e.g. cytokines) and expression (e.g. RNA), but it also speaks to the communication between different cells. For example, one of the main findings described herein involves the unexpected discovery that the endothelial cells behave differently in response to neuroinflammation in the brain compartment of the chip vs. when endothelial cells are inflamed by circulating inflammatory compounds. Thus, results shown herein provide a clear demonstration that neuroinflammation responses are different than systemic responses, albeit some overlapping characteristics. Moreover, microglia cells were discovered to contribute to neuroinflammation responses in microfluidic Brain Chips.

FIGS. 34A-B shows exemplary schematic diagrams demonstrating contemplated embodiments of drug targets and biomarkers for normal (noninflammatory) and disease associated inflammatory conditions.

FIG. 34 shows exemplary systemic inflammation cause a breach in the blood brain-barrier (BBB) thereby allowing for the entry of immune/inflammatory cells and proinflammatory cytokines into the brain.

FIG. 34B shows exemplary neuroinflammation induces and accelerates pathogenesis of Parkinson's disease (PD), Alzheimer's disease (AD) and Multiple sclerosis (MS). Smyth et al., Journal of Neuroinflammation 2018.

A. Neuroinflammation.

Neuroinflammation is specifically implicated in PD, Alzheimer's, ALS, traumatic brain injury and a host of other diseases and conditions. In some embodiments, cellular secretions are contemplated for use as biomarkers (e.g. soluble markers released by the cells that would indicate the presence, extent or nature of the neuroinflammation). As diagrammed in FIGS. 33A-B, contemplated inflammatory inducing cytokines; proinflammatory cytokine responses, responses including immune cell recruitment; etc.; are shown. Further, diseases related to inflammation such as infections and Epileptogenesis (referring to the gradual process by which a normal brain develops epilepsy) may be modeled using brain chips and methods described herein. Epileptogenesis is associated with subtle neuronal damage, gliosis, and microgliosis, with an increased, strong, and persistent inflammatory state in the microenvironment of neural tissue.

FIG. 14 shows exemplary florescent images of one embodiment of a Brain-Chip exposed to TNF-alpha showing Neurons (MAP2-green), Astrocytes (GFAP-pink) and staining for Nuclei: Hoechst (blue). Scale bars: 50 micron. Chart demonstrates % of specific cell subtypes over total brain cells. Data are means±SEM, n=6 Chips, *P<0.05.

B. TNF-α-Mediated Neuroinflammation Responses in Embodiments of a Microfluidic Brain-Chip.

Neuroinflammation incorporates a broad spectrum of complex cellular responses that often contribute to the pathogenesis and progression of various neurological disorders. To study the effects of neuroinflammatory specific signals on the Brain-Chip, TNF-α was perfused within the brain channel at a pathophysiological concentration of 100 ng/mL (FIG. 36A and FIG. 35). TNF-α treatment significantly increased the proportion of the GFAP-positive cells and decreased the proportion of the MAP2-positive cells (FIG. 36B and FIG. 36F). As demonstrated by CD68 expression, activated microglia were also observed following two days of exposure with TNF-α (FIG. 36B and FIG. 36G). These observations are consistent with previous studies that show that glial cells such as microglia and astrocytes in the brain become reactive under pathological conditions. In addition, excessive TNF-α levels have been reported to have an inhibitory effect on glutamate transporters, resulting in increased glutamate concentration in the CNS parenchyma, linking the central inflammatory response to glutamate-mediated toxicity. To test this hypothesis in this system, secreted glutamate levels were measured by enzyme-linked immunosorbent assay (ELISA). Levels of glutamate detected were significantly higher in the media of the brain channel following two days of exposure with TNF-α compared to the untreated group (FIG. 36H), supporting findings that showed a decrease in the number of neurons upon TNF-α treatment, likely due to glutamate-mediated toxicity.

In one embodiment, a model of neuroinflammation was provided by treating cells as a neurovascular unit (brain side) with TNF-alpha added to the brain channel. TNFα, was previously reported to mediate neuroinflammation and detected in patients with neurological. disorders. On days 0 and 1, cells were seeded on the top and bottom channel of one embodiment of a Brain-Chip, respectively. The Brain-Chip is then allowed to mature under flow conditions for 4 more days and on day 5, cells on the brain side are treated with TNFα, for 48 hrs. During exposure, effluent is collected, and on the last day of the experiment, cells in chips may be imaged, stained for immunohistochemistry and imaged. In some embodiments, cells are lysed for transcriptomic analysis. Thus, modeling neuroinflammation comprises Brain chips and methods of use where TNF-a and/or other inflammatory compounds are added to the Brain channel. In some embodiments, such neuroinflamed brain chips are used in methods for developing treatments in addition for testing compounds for efficacy and safety.

FIG. 35 shows an exemplary schematic experimental timeline of one embodiment of a neuroinflammation culture model.

To determine whether the level of other inflammatory cytokines increases in this TNF-α model, levels of IFNγ, interleukin-1β (IL-1β) and IL-6, were measured following two days of exposure within the brain channel media. These cytokines are associated with the increase of BBB permeability. Levels of IFNγ, IL-1β, and IL-6 were significantly higher upon exposure to TNF-α compared to the untreated group (FIG. 36c, FIG. 36d, and FIG. 36e). Surprisingly, the levels of IL-6 were not stigmatically changed upon exposure to TNF-α in the absence of microglia in our culture system, indicating that IL-6 is a significant contributor to the microglia-induced exaggeration in neuroinflammation in our model (FIG. 36E).

We further investigated this inflammatory response of the Brain-Chip at a transcriptomic level. We collected RNA samples from the brain channel of both treated and untreated groups (n=4 per condition). We performed Differential Gene Expression analysis, where we identified 801 up-regulated genes (magenta dots)- and 373 down (cyan dots)-regulated genes in the treated Brain-Chip compared to the untreated samples after 48 hs of exposure to TNF-α (FIG. 38A). Next, functional enrichment analysis was performed utilizing the PANTHER classification system to highlight biological processes that are significantly enriched gene ontology (GO) terms within these gene sets. The majority of differentially expressed genes belonged to pathways related to inflammatory responses, such as cytokine production, astrocyte, and microglia activation, as well as cell death, confirming our functional findings described above (FIG. 38B). Neuroinflammation is a hallmark of several neurological disorders associated with cognitive loss. Activated microglia and secreted factors are mediators of neuroinflammation and may contribute to neuronal dysfunction. Notably, tumor necrosis factor-alpha (TNF-α) was shown to be markedly elevated in patients with Alzheimer's disease (Alsadany et al., 2013, Heneka and O'Banion, 2007, Liu et al., 2014), suggesting a central inflammatory state that intensifies the secretion of associated cytokines and the duration of the immune response, which together may impact neuronal health. TNF-α can directly impair neuronal function and suppress long-term hippocampal potentiation (LTP), a mechanism essential for memory storage and consolidation (Cunningham et al., 1996; Tancredi et al., 1992). Using our Brain-Chip, we were able to recapitulate central inflammation pathogenesis using TNF-α. We report that exposure of the brain channel of the Brain-Chip to TNF-α induces time-dependent changes in BBB function, glia and cytokine activation, as well as a global change of the transcriptomic profile on both the vascular and brain channels. This data shows that TNF-α induce multiple inflammation-related pathways associated with biological processes in brain parenchymal cells, including glial cell activation, microglial cell activation, and regulation of phagocytosis, astrocyte cell migration, cell death, calcium-mediated signaling, and regulation of IL-6 production. This is in line with the well-known effect of TNF-α on glia cell activation and neuronal death; pathological events applied to several neurodegenerative disorders.

Further, the initial response to TNF-α stimulation on the brain side of the Brain-Chip resulted in BBB leakiness, increased ICAM-1 expression, and junctional alterations. Increased ICAM-1 expression was shown to promote the recruitment of immune cells in the brain parenchyma, leading to an amplification of the initial innate immune response. A functional interpretation of these responses was provided by surveying the effects of TNF-α on predefined sets of genes from the Gene Ontology (GO) enrichment analysis. Several GO terms associated with inflammation, including inflammatory response, cytokine production, and leukocyte chemotaxis, were highly upregulated in endothelial cells following central inflammation.

C. Neuroinflammatory Stimulation Effects on Blood-Brain Barrier: Blood-Brain Barrier Disruption During Neuroinflammation.

Brain barriers are uniquely poised to communicate signals between the CNS and peripheral compartments. Cells of the BBB respond to signals that arise from the CNS (including brain) or blood (systemic) compartments, which may stimulate alterations in their barrier, transport, and secretory functions (Verma et al., 2006; Krasnow et al., 2017). However, the contribution of these interface functions to BBB pathology and the mechanisms involved in these processes are vastly unknown.

Emerging evidence suggests a complex pathological impact of TNF-α on BBB structure and function that involves both direct effects on the endothelial cells and indirect paracrine responses manifested by increased pro-inflammatory stimuli in the brain.

To demonstrate the Brain-Chip utility for probing the effect of neuroinflammation on the BBB, the brain channel was perfused with TNF-α at 100 ng/mL for up to two days. To directly measure how BBB permeability changed over time in response to TNFα, the transport of 3-kDa Cascade Blue dextran was evaluated across the BBB. Fluorescent dextran molecule was introduced in the vascular channel. After 24 hrs and 48 hrs of exposure, the amount of fluorescent dextran collected in the Brain-side was measured. Data showed significantly increased permeability to 3 kDa dextran in the brain channel of the Brain-Chip in time depended manner (FIG. 36A).

TNF-α provoked the loss of ZO-1 and caused an excessive increase in intercellular adhesion molecule 1 (ICAM-1), a hallmark of inflammation, which functions in promoting adhesion and transmigration of circulating leukocytes across the blood-brain barrier (FIG. 44A). BBB permeability assays were done on the Brain-Chip upon exposure to TNF-α compared to the untreated group.

To further characterize the endothelium in neuroinflammation in a Brain-Chip model and to determine whether the exposure to TNF-α leads to transcriptomic changes in these cells, RNA-Seq analysis was used. Differential Gene Expression analysis of cells harvested from the brain channel resulted in the identification of 1174 DE genes, either significantly up-regulated (801 genes) or down-regulated (373 genes) (FIG. 38A) in the TNF-α treated group compared to untreated Healthy Brain chips.

However, in the vascular channel, 16 genes were upregulated while 371 genes were downregulated. (FIG. 41A). Notably, biological processes enriched in this gene set were associated with BBB functions such as leukocyte chemotaxis, leukocyte migration involved in inflammatory response, regulation of interferon-gamma production, cytokine production, regulation of transport, regulation of cell adhesion, and regulation of angiogenesis (FIG. 41B). These data support the concept that neuroinflammation mediated by TNF-α increases BBB permeability, demonstrating that this model has a use as a platform to evaluate the destructive effects of various neuroinflammatory mediators on the BBB integrity.

Thus, one embodiment of a Brain-Chip supports the survival, function, and interaction of iPS-derived cortical neurons, human primary astrocytes, microglia and pericytes as well as BBB integrity for 7 days in culture. Using TNFα, a human relevant inflammatory response of one embodiment of a Brain-Chip successfully demonstrated release of major proinflammatory cytokines as well as significant damage of the endothelial tight monolayer resulting in the breakdown of the BBB.

FIG. 40 shows an exemplary chart of comparative apparent permeability measured after 24 hrs and 48 hrs of exposure to TNFα (100 ng/mL) vs. vehicle control, introduced on the brain side. Transport through the BBB was significantly increased by three times over the control. Data are means±SEM, *p<0.5 (n>4 chips).

FIG. 43A shows exemplary secretion of pro-inflammatory cytokines. One embodiment of a brain channel of a Brain-Chip was exposed to continuous flow of media containing 100 ng/mL TNFα for 48 hrs. The effluent was collected at the 48 h timepoint; cytokines were measured and analyzed using MSD human proinflammatory panel. Data are means±SEM, ***p<0.001, ****p<0.0001 (n>4 chips).

FIG. 44A shows exemplary representative immunofluorescent staining of endothelial and tight junction markers after 48 hrs of exposure to TNFα on the brain-side showing a decreased expression of tight junction protein, ZO-1, and increased expression of intercellular adhesion molecule-1 (ICAM-1).

D. Contribution of Microglia to Neuroinflammation.

Microglia Responses to Pro-Inflammatory Stimuli. TNF-α activates microglia as shown by phenotypic changes and induction of phagocytosis. Activated microglia is an emerging feature in the pathogenesis of neurodegenerative diseases.

Presence of microglia in the Brain-Chip induces significantly the TNFα-mediated cytokine response in a time-dependent manner.

FIG. 15 shows exemplary florescent images of one embodiment of a healthy Brain-Chip exposed to TNF-alpha (100 ng/ml) (left panel Microglia (Iba1)-red and Neurons (MAP-2)-green. Live Imaging (right panel) of an Inflamed Brain-Chip (right panel Microglia (Cell Tracker Red CMPTX dye) and green Fluorescent latex beads. Phagocytosis of beads are an indication of activation of glial cells.

TNF-alpha was used for testing two different embodiments of Brain chips, one embodiment having pericytes, astrocytes and neuronal cells without microglial cells and one embodiment with microglial cells, pericytes, astrocytes and neuronal cells. Both embodiments contained MBECs in the opposite channel. Although TNF-alpha induced significant IL-6 production in both embodiments, there was an unexpected significant increase in IL-6 secreted after both 24 and 48 hours in embodiments of Brain chips including microglial cells over those without.

FIG. 16 shows an exemplary comparison of IL-6 secretion in pg/ml from Brain-Chip (−Microglia) and Brain-Chip (+Microglia) at 24 and 48 hours. Data are means±SEM (n=6 chips), Anova with Tukey's post-hoc test, **P<0.01, ***P<0.001.

Additionally, TNF-alpha induced neuroinflammation in Brain chips comprising microglia demonstrated increased secretion of IFN-γ, IL-1β along with IL-6 in effluents sampled from the Brain channel. See, FIGS. 36C, 36D, and 36E which show exemplary results of TNF-alpha induced neuroinflammation by secreted levels of proinflammatory cytokines (IFN-γ, IL-1β and IL-6) in the healthy or 48 hr TNF-α treated Brain-Chips in the presence or absence of microglia (n=4˜7 independent chips, **p<0.01, ****p<0.0001, NS, not significant). Data are mean±S.E.M. Statistical analysis was by Student's t-test.

E. TNF-α-Mediated Systemic Inflammation Responses in Embodiments of a Microfluidic Brain-Chip.

In one embodiment, a model of systemic inflammation was provided by treating cells with TNF-alpha added to the vascular channel. Thus, modeling systemic inflammation effects on brain cells comprises Brain chips and methods of use where TNF-a and/or other inflammatory compounds are added to the vascular channel. In some embodiments, such systemic inflamed brain chips are used in methods for developing treatments in addition for testing compounds for efficacy and safety.

To evaluate whether systemic inflammation had any effect on the BBB, TNF-α was perfused within the vascular channel at a pathophysiological concentration of 100 ng/mL (FIG. 37A) Immunofluorescence analysis showed that expression of BBB tight junction protein ZO-1 was significantly attenuated, while the expression of ICAM-1 was increased in TNF-α treated group compared to the control (FIG. 44B). Moreover, data showed significantly increased permeability to 3 kDa dextran with heightened systemic inflammation (FIG. 37B). These findings were complemented by RNA-Seq, indicating inflammation-associated pathways such as leukocyte proliferation and activation, neutrophil migration, regulation of cytokine secretion, as well as nitric oxide biosynthetic process, and cell death to be significantly upregulated in the endothelium (FIG. 42B).

FIG. 37A shows an exemplary schematic illustration of the Brain-Chip showing the TNF-α perfusion within the vascular channel as a systemic inflammation culture model. On days 0 and 1 cells were seeded on the top and bottom channel, respectively. The Brain-Chip is then allowed to mature under flow conditions for 4 more days and on day 5, cells on the vascular side are treated with TNFα, for 48 hrs. During exposure, effluent is collected, and on the last day of the experiment, cells in chips may be imaged, stained for immunohistochemistry and imaged. In some embodiments, cells are lysed for transcriptomic analysis.

FIG. 37B shows an exemplary Quantitative barrier function analysis via permeability to 3 kDa fluorescent dextran, over 48 hrs of treatment with TNF-α perfused at the vascular channel, showing time-depended BBB disruption (n=3-4 independent chips, *P<0.05, vehicle compared to TNF-α treated group after 48 hrs of treatment). Data are mean±S.E.M. Statistical analysis is two-way ANOVA with Tukey's multiple comparisons test.

F. Brain Channel Vs. Vascular Channel: Neuroinflammation Vs. Systemic Inflammation Using Healthy Brain Chips as a Baseline.

In some embodiments, neuroinflammation results were compared to untreated Healthy Brain Chips. In some embodiments, systemic inflammation results were compared to untreated Healthy Brain Chips.

FIGS. 38A-B shows exemplary Differential Gene Expression analysis for Neuroinflammation of the Brain channel: between TNFα exposed Brain-Chips and Healthy Brain-Chips on day 7 using a volcano plot of DGE Analysis (Neuroinflammation) in one embodiment of a Brain Channel. Differential Gene Expression Analysis was applied to RNA expression date (RNA Seq) between inflammed Brain-Chips vs Healthy Brain-Chips on Day 6. See Table 1.

FIGS. 39A-B shows exemplary Differential Gene Expression analysis for Systemic inflammation of the Brain channel: between TNFα exposed Brain-Chips and Healthy Brain-Chips on day 7 using a volcano plot of DGE Analysis (Neuroinflammation) in one embodiment of a Brain Channel. Differential Gene Expression Analysis was applied to RNA expression date (RNA Seq) between inflammed Brain-Chips vs Healthy Brain-Chips on Day 6. See Table 2.

1. Brain Channel: Neuroinflammation vs Healthy Brain-Chip. Neuroinflammed Brain-Chips vs Healthy on Day 6, 1174 genes were found significantly differentially expressed in cells within the Brain Channel, each contemplated for use as biomarkers, see Table 1 and FIG. 38A-B including GO gene categories.

TABLE 1
Neuroinflammation vs Healthy Brain-Chips.
Brain Channel: Neuroinflammation.

| Condition | # DE Genes | # up-regulated | # down-regulated |
|---|---|---|---|
| Neuroinflammation vs | 1174 | 801 | 373 |
| Healthy Brain-Chips | | | |

FIGS. 38A-B shows exemplary volcano plot of DGE Analysis (Neuroinflammation) in one embodiment of a Brain Channel. Differential Gene Expression Analysis was applied to RNA expression date (RNA Seq) between Brain channels of neuroinflammed Brain-Chips vs Healthy Brain-Chips on Day 6. See Table 1.

FIG. 38A shows an exemplary volcano plot illustrating the number of the differentially expressed (DE) genes (up-373 and down-801 regulated) and how they stratify based on their expression changes. Green dots: DE genes significantly up-regulated; Red dots: DE genes significantly down-regulated (adj.p-value<0.01 and |log 2FoldChange|>1); black dots: non-DE expressed genes. In total, 1174 genes were found significantly DE in cells of brain parenchyma, 801 up-regulated (in the inflamed Brain-Chips) and 373 down-regulated (in the inflamed Brain-Chips). FIG. 38B shows an exemplary Gene Ontology (GO) enrichment analysis based on the 801 up-regulated DE genes between the TNFαBrain-Chips and Healthy Brain Chips. Bar plot presents a subset of the significantly enriched biological processes identified by the enrichment analysis.

2. Brain Channel: Systemic Inflammation Vs Healthy Brain-Chips.

Differential Gene Expression Analysis of brain channel cells between systemically inflamed Brain-Chips vs Healthy on Day 6. 528 genes were found significantly differentially expressed in cells within the Brain Channel, each contemplated for use as biomarkers. DE genes selection criteria using DESeq2 R package: adj.pvalue<0.01; |log 2FoldChange|>=1. See Table 2.

TABLE 2
Brain Channel: Systemic Inflammation vs Healthy Brain-
Chips. Systemic Inflammation. 528 genes were found significantly
differentially expressed in cells within the Brain Channel,
each contemplated for use as biomarkers.

| Condition | # DE Genes | # up-regulated | # down-regulated |
|---|---|---|---|
| Systemic | 528 | 473 | 55 |
| Inflammation vs | | | |
| Healthy Brain-Chips | | | |

FIG. 39A shows an exemplary volcano plot illustrating the number of the differentially expressed (DE) genes (up473- and down-55 regulated) and how they stratify based on their expression changes. Green dots: DE genes significantly up-regulated; Red dots: DE genes significantly up- or down-regulated (adj.p-value<0.01 and |log 2FoldChange|>1); black dots: non-DE expressed genes. In total, 528 genes were found significantly DE in cells of brain parenchyma, 473 up-regulated (in the inflamed Brain-Chips) and 55 down-regulated (in the inflamed Brain-Chips). See Table 2.

Significantly Enriched GO terms from the list of the 473 DE up-regulated genes upon TNF-α exposure.

FIG. 39B shows an exemplary Gene Ontology (GO) enrichment analysis based on the 473 up-regulated DE genes between the TNFα Brain-Chips and Healthy Brain Chips. Bar plot presents a subset of the significantly enriched biological processes identified by the enrichment analysis.

3. Vascular Channel: Neuroinflammation vs Healthy Brain-Chip.

DGE Analysis (Neuroinflammation): 387 genes were found significantly regulated in endothelial cells.

Differential Gene Expression Analysis between Inflamed Brain-Chips vs Healthy on Day 6

DE genes selection criteria using DESeq2 R package: adj.pvalue<0.01; log 2FoldChange|>=1.

TABLE 3
Neuroinflammation vs Healthy Brain-Chips. Vascular
Channel: DGE Analysis (Neuroinflammation).

| Condition | # DE Genes | # up-regulated | # down-regulated |
|---|---|---|---|
| Neuroinflammation vs | 387 | 371 | 16 |
| Healthy Brain-Chips | | | |

FIGS. 41A-B Vascular Channel: DGE Analysis (Neuroinflammation). See Table 3.

FIG. 41A shows an exemplary volcano plot illustrating the number of the differentially expressed (DE) genes (up- and down-regulated) and how they stratify based on their expression changes. Red dots: DE genes significantly up- or down-regulated (adj.p-value<0.01 and |log 2FoldChange|>1); back dots: non-DE expressed genes. In total, 387 genes were found significantly DE in endothelial cells, 371 up-regulated (in the inflamed Brain-Chips) and 16 down-regulated (in the inflamed Brain-Chips). Significantly Enriched GO terms are provided from the list of the 387 DE up-regulated genes upon TNF-α exposure.

FIG. 41B shows an exemplary Gene Ontology (GO) enrichment analysis based on the 371 up-regulated DE genes between the TNFα treated Brain-Chips and Healthy Brain Chips. Bar plot presents a subset of the significantly enriched biological processes identified by the enrichment analysis.

4. Vascular Channel: Systemic Inflammation Vs. Healthy Chip.

Vascular Channel: Vascular Channel: DGE Analysis (Systemic Inflammation) 371 genes were found significantly regulated in endothelial cells.

Differential Gene Expression Analysis between Inflamed Brain-Chips vs Healthy on Day 6. DE genes selection criteria using DESeq2 R package: adj.pvalue<0.01; log 2FoldChange|>=1.

Vascular Channel: Significantly Enriched Biological Processes: Significantly Enriched GO terms from the list of the 371 DE up-regulated genes upon TNF-α exposure

TABLE 4
Neuroinflammation vs Healthy Brain-Chips. Vascular
Channel: DGE Analysis (Neuroinflammation).

| Condition | # DE Genes | # up-regulated | # down-regulated |
|---|---|---|---|
| Systemic | 422 | 371 | 51 |
| Inflammation vs | | | |
| Healthy Brain-Chips | | | |

Significantly Enriched GO terms from the list of the 371 DE up-regulated genes upon TNF-α exposure Vascular Channel: Neuroinflammation vs. Systemic Inflammation.

FIGS. 42A-B Vascular Channel: Systemic Inflammation vs. Healthy Chip shows exemplary Differential Gene Expression analysis between TNFα exposed Brain-Chips and Healthy Brain-Chips on day 6. See Table 4.

FIG. 42A Vascular Channel: Systemic Inflammation vs. Healthy Chip shows an exemplary volcano plot illustrating the number of the differentially expressed (DE) genes (up- and down-regulated) and how they stratify based on their expression changes. Red dots: DE genes significantly up- or down-regulated (adj.p-value<0.01 and |log 2FoldChange|>1); black dots: non-DE expressed genes. In total, 422 genes were found significantly DE in endothelial cells, 371 up-regulated (in the inflamed Brain-Chips) and 51 down-regulated (in the inflamed Brain-Chips).

FIG. 42B Vascular Channel: Systemic Inflammation vs. Healthy Chip shows an exemplary Gene Ontology (GO) enrichment analysis based on the 371 up-regulated DE genes between the TNFα Brain-Chips and Healthy Brain Chips. Bar plot presents a subset of the significantly enriched biological processes identified by the enrichment analysis.

FIG. 42C Brain Channel: Systemic Inflammation vs. Healthy Chip shows an exemplary Gene Ontology (GO) enrichment analysis based on the 371 up-regulated DE genes between the TNFα Brain-Chips and Healthy Brain Chips. Bar plot presents a subset of the significantly enriched biological processes identified by the enrichment analysis.

### IV. Similarities and Differences Between Neuroinflammation and Systemic Inflammation.

In one embodiment, comparisons were made between neuroinflammation vs. systemic inflammation and in the Brain-Chip which identified cues on similarities and differences. Thus, in some embodiments, in order to model neuroinflammation, TNFa was flowed into the brain channel of a Brain chip for inducing neuroinflammation. In some embodiments, in order to model systemic inflammation, TNFa was flowed into the vascular channel of a Brain chip for inducing systemic inflammation. Effluent and cells were sampled or visualized from both the upper brain channel and lower vascular channel for each type of inflammation. In some embodiments, neuroinflammation results were compared to Systemically inflamed chips. See sections below for comparative results.

In fact, bot neuronal inflammation and systemic inflammation cause morphological changes in the BBB of brain chips including increasing cell adhesion molecules for white bloods cells, e.g. ICAM-1. FIG. 44A shows exemplary immunofluorescence of ICAM-1 and ZO-1 on neuroinflammed endothelial cells neuroinflammation vs. FIG. 44B systemic inflammation.

Since systemic inflammation also compromises BBB integrity (FIG. 37A), it was contemplated that TNF-α perfused within the vascular channel might amplify inflammatory responses in the brain channel. To this end, levels of IFNγ, IL-1β, and IL-6 following two days of exposure within the brain channel media were measured after neuroinflammation and systemic inflammation. We observed significantly higher levels of brain cytokines IFNγ, IL-1β, and IL-6, as consequence of the systemic inflammation compared to the untreated group. FIG. 43B Brain channel: systemic inflammation. These findings in microfluidic Brain-chips support previous studies that showing TNF-α crosses the BBB and acts on microglia to induce brain inflammation, including the release of pro-inflammatory cytokines, thus augmenting inflammation processes in the CNS (Qin et al. 2007; Joshi et al., 201; Tangpong et al. 2006, 2007).

However, it was not known whether these observations would be mirrored on the transcriptional level so RNA seq comparisons were done. Surprisingly, the majority of differentially expressed genes belonged to pathways similar to neuroinflammatory state such as cytokine production, astrocyte, and microglia proliferation, as well as cell death (FIG. 41B).

Despite the overall similarities in neuroinflammation and systemic inflammation on morphology, we found differentially expressed genes between these two conditions on brain cells FIG. 45. This analysis in the brain channel resulted in the identification of 53 differentially expressed genes, either significantly up- (26 genes) or down-regulated (27 genes) (FIG. 45). Surprisingly, the glia-associated genes GFAP, XYLT1, H19, RGS4, TREM2, PADI2 and PADI4 were found to be increased in systemic inflammation. Given the importance of these genes in astrocytic scar formation (GFAP, XYLT1, H19, RGS4), microglia phagocytosis (TREM2), and destabilization of myelin (PADI2 and PADI4), it is plausible that their increased expression may underlie the lack of the ability of the CNS in self-repair and regeneration since the glial scar is considered the main hindrance to axonal regeneration and neuronal connectivity recovery, due to the production of growth-inhibitory components and the formation of physical and chemical barriers that hinder axon elongation.

Moreover, elevated levels of astrocyte intermediate filaments appear to be related to perturbances in the barrier function. On the other hand, regional applied stimuli (neuroinflammation) led to a strong induction of several pro-inflammatory cytokines and MMPs genes that can directly degrade endothelial tight junction-related proteins and ECM molecules, which promotes angiogenesis whereas simultaneously increases BBB permeability.

We applied a similar type of comparative analysis in the vascular channel after induced neurostimulation vs. systemic inflammation. This analysis resulted in the identification of 25 differentially expressed genes, either significantly up- (8 genes) or down-regulated (17 genes) (FIG. 46A). In contrast, all of the 25 genes with significant differential expression between the two states (neuroinflammation vs. systemic inflammation) showed not be involved in BBB permeability, cellular function, and endothelial junctions (FIG. 46B). Taken together, these findings indicate that systemic inflammation induces distinct changes in the brain; however, the contribution of these changes to the development of neurodegenerative disorders is still an open question.

In order to discover whether there was a difference between neuroinflammation vs. systemic inflammation, differential Gene Expression analysis and morphological comparisons were done comparing neuroinflammation vs. systemic inflammation for brain channel cells. An additional DE expression analysis was done comparing neuroinflammation vs. systemic inflammation for vascular cells. DE genes selection criteria using DESeq2 R package: adj.pvalue<0.01; |log 2FoldChange|>=1.

In other words, in some embodiments, at least one inflammatory inducing compound was added to either the brain channel or the vascular channel. Thus, these methods may be used for identification of potential drug targets for therapies against neuroinflammation and/or systemic inflammation.

FIG. 47A brain channel: Neuroinflammation vs. Systemic inflammation, each using healthy brain-chips as baseline, shows an exemplary Venn diagram demonstrating an overlap of 508 DE genes expressed in cells of the brain channel associated with inflammation. Inflamed Brain-Chips (Neuroinflammation) Vs. Healthy on Day 6 (blue). Inflamed Brain-Chips (Systemic Inflammation) Vs. Healthy on Day 6 (pink).

FIG. 47B vascular channel: Neuroinflammation vs. Systemic inflammation, each using healthy brain-chips as baseline, shows an exemplary Venn diagram demonstrating an overlap of 301 DE genes expressed in cells of the vascular channel. Inflamed Brain-Chips (Neuroinflammation) Vs. Healthy on Day 6 (blue). Inflamed Brain-Chips (Systemic Inflammation) Vs. Healthy on Day 6 (pink).

While it was shown that TNF-α influences the BBB function in several ways, few studies have considered its effects on the different sides of the barrier. Studies have shown that systemic inflammation exacerbates neuroinflammation and neurodegeneration in the brain (Perry, 2010; Villarán et al., 2010; Machado et al., 2011; Hernandez-Romero et al., 2012; Träger and Tabrizi, 2013). Systemically produced TNF-α can enter the circulation and cross the BBB through active transport or passively after pathologic BBB disruption. TNF-α was shown to affect synaptic transmission and synaptic scaling, as well as regulate the production of other cytokines and chemokines to impact neuronal function. However, current knowledge of the interactions between the peripheral immune and inflammatory components with the neuroinflammatory mechanism is still limited (Holmes and Butchart, 2011).

To study the effect of systemic inflammation in the 1 Brain-Chip, TNF-α was perfused through the vascular channel. BBB was characterized by reduced tight junction formation and increased membrane permeability. Using RNA seq analysis, investigation of transcriptomic changes due to central inflammation and systemic inflammation were evaluated in the Brain-Chip. Despite the overall similarities in BBB permeability changes in central inflammation and systemic inflammation, distinct transcriptomic signatures were identified between the two conditions, suggesting a different pattern of responses.

Despite the complexity of these expression profiles, each cell type responds in a different manner. Future studies should include investigation of single-cell transcriptomes underlying subtype specificity associated with inflammatory responses. Collectively these findings suggest a potential molecular mechanism by which inflammation originating in the periphery can induce transcriptional modulation in the brain. Moreover, understanding the control of microglial and astrocytic response during systemic inflammation opens new targets for glia modulation in neurodegenerative diseases. Therefore, developing methods to modulate these pathways to control the timing, spatial distribution, and amount of BBB dysfunction are contemplated to contribute to developing drug treatments and evaluating therapeutics, including for personalized medicine.

Overall, this platform could be useful in studying the roles of BBB in various diseases and screening drug candidates to modulate central or systemic inflammation and its consequences on BBB and how the diversity in regulatory strategies employed at inflammatory genes provides novel opportunities for therapeutic intervention.

### V. Complement Proteins in Neurological Disorders.

The complement system is a part of the immune system that enhances (complements) immune responses. The complement system refers to a series of >20 proteins, circulating in the blood and tissue fluids. Most of the proteins are normally inactive, but in response to the recognition of molecular components of microorganisms or other proteins, they become sequentially activated in an enzyme cascade, where the activation of one protein enzymatically cleaves and activates the next protein in the cascade. The cascade of interacting proteins forms at least 3 cascade pathways, where subsets of some of the complement proteins are involved. In other words, activation of one set of complement proteins does not always involve all of the complement proteins. Deficiencies or altered functioning of some complement proteins may be an etiologic factor in the development of disease, e.g. autoimmune disease.

C1 protein complex is an initial responder of a classical pathway of the innate immune system, composed of 3 subunits designated as C1q, C1r, and C1s. Merely for example; C1q recognizes and binds to immunoglobulin complexed to antigen for initiating activation of at least some proteins in the complement cascade, e.g. C1 to C4.

In particular, C1q, which modulates the immune responses of a variety of cells, interacts with diverse ligands, which can perform various functions in physiological and pathophysiological conditions. C1q has a broad neuroprotective role during the inflammatory response to pathogens. However, C1q may also have deleterious interaction with abnormal protein aggregates and may be involved in the progression of neurodegenerative diseases. C1q is also produced by cells of the central nervous system (CNS). Cho, “Emerging Roles of Complement Protein C1q in Neurodegeneration.” Aging and Disease, 10(3): 652-663. June, 2019, reports that normal C1q may have deleterious interaction with abnormal protein aggregates and thus may be involved in the progression of neurodegenerative diseases.

FIG. 17 shows an exemplary schematic diagram of examples of C1q with neurodegenerative diseases. Cho, “Emerging Roles of Complement Protein C1q in Neurodegeneration.” Aging and Disease, 10(3): 652-663. June, 2019.

A. Targeting of Complement to Treat Neuroinflammation in the Brain-Chip.

In some embodiments, a BBB chip and Brain chip are contemplated for determining whether blockade of complement activation would reduce neuroinflammation similar to its known in vivo actions. In some embodiments, a BBB chip and Brain chip are contemplated for determining whether blockade of complement activation would reduce neurodegeneration triggered by neuroinflammation. In one embodiment, measuring cytokine levels, e.g. IL-6, is used for determining a reduction in neuroinflammation.

FIG. 18 shows an exemplary chart demonstrating that Treatment with C1q neutralizing antibody attenuates TNF-mediated inflammation, as indicated by IL-6 levels. Data are means±SEM (n=6 chips), Anova with Tukey's post-hoc test, **P<0.01, ***P<0.001.

B. Embodiments of Inflammation Models.

As described herein, at least two types of neurotoxicity were discovered using embodiments of Brain chips: neuroinflammation with increased permeability of the BBB, and systemic inflammation also associated with increased permeability of the BBB. Thus, in some embodiments, systemic inflammation of a brain chip is contemplated for use in determining whether targeting complement might be an effective treatment.

### VI. Pathology of Parkinson's Disease.

Parkinson's disease (PD) is the second most common degenerative neurological disorder after Alzheimer's disease. Overall, as many as 1 million Americans are living with PD, and approximately 60,000 Americans are diagnosed with PD each year. There is no standard treatment for Parkinson's disease (PD). Loss of substantia nigra (SN) neurons causes Parkinson's disease. Some of the remaining neurons in PD contain insoluble cytoplasmic protein aggregates (Lewy Bodies) that are made of aggregated alpha-synuclein. FIGS. 47A-E.

FIGS. 54A-E shows images from a pathological examination of a healthy patient (FIG. 54A) reveals typical pigmented DA neurons in the SN (arrows); in contrast, loss of SN neurons matrices (ECM), combined with the application of perfusion and other in vivo microenvironment relevant cues. Existing in vitro human BBB-Chip models have been designed to reconstitute the cerebrovascular interface. However, they have not included combinations of essential cell types, such as region-specific neurons, astrocytes, and microglia, to simulate the complex physiology of NVU milieu³⁰⁻³⁴.

As described herein, a novel human Brain-Chip with dopaminergic neurons of the Substantia Nigra (SN), a predominantly affected area in PD (referred to as “SN Brain-Chip”) was provided. This embodiment of SN Brain-Chip recreated a vascular-neuronal interface using iPS-derived human brain endothelial cells, pericytes, astrocytes, microglia, and dopaminergic neurons. To model states of exposure to abnormal αSyn aggregation and confirm the capability of the SN Brain-Chip to generate clinically relevant endpoints, a model of synucleinopathy was induced by introducing human αSyn pre-formed fibrils (PFFs), referred as “αSyn fibrils”, within the brain channel. Evidence that this model can replicate pathological hallmarks observed in human PD brains, included pSer129-αSyn accumulation, mitochondrial dysfunction, and progressive neuronal death³⁵. In parallel, activation of astrocytes and microglia was observed in line with the active inflammatory process operating in the SN in patients with PD³⁶. Further, evidence that the worsening of the brain pathology over time affects the whole neurovascular unit, was unexpectedly observed by compromised BBB permeability.

When taken together, these data suggest that a human αSyn fibril-induced disease model on the SN Brain-Chip provides a valid model for dissecting complex pathophysiological features of PD, including the BBB dysfunction. It provides a platform for evaluating the efficacy of new therapies against PD and other synucleinopathies and can potentially be utilized for the evaluation of new disease biomarkers and preclinical testing of therapeutic compounds. Additionally, embodiments of Brain chips may be used for safety and efficacy testing of currently known therapeutics used for treating neurodegenerative diseases.

A. Modeling Parkinson's Disease.

Parkinson's Disease (PD): A progressive neurodegenerative disease often lethal first targeting dopaminergic (DA) neurons. The clinical pathology in humans is Lewy Body formation, consisting of abnormal aggregates of a-synuclein (alpha-Syn), a protein expressed in healthy and diseased states. Trigger of pathology initial event still unclear (Phosphorylation of a-synuclein involved). Current hypotheses for pathogenesis: intestine-originated, neuroinfection-driven, genetic involvement, prion-like disease etc. Currently, research on PD-associated BBB impairment in a cell culture system is done using conventional cell culture systems, culturing endothelial cell lines after a short static incubation period with αSyn. However, such models lack many features of the human brain microenvironment in PD and are therefore of limited value. Despite the promise of microfluidic technology for modeling complex neurodegenerative diseases such as PD, no microfluidic models of PD are reported in the literature that includes 5 cellular components of the brain's neurovascular unit.

1. Successful Incorporation of Dopaminergic Neurons in the Brain-Chip.

Demonstrated survival of iPS-derived dopaminergic neurons over 10 days of culture on the Brain-Chip. Confirmed functionality as indicated by the sustained dopamine release.

FIG. 19 shows an exemplary immunostained Brain-Chip on Day 10 demonstrating iPS-derived Dopaminergic Neurons double positive (yellow) for a MAP2: Neuronal Marker (green) and a TH: Selective Marker for Dopaminergic Neurons (red). Scale bar: 50 μm. Shows an exemplary chart demonstrating Neurotransmitter Secretion, e.g. Dopamine in the range of pg/mL, at Day 7 and Day 10 (n=6 chips).

2. Comparing Some Pathological Similarities and Differences Between Parkinson's Disease and Alzheimer's Disease.

For Parkinson's disease, red shade indicates sites of major cell loss and α-synuclein pathology, e.g. near the brain stem. For Alzheimer's disease, green shade throughout the cortex indicates major regions of cell loss and β-amyloid plaques and tau pathology.

FIG. 20 shows an exemplary schematic diagram of a human brain cortex containing GABAergic and glutamatergic neurons representing two neuronal classes, which establish inhibitory and excitatory synapses, respectively. Human Dopaminergic neurons are localized in the substantia nigra (SN). In some embodiments for comparing some pathological similarities and differences between Parkinson's disease and Alzheimer's disease. For Parkinson's disease, red shade indicates sites of major cell loss and α-synuclein pathology, e.g. near the brain stem. For Alzheimer's disease, green shade throughout the cortex indicates major regions of cell loss and β-amyloid plaques and tau pathology.

B. Microfluidic Platforms for Parkinson's Disease

The following describe exemplary publications that do not provide the benefits of the microfluidic devices described herein. A Novel Microfluidic Cell Co-culture Platform for the Study of the Molecular Mechanisms of Parkinson's Disease and Other Synucleinopathies. Frontiers in Neuroscience 2016, uses H4 neuroglioma cells—Cell line (Human), N9 cells—Cell line (mouse). A microfluidic platform for continuous monitoring of dopamine homeostasis in dopaminergic cells. Microsystems and Microengineering 2019, uses a SH-SY5Y-Cell line (Human Cell in widely used for a number of different disease states. Lacks specificity). 3D Cultures of Parkinson's Disease-Specific Dopaminergic Neurons for High Content Phenotyping and Drug Testing. Advanced Science. 2018 uses Human iPS-derived Dopaminergic Neurons from PD patients. Automated microfluidic cell culture of stem cell derived dopaminergic neurons. Scientific Reports 2019 (Mimetas) uses Automating the differentiation of human neuroepithelial stem cell into dopaminergic neurons (Healthy and Mutated). They showed Immunostaining and calcium imaging of iPS-derived dopaminergic neurons, however no glia cells were included in these studies. In addition, this model lacks the BBB module. Modeling Parkinson's disease in midbrain-like organoids. NPJ Parkinson's Disease 2019. The main focus of this study is to recapitulate disease-relevant phenotypes using organoids from healthy individuals and patients. Main finding: FOXA2, for dopaminergic neurons generation, increases in PD patient-derived midbrain organoids, suggesting a neurodevelopmental defect in dopaminergic neurons expressing LRRK2-G2019S (mutant). No glia cells were included in these studies. In addition, this model lacks the BBB module. LRRK2 is not particularly a strong model as it shows minimal levels of neurodegeneration, and does not cover other facets of the disease.

1. Major Sites of Brain Pathology.

In the human cortex, GABAergic and glutamatergic neurons represent 2 major neuronal classes, which establish inhibitory and excitatory synapses, respectively. Human Dopaminergic neurons are localized in the substantia nigra (SN). For Alzheimer's disease, green shade indicates major regions of cell loss and β-amyloid plaques and tau pathology, while in Parkinson's disease, red shade indicates sites of major cell loss and α-synuclein pathology. In some embodiments, an innervated Brain-chip may be used for in vitro parallel of in vivo brain pathology comprising hiPSC-derived neuronal cultures using specific markers for discriminating neurons from astrocytes and pericytes, showing neurons (MPA2+, green) in direct contact with astrocytes (GFAP+, pink) and pericytes (NG2+, red), after 10 days of co-culture. Blue represents Hoechst-stained nuclei.

FIG. 21 shows exemplary schematic diagrams depicting the progression of Parkinson's Disease in one embodiment of a Brain-Chip. Healthy alpha-synuclein (alpha-Syn) (monomeric) becomes phosphorylated at P Ser-129 (amino acid 129) forming alpha-Syn oligomers which aggregate into fibril aggregates with pathologic alpha-Syn (PFFs). Dopaminergic neurons and other brain cells take up extracellular PFFs inducing on a Brain-chip one or more of neuronal dysfunction, e.g. Impaired Calcium activity; impaired Mitochondrial Function e.g. Expression measured by JC-1; Neuroinflammation, e.g. Increased IL-6 secretion, Microglia activation, Astrocyte proliferation; and Neuronal Loss e.g. reduced number of cells after staining with MAP2, symptoms and pathology also observed in clinical/pathology of a PD brain. Exposure to pathogenic alpha-Syn Drives Disease-Relevant Mechanism of Action 90% of α-Syn deposition in Lewy bodies (PD brain) is phosphorylated at Ser129, as opposed to no more than 4% in the healthy brain.

Increase in alpha-Syn phosphorylation upon exposure to PFFs (pathogenic form of alpha-Syn) in the Brain-Chip indicates induction of in vivo relevant mechanism of disease pathogenesis on Chip in a tightly, concentration- and time-controlled manner. To our knowledge this is the first report that a complex human platform model this seminal aspect of development of human PD.

FIG. 22 shows exemplary fluorescently stained micrographs and a chart demonstrating a dose response of pathogenic alpha-Syn PFFs contacting neurons in one embodiment of a microfluidic brain-chip over time for inducing an increasing amount of pSer129 within neurons simulating a-Syn deposition in Lewy bodies of a PD brain. Dose response is 400 ng/ml vs. 4000 ng/ml of alpha-Syn, e.g. alpha-Syn PFFs at Day 3 and Day 6 of exposure. Panels show results of cellular exposure to monomers (normal alpha-Syn) in a brain chip in contrast to panels showing exposure of cells in a Brain-Chip to PFFs (Pathogenic alpha-Syn). pSer129-αSyn (green) and DAPI stained nuclei (blue). Scale bar: 50 μm.

An exemplary chart shows increasing amounts of a toxic form of Ser129-αSyn activity (Fold change vs monomers) where at Day 3 there is a similar amount with 400 ng/ml vs. 4000 ng/ml (NS—not significant).

2. Mitochondria Impairment Following Exposure to αSyn PFFs.

aSyn PFFs induced mitochondria damage in a concentration and time dependent manner, in line with the in vivo findings. JC-1 dyes can be used as an indicator of mitochondrial membrane potential in a variety of cell types, including myocytes and neurons.

FIG. 23 shows exemplary florescent micrographs of fluorescently stained embodiments of Brain Chips and a chart demonstrating a dose response of pathogenic alpha-Syn PFFs contacting neurons in one embodiment of a microfluidic brain-chip over time for inducing an increasing amount of JC-1 within neurons simulating JC-1 staining of a PD brain. Dose response is 400 ng/ml vs. 4000 ng/ml of JC-1, e.g. alpha-Syn PFFs at Day 3 and Day 6 of exposure. Red fluorescence indicated normal mitochondrial potential, whereas green fluorescence indicated damage to mitochondrial potential. Panels show results of cellular exposure to monomers (normal alpha-Syn) in a brain chip in contrast to panels showing exposure of cells in a Brain-Chip to PFFs (Pathogenic alpha-Syn). JC-1 (green) and DAPI stained nuclei (blue). Scale bar: 50 μm.

3. Impairment of Ca 2+ Transients Upon Exposure to αSyn PFFs.

αSyn PFFs caused progressive impairments in neuronal network function and excitability that culminate in neuron death, as reported in human PD brains.

FIG. 24 shows exemplary loss of transient Ca++ signaling (no change in Ca++ levels) over time after Alpha-Syn PFFS treatment compared to Alpha-Syn monomer treatment (signaling off and on, see insets). FUOR-4AM fluorescent staining of Brain chips after 6 Days of Exposure to Monomer and PFFS, 4000 ng/ml. Column of panels, left to right, 0 sec 10 sec 20 sec 30 sec.Scale bar: 50 μm. Electrical read-outs show an almost complete loss of transient signaling after Alpha-Syn PFFS treatment, lower charts.

4. Inflammatory Responses Following Exposure to αSyn PFFs.

Alpha-Syn PFFs induced astrogliosis, microglia activation and neuronal loss, similarly to the findings in human PD patients.

Concentration-dependent induction of IL-6 secretion, demonstrates support for a role of neuroinflammation in PD pathogenesis.

FIG. 25 shows exemplary florescent micrographs and charts comparing fluorescently stained Neurons (MAP2), Astrocytes (GFAP), Activated Microglia (CD11b), Nuclei (DAPI) 6 Days of Exposure after Alpha-Syn PFFS treatment compared to Alpha-Syn monomer treatment, 4000 ng/ml. Left chart demonstrates % of specific cell subtypes over total brain cells (normalized to DAPI stained nuclei). n=6 chips means±SEM.*P<0.05, **P<0.01, ***P<0.001. Right chart demonstrates IL-6 levels pg/mL in neuronal IL-6 channels. ** indicates a significant difference.

5. aSyn PFFs—Mediated Cytotoxicity in the Brain-Chip.

Prolong exposure to aSyn PFFs resulted in clinically relevant progressive cytotoxicity in a concentration dependent manner. In some embodiments methods are provided herein, for testing therapeutics for their efficacy in reducing or eliminating this toxicity.

FIG. 26 shows exemplary results of LIVE-DEAD assay comparisons indicating neuronal death after 3 days of exposure to Monomer and PFFS, 4000 ng/ml along with charts showing LIVE/DEAD Ratios after 3 and 10 days of exposure. n=6 chips means±SEM. **P<0.01, ***P<0.001.

6. Blood-Brain Barrier Pathology in Parkinson's Disease.

It was initially assumed that BBB remained unaltered during the development of the pathology, as observed in animal models and permeability studies of PD drugs such as levodopa and benserazide (Kurkowska-Jastrzebska et al., 1999; Haussermann et al., 2001). More recently, clinical studies have presented evidence of BBB disruption in PD patients (Kortekaas et al., 2005; Hirano et al., 2008; Ohlin et al., 2011; Lee and Pienaar, 2014). For example, an early study (Kortekaas et al., 2005) pointed out an increase in the brain uptake of drugs that usually do not cross the BBB including benzerazide and [11C] verapamil in PD patients and rat models, suggesting a possible BBB breakdown.

However, it was discovered as described herein, that Syn PFFs caused an increase in blood-brain barrier permeability after 6 days of exposure.

FIG. 27 shows exemplary results of a loss of barrier function by an Alpha-Syn PFFS treated a Brain chip compared to alpha-Syn monomer treatment. n=8 chips. means±SEM. ****P<0.0001.

Thus, a Brain-Chip including a unique combination of brain microenvironment and cell features was used to recapitulate physiological relevance not previously achieved with similar in vitro and ex vivo models. We provided proof-of-concept that the Brain-Chip be used as a reliable and reproducible system for assessment of drug delivery to the brain across the BBB, as well as for recapitulating features of neuroinflammation by an in vivo—relevant manner. Proof of concept was also provided for recapitulating of aspects of a human PD model in the Brain-Chip by utilizing in vivo relevant neuronal inflammation, neurodegeneration and neuronal death.

Thus, an inflammatory microfluidic cell model was developed wherein altered neuronal proteins associated with pathogenic neuronal aggregates were used for inducing inflammation in a microfluidic Brain chip. Because embodiments of microfluidic Brain chips include multiple cell types, such induced inflammation using types of abnormal proteins found in vivo neurologically associated diseases, allows for the identification of new drug targets and also allows for preclinical testing of drugs and other compounds using the same type of microfluidic systems. The use of patient derived cells for drug testing using compositions and methods described herein also may be applied to personalized clinical treatments.

Further, a sensory neuron ECM was developed and tested for increasing the quality of neuronal cells that are matured and differentiated on microfluidic chips after seeding with iPSC derived neuronal progenitor cells. In one embodiment, sensory neuron ECM was used for coating chips prior to seeding iPSC derived neuronal progenitor cells on Brain chips. In one embodiment, sensory neuron ECM was used in combination with immune cells and intestinal cells in microfluidic chips.

Moreover, methods were developed for modulating complement activity in a microfluidic chip. In one embodiment, an inflamed Brain chip was treated with an anti-C1q antibody for decreasing IL-6 secretion.

As described herein, one contemplated part of neuronal inflammation is proteins released by intestinal cells. Exemplary embodiments of Intestine-chips including inflamed Intestine-chips are described herein. Thus, in one embodiment, effluent from in an inflamed intestine chip is flowed into a healthy Brain chip for identifying new drug targets and preclinical evaluation of drug treatments. in one embodiment, effluent from in an inflamed intestine chip is flowed into an inflamed Brain chip for identifying new drug targets and preclinical evaluation of drug treatments. In some embodiments, the present invention provides a human in vitro cellular model to study the role of alpha-Synuclein in the pathogenesis of Parkinson's disease.

In some embodiments, neuronal cells in the brain compartment comprise dopaminergic brain cells. In some embodiments, neuronal cells in the brain compartment comprise cortical brain cells.

### VI. SN Brain-Chip: Substantia Nigra Module.

As described herein, embodiments of a novel human Brain-Chip were developed in an attempt to recapitulate a complex neurovascular unit by creating a vascular-neuronal tissue in a microfluidic device for interface mimicking in vivo brain tissue. See FIGS. 30A-F for one example. Models of dopamine neuron vulnerability are hindered by the lack of dopaminergic cell death in α-synuclein models. In preferred embodiments, a SN Brain-chip neuronal cell comprises dopaminergic neuronal cells. In further embodiments of a SN Brain-chip neuronal cells comprise dopaminergic neuronal cells and cortical nerve cells.

Surprising advantages of using these embodiments of a Brain-chip include providing a better brain tissue model when compared to Transwells, i.e. devices lacking flowing fluids. In fact, data provided herein demonstrates more gene expression similarities between one embodiment of a Brain-Chip to in vivo brain tissue than Transwell based cell cultures compared to in vivo brain tissue. Further, evidence is provided herein demonstrating a surprising discovery of cross-talk between endothelium and brain neuronal tissue across a blood-brain barrier in a Brain-chip. In part, evidence includes changes in regulation of endothelial genes in response to changes within the adjacent neuronal (brain) compartment.

Moreover, a model of substantia nigra brain chip comprising a vascular-neuronal tissue interface comprising dopaminergic neurons was developed and used as described herein. Dopaminergic neuron function is reduced in the substantia nigra along with the formation of Lewy body inclusions containing aggregated α-synuclein in Parkinson's disease (PD). Defects of α-synuclein are associated with both familial and idiopathic cases of Parkinson's disease. However, there are current challenges with therapies that target α-syn. For some examples, there are difficulties in identifying which variation of target α-syn conformations are present between different individuals; identification of additional contributing gene products. Then after therapeutics are developed and use clinically, there are patient safety concerns over long-duration large clinical trials and usage. Further, some in vitro models showing dopamine neuron vulnerability are hindered by the lack of dopaminergic cell death when modeling α-synuclein effects for testing potential use of α-synuclein targeted therapeutics.

Progressive misfolding and accumulation of α-syn are related to an imbalance in levels of α-syn synthesis, aggregation, and clearance. Because α-syn is the major defected molecule implicated in the progression of PD, more effective α-syn-directed (targeted) therapeutics are needed. Current treatments for PD are focused merely on symptom-controlling and supplementing dopamine deficiency by using dopamine replacement therapy with levodopa with or without related compounds. Moreover, there are no effective therapeutics for halting the underlying degeneration, nor to treat symptoms due to non-dopaminergic neuron damage. Current treatments are also limited by a lack of disease-modifying therapeutic compounds aimed at reducing α-syn toxicity with a lack of alleviating the neurotoxic gain of α-syn aggregation. Hence, successful α-syn-directed therapeutics might alleviate the neurotoxic gain of α-syn via drug targets associated with one or more of: a reduction of α-syn synthesis, inhibition of α-syn aggregation, and an increase of α-syn clearance. These types of targeted α-syn-directed therapeutics are contemplated to induce a healthier neuronal balance between α-syn synthesis, aggregation, and clearance. αSyn released into extracellular space may interact with lipoprotein particles released by microglia and astrocytes so that this type of complex may also be a drug target.

Thus, embodiments of methods using SN Brain-Chips for providing new drug targets associated with inducing α-syn damage to the cells in a SN Brain-Chip are described herein. Examples of additional drug targets include but are not limited to test compounds that target such as binding-proteins, β-wrapins, e.g. beta-wrapin AS69, for preventing α-synuclein from aggregating by preventing elongation and formation of new protein fibrils in nerve tissue and/or cells; and test compounds such as protease stimulators and autophagy stimulators for increasing degradation of misfolded alpha-synuclein.

A. Alpha-Synuclein and Synucleinopathies.

Alpha-synuclein (aSyn or a-syn) is found as a major component of Lewy bodies and Lewy neurites of neurons in patients showing symptoms of PD and patients with a related dementia with Lewy Bodies (DLB). Further, neocortical distribution of α-synuclein pathology is found in patients' with PD dementia. These protein inclusions made up primarily of insoluble and fibrillary aSyn protein. aSyn also accumulates in Lewy bodies of multiple system atrophy (MSA) patients. In MSA patients, aSyn is found predominantly within oligodendrocytes as cytoplasmic inclusions. These disorders share the accumulation of aSyn aggregates as a pathological feature so are collectively known as synucleinopathies. FIGS. 45A-B.

Human cells endogenously express α-syn. Normal human α-syn refers to an abundant 14-kDa protein having around 140 amino acids, comprising 3 domains: (1) an N-terminal lipid-binding α-helix, (2) a non-amyloid-β component (NAC) domain, and (3) an unstructured C-terminus. The N-terminal, having seven 11 amino acid repeats, plays a role in binding to membranes, upon which it adopts an α-helical secondary structure. When abnormal, it misfolds into aggregates. When this occurs, a random coil of the NAC region, a highly hydrophobic sequence underlying the aggregate nature, forms β-sheets and leads to protofibrils and fibrils. The unstructured C-terminus contains a large number of charged residues, that contribute to inhibiting this fibril formation, and may have significant abnormal post-translational modifications that alter aSyn.

Human SNCA protein has the following amino acid sequence (SEQ ID NO: 1):

1 MDVFMKGLSK AKEGVVAAAE KTKQGVAEAA* GKTKEGVLYV
GSKTKE*GVVH* GVA*TVAEKTK EQVTNVGGAV VTGVTAVAQK
TVEGAGSIAA ATGFVKKDQL GKNEEGAPQE GILEDMPVDP
DNEAYEMPSE EGYQDYEPEA 140

Amino acid tandem repeats of [Glu/Gly/Ser]-Lys-Thr- Lys-[Glu/Gln]-[Gly/Gln]-Val-X-X-X-X are highlighted. The star sign shows mutation sites in α-syn including A30P, E46K, H50Q and A53T, which are associated with PD, and occur in the N-terminal of α-syn in tandem repeats.

Further, α-synuclein refers to a SNCA gene and expressed protein that is a member of a protein family of synucleins, together with beta (β)-synuclein and gamma (γ)-synuclein. Proteins without coding mutations in this region share a characteristic consensus amino acid sequence (KTKEGV) that is repeated about six times, more or less, at the N-terminal part of the protein. β-synuclein shares the closest homology (90% homology in the N-terminus and 33% homology in the C-terminus) with α-syn (aSyn). Point mutations in a human SNCA gene, encoding for αSyn, and multiplications of the SNCA locus were identified in families with autosomal-dominant forms of Parkinson's disease (PD). Genome-wide association studies linked other single-nucleotide polymorphisms in the SNCA gene with increased susceptibility to sporadic PD. Moreover, several SNCA gene polymorphisms were associated with increased risk of multiple system atrophy (MSA).

Within cells, α-syn normally adopts α-helical conformation (FIG. 44A, left) for mediating a range of neuronal cellular functions (FIGS. 44A right and 44B), examples described herein. Under certain circumstances, such as a point mutation or post-translation modifications, to provide modified monomers that undergo a profound conformational transition into oligomers, and a β-sheet-rich fibril structures that polymerizes to form toxic oligomers, amyloid fibrils and Lewy bodies. (FIG. 44A, middle and right). In fact, α-syn is also considered an apolipoprotein. Merely as one example, when the structure of the monomer is changed, such as with at least one point mutation in certain positions, then instead of an alpha-helical structure the altered aSyn monomer forms mutant oligomers and fibrils ((FIG. 44A, middle and right).

FIG. 44A shows exemplary schematic diagrams demonstrating native and toxic conformations of α-syn. Alpha-synuclein transforms into multiple different conformations, including monomers (predominant in a α-helical confirmation), tetramers, higher-level oligomers (soluble conformations), and fibrils (highly ordered insoluble conformations characterized by β-sheet conformation). Alpha-synuclein exists in a native conformation as monomers as well in a dynamic equilibrium with tetramers. The tetramer, less likely to form aggregate, may form an aggregate after disrupted into monomers in order to misfold. Many factors, such as the posttranscriptional modification and SNCA mutations in A53T and E46K promote formation of pathological oligomers, presently considered to be the most toxic structure of α-syn, which is further folded to form amyloid fibril (rich in β-sheet structure), the accumulation of which leads to the formation of intracellular inclusions called Lewy Body.

FIG. 44B shows exemplary interactions between α-syn and cellular components contemplated as drug targets for use in drug screening methods as described herein. Misfolded α-syn is degraded through the autophagy-lysosomal pathway (ALP) and the ubiquitin-proteasome system (UPS). Certain oligomeric species present toxicity via interactions with cellular components by mechanisms that include: (1) alteration of cytoskeletal integrity; (2) membrane disruption and pore formation; (3) nuclear dysfunction; (4) inhibition of vesicle docking; (5) UPS dysfunction; (6) ALP impairment; (7) reduction of mitochondrial activity; and (8) chronic ER stress. UPS, ubiquitin-proteasomal system; ALP, autophagy-lysosomal pathway; ER, endoplasmic reticulum.

FIG. 44C shows exemplary schematic summary of interactions between α-synuclein and cellular components, such interactions are contemplated for use as drug targets in methods of use for microfluidic Brain-Chips as described herein. At least six different exemplary intracellular pathways are affected by α-synuclein (α-syn). The protein α-syn is enriched at the pre-synaptic terminals of the majority of types of neurons in the brain, where it participates in the vesicle recycling, thereby modulating synaptic function. α-syn can be degraded by the ubiquitin-proteasome system (UPS) and inside the lysosomes. α-syn interacts strongly with membranes, such as plasma membrane and mitochondrion. When misfolded, α-syn forms distinct structures that are prone to aggregation, into oligomers, then into larger structures. α-syn oligomers in a toxic form may impair basic neuronal processes, such as ER-Golgi trafficking, lysosome and UPS functions, reduced mitochondrial activity and alter the plasma membrane through the pore/perforations that can dysregulate calcium and cation homeostasis. In fact, many of these pathways were identified as GO categories of Genes that were upregulated genes in Brain-Chips.

FIG. 44D shows exemplary autophagy-lysosomal pathway (ALP) and ubiquitin-proteasome system (UPS) pathways under normal and pathological conditions. Proteins are tagged with ubiquitin conjugates through a sequential enzymatic mechanism involving three classes of enzymes, E1, E2 and E3. Under normal conditions, ubiquitylated substrates are recognized by ubiquitin receptors present in ALP and UPS pathways and efficiently eliminated. In the UPS, substrates are subsequently deubiquitylated by RPN11, a step for substrate degradation and amino acid recycling. Free-Ub chains formed by RPN11 activity promote ALP function. Ubiquitin receptors in the ALP, in contrast to the UPS, form oligomers to facilitate substrate recognition and autophagosomal recruitment. Under aging and Alzheimer's disease conditions there is a decrease in the function of the ALP and the UPS that reduces substrate degradation and amino acid recycling. Downregulation of RPN11 in Alzheimer's disease (AD) decreases free-Ub chains disrupting substrate recognition, their recruitment into autophagosomes and their final degradation by the ALP. Altogether, leading to the accumulation of deleterious protein aggregates. Transcriptional regulation (Nrf1/2) and phosphorylation (kinases/phosphatases) play a crucial role in ALP and UPS function whereas their dysregulation is the focus of intense studies in aging and Alzheimer's disease

FIG. 45A shows exemplary schematic diagrams depicting steps towards accumulation of Alpha-synuclein protein (SNCA). Natural SNCA becomes misfolded under stress and becomes oligomers, oligomers, profibril oligomers that form fibril aggregates that form Lewy bodies in affected neurons of a patient's PD brain leading to dopamine (DA) neuronal loss. Parkinson's disease (PD) is part of a larger group of Lewy body disorders. FIG. 45B shows an exemplary schematic depiction of α-synuclein fibril contributions to Alpha/Beta plaques, Tau tangles and α-synuclein Lewy bodies found in degenerating neurons. In some embodiments, test compounds are added to Brain-chips comprising neurons having α-syn Lewy bodies for identifying compounds for use in reducing the size and or number of α-syn Lewy bodies for use in treating α-syn Lewy body associated diseases. In some embodiments, test compounds are added to Brain-chips comprising neurons having alpha-beta plaques for identifying compounds for use in reducing the size and or number of alpha-beta plaques for use in treating alpha-beta plaque associated diseases. In some embodiments, test compounds are added to Brain-chips comprising neurons having Tau tangle for identifying compounds for use in reducing the size and or number of Tau tangle for use in treating Tau tangle associated diseases.

Synergistic effects of α-synuclein, hyperphosphorylated tau, amyloid-β, and other pathologic proteins include induction and spread of protein aggregates in neurons. In some embodiments, α-synuclein induces hyperphosphorylation of tau protein. Under certain conditions mutant (changed) aSyn protein may further induce formation of or comprise an α-syn Lewy body, an alpha-beta plaque, or a Tau tangle. Thus in some embodiments, Brain-Chips are contemplated for use in testing of α-syn molecules described herein to form any one or more of an α-syn Lewy body, an alpha-beta plaque, or a Tau tangle associated to Synucleinopathy disorders. One common pathological link connecting these disorders is the intracytoplasmic accumulation of misfolded and aggregated forms of aSyn in neurons and in glial cells. FIG. 45B.

FIG. 45B shows an exemplary schematic depiction of α-synuclein fibril contributions to Alpha/Beta plaques, Tau tangles and α-synuclein Lewy bodies found in degenerating neurons.

As described herein, in one embodiment, methods are provided for characterization of the effects of aSyn fibrils on the cells in the brain channel of SN Brain-Chips. Thus, in one embodiment, there is a characterization of the effects of aSyn fibrils on the permeability of the Blood-Brain Barrier with comparisons of exposure to a monomeric form of aSyn. However, such methods of testing aSyn are not limited to aSyn fibrils. Indeed, nonlimiting types of aSyn molecules and structures are contemplated for comparative testing for neuronal effects. Examples of aSyn molecules and structures contemplated for testing include but are not limited to oligomers, profibrils, and variants including those described herein.

More examples of variants of α-syn contemplated for toxicity testing in a Brain-Chip include but are not limited to dinucleotide repeat REP1 located in the SNCA promoter (SNCA-REP1) and the 3′ untranslated region (UTR) variants. Variations in these regions may increase susceptibility to PD by interfering with transcription factor binding sites and creating or destroying microRNAs target sites, which in turn modifies gene expression. Examples of missense mutations in SNCA locus were identified in familial forms of PD (A53T, A30P, E46K, and H50Q) and sporadic PD patients (A18T and A29S). Further, duplications and triplications of the SNCA locus cause familial parkinsonism and correlate with disease severity.

Single nucleotide polymorphism (SNP) analyses of patients may be used for providing α-syn variants for toxicity testing, both for obtaining general information and personal information for pre-clinical use then use in patients, including the donor. At least thirty-nine different SNPs in the SNCA gene showed a statistically significant effect on PD susceptibility: nine variants in the 5′ end, nine variants near the 3′ end, and 25 intron variants. Moreover, at last 28 distinct loci that modify the individual risk to PD. AT least 2 genes whose variants contribute to susceptibility to sporadic PD are α-synuclein (SNCA) and microtubule-associated protein tau (MAPT) genes, which can exert independent or joint effects on the risk of PD. Variants in other genes previously linked with autosomal forms (LRRK2, PARK16-18, and GBA) were shown association with PD risk. Campêlo, et al., Genetic Variants in SNCA and the Risk of Sporadic Parkinson's Disease and Clinical Outcomes: A Review. 2017.

Alpha-synuclein (aSyn) aggregation from monomers may also form amyloid Amyloidosis refers to when abnormal amyloid protein including amyloid fibrils builds up in tissues and organs. Cerebral amyloid angiopathy (CAA) refers to a condition in which amyloid proteins build up on the walls of the arteries in the brain. CAA increases the risk for stroke caused by bleeding and dementia. aSyn was identified as a component of amyloid from brain tissues of Alzheimer's disease (AD) patients. The presence of a hydrophobic 12 amino-acid sequence in the central part of the protein allows oligomerization and fibrillization of aSyn. Deletion or disruption of this domain blocks the capacity of aSyn to form amyloid fibrils. The process of aSyn aggregation was studied in detail in an attempt to identify the toxic species responsible for neuronal dysfunction and death. However, it is still unclear what is/are the toxic forms of the protein. There is evidence showing that inhibition of aSyn aggregation process is associated with a decrease of aSyn toxicity. and B-pleated sheets

Similarly to the case of amyloid-beta (Aβ) plaques in AD, fibrillar forms of aSyn might one of many toxic aSyn species. Additionally, pre-fibrillar, soluble oligomeric species (comprising multiple aSyn molecules) may also be a toxic aSyn species, with amyloid aggregates serving as a reservoir.

Neurotoxic effects of aSyn oligomers were studied in vivo, using animal models of synucleinopathies. In these studies, aSyn mutant variants that promote oligomer formation were designed and tested for toxicity in vivo. The increasing inability of the mutants to form fibrils was directly correlated with toxicity and neurodegeneration in some in animal, worm and insect in vivo models. In another study, aSyn variants that were shown to promote oligomer formation caused the most prominent dopaminergic cell death upon lentiviral injection into rat substantia nigra (SN). Together, these studies provide evidence for the involvement of soluble oligomers as one of the toxic species in synucleinopathies, although the precise size and type of additional toxic oligomeric species remains to be determined. Further, in vitro studies showed that the acceleration of oligomerisation, and not fibrilization, may be a distinctive shared property of the A53T and A30P αSyn mutations linked to early-onset human PD.

Thus, α-syn molecules contemplated for use in determining effects upon the BBB include but are not limited to forms/structures of α-syn molecules considered normal and altered forms structures of α-syn molecules. Normal forms of α-syn molecules themselves are considered nontoxic, such as monomers endogenously expressed as functional α-syn in normally functioning neuronal cells not associated with a disease phenotype. Some forms of altered α-syn molecules are considered toxic to neurons in other systems, such as altered α-syn molecules that form fibrils. In some embodiments, both types of α-syn molecules are contemplated for testing BBB responses in microfluidic Brain-Chips, including SN Brain-Chips. In some embodiments, altered α-syn molecules, such as those with point mutations that change structure, binding to other molecules, etcs., are contemplated for use in testing in Brain-Chips in order to determine whether a particular change will alter neuronal function, e.g. whether it will show toxic effects. Some changed α-syn molecules may not show effects until combined with another alteration in physiology, for example, when dopaminergic neurons express higher than normal amounts of dopamine.

In some embodiments, toxic effects of α-syn molecules may induce cell degeneration or death of dopaminergic neurons in microfluidic Brian-Chips over time. Thus, in some embodiments, a microfluidic Brain-Chip is contemplated for use in comparative testing for neurotoxity of a range of aSyn mutations, aSyn allele variants, aSyn structural variants, etc., for identifying additional forms of aSyn as drug targets for testing aSyn therapeutics.

### VII. Exemplary Methods of Modeling Neurodegenerative Diseases.

Example A—In some embodiments, a microfluidic SN Brain-Chip is exposed to a mutant variant of α-synuclein.

Parkinson's disease is characterized clinically by a triad of cardinal motor symptoms (bradykinesia, rigidity and tremor) resulting from the loss of dopaminergic neurons in the substantia nigra. Currently, these symptoms may be alleviated by drugs that restore dopaminergic neurotransmission, and/or by deep brain stimulation in some patients. Parkinson's disease (PD) is characterized by the accumulation of misfolded fibrillar α-synuclein (α-syn) that significantly features Lewy bodies and Levvy neurites (LBs/LNs).

In one embodiment, α-syn molecules, such as fibrils, as opposed to α-syn molecules, such as monomers, were used for inducing breakdown of a healthy BBB. In one embodiment, α-syn as fibrils, were used for inducing IgG penetration through a previously healthy BBB, as opposed to α-syn monomers. In other embodiments, other forms of α-syn molecules are contemplated for use to determine their effects on BBB permeability and/or IgG penetration from the vascular channel comprising endothelial cells into the brain channel. Thus in preferred embodiments, IgG is added to the endothelial channel then after chosen time points the amount of the same type of IgG is measured in effluent collected from the brain channel. See, FIGS. 64A-B, for examples.

Example B—In some embodiments, a microfluidic SN Brain-Chip is exposed to α-synuclein comprising an A53T mutation associated with PD.

In some embodiments, iPS cells were generated from a healthy individual to provide normal derived dopaminergic neurons in a healthy brain chip. In some embodiments, iPS cells were generated from a PD patient known to have α-synuclein comprising an A53T mutation to provide disease (mutant) derived dopaminergic neurons in an A53T Brain-chip.

After culturing on-chip, brain channel cells were harvested for RNA Sequencing analysis (RNA-Seq). See, FIG. 50A. Samples using the information from the first 2 Principal Components that explain 36.36% of the total variance in the data. Brain-Chips are clearly separated from the Transwell cultures.

Samples of the RNA Seq data were plotted using information from the first 3 Principal Components (PC) that explain 71.19% of the total variance in the data. Differential Gene Expression Analysis was applied between Brain-Chip (A53T) vs Brain-Chip (Healthy). DE genes selection criteria using DESeq2 R package: adj.pvalue<0.05. |log₂FoldChange|>=0.5. As plotted in FIG. 50B, healthy samples are clearly separated from PD Disease (A53T).

Brain Chips. Control samples (i.e. Healthy and Monomers) are clearly separated from the Disease (Fibrils and A53T Fibrils). Healthy and Monomer exposed Transwell cultures do not appear to have significant differences, i.e. they have overlapping clusters of expressed genes). Fibril and A53T Fibril exposed Transwell cultures show different responses. Healthy and Monomer exposed Brain Chips appear to have wider variances in responses which are clearly different than Transwell culture responses. However Fibril and A53T Fibril exposed Brain Chips do not seem have significant differences, as they show overlapping clusters of expressed genes.

Transwells. Control samples (i.e. Monomers) overlap with the Fibrils. A53T samples are clearly separated from Monomers and Fibrils.

TABLE 5
Brain Channel: DGE Analysis (A53T vs Healthy) 482 genes
were found significantly differentially expressed.

| Condition | # DE genes | # up-regulated | # down-regulated |
|---|---|---|---|
| Brain-Chips | 482 | 320 | 162 |
| (A53T) Vs Brain- | | | |
| Chip (Healthy) | | | |

FIG. 50A shows exemplary schematics for providing embodiments of a Brain Chip: comprising Neurons, Astrocytes, microglia, pericytes, and endothelial cells.

FIG. 50B shows an exemplary Principal Components Analysis (PCA) of healthy vs. PD disease associated brain channel cells.

FIG. 51A Brain Channel: Volcano plot: Brain-chip A53T vs. healthy.

FIG. 51B Brain Channel: GO-terms Enrichment Analysis Results. Go-term enrichment analysis results using the 320 up-regulated genes in A53T brain-Chips.

FIG. 52 Brain Channel: Principal Components Analysis (PCA) comparing the same brain cells cultured in either Transwell cultures or Brain Chips, as healthy cultures without exposure to a monomer or fibril, or exposed to monomers, fibrils or fibrils comprising an A53T mutation.

Example C— In some embodiments, a microfluidic SN Brain-Chip demonstrating altered physiology, e.g., from a mutation in at least one gene that is not α-synuclein, is exposed to a mutant variant of α-synuclein, e.g., A53T α-synuclein. In some embodiments, both dopamine levels and α-synuclein expression are manipulated for observing interactions as contemplated drug targets for therapeutic testing.

One example is shown in FIG. 53. Nigrally targeted expression of mutant tyrosine hydroxylase with enhanced catalytic activity increased dopamine levels without damaging neurons in non-transgenic mice. Mice overexpressing dopamine in the presence of normal human α-syn do not show neuronal degeneration. In contrast, raising dopamine levels in mice also expressing human A53T mutant α-synuclein induced progressive nigrostriatal degeneration and reduced locomotion. Some loss of synapse function around 2.5 months after injection of mutant A35T human α-syn, and over another 2.5 months shows greater loss of synapse function with locamotor impairment and pathological signs of neuronal cell degeneration. Thus, toxic effects of α-syn molecules in vitro may reduce excitability of dopaminergic neurons resulting in loss of synapse function and over time may induce neuronal degeneration and death.

For another example, a microfluidic SN Brain-Chip demonstrating increased dopamine levels from a gene mutation or gene transgenic expression of a dopamine inducing gene, is exposed to α-synuclein proteins or mutant α-synuclein proteins. Thus both dopamine levels and α-synuclein may be tested together for identifying effective aSyn therapeutics on altered dopamine expressing neurons. Nigrally targeted expression of mutant tyrosine hydroxylase with enhanced catalytic activity may increase dopamine levels without damaging neurons in as in non-transgenic mice. In contrast, raising dopamine levels, as in mice expressing human A53T mutant α-synuclein, may also induce progressive nigrostriatal degeneration and reduced locomotion as observed in mice. Dopamine elevation in A53T mice increased levels of potentially toxic α-synuclein oligomers, resulting in conformationally and functionally modified species. Moreover, in genetically tractable Caenorhabditis elegans models, expression of α-synuclein mutated at the site of interaction with dopamine prevented dopamine-induced toxicity. These data suggest that a unique mechanism links two features of PD found in patients: dopaminergic cell death and α-synuclein aggregation. Thus, SN Brain-Chips may be used for testing α-synuclein and α-synuclein mutated at the site of interaction with dopamine for identifying changes in interactions with dopamine. In particular, α-synuclein/dopamine interactions are contemplated for use in developing and testing αSyn therapeutics.

FIG. 53 shows exemplary schematic depictions of dopaminergic neurons with synaptic terminals. While increasing dopamine (blue dots) in the wild-type (WT) setting is benign (left), similar increases in the setting of human mutant (A53T) α-synuclein (α-syn) lead to progressive neurodegeneration (middle and right) in mice. Synaptic loss (red X marks) in presynaptic striatal terminals precedes somatic degeneration, and toxicity is thought to be mediated by α-synuclein (red) oligomers in the presence of dopamine.

### VIII. Contemplated Drug Targets.

The ability to model cellular characteristics of synucleinopathies in embodiments of Brain-Chips provides myriad opportunities for studying pathogenic mechanisms and aid the development and validation of future pharmacological interventions. In particular for use in targeting α-Syn and/or associated molecules for exposing to therapeutics for treating neurodegenerative diseases. Thus, in some embodiments, a microfluidic Brain-Chip is used for screening drug compounds, e.g. test compounds. These disorders include Parkinson's disease (PD), dementia with Lewy bodies (DLB), a Lewy body variant of Alzheimer's disease and multiple system atrophy (MSA). Successful test compounds for halting neurodegenerative effects in microfluidic Brain-Chips are contemplated for further testing as therapeutics for patients at risk of neuronal degeneration disorders related to Lewy body formation.

Contemplated drug targets present in cells of Brain-Chips may be used for test compound evaluation for preventing, slowing or halting neurological diseases, e.g. neurodegenerative diseases such as PD, include but are not limited to α-synuclein associated oxidation, nitration, proteasomal degradation, Golgi trafficking, exocytosis, endocytosis, cellular trafficking, neuro-inflammation, in addition to intercellular α-synuclein, intracellular α-synuclein, etc. Contemplated drug targets include but are not limited to one or more function such as blocking cellular uptake of exogenous α-syn fibrils, transport of exogenous and/or endogenous α-syn fibrils through a neuronal cell and release of α-syn fibrils from cells into the intercellular areas. Thus, contemplated drug targets include but are not limited to blocking movement of α-syn through axonal projections to the synapsis area.

Contemplated drug targets include but are not limited to blocking exogenous entry of α-syn into neuronal cells or other cells involved with an alteration in neural function.

Because some sporadic forms of PD are associated with genes other than those encoding α-syn may also result in abnormal α-syn accumulation. This suggests that at least one or more mutations in at least one of these other genes might exacerbate the role of α-syn function associated upstream factors such as oxidation, nitration, and decreased proteasomal and lysosomal functions. α-syn oligomers may also induce microglia-mediated inflammation. Glial neuro-inflammation, induced by α-syn oligomers or other factors, as well as Golgi trafficking and calcium buffering, might lead to normal and/or abnormal α-syn accumulation through an independent pathway specific to at least one other gene.

In addition, α-syn may spread from neuron to neuron or neuron to glia via self-amplification and for propagating dysregulation of neuronal function in a stereotypical and topographical pattern among neighboring cells. Moreover, aberrant protein deposition within dopaminergic neurons could be related to the dysregulation of the lysosomal autophagy pathway.

Contemplated drug targets include but are not limited to one or more function such as blocking cellular uptake of exogenous α-syn fibrils, transport of exogenous and/or endogenous α-syn fibrils through a neuronal cell and release of α-syn fibrils from cells into the intercellular areas. Thus, contemplated drug targets include but are not limited to blocking movement of α-syn through axonal projections to the synapsis area.

Contemplated drug targets include but are not limited to blocking exogenous entry of α-syn into neuronal cells or other cells involved with In one embodiment, a treatment compound is contemplated for use in blocking cellular uptake of α-syn, e.g. a neuronal cell such as a dopaminergic cell, cortex cell, glial cell, and any other cell within a Brain-Chip. α-syn can be taken up by primary human cortical neurons and astrocytes in vitro. Brain cells exposed to α-syn may lead to impaired mitochondrial function, in turn leading to cellular degeneration and cell death. Thus, mitochondrial dysfunction may also be a drug target related to toxicity of α-syn in human cells.

α-syn fibrils may also be released via exosomes into intercellular spaces. In some embodiments, contemplated drug targets inhibit exosomal release of α-syn fibrils. Aggregated α-syn is secreted by neurons by non-canonical pathways implicating various molecular chaperones including USP19 and the DnaJ/Hsc70 complex. In some embodiments, contemplated drug targets are aggregated α-syn molecular chaperones.

Extracellular α-syn fibrils may diffuse from neuron-to-neuron or actively transmitted from neuron-to-neuron by tunneling nanotubes. In some embodiments, contemplated drug targets block extracellular movement and/or entry into other cells, e.g. using α-syn fibril blocking antibodies.

In some embodiments, cell surface receptors that regulate the uptake of α-syn fibrils are contemplated drug targets. Aggregates that enter cells are then transported along axons, both in the anterograde and retrograde direction. However, some aSyn variants are associated with defects in vesicle trafficking. aSyn associated trafficking molecules may also be drug targets. An inhibitory effect of aSyn on ER-to-Golgi complex trafficking in mammalian kidney and neuroendocrine cells, with the A53T aSyn mutant causing stronger inhibition then the wild-type form was reported. This aSyn-elicited trafficking defect can be rescued by the co-overexpression of Ykt6p, a vesicle-associated SNARE that promotes vesicle fusion In neuronal cells, aSyn co-localizes and interacts with prenylated Rab acceptor protein 1 (PRAT) in turn associated with multiple prenylated Rab GTPases involved with vesicle trafficking and recycling. aSyn overexpression resulted in accumulation of cytoplasmatic vesicles. Thus contemplated aSyn therapeutics may normalize trafficking when aSyn causes defects in vesicle trafficking steps, including impaired vesicular movement within a cell and recycling of molecules within vesicles.

Exemplary drug targets include but are not limited to, targeting α-syn, e.g., targeting oligomeric α-syn by immunotherapies. In fact, excessive microglial activation is known to increase the production of proinflammatory cytokines including tumor necrosis factor alpha (TNF-α), interleukin-1-β (IL-1β), interleukin-6 (IL-6), and interferon-γ (TNF-γ). Thus contemplated drug targets include blocking α-syn induction of microglial activation. Contemplated readouts include but are not limited to reduction of at least one or more proinflammatory cytokine.

In some embodiments, methods of using Brain-Chips are contemplated for testing compounds for neuroprotective (prophylactic) treatments for patients at risk of developing neurodegenerative disorders, e.g. patients having known genetic mutations associated with onset of disease. In some embodiments, methods of using Brain-Chips are contemplated for testing compounds for reducing or slowing neurodegeneration.

In some embodiments, Brain-Ships including SN Brain-Chips comprise individual human biopsy derived cells, as primary cells, cultured cells or iPS cell derived cells from patient's biopsies for seeding into microfluidic devise for use in general and individualized personal medicine. IN some embodiments, therapeutics currently being used for treating patients are used for treating Brain-Chips for comparing clinical results to in vitro results on chips. In particular for individualized medicine, known or test compounds as candidate therapeutics may be used for treating brain cells and/or endothelial cells for determining the safety and predicting efficacy for the use of the known compound in the patient donating the original biopsy. Such test compounds for use as therapeutics for treating PD or other types of synchipathies, include but are not limited to known compounds such as Levodopa (L-Dopa), which crosses the blood-brain barrier and increases dopamine levels in the substantia nigra.

Commonly used treatments for PD, such as Levodopa (L-Dopa), are partially or transiently effective and are available or applicable to a minority of patients. These therapies neither restore the lost or degenerated dopaminergic neurons, nor prevent or delay the disease progression, driving the need for more effective therapeutics to protect or rescue damaged dopaminergic neurons.

Further, a gene editing technique, clustered regularly-interspaced short palindromic repeats-associated protein 9 (CRISPR-Cas9), may prove useful for treating PD by preclinical testing in microfluidic Brain-Chips.

Examples of test compounds for use individually or in combinations on Brain-Chips include but are not limited to DA targeting drugs; non-DA targeting drugs; α2-adrenergic antagonists, serotonergic, and adenosine Ata antagonists, novel formulae for levodopa/carbidopa drugs (e.g. use of IPX066, XP21279, and Opicapone), MAO-inhibitors (e.g. safinamide); micro-RNA or Si-RNA approach to inhibit mRNA of misfolded protein aggregates; small molecule-based compounds, active compounds found in foods suggested for PD patients including but not limited to hydroxytyrosol, curcuminoid, isoflavone, caffeine, resveratrol indicated for use as antioxidants, for decreasing SNCA aggregation, anti-inflammatory, etc. Ab against the N-terminal or central region of SNCA, Monoclonal Ab: Syn303 (binds pathological conformations of human and mouse SNCA) targeting N-terminus; Single-chain fragment variables against oligomeric SNCA fused to the low-density lipoprotein receptor-binding domain of APOE-B; SNCA protofibril-selective monoclonal Ab (mAb47); C-Terminus SNCA Ab: 1H7, 9E4, 5C1, and 5D12; etc.

Biomarkers may be used for determining effects of aSyn variants and effects of test compounds. Biomarkers that may find use include but are not limited to gene expression profiling, metabolomics, protein profiling (e.g. Aβ and tau) and inflammatory markers (e.g. IL-6).

FIG. 65 shows exemplary schematic depictions toxicity of α-syn as a therapeutic target. Toxicity of alpha-synuclein to neurodegeneration is associated tightly with the dynamic equilibrium of the protein synthesis, aggregation, and clearance. Levels of specific conformations (oligomers and protofibrils) vary in different stages of PD. Disease-modifying therapeutic strategies are mainly focused on these processes as well as inhibiting cell-to-cell propagation: (i) reducing a-syn synthesis with small interfering RNA (siRNA), microRNA (miRNA), small hairpin RNA (shRNA), and transcription inhibitors; (ii) increasing degradation of a-syn via UPS and ALP; (iii) reducing aggregation of a-syn via heat-shock proteins (hsp40/70/104), aggregation inhibitors, antioxidant, and posttranslational modification approaches (oxidation, nitration, phosphorylation, and C-terminal cleavage); (iv) blocking the propagation of a-syn with immunotherapies by targeting extracellular a-syn or exosome and by blocking putative receptors in recipient cells; and (v) seeking neuroprotective strategies including anti-inflammation and antioxidant.

FIG. 66A shows exemplary potential mechanisms involved in propagation of α-syn. Spreading mechanisms of α-syn in neighboring cells are multiple and can occur via (1) passive transmission through membrane fusion; (2) classical exocytosis and endocytosis; (3) packaged-exosomes; (4) tunneling nanotubes (a direct connection between two cells); (5) axonal transport and transsynaptic junction; and (6) receptor-mediated internalization.

FIG. 66B shows exemplary molecules and signaling pathways involved in α-syn-mediated microglial activation. Excessive microglial activation can increase the production of pro-inflammatory cytokines (TNF-α, IL-1β, IL-6, and TNF-γ), and induce an oxidative stress response, including the release of reactive oxygen species (ROS) and nitric oxide (NO) as well as the production of NADPH oxidase. Toll-like receptors (TLRs) play a vital role in recognizing pathogen-associated molecular patterns (PAMPs) and initiating innate immune responses via distinct signaling pathways, including NF-κB and MAPK activation. Activation of TLR2 resulted in the accumulation of α-syn as a result of the inhibition of autophagic activity through regulation of the AKT/mTOR pathway. Other receptors that are involved in the α-syn-induced microglial response include FcγRs/CD36/P2×7R/EP2/Mac-1/Ion channels. Also, α-syn induced the expression of matrix metalloproteinases (MMPs) and stimulated the activities of MAPK, NF-κB, and AP-1. In addition, MMPs may activate microglial protease-activated receptor-1 (PAR-1) in an autocrine or paracrine manner and increase microglial inflammatory signals (not shown in the diagram). Furthermore, major histocompatibility complex II (MHC-II) and Th1 cells were targeted recently for the activation of microglia. Exosomes are specifically and efficiently taken up by microglia via a macropinocytotic mechanism and are released via activation of 5-hydroxytryptamine (5-HT2a, 2b, and 5-HT4) receptors. Activated exosomes expressed a high level of MHC-II, which may be a potentially pathway for the activation of microglia. In contrast, regulator of G-protein signaling 10 (RGS10), RING finger protein 11 (RNF11), and NF-κB essential modulator (NEMO) inhibitors exert negative regulation on NF-κB signaling, producing a dampened immune response. Finally, microglial cells are also able to phagocytose different forms of extracellular α-syn, via ubiquitin-proteasomal system (UPS) and autophagy-lysosomal pathway (ALP), presenting a mechanism of clearance that might be even beneficial for neuronal survival. The CD36 (a scavenger receptor), FcγRs (Fc gamma receptors), Mac-1 (macrophage antigen-1 receptor), EP2 (prostaglandin E2 receptor subtype 2), P2×7R (purinergic receptor P2×, ligand-gated ion channel 7), and plasma membrane ion channels.

FIG. 66C shows exemplary internalization of α-synuclein fibrils and aggregation of endogenous α-syn protein. Recombinant α-syn fibrils are transported into the cell through endocytosis. This process is facilitated by the binding of α-syn PFFs to the cell membrane through interactions with cell surface molecules. In particular, the cell surface receptor LAGS (lymphocyte activation gene 3) can bind and mediate the endocytosis of fibrillary α-syn. Additionally, α-syn fibrils can bind and cluster a number of other surface receptors at the plasma membrane. It is currently unknown whether any of these cell surface proteins can regulate the uptake of α-syn as well. Heparan sulfate proteoglycans (HSPG), abundant extracellular glycoproteins that are able to interact with a large number of extracellular proteins and ligands, are able to bind α-syn fibrils and promote their uptake. Internalized PFFs travel through the early and late endosomal compartment to the lysosome, where they are destined for degradation. Through some unknown process, α-syn PFFs can escape the lumen of the endosomal compartment and template the misfolding of soluble endogenously expressed α-syn in the cytoplasm. (??) indicates additional mechanisms and molecular players.

### IX. Developing a Human SN Brain-Chip.

Embodiments of a Brain-Chip, such as a SN Brain-Chip, may be related to Organ-Chip designs.²⁵ In some embodiments, an organ-chip has two microfluidic channels fabricated from polydimethylsiloxane (PDMS) elastomer separated by a thin (50 μm) PDMS membrane containing multiple pores (7 μm diameter, 40 μm spacing). Each channel has dedicated inlet and outlet ports for the inoculation of cells. In some embodiments, cells are maintained under precisely controlled laminar flow applied independently to each channel (FIG. 30A). Moreover, inlet ports may be used for adding inflammatory modulators, e.g. TNF-alpha, etc., with outlet ports allowing the sampling of effluent fluids.

A membrane separating two channels of one embodiment of a brain chip is coated on both sides with a tissue-specific ECM cocktail, e.g., optimized for a Brain-Chip to contain collagen type IV, fibronectin, and laminin.

One brief example of methods for seeding cells follows. First (Day (D) 0), the brain channel was seeded with human iPS-derived dopaminergic (DA) neurons derived from a healthy donor, as well as human primary brain astrocytes, microglia, and pericytes, at respective seeding densities, as described herein. The next day (D1), human iPS-derived brain microvascular endothelial cells (HBMECs) were seeded on the opposite surface of the membrane from neuronal cells (FIG. 30B). Glia (astrocytes and microglia) and pericytes cultured in the upper channel support the proper development and maintenance of the BBB function, as previously reported^{33,37,38}. The lower and apical channels were perfused with endothelial cell medium supplemented with 2% platelet-poor plasma-derived serum, and specific Dopaminergic Neurons Media, respectively (see Methods herein). The SN Brain-Chip was maintained for two days (D1-D2) in static culture to promote the formation of the endothelial lumen and acclimate cells to the microenvironment before switching to continuous medium flow (60 μL h⁻¹).

Double-label immunofluorescence with antibodies against Tyrosine Hydroxylase (TH) and Microtubule-Associated Protein 2 (MAP2) after 8 days in culture, revealed the vast majority of neurons as TH-positive (˜80%), confirming their identity as midbrain dopaminergic neurons (FIG. 30C).

Development of tight junctions in the endothelial monolayer in the vascular channel of the SN Brain-Chip was shown by the expression of Claudin-1, Claudin-5, Occludin, ZO-1 as well as the cell-cell adhesion protein CD31 (FIG. 30D, FIG. 30E), as described for the cerebral endothelial cells of the human blood-brain barrier^{33,39}.

The SN Brain-Chip sustained the barrier integrity for up to 8 days in culture under continuous perfusion, as assessed by low passive diffusion of dextran Cascade Blue (Mw: 3 kDa), and Lucifer yellow (Mw: 0.5 kDa), FIG. 30F. Specifically, the apparent permeability of the BBB in the SN Brain-Chip was maintained at values within a range of 1-3×10⁻⁶ cm s⁻¹ and 4-6×10⁻⁶ cm s⁻¹, for dextran (3 kDa) and luciferin yellow (0.5 kDa) respectively, evidence of the size-dependent transport across the BBB on SN Brain-Chip. Notably, the low permeability of the Brain-Chip to dextran were comparable to the previously reported in vivo values^{40,41}.

FIGS. 30A-F shows exemplary schematic depiction of one embodiment of a microfluidic human Substantia Nigra (SN) Brain-Chip having dopaminergic neurons and 4 additional cell types, and immunohistochemistry of iPS-derived brain endothelial cells cultured on 4 surfaces of the lower vascular channel, and iPS-derived dopaminergic neurons, primary human brain astrocytes, microglia and pericytes on the upper surface of the central horizontal membrane in the apical brain channel.

FIG. 30A shows a schematic depiction of one embodiment of a SN Brain-Chip of a 2-channel microfluidic Organ-Chip comprising 5 cell types. In one channel (brain channel) is a co-culture of microglia, astrocytes, dopaminergic neurons and pericytes. In an opposing channel, separated by a porous membrane, are endothelial cells (vascular channel).

FIG. 30B shows a 3D reconstruction of a confocal z-stack of fluorescent images showing the organization of five cell types in one embodiment of a SN Brain-Chip. Nuclei (blue); GFAP+(pink); pericytes (light blue); and endothelial cells stained for a tight junction protein (ZO-1: red) as shown in cross section.

FIG. 30C shows a representative image of iPS-derived dopaminergic neurons that are stained with DAPI (colored blue) MAP2 (green), TH (red), and a merged image on day 8. Scale bars: 100 μm.

FIG. 30D shows an immunofluorescence micrographs of the human brain endothelium cultured on the vascular channel of Brain-Chip for 7 days post-seeding (D8) labeled with Claudin-1 (red), Claudin-5 (cyan), Occludin (yellow), and CD31 (white). Scale bars: 100 μm. BBB integrity was observed for up to 8 days in one embodiment of a Brain-Chip.

FIG. 30E shows immunofluorescence micrographs demonstrate high levels of expression of ZO-1 (red) across the entire endothelial monolayer. Scale bars: 100 μm.

FIG. 30F shows a quantitative barrier function analysis of a five cell type Brain-Chip via permeability to 3 kDa fluorescent dextran, and 0.5 kDa Lucifer yellow crossing through the vascular to the neuronal channel on day 5 and 8 (n=6-9 independent chips). Error bars present mean±SEM.

### Dopaminergic Neurons.

Functionality of a Brain-Chip was indicated by the sustained dopamine release. Secreted levels of dopamine were accessed via enzyme-linked immunosorbent assay (ELISA) to confirm the functionality of the dopaminergic neurons in the Brain-Chip (FIG. 31A). Similarly, other cells of the co-culture in the upper channel of the SN Brain-Chip assessed on D8 of the culture, were found to express the cell-specific markers glial fibrillary acidic protein (GFAP; astrocytes), transmembrane protein 119 (TMEM119; resting microglia), and proteoglycans (NG2; pericytes) (FIG. 31B). Thus, incorporation of Dopaminergic Neurons in the Brain Channel demonstrated survival of iPS-derived dopaminergic neurons over 8 days of culture on the Brain-Chip (FIGS. 30F and 31A).

FIGS. 31A-F shows exemplary characterization of neurons and endothelial cells in one embodiment of a Human Substantia Nigra Brain-Chip.

FIG. 31A Graph shows neurotransmitter release over time between 5 and 8 days of co-culture. Neurotransmitter release is shown as ELISA results for dopamine secreted into the medium of the brain channel on days 5 and 8. (n=3 independent chips with duplicate technical replicates assayed per condition). n=6 chips. Error bars present mean±SEM.

FIG. 31B shows exemplary immunofluorescent microphotographs (left) validate the dopaminergic neurons with MAP2+(green), astrocytes with GFAP (magenta) and pericytes (red), and the DAPI (blue) for cell nuclei. Immunofluorescent microphotograph (right) validates the glia culture: astrocytes (magenta, GFAP staining), and resting microglia (yellow, TMEM119). Scale bars: 50 μm.

FIG. 31C shows exemplary immunofluorescent images of MAP2+(green); TH (red); Hoechst stained nuclei (blue) of iPS-derived dopaminergic neurons. Scale bar=10 μm.

FIG. 31D shows exemplary Iimmunocytochemical analysis of iPS-derived neuronal cultures in direct contact with astrocytes and pericytes. Specific markers were used to identify neurons (MAP-2), astrocytes (GFAP), and pericytes (NG2). Blue represents Hoechst-stained nuclei.

FIG. 31E shows exemplary immunocytochemical analysis that demonstrated endothelial monolayer tightness and brain specificity using ZO-1, GLUT-1, CD31, and Occludin markers at day 7 in culture.

FIG. 31F shows exemplary representative merged confocal image of the brain channel co-stained for iPS-derived cortical neurons (MAP2, green) and vesicular Glutamate transporter 1 (VGLUT1, red) (bar, 100 μm).

### X. Embodiments of Brain Chips are Superior to Conventional Cell Culture/Transwell Cultures.

To examine whether embodiments of a Brain-Chip, as described herein, faithfully recreates the human cortex tissue and better understand how much it differs from the conventional culture in vitro systems (transwells), RNA seq analysis was used for comparisons. Both the Brain-Chips and the Transwell cultures were seeded using the same cell composition and experimental conditions.

A. Superior Gene Expression of Cells in the SN Brain-Chip.

Transcriptomic profiling of the SN Brain-Chip shows gene expression that is closer to in vivo brain tissue than conventional 2D Cell Cultures. Moreover, gene expression in SN Brain-Chips is significantly different than in Transwell cultures of brain cells.

Expression analysis demonstrated that brain channel co-cultures of cells a Brain-Chip transition toward a more matured and/or differentiated state compared with a more proliferating state observed in conventional cell culture (CCC: Conventional Cell Culture) or Transwell cultures, neither of which have flowing fluids). Surprisingly, differential gene expression analysis showed that a brain channel of a SN Brain-Chip shows gene expression closer to in vivo brain tissue than conventional 2D Cell Cultures. More specifically, RNA seq (sequencing) data was obtained when embodiments of Brain-chips were compared to Transwell brain tissue/cell cultures.

Global RNA-sequencing (RNA-seq) profile data was obtained from embodiments of neural vascular units (NVU) constructed using Conventional Cell Culture (CCC) (n=4) (no flow) and SN Brain-Chip culture under constant flow (n=4), and human adult brain-derived SN (n=8) retrieved from the Genotype-Tissue Expression (GTEx) Portal⁴². The CCC and the SN Brain-Chip cultures were constituted by the same cell-type composition and subjected to the same experimental conditions (FIG. 32A). In other words, the same experimental protocol (all cell types included) was used for both conventional cell culture (CCC)/Transwell cultures and on-chip as a microfluidic Brain-Chip.

B. Differential Gene Expression (DGE) Analysis Between the SN Brain-Chip and Conventional 2D Cell Culture.

To select the differentially expressed (DE) genes, the following thresholds were applied: adjusted p-value<0.05 and |log 2FoldChange|>1. Out of the 38,887 genes annotated in the genome, 1316 were significantly differentially expressed, with 646 and 670 genes respectively up- and down-regulated in the SN Brain-Chips (FIG. 32A). Gene Ontology analysis utilizing the Gene Ontology knowledgebase was used to highlight the biological processes significantly enriched within these gene sets. Among the up-regulated genes in the SN Brain-Chip samples, functional gene sets were identified that significantly clustered under 669 GO terms. These functional gene sets were part of several relevant biological processes, including synaptic transmission, ion transport, metabolic and immune processes, extracellular matrix organization, cell adhesion, tissue development, and stimuli-evoked responses (FIG. 32B). FIG. 32B and FIG. 32C shows lists of biological processes identified by Gene Ontology (GO) enrichment analysis using the up- and down-regulated genes respectively resulted by the differentially gene expression analysis between SN Brain-Chip and CCC, respectively.

C. Differential Gene Expression Analysis: Brain-Chip vs. Conventional 2D Cell Culture.

The relative differences following comparisons between SN Brain-Chip or the adult SN tissue and CCC (SN Brain-Chip versus CCC and adult SN versus CCC) were assessed. This assessment was to determine which sets of genes underlie the closer similarity of the SN Brain-Chip to the adult SN tissue, as compared the CCC.

Compared to the SN Brain-Chip, the transcriptome of the CCC was enriched in genes involved in cell division, microtubule cytoskeleton organization implicated in mitosis, and cell cycle processes (FIG. 32C). These findings indicate that in the SN Brain-Chip, the cells acquire a more mature and/or differentiated state compared to the cells in the CCC, which seems to favor the cell proliferating state. These results are in line with previous studies showing that stem cell-based tissue models exhibit a higher resemblance to the biological properties of the mature tissue when developed in Organ-Chips as compared to CCC^{43,44}.

Additional DGE analysis revealed specific gene sets that may underlie a closer similarity between the SN Brain-Chip and the adult SN tissue, as compared to the CCC. Therefore, differences between the SN Brain-Chip or the adult SN tissue and the CCC (SN Brain-Chip versus CCC and adult SN versus CCC) were further evaluated. 1316 and 680 DE genes, respectively, were identified from each of the above comparisons, with 209 genes at the intersection of the two (FIG. 32D).

These 209 overlapping genes differentially expressed in SN Brain-Chip and human adult SN tissue versus the CCC, were contemplated to identify the biological processes that are exclusively enriched in SN Brain-Chip and human SN tissue.

Therefore, a Gene Ontology enrichment analysis was done in order to examine biological processes that are enriched in this gene list. As a result, the list of 209 overlapping genes was associated with 25 significant GO terms. The biological processes enriched in this gene set were associated with essential functions such as secretion, transport, as well as tissue and system development (FIG. 32D). This data indicates that gene expression patterns characterizing the primary SN brain tissue are recapitulated by the SN Brain-Chip but not by the CCC. In other words, at the transcriptomic level the SN Brain-Chip culture maintains better than the CCC patterns observed in the primary SN brain tissue.

FIGS. 32A-D shows exemplary Differentially Expressed (DE) genes and enriched gene ontology (GO) categories in SN Brain-Chip and conventional cell culture (CCC) system, as compared to the adult in vivo substantia nigra.

FIG. 32A shows schematic drawings of devices, Transwell and microfluidic brain-chips, along with a volcano plot resulting from DGE analysis between SN Brain-Chip and CCC. For the selection of the DE genes, the following thresholds were used: adjusted p-value<0.05 and |Log 2(foldchange)|>1. The identified up- (down-) regulated genes are highlighted in cyan (magenta) color respectively. Sample sizes were as follows: SN Brain-Chip, n=4, conventional cell culture system, n=4.

FIG. 32B and FIG. 32C shows exemplary list of biological processes identified by Gene Ontology (GO) enrichment analysis using the up- and down-regulated genes respectively resulted by the differentially gene expression analysis between SN Brain-Chip and CCC.

FIG. 32B shows exemplary GO Term Enrichment Biological Processes Upregulated Genes in Brain Chip.

FIG. 32C shows exemplary GO Term Enrichment Biological Processes Upregulated Genes in Conventional Cell Culture.

FIG. 32D shows exemplary DGE analysis identified up- and down-regulated genes in SN Brain-Chip compared to CCC (cyan circle), and human adult substantia nigra compared to CCC (yellow circle). Sample sizes were as follows: SN Brain-Chip, n=4, Conventional cell culture system, n=4, and adult substantia nigra, n=8 (independent biological specimens). Culture in Brain-Chips and CCC were done in parallel. Samples were collected and processed for analyses 8 days post-seeding (D8).

FIG. 32E shows exemplary SN Brain-Chip exhibits higher transcriptomic similarity to adult substantia nigra than conventional cell culture. The results of the GO terms analysis using the 209 DE genes showed 6 significantly enriched (FDR adjusted p-value<0.05) biological processes related to tissue development, response to a stimulus, biological adhesion, and cell surface receptor signaling pathway. The size of the bars indicates the fold-enrichment of the corresponding pathways.

FIGS. 33A-D shows exemplary embodiments of a microfluidic Brain-Chip that exhibits higher transcriptomic similarity to adult cortex tissue than Transwell cultures (not under flow).

FIG. 33A shows an exemplary Principal component analysis (PCA) generated using RNA-seq data generated by the samples collected from the brain channel of the Brain-Chips and transwells on days 5 and 7 in culture (n=4 per condition), as well as human brain cortex. A 2D-principal component plot is shown with the first component along the X-axis and the second along the Y-axis. The proportion of explained variance is shown for each component.

FIG. 33B shows an exemplary Quantitative analysis on the distances of the Brain-Chip or Transwell culture from Human Brain Cortex on days 5 and 7 of culture.

FIG. 33C shows an exemplary Differential Gene Expression (DEG) analysis identified up- and down-regulated genes in the Brain-Chip compared to conventional cell cultures (blue circle), and human adult cortex brain tissue compared to conventional cell cultures (yellow circle). Gene lists summarized in the Venn diagram are provided in Extended Data. Sample sizes were as follows: Brain-Chip, n=4, transwells n=4, and adult cortex tissue, n=8 (independent biological specimens). Culture in Brain-Chips and conventional cell cultures were done in parallel.

FIG. 33D shows an exemplary Curated heatmap generated to examine particular genes that belong to the enriched KEGG pathways and to show the expression levels log 2(FPKM) of these genes across different samples. Genes belonging to four different pathways, including: intermediate filament cytoskeleton organization, neuronal action potential, axon guidance, and extracellular matrix organization, are shown. Each heatmap has its own color scale, which corresponds to a different range of log 2 (FPKM) values, as indicated on the color bars located to the left.

Further analysis of the differences were done between the Brain-Chip or the human cortex tissue and the conventional cell cultures (Brain-Chip versus conventional cell cultures and human cortex tissue versus conventional cell cultures). Samples of RNA were taken from the brain channel of the brain-Chip and Transwell cultures on days 5 and 7 of culture. Principal component analysis (PCA), identified spacial distances within the three models (FIG. 33A). Quantified distances between the human brain cortex tissue and the transwells and Brain-Chips surprisingly showed that the Brain-Chip on days 5 and 7 in culture have a much closer transcriptomic profile to the human cortex tissue compared to that of the conventional cell culture system (transwell) (FIG. 33B). 2100 and 128 DE genes, respectively, from each of the above comparisons, with 605 genes at the intersection of the two, showing the overlapping genes (FIG. 33C). To further examine these specific genes and pathways identified in this system which may provide a competitive advantage over the conventional culture system, curated heatmaps were generated (FIG. 33D).

The majority of differentially expressed genes were found to relate to pathways specific to essential neuronal function, structure, organization, and maturity of the human brain. Genes related to the intermediate filament cytoskeleton organization, whose regulation is crucial for neuronal cell function and associated with neuronal dysfunction were altered in expression. Upregulation of genes were observed involved with neuronal action potential and axon guidance pathways, both of which play a central role in cell-to-cell communication. Also, upregulation of genes involved in the extracellular matrix organization, affecting virtually all aspects of nervous system development and function were observed. These noteworthy findings demonstrate that the cortical Brain Tissue is more closely recapitulated by the Brain-Chip than the conventional cell cultures. Cumulatively, these data demonstrated an increased similarity in the global gene expression profile between Brain-Chip and human adult cortex tissue, compared to that of Transwell cultures.

D. Establishment of an αSyn Fibril Model in the SN Brain-Chip.

Loss of substantia nigra (SN) dopaminergic neurons of the substantia nigra, part of the brain basal ganglion, leads to Parkinson's disease. Associated with this loss are accumulations of aSyn. Although therapeutics must cross the BBB to reach these affected areas, there is little known about the relationship between the BBB and aSyn pathology in PD or other neurodegenerative brain disorders and diseases. Research on PD-associated BBB impairment in a cell culture system is done using conventional cell culture systems with a short static incubation time of culturing endothelial cell lines with aSyn. However, such models lack many features of the human brain microenvironment in PD so are of limited value. Despite microfluidic models of PD reported the in the literature, none includes the cellular components of the brain's neurovascular unit for accurate modeling complex neurodegenerative diseases such as PD.

FIGS. 55A-B shows exemplary schematic depictions of a comparison of cellular interactions contemplated to provide an intact BBB (healthy) in FIG. 55A vs. contemplated cellular interactions in the brain resulting in breakdown of BBB in Parkinson's Disease. aSyn can be taken up to form inclusions in microglia, pericytes and astrocytes.

FIG. 56 shows exemplary schematic depictions of a model of aSyn actively secreted or released by dying neurons, e.g. neuron 1, into the extracellular space. Extracellular aSyn can then activate surrounding astrocytes and microglia, eliciting glial pro-inflammatory activity. Upon activation microglia produce pro-inflammatory cytokines, nitric oxide, and reactive oxygen species, which may be toxic to neurons. aSyn can be directly transferred between neurons, e.g. neuron 1 to neuron 2, and so on, leading to propagation of an aberrant aSyn aggregation process.

There was a question as to whether one embodiment of a SN Brain-Chip would respond to abnormal, toxic protein species similar to those found in synucleinopathies, as reflected in disease-relevant endpoints. Altered aSyn may act as a nucleation molecule for causing additional aSyn aggregation. Accumulation of extracellular aberrant α-synuclein (aSyn) fibrils within cells induces phosphorylation of endogenous alpha synuclein at residue S129 in a time-dependent manner. To this end, αSyn fibrils were obtained for use in testing because these fibrils are found in Lewy bodies and were shown to exert toxicity in DA neurons^{45,46}.

An exemplary experimental method and timeline is shown in FIG. 57A. Thus, the capability of exogenously added fibrils was accessed for accumulation and processing cells of a SN Brain-Chip. Recombinant αSyn fibrils (4 μg/mL) was added in the culture medium of the brain channel under continuous flow, on Day 2 of the culture (FIG. 57A).

After three- and six-days upon-exposure to αSyn fibrils (D5 and D8 of the experiment, respectively), the exposed chips underwent immunostaining in order to determine the abundance of phosphoSer129-alph α-synuclein (phospho-αSyn129), a post-translational modification characteristic of pathogenic αSyn species^{47,48}. These results show that exposure of the SN Brain-Chip to αSyn fibrils was sufficient to induce phosphorylation of αSyn in a time-dependent manner (FIG. 57B, FIG. 57C, and FIG. 58A).

Induction of phospho-αSyn129 was surprisingly evident following exposure to αSyn fibrils, as the same amount of αSyn monomer or PBS did not lead to the induction of detectable phospho-αSyn129 in the culture. Immunofluorescence staining verified phospho-αSyn129 accumulation within the TH positive neurons in the SN Brain-Chip (FIG. 57D).

FIGS. 57A-D shows exemplary pathological αSyn accumulation in the brain channel was observed following exposure to human αSyn fibrils.

FIG. 57A shows exemplary schematic depiction of one embodiment of an Experimental design for assessing the effects of αSyn toxic aggregates (fibrils) in the SN Brain-Chip, including the seeding in the Brain-Chip, the timeline for medium changes, as well as sampling times.

FIG. 57B Immunofluorescence micrographs show the accumulation of phosphorylated αSyn (green, phospho-αSyn129 staining; blue, DAPI) at day six post-exposure (D8). Pathology is absent in the brain channel following exposure to monomer or PBS. Scale bars: 100 μm.

FIG. 57C Quantitative analysis of fluorescence intensity in each group at day three and six post-exposure (D5 and D8, respectively).

FIG. 57D Immunofluorescence staining shows phospho-αSyn129 (green) accumulation within the TH (red) positive neurons in the SN Brain-Chip. yellow indicates co-localization of phospho-αSyn129 and TH. Statistical analysis is two-way ANOVA with Tukey's multiple comparisons test (n=3˜4 independent chips with 3˜5 randomly selected different areas per chip, *P<0.05, ****P<0.0001 compared to monomeric or PBS group). Error bars present mean±SEM.

FIGS. 58A-B shows exemplary accumulation of phosphorylated αSyn and mitochondrial impairment in the αSyn fibril model at day 5.

FIG. 58A shows exemplary assessment of phosphorylated αSyn resulting from a three day post-exposure to αSyn fibrils shown in lower row of panels. Immunofluorescence micrographs show the accumulation of phosphorylated αSyn (green, phospho-αSyn129 staining-vertical middle panels; blue, DAPI stained nuclei-vertical left panels) and merged images-vertical right panels. PBS treated controls upper row of panels, αSyn monomer treated middle row of panels. Scale bars: 100 μm.

FIG. 58B shows exemplary effects of αSyn fibrils on mitochondrial membrane potential at three days after exposure. Mitochondrial membrane potential assessed by JC-1 staining on the brain side. Dual emission images (527 and 590 nm) represent the signals from monomeric (green) and J-aggregate (red) JC-1 fluorescence. Scale bars: 100 μm.

E. Effects of αSyn Fibrils in Mitochondria and ROS Production in the SN Brain-Chip.

Some evidence indicates a role of mitochondrial dysfunction and increased reactive oxygen species in the development of neurodegenerative diseases, including sporadic PD^{31,49}. To assess the mitochondrial membrane potential in the cells in the SN Brain-Chip, JC-1, a staining probe for the detection of mitochondrial damage was used. JC-1 in the form of a green monomer enters the cytoplasm and accumulates in the healthy mitochondria, where it forms numerous red J-aggregates. The transition of fluorescence signal from red to green indicates loss of mitochondrial membrane potential, as in cases of significant mitochondrial damage⁵⁰. Exposure to αSyn fibrils led to a lower intensity of red and increased of the green fluorescence, in a time-dependent manner. Reduction in the red-to-green fluorescence intensity ratio was found in the αSyn fibrils-exposed SN Brain-Chip and not following exposure to the monomeric αSyn species (FIG. 59A, FIG. 59B and FIG. 58B). Red fluorescence indicated normal mitochondrial membrane potential, whereas green fluorescence indicated damage to mitochondrial membrane potential. aSyn fibrils caused mitochondrial impairment in a time-dependent manner, as evidenced by both reduced red fluorescence (normal mitochondria) and increased green fluorescence (damaged mitochondria).

Therefore, data demonstrated that the number of caspase 3-positive apoptotic dopaminergic neurons was increased on day 8 post exposure to aSyn fibrils compared to monomeric aSyn.

The intracellular ROS levels in the brain channel of the SN Brain-Chip was measured on day 8 of the culture, using CellROX reagent. As shown (FIG. 59C, FIG. 59D), exposure to αSyn fibrils led to a significant increase in ROS production, as compared to the exposure to αSyn monomers.

FIGS. 59A-D shows exemplary reduction of mitochondrial activity and increase in ROS production in the αSyn fibril model.

FIG. 59A shows exemplary mitochondrial membrane potential assessed by JC-1 staining in the brain side at day six post-exposure. Dual emission images (527 and 590 nm) represent the signals from monomeric (green) and J-aggregate (red) JC-1 fluorescence. Scale bars: 100 μm.

FIG. 59B Quantitative analysis of the ratio of Red/Green fluorescence intensity in each group at day three and six post-exposure (D5 and D8, respectively). Statistical analysis is two-way ANOVA with Tukey's multiple comparisons test (n=3 independent chips with 3-4 randomly selected different areas per chip, *P<0.05, ****P<0.0001 compared to monomeric group).

FIG. 59C shows exemplary representative images of ROS levels (green, CellROX) show higher levels of intercellular ROS in the cells of the brain channel exposed to αSyn fibrils than those exposed to αSyn monomer at day six post-exposure. Scale bars: 100 μm.

FIG. 59D shows exemplary quantification of the number of CellROX-positive events per field of view in each group. Statistical analysis is Student's t test (n=3 independent chips with 3-4 randomly selected different areas per chip, ****p<0.0001 compared to monomeric group). Error bars present mean±SEM.

F. αSyn Fibrils Induce Cell Death and Neuroinflammation in the SN Brain-Chip.

Several studies showed that αSyn fibrils initiate a series of secondary processes leading to neuroinflammation, neurodegeneration, and cell death^{45,51}. Therefore, it was questioned whether the cells in the SN Brain-Chip would respond to αSyn fibrils by induction of apoptosis. Three days (experimental D5) following exposure to αSyn, either monomeric or fibrillar, no effect in cell survival was detected, as reflected by the similar percentage of live cells under these experimental conditions. In contrast, six days upon treatment (experimental D8), there was a significant reduction in live cells in the SN Brain-Chip exposed to αSyn fibrils compare to αSyn monomers or PBS (50.63±3.9 vs 91.2±1.05 vs 87.02±0.87) (FIG. 53A, FIG. 53B). Confocal immunocytochemical analysis using antibodies against microtubule-associated protein 2 (MAP2) and cleaved caspase-3 (CC3), confirmed the increase in caspase 3-positive neurons on D8 post-exposure to αSyn fibrils, as compared to those exposed to monomeric αSyn (FIG. 61A and FIG. 61B).

The extent of inflammatory response mediated by αSyn fibrils in the SN Brain-Chip was then measured. An increase of GFAP staining observed in the αSyn fibrils-exposed chips was suggestive of reactive astrogliosis (FIG. 60C) and (FIG. 60D), a known component of the brain inflammatory response.³⁶ A significant increase in ROS production in cells exposed to aSyn fibrils was observed compared to cells exposed to aSyn monomers (FIG. 59C).

aSyn fibrils caused mitochondrial impairment in a time-dependent manner, as evidenced by both reduced red fluorescence (normal mitochondria) and increased green fluorescence (damaged mitochondria)

In parallel, there was an activation of microglia, as indicated by the increase in CD68 immunoreactivity (F FIG. 60C and FIG. 60D). The observation in a Brain-Chip that aSyn fibrils induced astrogliosis, microglia activation, and IL-6 secretion was similar to findings in human PD patients.

Secreted levels of interleukin-6 (IL-6), and tumor necrosis factor-alpha (TNF-α) induced in the effluent of the neuronal channel, were significantly increased following exposure to αSyn fibrils versus monomeric αSyn (FIG. 60E and FIG. 60F).

FIGS. 60A-F shows exemplary αSyn-induced caspase-3 activation and neuroinflammation.

FIG. 60A shows exemplary representative merged images showing double immunostaining for MAP2 (grey) and Cleaved Caspase-3 (red, CC3) in the brain channel at six-days post-exposure. Scale bars: 50 μm.

FIG. 60B shows exemplary quantitative data on the number of CC3 positive neurons. Statistical analysis is Student's t test (n=3 independent chips with 3-4 randomly selected different areas per chip, ***p<0.001 compared to monomeric group).

FIG. 60C shows exemplary immunostaining of the astrocyte marker GFAP (magenta) demonstrated activation of astrocytes at day 8 following exposure to αSyn fibrils compared to monomeric αSyn. Scale bar, 100 μm.

FIG. 60D shows exemplary immunostaining of the microglial CD68 (red) demonstrated activation of astrocytes and microglia at day 8 following exposure to αSyn fibrils compared to monomeric αSyn. Scale bar, 100 μm.

FIG. 60E shows exemplary secreted levels of TNF-α in the αSyn fibril model. Statistical analysis was by Student's t-test (n=6-7 independent chips, **p<0.01).

FIG. 60F shows exemplary secreted levels of proinflammatory cytokine IL-6 in the αSyn fibril model. Statistical analysis was by Student's t-test (n=4˜7 independent chips, ****p<0.0001). Error bars present mean±SEM.

FIGS. 61A-D shows exemplary αSyn-induced cell death and neuroinflammation.

FIG. 61A shows exemplary cell viability (live/dead) assay following exposure to human αSyn fibrils. Live/Dead cell staining assay was designed to test the potential cytotoxicity of αSyn fibrils at days 5 and 8 of culture. Scale bars: 100 μm.

FIG. 61B shows exemplary data are expressed as the average live cells/total number of cells (sum of calcein AM positive and ethidium homodimer positive cells). Statistical analysis is two-way ANOVA with Tukey's multiple comparisons test (n=3 independent chips with 3˜5 randomly selected different areas per chip, ****P<0.0001 compared to monomeric or PBS group, NS: Not Significant).

FIG. 61C shows exemplary quantification of the number of GFAP-positive events per field of view. Statistical analysis is Student's t test (n=3 independent chips with 3˜4 randomly selected different areas per chip, ***p<0.001 compared to monomeric group). FIG. 61D shows exemplary quantification of the number of CD68-positive events per field of view. (n=3 Brain-Chips with 3˜5 randomly selected different areas per chip, ****P<0.0001 compared to the monomeric group). Error bars present mean±SEM.

G. Blood-Brain Barrier Disruption in αSyn-Associated PD Pathology.

As there is evidence of extraneuronal manifestations of PD, attention is drawn to the effects of the disease on the BBB. Measurable levels of αSyn were identified in the brain and in the systemic circulation. Therefore they are implicated in disease onset and/or progression. The origin of peripheral αSyn remains a subject of discussion, as well as the possibility that αSyn crosses the BBB in either direction¹². Recent data suggests there is BBB dysfunction in PD as in other neurodegenerative diseases and that it may have a role in the pathogenesis and progression of PD⁵². Thus, BBB permeability assays were measured on the SN Brain-Chip after exposure to αSyn fibrils, as compared to exposure to αSyn monomers or PBS. A model for measuring BBB breakdown is shown schematically in FIG. 62.

FIG. 62 shows an exemplary schematic model for measuring BBB breakdown. In one embodiment modeling neuroinflammation when a-Syn is added to the brain channel. In one embodiment modeling systemic inflammation when a-Syn is added to the vascular channel.

Data indicate significantly increased permeability to 160 kDa Immunoglobulins (IgG), 3 kDa dextran, and 0.5 kDa lucifer in the brain channel of the SN Brain-Chip six days after exposure to αSyn fibrils (FIG. 5, 6, and FIGS. 64A-B). Thus, aSyn fibrils significantly increased the paracellular permeability of BBB in a time-dependent manner.

Further, accumulation of aSyn in endothelial cells is accompanied by loss of tight junction formation compared to the aSyn monomeric group (FIG. 51B). Therefore, endothelial cells represents a new pathogenic mechanism and contemplated as a new drug target for therapeutic intervention in PD.

To further characterize the endothelium in the SN Brain-Chip model and to determine whether the exposure to αSyn fibrils leads to transcriptomic changes in these cells, RNA-seq analysis was used. This analysis resulted in the identification of 1280 differentially expressed genes, either significantly up- or down-regulated.

Principal components analysis (PCA) revealed differences in the transcriptome profiles between the two conditions, αSyn fibrils and αSyn monomers (FIG. 63C). This analysis resulted in the identification of 1280 DE genes, either significantly up-regulated (739 genes) or down-regulated (541 genes) (FIG. 63D) in the αSyn fibril-exposed SN Brain-Chips.

FIGS. 63A-D shows exemplary Blood-Brain Barrier dysfunction in the αSyn fibril model.

FIG. 63A and FIG. 63B shows exemplary quantitative barrier function analysis via permeability to 0.5 kDa lucifer yellow and 3 kDa fluorescent dextran at day 5 and 8 following exposure to αSyn fibrils or αSyn monomers. Statistical analysis is two-way ANOVA with Tukey's multiple comparisons test (n=6˜9 independent chips, ****P<0.0001 compared to monomeric group, NS: Not Significant).

FIG. 63C shows exemplary principal component analysis generated using the RNA-seq data generated by the samples collected from the vascular channel of the SN Brain-Chip upon exposure to αSyn monomers or αSyn fibrils (n=4 per condition). A 2D-principal component plot is shown with the first component along the X-axis and the second along the Y-axis. The proportion of explained variance is shown for each component.

FIG. 63D shows exemplary volcano plot indicating DE genes between αSyn fibrils and αSyn monomers, as identified by the RNA-sequencing analysis. For the selection of the DE genes the following thresholds were applied: adjusted p-value<0.05 and |Log 2(foldchange)|>0.5. The identified up- (down-) regulated genes are highlighted in cyan (magenta) color. Sample sizes were as follows: Brain-Chip (αSyn monomers), n=4, Brain-Chip (αSyn fibrils), n=4.

This set of 1280 DE genes includes several genes that were implicated in BBB dysfunction in a number of diseases⁵³ in addition to new genes. Multiple members of specific gene families were up-regulated, such as extracellular proteases of the Serpin family (SERPINA1), collagens (COL3A1), centromere proteins (CENPE), and kinesins (KIF15). In addition, multiple new genes in the αSyn fibrils-exposed chips implicated in cellular processes were associated with PD pathogenesis (Table 6 and FIG. 64B) such as autophagy, oxidative stress, mitochondrial function, inflammation, and vesicular trafficking, highlighting the potential for brain endothelial cells to contribute to molecular mechanisms and functional deficits in PD. Examples of PD associated genes include leucine-rich repeat kinase 2 (LRRK2)⁵⁴, synphilin-1 (SNCAIP)⁵⁵, monoamine oxidase A (MAOA)⁵⁶, complement 5 (C5)⁵⁷, and apolipoprotein A-1 (APOA1)⁵⁸. BBB-related genes with altered expression are low-density lipoprotein receptor-related protein 1B (LRP1B)⁵⁹ and ATP-binding cassette (ABC) transporters (ABCB1)⁶⁰. The upregulation of the LRP1 gene is consistent with previous findings, where dysfunction of LRP1B was associated with PD⁶¹. Further, positive and negative associations between specific ABCB1 haplotypes associated with P-glycoprotein activity and PD incidence were reported.

Unexpectedly, endothelial genes down-regulated upon exposure to αSyn fibrils included the tight junctions claudin gene family (CLDN1, CLDN4, and CLDN9), and the gap junction protein alpha 4 (GJA4).

This analysis resulted in the identification of 1280 differentially expressed genes, either significantly up- or down-regulated

Additional changes in aSyn transporter molecules for transporting, e.g. trafficking of α-syn, are contemplated in the endothelial cells of SN Brain-Chips, e.g. exposed to α-syn variants, overexpression of α-syn, etc., in SN Brain-Chips and SN Brain-Chips compromising cells derived from PD patients. Thus, treatments that prevent vascular degeneration are contemplated to improve vascular remodeling in the brain and provide a novel target to ameliorate the disease burden in PD.

FIGS. 64A-B shows exemplary Blood-Brain Barrier dysfunction in the αSyn fibril model. IgG Penetration through BBB. See, Table 5.

FIG. 64A shows exemplary quantitative barrier function analysis via permeability to immunoglobulin G (IgG1) at day 5 and 8 following exposure to αSyn fibrils, αSyn monomers or PBS. Statistical analysis is two-way ANOVA with Tukey's multiple comparisons test (n=5˜8 independent chips, ****P<0.0001 compared to monomeric group, NS: Not Significant). Error bars present mean±SEM.

FIG. 64B shows exemplary selection of the 739 up-regulated and 541 down-regulated genes identified after performing DGE analysis between αSyn fibrils and αSyn monomers. The size of the bars indicates the log₂(Fold-Change) of the corresponding gene expressions and the colors the statistical significance (FDR adjusted p-values) of the corresponding changes.

In some embodiments, 739 up-regulated and 541 down-regulated genes identified after performing DGE analysis between αSyn fibrils and αSyn monomers may be used as biomarkers in microfluidic Brain-Chips. In some embodiments, proteins expressed from 739 up-regulated and 541 down-regulated genes identified after performing DGE analysis between αSyn fibrils and αSyn monomer may be used as biomarkers in microfluidic Brain-Chips.

As examples, APOA1, apolipoprotein A1, Adenosine Triphosphate ATP-Binding Cassette (ABC) transporter 1 (ABCB1) are potential biomarkers for PD in microfluidic Brain-Chips along with other biomarkers listed in Table 1. Mutations in genes encoding leucine-rich repeat kinase 2 (LRRK2) and α-synuclein are associated with both autosomal dominant and idiopathic forms of Parkinson's disease (PD) so that neurons comprising LRRK2 induced or endogenous mutations may be tested for recovery of BBB permeability in the presence of a-Syn treated cells and a test compound. LRRK2 co-localizes with the α-synuclein inclusions, and knocking down LRRK2 increases the number of smaller inclusions in other in vitro models.

GJA4 (Gap Junction Protein Alpha 4) expressed by brain neurons and glial cells as Cx37 protein was downregulated in a SN Brain-Chip so when present may be also be used as a biomarker for healthy microfluidic Brain-Chips. Alternatively as this marker is lost it may be indicative of degenerating cells in the brain channel of a Brain-Chip.

H. Exemplary Evaluation of Exposing a Brain-Chip to a Test Compound for Potential Use as a Treatment Compound for a Neurodegenerative Disease.

Several models show contemplated drug targets present in α-syn treated co-cultures depicting neuronal dysfunction and cell death, FIG. 66A. Contemplated microglial activation and immunological targets are shown in FIG. 66B. Contemplated trafficking targets are shown in FIG. 66C.

Merely for an example of a method of use of a SN Brain-Chip exposed to α-syn preformed fibrils (PFFs) for test compound evaluation, the following is an exemplary method of use, e.g. trehalose as an exemplary autophagy modulator, for potential use as a treatment for a disease, such as PD, as described herein. See FIG. 67 for contemplated modeling of α-syn and α-syn preformed fibrils (PFFs). In one embodiment, an exemplary test compound for rescuing BBB permeability induced by a-Syn fibrils is trehalose. Trehalose refers to a non-reducing disaccharide (O-α,-d-glucopyranosyl-[1→1]-α-d-glucopyranoside) with two glucose molecules linked through an α, α-1,1-glucosidic bond. Trehalose also refers to a natural disaccharide that is considered a candidate for the treatment of neurodegenerative diseases. Trehalose has a chaperone-like activity for preventing/reducing protein misfolding or aggregation, i.e. protein stabilization, and by promoting autophagy of misfolded or aggregates for contributing to the removal of accumulated proteins and anti-neuroinflammatory effects. See, FIG. 67.

FIG. 67 shows an exemplary schematic diagram contemplating a-Synuclein fibrils (PFF) (red circles) recruiting endogenous a-Synuclein (aSyn) (yellow circles) to form aggregates and induce neuron death. At least some aggregates are released and propagate to neighboring cells and further pathological damage to the brain. Enhanced lysosomal efficiency/hydrolytic capacity through increased Cathepsin D or enhanced autophagosome production through trehalose treatment may promote the sequestering and degradation of toxic aSyn species. Redmann et al., Aging and Disease, 2016.

As described and shown herein, embodiments of a SN Brain-Chip are contemplated for use to assess novel treatments that protect from vascular dysfunction or improve vascular remodeling in the brain. To this purpose, a SN Brain-Chip was tested to see whether the disrupted BBB on the SN Brain-Chip in the αSyn-fibril model could be restored by a therapeutic agent inducing autophagy, thereby clearing accumulated αSyn protein aggregates, i.e. targeting the autophagy pathway. Autophagy refers to a lysosome-mediated degradation process to remove damaged cellular components. These include damaged organelles, such as mitochondria, endoplasmic reticulum (ER), and peroxisomes, as well as misfolded or aggregated proteins and intracellular pathogens. At least three types of autophagy are known, macroautophagy, chaperone-mediated autophagy (CMA), and microautophagy.

Recent reports of trehalose, a disaccharide approved by FDA, have shown beneficial effects against the accumulation of neurotoxic, aggregated proteins, and neurodegeneration⁶². To date, the effects of trehalose were evaluated in vitro by neuronal cell lines and animal models^{63,65,66}. One study of Hoffmann et al. provided evidence that trehalose prevents or halts the propagation of αSyn pathology by targeting lysosomes⁶³. Studies in aged mice suggest that oral supplementation of the autophagy-stimulating disaccharide trehalose restored vascular autophagy and ameliorated age-related endothelial dysfunction⁶⁴. On the basis of these results, it was contemplated that trehalose may disturb lysosome integrity and its function, which might subsequently hinder BBB disruption induced by αSyn fibrils.

The results reported herein, unexpectedly demonstrate that brain pathology induced by αSyn fibrils may drive BBB dysfunction. Further, a treatment for reducing αSyn fibril accumulation will improve or rescue this αSyn fibril induced BBB dysfunction.

FIG. 68A shows an exemplary timeline for methods of use in testing therapeutic test compounds, such as trehalose. Trehalose was added to the SN Brain-Chip, via the brain channel on experimental day 5 (three days after adding αSyn fibrils). As shown herein, differential gene expression data shows downregulation of CLAUDIN and other tight junction proteins induced when brain cells are exposed to αSyn fibrils.

FIG. 68A shows an exemplary timeline for methods of use in testing therapeutic test compounds.

FIG. 68B shows exemplary morphological analysis of tight junctions in endothelial cells in the αSyn fibril model with or without trehalose treatment. The reduction of ZO-1 by αSyn fibrils and restoration of junction protein expression of ZO-1 was visualized by immunofluorescence staining with a ZO-1 antibody. Scale bars: 50 μm.

After 72 hours (experimental D8), BBB permeability was assessed by introducing 3 kDa dextran into the vascular channel. Trehalose-treated αSyn fibril damaged SN Brain-Chips showed significantly decreased BBB permeability (FIG. 62A) rescuing damaged tight junctions (FIG. 68B and FIG. 69B). Thus trehalose, at a 10 mM dose, diminished the accumulation of αSyn within the cells of the brain channel when compared to cells exposed to αSyn fibrils alone.

FIGS. 69A-B shows an exemplary effect of a test compound as an autophagic inducer, trehalose on BBB integrity.

FIG. 69A shows an exemplary quantitative barrier function analysis via permeability to 3 kDa fluorescent dextran at day 8 in the αSyn fibril model with or without trehalose treatment. Statistical analysis is Student's t test (n=5˜8 independent chips, ****P<0.0001 compared to monomeric group, ***P<0.001 compared to αSyn fibrils). Error bars present mean±SEM.

FIG. 69B shows an exemplary morphological analysis of tight junctions in endothelial cells in the αSyn fibril model with or without trehalose treatment. The junction protein expression of Claudin-5 was visualized by immunofluorescence staining with a Claudin-5 antibody and DAPI for cell nuclei. Scale bars: 50 μm.

In summary, a novel microfluidic SN Brain-Chip comprising human brain cells was developed to recapitulate the complex neurovascular unit found in vivo by recreating the vascular-neuronal tissue interface in vitro.

A microfluidic SN Brain-Chip described herein successfully recreates certain aspects of idiopathic PD after the exogenous administration of α-syn fibrils, and serves as a well-controlled platform to understand and test physiological and pathological mechanisms of BBB dysfunction in PD. In some embodiments, recreate several pathological hallmarks observed in PD patients: accumulation of Alph α-synuclein; Mitochondrial dysfunction; ROS production; neuroinflammation; neuronal death; and BBB dysfunction. Surprisingly, α-syn dysregulation within the Brain channel, i.e. neuronal area unexpectedly dysregulated endothelial cells as shown herein by a loss of barrier function of the BBB. Therefore results demonstrated herein provide unexpected evidence of cross-talk between the neuronal compartment and the endothelial cells.

Embodiments of microfluidic Brain-Chip models are contemplated to enable the identification of biomarkers for α-syn regulation related to neuronal dysfunction. Embodiments of microfluidic Brain-Chip models are contemplated to enable the discovery of new targets of significant value for use in testing new compounds. Additionally, embodiments of microfluidic Brain-Chip models are contemplated to enable translation of findings from other systems testing new compounds in preclinical development.

TABLE 6
Identification of multiple genes in the alpha-Syn fibrils
condition implicated in a variety of cellular processes
contemplated as biomarkers. See, FIG. 57B.

| Biological Functions | Genes | Regulation |
|---|---|---|
| Endothelial active efflux | ABCB1 | Up |
| Lipoprotein receptors | LRP1B, LRP2, APOA1 | Up |
| Solute carrier-mediated | SLC16A6, SLC2A6 | Down |
| transport | | |
| Tight junctions | CLDN1, CLDN4, and CLDN9 | Down |
| Gap junctions | GJA4 (commonly, Cx37) | Down |
| Mitochondrial oxidation | MAOA | Up |
| Inflammation | C5, IL1R1, SERPINA1 | Up |
| Autophagy and proteasome | LRRK2, SNCAIP | Up |
| system | | |
| Vesicular trafficking | CENPE, KIF15 | Up |

### XI. Brain-Chip in Space.

Modelling disease states, e.g. autoimmune diseases and diseases associated with neuronal inflammation in Space could enable us to improve human health on earth. As such, compositions and methods described herein, may also be done during space flight or on a space station. In such embodiments, additional variables such as acceleration, deceleration, micro-gravity and zerogravity may be evaluated, in particular for induction of inflammation and resolution of inflammation under nonsurface level gravity conditions. Readouts are contemplated as described herein. In some embodiments, methods are conducted within Space Tango's CubeLab.

### XII. Summary

PD is characterized by an array of premotor and CNS symptoms, together with degenerative changes in the SN, which expand to more brain areas as the disease advances. Pathology findings reveal the existence of the characteristic Lewy bodies, proteinaceous aggregates containing αSyn1-3. Experimental models have significantly contributed to comprehension of the pathogenesis of PD and other synucleinopathies, by demonstrating aspects of αSyn biology, such as intracellular aggregation and neuronal death67. Despite the strong experimental and clinical evidence, the course of events driving the detrimental pathology in synucleinopathies remains unknown, as the diagnosis of the disease usually is made in later stages when the damage has advanced. Further, the existing animal models are limited in their relevance to human disease in terms of disease induction and progression in time. Given the complexity of the etiology and progress of synucleinopathies and the lack of in vivo models representative of the human disease, there is an urgent need for human cell-based models able to uncover the cell-cell interactions driving the tissue pathology.

To address this need, an engineered human microphysiological system was developed as described herein in order to capture the dynamic interactions in the human neurovascular unit, composed of iPSCs-derived brain endothelium and dopaminergic neurons, and primary astrocytes, pericytes, and microglia. A semipermeable membrane, coated with tissue-relevant ECM, separates the endothelial from the parenchymal cells cultured independently in specific media, under continuous medium flow. This setup is amenable to imaging, and it enables frequent sampling of the effluent from either side of the membrane for assessment of barrier permeability, and characterization of the secretome at different time points. Exposure of the SN Brain-Chip to αSyn fibrils led to progressive accumulation of phosphorylated αSyn and the associated induction of specific aspects of αSyn toxicity, such as mitochondrial dysfunction and oxidative stress. Compromised mitochondrial function, as reflected in mitochondrial complex I levels and development of oxidative stress, are central contributors in the neurodegenerative process in PD49. Additionally, exposure of the SN Brain-Chip to αSyn fibrils, resulted in microglia activation, astrogliosis, and a time-dependent neuronal loss, as was described in PD patients36. The microfluidic-based, controlled microenvironment of the SN Brain-Chip may underlie the gradual development of αSyn fibrils-induced toxicity. Another contributing factor might be that the cells on the Brain-Chip transition towards a more mature state, recapitulating aspects of the brain responses that have not been captured by the conventional. cell culture systems. This hypothesis is supported by this transcriptomic data, and is in agreement with reports showing that maturation of neurons/astrocytes promotes the propensity of αSyn aggregation46,68.

The first link between synucleinopathies and inflammation was provided by findings on activated microglia in the SN of PD patients69. Further, marked upregulation of TNF-α and IL-6 mRNA levels was found in the SN of MPTP-treated animals compared to controls70 as well as increased levels of inflammatory mediators in brain tissue from PD patients71,72, Similarly activated microglia and increased levels of secreted cytokines was detected in the SN Brain-Chip effluent following exposure to αSyn fibrils. Although microglia are drivers of the neuroinflammatory responses that propagate the neuronal cell death in PD, additional role(s) for microglia in the progress of synucleinopathies have been suggested73. Therefore, embodiments of SN Brain-chip provides unprecedented opportunities to identify the exact interactions between microglia and other CNS cell types and how they could be targeted to modify the spread of αSyn pathology. A potential caveat of the current design is the lack of recruited peripheral immune cells, a component of the disease. The perfusion capacity of this platform may be leveraged in the future to model the recruitment of disease-relevant immune cell subsets across the BBB, similar to previous reports74.

BBB dysfunction was recently increasingly viewed as an inherent component of PD progression10-13. In PD animal models, including MPTP-treated mice75 and 6-hydroxydopamine (6-OHDA)-treated rats14, BBB disruption has also been found, in agreement with the clinical data. Additional studies have suggested that αSyn deposition increases BBB permeability76 and PD development77. Despite the strong experimental and clinical evidence on the BBB disruption in PD, the underlying mechanisms remain unclear, whereas it is suggested that BBB involvement might even precede the dopaminergic neuronal loss in substantial nigra78. Finally, late studies on the peripheral origin of PD propose that BBB is involved in the gut-derived signaling that induces brain pathology and is considered as a likely early mechanism in PD pathogenesis79. Results show signs of tight junctions derangement and progressively compromised BBB permeability in response to αSyn fibrils. This is in line with previous studies showing deregulation of claudin as a determinant of the BBB integrity and paracellular permeability80. Transcriptomic analysis of the BBB endothelium from the SN Brain-Chip revealed that αSyn fibrils alter the expression of genes associated with distinct biological processes implicated in PD, including autophagy, oxidative stress, mitochondrial function, and inflammation. Excitingly, control over the amount of αSyn accumulation by treatment with the autophagy inducer trehalose, rescued the compromised BBB permeability and the derangement of the tight junctions, suggesting a prospective therapeutic approach for treating compromised BBB implicated in PD.

In conclusion, the development of a novel SN Brain-Chip reproduces in vivo-relevant aspects of synucleinopathies upon exposure to αSyn fibrils. The SN Brain-Chip provides a promising platform for the identification of the specific operating networks underlying this unmet medical need, including the dynamics of BBB dysfunction. Moreover, this platform may be useful to characterize the response to new PD therapies and identify associated biomarkers among different patients.

### XIII. Blood-Brain Barrier-Chip Culture Guideline.

This exemplary guideline describes a monoculture and co-culture models, including but not limited to monocultures of one cell type, wherein embodiments for co-culturing may be isolated for seeding one cell type, two cell types, three cell types, four cell types or more, e.g. adding immune cells, such as isolated PBMCs, partially purified and purified cell types, with or without preactivation prior to seeding on chip.

Brain endothelial cells can be sourced from iPSCs, primary, or immortalized human primary brain microvascular endothelial cells from different vendors and sources. Different cell sources and vendors can greatly affect the cell quality and lead to variability. We are currently working to identify a cell source that provides the highest functionality and yet is reproducible and does not lead to large variability between lots/donors. There are still a high level of inconsistency in the cell quality depending on the cell source. Culturing these cells is not currently standardized and there are no established media compositions or growth conditions. Additionally, cells from a particular donor or source can be evaluated after the cells have completely differentiated which takes 10 days for iPSCs. These issues present a challenge when working with brain endothelial cells and should be recognized before attempting this guideline. Note that success of the chips is highly dependent on the quality of the cells.

The microenvironment created within each Chip-S1 includes tissue-specific cells in the top channel and vascular cells in the bottom channel. Top and bottom channels are separated by a porous membrane that allows for cell-cell interaction like those that are seen in vivo. These two channels are fluidically independent. Cells in each channel are seeded with organ- and cell-specific extracellular matrix proteins (ECM), and can be maintained in static culture for up to four days, depending on cell type, and may also be under continuous flow of cell culture media.

Proper gas equilibration of media is essential for successful Organ-Chip culture. As the system is sensitive to bubbles, media should be equilibrated to 37° C. and excess gas removed prior to use in the chip.

Day −5 to −3: Prepare hBMEC, astrocyte and pericyte culture media and flasks. Activate the inner surface of the chip channels for proper ECM attachment. Coat inner channels with ECM proteins as described herein. For both the channels of the Blood-Brain Barrier—Chip: ECM solution: Collagen IV: 400 μg/mL Fibronectin: 100 μg/mL Laminin: 10 μg/mL

Day 0: hBMEC, Astrocytes and Pericytes to Chips. Seed hBMECs on the upper surface of the bottom channel, DMEM/F12+10% FBS for hBMECs. Invert the chips and incubate at 37° C. for 2-3 hours. Then seed astrocytes and pericytes in the top channel.

Days 2-5: Maintenance and Experiments. Media replenishment and sampling.

For transcytosis applications, it is not necessary to seed the lower surface of the bottom channel. Confirm cell attachment under a brightfield microscope. Once it was confirmed that cells have attached to the upper surface of the bottom channel (they are not expected to be forming cell-cell junctions yet), rinse the bottom channel once with hBMEC culture medium and the top channel with a DMEM/F12 10% FBS culture medium. Hereafter, we will refer to this media as “top channel medium”.

### IVX. Innervated Intestine (Gut)-Chip.

Intestinal homeostasis and barrier function are maintained by a continuous interaction between epithelial enterocytes, enteric neurons and glia, and immune cells in close proximity with each other. In addition, the GI immune system is called into operation whenever the mucosa is affected by microbial infection, allergen exposure, inflammation and other types of injury. Thus, inflammation that disrupts barrier function in the gastrointestinal tract provides a system for evaluating microfluidic intestine-chips innervated with sensory neurons as a platform for in vivo relevant responses to neuroinflammation with and without enteric infections. Intestine includes small intestine and large intestine, e.g. colon.

Embodiments of microfluidic Innervated Intestine-Chip model incorporate epithelial cells, immune cells, iPSC-derived sensory neurons, and human large intestinal microvascular endothelial cells showed closer characteristics and function over other intestinal organ models. Barrier function evaluated by apparent permeability is maintained and specific cellular markers for nociception are maintained by neurons. Immune cell viability was maintained and incorporation of differentiated macrophages was successful within the chip. In summary, findings described herein, demonstrate the optimization and functionality of an innervated Intestine-Chip to develop gastrointestinal (GI) disease and infection for investigating associated responses of the human immune system.

In one embodiment, an Intestine chip is seeded with epithelial cells including but not limited to primary epithelial cells, iPSC derived cells, biopsy derived cells, cell lines, commercial sources of cells, etc.

In one embodiment, a Caco-2 Intestine-Chip model, human microvascular endothelial cells are seeded in the bottom compartment and Caco-2 epithelial cells in the top compartment to emulate intestinal function including dynamic stretch and continuously flow producing shear forces and nutrients/waste cycling. In another embodiment, an innervated Intestine-Chip as described herein, further comprises resident immune cells and sensory neurons are incorporated in a gel in between the intestinal epithelial cells and the chip membrane, to recapitulate intestinal lamina propria.

In one embodiment, as an exemplary capture of the interaction between immune cells and sensory neurons of the GI Tract, macrophages and iPSC-derived Sensory Neuron Progenitors will be embedded in a matrix microenvironment layer within the chip (plate images shown). Caco-2 intestinal epithelial cells establish a tight barrier by day 3 and maintain a villi-like morphology under the presence of continuous flow and stretch. The bottom channel of the chip is seeded with Human Large Intestinal Microvascular Endothelial Cells (cHIMECs) to model the vascular endothelium.

ECM Characterization and Immune Cells embedded within the Chip. Optimization of the extracellular matrix (ECM) layer established within the chip was assessed by immunofluorescent staining. Optimized ECM conditions were then used for embedding immune cell types within the chip for further experiments.

Immune Cell Co-culture Optimization and Characterization. To model the intestinal immune response, both CD4 T-cells and macrophages were embedded in the Innervated Intestine-Chip. Immune cell proliferation was evaluated by fluorescent dilution of labelled cells over 14 days in the presence of continuous flow and when co-cultured with epithelial cells. FACs analysis confirmed the differentiation of macrophages from blood-derived monocytes.

Optimization of iPSC-derived Sensory Neuron Progenitors. Incorporation of the neuronal component on the Innervated Intestine-Chip has included optimization of different extracellular matrix coating compositions and varied seeding density. We have established new methods to culture iPSC-derived sensory neurons differentiation within the chip.

A sensory neuron biomarker, e.g. nociception specific marker, TRPV1, was expressed on the Innervated Intestine-Chip by day 7. Positive immunofluorescent staining for a marker of sensory neuron differentiation indicates maturation and functionality is present within the chip.

In one embodiment, an exemplary innervated Intestine-Chip model incorporates epithelial cells, immune cells, iPSC-derived sensory neurons, and human large intestinal microvascular endothelial cells. Therefore, cross-talk between cells may be used for providing new drug targets using inflammatory models as described herein, including but not limited to responses to neuronal molecules, altered neuronal molecules, and complement modulating treatments.

FIG. 28A-I shows exemplary embodiments of schematics, FIG. 28I, and images of Intestine-chips, FIG. 28F. Villi-like formations in the Intestine-Chip. Morphology was characterized with immunofluorescence cross-sectional view FIG. 28A of intestinal epithelial cells in the Caco-2 Intestine-Chip and Scanning Electron Micrograph (SEM) of Caco-2 FIG. 28B. Epithelial thickness is reduced after an inflammatory treatment FIG. 28D compared to control FIG. 28C. A representative whole-chip tile was taken showing expression of tight junction protein ZO-1 with immunofluorescence in the bottom image, FIG. 28E. Epithelial Layer Morphology and Barrier Function, FIG. 28F. Epithelial cells and iPSC-derived sensory neuron progenitors co-cultured within the chip in the presence of continuous flow for 14 days maintain barrier function, FIG. 28H. Maturation and differentiation of the epithelial morphology and villi-like structures were monitored via bright field imaging over 14 days. The overall viability of the epithelium was assessed by measurement of effluent LDH and found less than 5% leakage for each co-culture condition, FIG. 28G.

FIG. 29 shows exemplary Neuronal Immunofluorescence Staining on one embodiment of an Innervated Intestine-Chip by day 7-Chip, demonstrating interactions (i.e. merged image) between sensory neurons, e.g. nociception specific marker, TRPV1 (red), MAP-2 (green), nuclear stain (blue).

Characterization of iPSC-derived Sensory Neuron Progenitors. The sensory neuron nociception specific marker, TRPV1, was expressed on the Innervated Intestine-Chip by day 7. Positive immunofluorescent staining for this marker of sensory neuron differentiation indicates maturation and functionality is present within the chip.

Readouts for microfluidic devices as described herein are contemplated, including but not limited to: cellular cytoskeleton, barrier function, neuronal activity, RNA levels, cytokine protein levels, biomarkers, biochemical assays, including but not limited to those provided by commercially available kits, immunofluorescent images, such as digital images of cross-sectional and longitudinal areas of microfluidic channels, and high content imaging.

### XV. Exemplary Chip Activation.

A. Chip Activation (Functionalization) Compounds

In one embodiment, bifunctional crosslinkers are used to attach one or more extracellular matrix (ECM) proteins. A variety of such crosslinkers are available commercially, including (but not limited to) the following compounds:

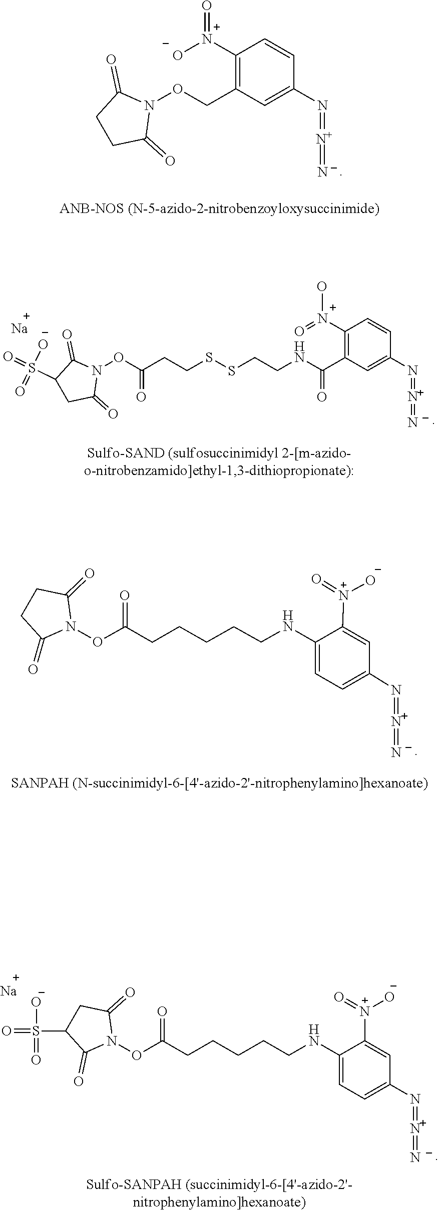

By way of example, sulfosuccinimidyl 6-(4′-azido-2′-nitrophenyl-amino) hexanoate or “Sulfo-SANPAH” (commercially available from Pierce) is a long-arm (18.2 angstrom) crosslinker that contains an amine-reactive N-hydroxysuccinimide (NHS) ester and a photoactivatable nitrophenyl azide. NHS esters react efficiently with primary amino groups (—NH₂) in pH 7-9 buffers to form stable amide bonds. The reaction results in the release of N-hydroxy-succinimide. When exposed to UV light, nitrophenyl azides form a nitrene group that can initiate addition reactions with double bonds, insertion into C—H and N—H sites, or subsequent ring expansion to react with a nucleophile (e.g., primary amines). The latter reaction path dominates when primary amines are present.

Sulfo-SANPAH should be used with non-amine-containing buffers at pH 7-9 such as 20 mM sodium phosphate, 0.15M NaCl; 20 mM HEPES; 100 mM carbonate/bicarbonate; or 50 mM borate. Tris, glycine or sulfhydryl-containing buffers should not be used. Tris and glycine will compete with the intended reaction and thiols can reduce the azido group.

For photolysis, one should use a UV lamp that irradiates at 300-460 nm. High wattage lamps are more effective and require shorter exposure times than low wattage lamps. UV lamps that emit light at 254 nm should be avoided; this wavelength causes proteins to photodestruct. Filters that remove light at wavelengths below 300 nm are ideal. Using a second filter that removes wavelengths above 370 nm could be beneficial but is not essential.

B. Exemplary methods of Chip Activation.
1. Prepare and sanitize hood working space
2. S-1 Chip (Tall Channel) Handling—Use aseptic technique, hold Chip using Carrier
   a. Use 70% ethanol spray and wipe the exterior of Chip package prior to bringing into hood
   b. Open package inside hood
   c. Remove Chip and place in sterile Petri dish (6 Chips/Dish)
   d. Label Chips and Dish with respective condition and Lot #
3. Surface Activation with Chip Activation Compound (light and time sensitive)
   a. Turn off light in biosafety hood
   b. Allow vial of Chip Activation Compound powder to fully equilibrate to ambient temperature (to prevent condensation inside the storage container, as reagent is moisture sensitive)
   c. Reconstitute the Chip Activation Compound powder with ER-2 solution
      i. Add 10 ml Buffer, such as HEPES, into a 15 ml conical covered with foil
      ii. Take 1 ml Buffer from above conical and add to chip Activation Compound (5 mg) bottle, pipette up and down to mix thoroughly and transfer to same conical
      iii. Repeat 3-5 times until chip Activation Compound is fully mixed
      iv. NOTE: Chip Activation Compound is single use only, discard immediately after finishing Chip activation, solution cannot be reused
   d. Wash channels
      i. Inject 200 ul of 70% ethanol into each channel and aspirate to remove all fluid from both channels
      ii. Inject 200 ul of Cell Culture Grade Water into each channel and aspirate to remove all fluid from both channels
      iii. Inject 200 ul of Buffer into each channel and aspirate to remove fluid from both channels
   e. Inject Chip Activation Compound Solution (in buffer) in both channels
      i. Use a P200 and pipette 200 ul to inject Chip Activation Compound/Buffer into each channel of each chip (200 ul should fill about 3 Chips (Both Channels))
      ii. Inspect channels by eye to be sure no bubbles are present. If bubbles are present, flush channel with Chip Activation Compound/Buffer until bubbles have been removed
   f. UV light activation of Chip Activation Compound Place Chips into UV light box
      i. UV light treat Chips for 20 min
      ii. While the Chips are being treated, prepare ECM Solution.
      iii. After UV treatment, gently aspirate Chip Activation Compound/Buffer from channels via same ports until channels are free of solution
      iv. Carefully wash with 200 ul of Buffer solution through both channels and aspirate to remove all fluid from both channels
      v. Carefully wash with 200 ul of sterile DPBS through both channels
      vi. Carefully aspirate PBS from channels and move on to: ECM-to-Chip
         VIX. Exemplary ECM-to-Chip: Coat Chips with ECM

In some embodiments, chip channels are coated with ECM, e.g. a mixture of Collagen IV, laminin and Fibronectin; organ-specific extracellular matrix proteins; cell-specific extracellular matrix proteins; Matrigel® (BD Corning); etc. ECM material may be diluted in Dulbecco's phosphate-buffered saline (DPBS) (without Ca²⁺, Mg²⁺).

A. Closed Top Microfluidic Chips without Gels.

In one embodiment, closed top gut-on-chips, or other types of organ-chips, do not contain gels, either as a bulk gel or a gel layer. Thus, in one embodiment, the device generally comprises (i) a first structure defining a first chamber; (ii) a second structure defining a second chamber; and (iii) a membrane located at an interface region between the first chamber and the second chamber to separate the first chamber from the second chamber, the membrane including a first side facing toward the first chamber and a second side facing toward the second chamber, wherein the first and second chambers are enclosed. The first side of the membrane may have an extracellular matrix composition disposed thereon, wherein the extracellular matrix (ECM) composition comprises an ECM coating layer. In some embodiments, an ECM gel layer e.g. ECM overlay, is located over the ECM coating layer.

Additional embodiments are described herein that may be incorporated into closed top chips without gels.

B. Closed Top Microfluidic Chips With Gels.

In one embodiment, closed top gut-on-chips do contain gels, such as a gel layer, or bulk gel, including but not limited to a gel matrix, hydrogel, etc. Thus, in one embodiment, the device generally comprises (i) a first structure defining a first chamber; (ii) a second structure defining a second chamber; and (iii) a membrane located at an interface region between the first chamber and the second chamber to separate the first chamber from the second chamber, the membrane including a first side facing toward the first chamber and a second side facing toward the second chamber, wherein the first and second chambers are enclosed. In some embodiments, the device further comprises a gel. In some embodiments, the gel is a continuous layer. In some embodiments, the gel is a layer of approximately the same thickness across the layer. In some embodiments, the gel is a discontinuous layer. In some embodiments, the gel has different thicknesses across the layer. In some embodiments, the first side of the membrane may have a gel layer. In some embodiments, a gel is added to the first side of the membrane without an ECM layer. The first side of the membrane may have an extracellular matrix composition disposed thereon, wherein the extracellular matrix (ECM) composition comprises an ECM coating layer. In some embodiments, an ECM gel layer e.g. ECM overlay, is located over the ECM coating layer. In some embodiments, the gel layer is above the ECM coating layer. In some embodiments, the ECM coating layer may have a gel layer on the bottom, i.e. the side facing the membrane. In some embodiments, the gel overlays the ECM gel layer.

Additional embodiments are described herein that may be incorporated into closed top chips with gels.

C. Closed Top Microfluidic Chips With Simulated Lumens.

A closed top gut-on-chip comprising a gel-lined simulated lumen may be used for generating a more physiological relevant model of gastrointestinal tissue. In some embodiments, closed top gut-on-chips further comprise a gel simulated three-dimensional (3-D) lumen. In other words, a 3-D lumen may be formed using gels by providing simulated intestinal villi (e.g. viscous fingers) and/or mimicking intestinal folds. In a preferred embodiment, the gel forms a lumen, i.e. by viscous fingering patterning.

Using viscous fingering techniques, e.g. viscous fingering patterning, a simulated intestinal lumen may be formed by numerous simulated intestinal villi structures. Intestinal villi (singular: villus) refer to small, finger-like projections that extend into the lumen of the small intestine. For example, healthy small intestine mucosa contains these small finger-like projections of tissue that are present along the lumen as folds of circular plica finger-like structures. A villus is lined on the luminal side by an epithelia cell layer, where the microvillus of the epithelial cells (enterocytes) faces the lumen (i.e. apical side). Viscous fingers may be long and broad, for mimicking villi in the duodenum of the small intestine, while thinner or shorter viscous fingers may be used for mimicking villi in other parts of the gastrointestinal tract. As one example, viscous fingers may be formed and used to mimic epithelial projections in the colon.

Methods to create three-dimensional (3-D) lumen structures in permeable matrices are known in the art. One example of a 3-D structure forming at least one lumen is referred to as “viscous fingering”. One example of viscous fingering methods that may be used to for form lumens, e.g. patterning lumens, is described by Bischel, et al. “A Practical Method for Patterning Lumens through ECM Hydrogels via Viscous Finger Patterning.” J Lab Autom. 2012 April; 17(2): 96-103, Author manuscript; available in PMC 2012 Jul. 16, herein incorporated by reference in its entirety. In one example of a viscous finger patterning method for use with microfluidic gut-on-chips, lumen structures are patterned with an ECM hydrogel.

“Viscous” generally refers to a substance in between a liquid and a solid, i.e. having a thick consistency. A “viscosity” of a fluid refers to a measure of its resistance to gradual deformation by shear stress or tensile stress. For liquids, it corresponds to an informal concept of “thickness”; for example, honey has a much higher viscosity than water.

“Viscous fingering” refers in general to the formation of patterns in “a morphologically unstable interface between two fluids in a porous medium.

A “viscous finger” generally refers to the extension of one fluid into another fluid. Merely as an example, a flowable gel or partially solidified gel may be forced, by viscous fingering techniques, into another fluid, into another viscous fluid in order to form a viscous finger, i.e. simulated intestinal villus.

In some embodiments, the lumen can be formed by a process comprising (i) providing the first chamber filled with a viscous solution of the first matrix molecules; (ii) flowing at least one or more pressure-driven fluid(s) with low viscosity through the viscous solution to create one or more lumens each extending through the viscous solution; and (iii) gelling, polymerizing, and/or cross linking the viscous solution. Thus, one or a plurality of lumens each extending through the first permeable matrix can be created.

In another embodiment, gel is added to a channel for making a lumen.

In some embodiments as described herein, the first and second permeable matrices can each independently comprise a hydrogel, an extracellular matrix gel, a polymer matrix, a monomer gel that can polymerize, a peptide gel, or a combination of two or more thereof. In one embodiment, the first permeable matrix can comprise an extracellular matrix gel, (e.g. collagen). In one embodiment, the second permeable matrix can comprise an extracellular matrix gel and/or protein mixture gel representing an extracellular microenvironment, (e.g. MATRIGEL®. In some embodiments, the first and second permeable matrixes can each independently comprise a polymer matrix. Methods to create a permeable polymer matrix are known in the art, including, e.g. but not limited to, particle leaching from suspensions in a polymer solution, solvent evaporation from a polymer solution, sold-liquid phase separation, liquid-liquid phase separation, etching of specific “block domains” in block co-polymers, phase separation to block-co-polymers, chemically cross-linked polymer networks with defined permabilities, and a combination of two or more thereof.

Another example for making branched structures using fluids with differing viscosities is described in “Method And System For Integrating Branched Structures In Materials” to Katrycz, Publication number US20160243738, herein incorporated by reference in its entirety.

Regardless of the type of lumen formed by a gel and/or structure, cells can be attached to these structures either to lumen side of the gel and/or within the gel and/or on the side of the gel opposite the lumen. Thus, three-dimensional (3-D) lumen gel structures may be used in several types of embodiments for closed top microfluidic chips, e.g. epithelial cells can be attached to outside of the gel, or within the gel. In some embodiments, stoma cells are added within the gel. In some embodiments, stomal cells are attached to the side of the gel opposite from the lumen. In some embodiments, endothelial cells are located below the gel on the side opposite the lumen. In some embodiments, endothelial cells may be present within the gel.

Additional embodiments are described herein that may be incorporated into closed top chips with simulated 3D lumens containing a gel.

### XVI. Exemplary Material and Methods.

Cell culture. Commercial human iPSC-derived dopaminergic neurons (iCell® Neurons), were purchased from Cellular Dynamics International (CDI, Madison, Wis.) and maintained in complete maintenance media (iCell DopaNeurons Media). iCell DopaNeurons Kit comprising M1010 100 ml iCell Neural Base Medium 1; M1029 2 ml iCell Neural Supplement B; and M1031 1 ml iCell Nervous System Supplement (Growth factor supplement for neuronal cell types). These neuronal cells were characterized by CDI to represent a pure neuronal population with >80% pure midbrain dopaminergic neurons.

Primary human astrocytes isolated from the cerebral cortex were obtained from ScienCell and maintained in Astrocyte Medium (ScienCell). Astrocytes refer to a subtype of glial cells observed as star shaped glial cells that reside in the brain and spinal cord. the most abundant glial cells in the brain that are closely associated with neuronal synapses. They regulate the transmission of electrical impulses within the brain. Astrocytes support neuronal function by producing antioxidants (glutathione), recycling neurotransmitters (glutamate and GABA), and maintaining the BBB (to sustain the microenvironmental equilibrium).

Primary human brain pericytes were also obtained from ScienCell and maintained in the Pericyte medium (ScienCell). Pericytes (PC) refer to cells found in vivo surrounding endothelial cells in small blood vessels. PCs show immune properties by responding to pro-inflammatory stimuli and engagement of functional pattern-recognition receptors. PCs secrete a variety of chemokines and express adhesion molecules such as ICAM-1 and VCAM-1 involved in the control of immune cell trafficking across vessel walls. Markers used to identify activated PCs include pan-macrophage marker CD68 (ED1) and CD11b (alpha chain of the integrin Mac-1/CR3). Additional markers include: PDGF receptor-β (PDGFR-β), nerve-glial antigen-2/chondroitin sulfate proteoglycan 4 (NG2), regulator of G-protein signaling-5 (RGS5), α-smooth muscle actin (αSMA), desmin, aminopeptidase N (CD13), endoglin (CD105), adhesion molecule CD146, Fc receptors, scavenger receptors, etc.

Microglia represent a specialized population of myeloid cells residing in vivo within the brain parenchyma.

Resting primary human brain microglia were purchased from ATCC, e.g. HMC₃ (ATCC® CRL-3304™), and cultured according to the manufacturer's instructions. The HMC₃ cell line was established through SV40-dependent immortalization of a human fetal brain-derived primary microglia culture. The primary cells were used at passage 2-4. Resting HMC₃ cells are strongly positive for the microglia/macrophage marker IBA1, positive for the endotoxin receptor CD14, but negative for the astrocyte marker GFAP. Markers of activated microglia, namely MHCII, CD68 and CD11b were negative in resting HMC₃ cells, but upregulated after activation by IFN-gamma (10 ng/ml, 24 h). Base medium for this cell line is EMEM (ATCC® 30-2003™). As one example for expansion and maintenance medium, for complete medium add 56 mL FBS (ATCC® 30-2020™) to a 500 mL bottle of the base medium. Primary sources of human microglia, include fetal tissue, biopsies from epileptic patients, normal tissue from brain tumor excisions, or postmortem brain tissue, e.g. for microglial-astrocyte or microglial-pericyte interactions

Differentiation of iPSCs into Brain Microvascular Endothelial Cells. Brain microvascular endothelial cell differentiation of hiPSCs. Human-induced pluripotent stem cells (hiPSCs) obtained from the Rutgers University Cell and DNA Repository (ND50028; RUCDR) were maintained on Matrigel-coated tissue-culture treated six-well culture plates (Corning) in mTeSR1 (Stem Cell Technologies). The established hiPSC colonies displayed a normal karyotype in culture. For each independent experiment, the same cell passage (P49) was used. Prior to differentiation, hiPSCs were singularized using Accutase (Invitrogen) and plated onto Matrigel-coated six-well culture plates in mTeSR1 supplemented with 10 μM Rho-associated protein kinase inhibitor Y27632 (ROCK inhibitor; Stem Cell Technologies) at a density between 25,000 and 50,000 cells cm-2. Directed differentiation of hiPSCs was adapted from a previously reported protocol³⁸. Briefly, singularized hiPSCs were expanded for three days in mTeSR1, then were treated with 6 μM CHIR99021 (Stem Cell Technologies) in DeSR1: DMEM/F-12 (Life Technologies), 1% non-essential amino acids (Thermo Fisher Scientific), 0.5% GlutaMAX (Thermo Fisher Scientific), 0.1 mM beta-mercaptoethanol (Sigma) to initiate differentiation (day zero). After one day, the medium was changed to DeSR2: DeSR1 plus 1×B27 (Thermo Fisher Scientific) and changed daily for five days. On day six, the medium was switched to hECSR1: hESFM (Thermo Fisher Scientific) supplemented with 20 ng/mL bFGF (R&D Systems), 10 μM all-trans retinoic acid (Sigma) and 1×B27. The medium was not changed for 48 hrs. On Day 9, the medium was switched to hESCR2: hECSR1 lacking RA and bFGF. On day ten, cells were dissociated with TrypLE™ (Thermo Fisher Scientific) and replated onto a human placenta-derived collagen IV/human plasma-derived fibronectin/human placenta-derived laminin-coated flasks. After 20 mins, the flasks were rinsed using a medium composed of human serum-free endothelial medium supplemented with 2% platelet-poor plasma-derived serum and 10 μM Y27632, as a selection step to remove any undifferentiated cells. Human brain microvascular endothelial cells (HBMECs) were then left in the same medium overnight to allow cell attachment and growth before seeded into the Organ-Chips.

In one embodiment, Human iPSCs (Donor 1: RUCDR; ND50028, Donor 2: iXcell; 30HU-002) were passaged onto Matrigel in mTeSR1 medium for 2-3 days of expansion. Colonies were singularized using Accutase (STEMCELL; 07920) and replated onto Matrigel-coated plates at a density 25-50×103 cells/cm2 in mTeSR1 supplemented with 10 mM Rho-associated protein kinase (ROCK) inhibitor Y-27632 (STEMCELL; 72304). Singularized Human iPSCs were expanded in mTeSR1 for 3 days. Cells were then treated with 6 mM CH1R99021 (STEMCELL; 72052) in DeSR1: DMEM/Ham's F12 (Thermo Fisher Scientific; 11039021), 1×MEM-NEAA (Thermo Fisher Scientific; 10370021), 0.5% GlutaMAX (Thermo Fisher Scientific; 35050061), and 0.1 mM b-mercaptoethanol (Sigma). On Day 1, the medium was changed to DeSR2: DeSR1 plus 1×B27 (Thermo Fisher Scientific) daily for another 5 days. On day 6, the medium was switched to hECSR1: hESFM (ThermoFisher Scientific) supplemented with bFGF (20 ng/mL), 10 mM Retinoic Acid, and 1×B27. On day 8, the medium was changed to hECSR2 (hECSR1 without R.A. or bFGF). On day 10 cells were dissociated with TrypLE™ and plated at 1×106 cells/cm2 in hESFM supplemented with 5% human serum from platelet-poor human plasma onto a mixture of collagen IV (400 μg/mL), fibronectin (100 μg/mL), and laminin (20 μg/mL) coated flasks at a density of 1×106 cells/cm2. After 20 mins the flasks were rinsed using hESFM with 5% human serum from platelet-poor human plasma with Y-27632 as a selection step to remove any undifferentiated cells and allowed to attach overnight (Qian et al., 2017).

Organ-Chip fabrication and culture module. Organ-Chips (Chip-S1, Emulate, Inc. Boston, Mass., USA) were used to recreate the human Brain-Chip. The chip, e.g. Chip-S1, is made of transparent, poly(dimethylsiloxane) (PDMS) flexible elastomer (elastomeric polymer). It consists of two channels (1×1 mm and 1×0.2 mm, “Brain” and “Vascular” channel, respectively) separated by a thin (50 μm), porous flexible PDMS membrane²⁵. The membrane has 7 μm diameter pores with 40 μm spacing, coated with E.C.M. (400 μg/mL collagen IV, 100 μg/mL fibronectin, and 20 μg/mL laminin, at the brain and vascular side). Flow can be introduced to each channel independently to continuously provide essential nutrients to the cells, while effluent containing any secretion/waste components from cells is excreted/collected on the outlet of each channel separately. This allows for channel-specific and independent analysis and interpretation of results. The Zoe culture module is the instrumentation designed to automate the maintenance of these chips in a controlled and robust manner (Emulate Inc.).

Human Brain-Chip and cell seeding. Prior to cell seeding, chips were functionalized using ER-1 protocols and ER-2 reagents. After surface functionalization, both channels of the human Brain-Chip were coated with collagen IV (400 μg/mL), fibronectin (100 μg/mL), and laminin (20 μg/mL) overnight both channels of the chip and then filled with DopaNeurons Media before seeding cells. A mixture of dopaminergic neurons, astrocytes, microglia, and pericytes was seeded in the upper brain channel of the Brain-Chips at the following concentrations: 2 million cells/mL for dopaminergic neurons, 2 million cells/mL for astrocytes, 0.1 million cells/mL for microglia, and 0.1 million cells/mL for pericytes.

In one embodiment, chips were seeded with human iPS-derived glutamatergic and GABAergic neurons (NeuCyte) at a density of 2×10⁶ cells/mL and 0.75×10⁶ cells/mL respectively, co-cultured with human primary microglia (ATCC; CRL3304) at a density of 1×10⁵ cells/mL, human primary astrocytes (NeuCyte), and primary pericytes (Sciencell; 1200) at a density of 1×10⁵ cells/mL, using “seeding medium” (NeuCyte), and incubated overnight. The next day, human iPS-derived Brain Microvascular Endothelial cells were seeded in the vascular channel at a density of 14-16×10⁶ cells/mL using human serum-free endothelial cell medium supplemented with 5% human serum from platelet-poor human plasma (Sigma) and allowed to attach to the membrane overnight. Chips were then connected to the culture module. At this time, the medium supplying the brain channel was switched to maintenance medium (Neucyte), and the serum of the vascular medium was lowered to 2%. Chips were maintained under constant perfusion at 60 μL/hr through all chips' brain and vascular channels until day 7. Two additional commercial endothelial cell sources were used in experiments. Human primary endothelial cells (Cell Systems) and hCMEC/D3 (Millipore) were cultured in media according to the manufacturer's instructions.

After cell seeding, the upper channel of the Brain-Chip was maintained in DopaNeurons Media and incubated overnight at 37° C. (Day 0). The following day (Day 1), the lower vascular channel was rinsed with human serum-free endothelial medium supplemented with 2% platelet-poor plasma-derived serum, 10 μM Y27632, and then BMECs were seeded at a concentration of 16-20 million cells/mL to ensure the very tight endothelial monolayer found in the human blood-brain barrier, and the chips were flipped immediately to allow BMECs to adhere to the ECM-coated part of the membrane. After 2 h incubation, the chips were flipped back to let the rest of BMECs sit on the bottom and sides of the channel to form a capillary lumen. The vascular channel of the Brain-Chip was maintained overnight. On Day 2, the Brain-Chips were connected to the culture module and perfused continuously through the brain and vascular channel at a flow rate of 30 μl hr⁻¹ and 60 μl hr⁻¹ respectively, using each channels' respective media.

TNF-α treatment. To mimic the inflammatory condition, cells were treated on either brain or vascular channel with TNF-α (Tumor Necrosis Factor-α, R&D Systems; 210-TA). The treatment was initiated after the formation of a confluent monolayer at ˜5 days in culture. Cells were further incubated in a culturing medium, including TNF-α (100 ng/ml) up to 48 hours.

Addition of exogenous alpha-synuclein to brain channel. Human recombinant αSyn monomers and pre-formed fibrils were purchased from Abcam (Monomers; ab218819, Fibrils; ab218819), and were diluted in DopaNeurons Media to a final concentration of 4 μg/mL. Fibrils are Recombinant Human Alpha-synuclein protein aggregate (Active) having endogenous alpha-synuclein phosphorylation capability and capable of phosphorylation at Ser-129; ‘non A-beta component of Alzheimer disease amyloid plaque’ domain (NAC domain) is involved in fibrils formation: WT human isoform NACP140, which may be induced to form fibrils as purchased from Abcam (SEQ ID NO: 1): MDVFMKGLSK AKEGVVAAAE KTKQGVAEAA GKTKEGVLYV GSKTKEGVVH GVATVAEKTK EQVTNVGGAV VTGVTAVAQK TVEGAGSIAA ATGFVKKDQL GKNEEGAPQE GILEDMPVDP DNEAYEMPSE EGYQDYEPEA.

In other embodiments, the use of other commercial types of alpha-syn fibrils and monomers are contemplated for use.

On the day of use, αSyn fibrils were sonicated, and their activity was verified by Thioflavin T assay. Endotoxin levels were evaluated by the Limulus amebocyte lysate assay (Endotoxin Testing Services, Lonza Europe), and the amount expressed was negligible.

For treatment, freshly prepared monomers and fibrils were used. On Day 2, the upper channel of the Brain-Chip was exposed to monomeric or fibrillar αSyn. After three days of exposure (D5), the media was changed, and the culture was maintained using DopaNeurons Media (αSyn free) for three more days (D8). Effluents, lysates, and staining were collected/fixed at day three- and day six post-exposure (D5 and D8 respectively), and were analyzed by a microplate reader, ELISA kits, and immunofluorescence microscopy.

Permeability Assays, e.g., evaluate the establishment and integrity of the barrier. Apparent permeability (Papp) of the barrier was calculated by following a previously described method⁸¹. Briefly, 100 μg mL⁻¹ (0.1 mg/mL) of dextran (3 kDa, e.g. Cascade Blue) and/or 20 μg mL⁻¹ of Lucifer (0.5 kDa) tracers were dosed (flowed) through the vascular channels for 24 hrs. After 24 hrs, effluent from both channels was sampled to determine the dye's concentration (amount) that had diffused through the membrane. Concentration of the dextran and Lucifer tracers in the outlet samples from both vascular and brain channels was determined by using BioTek (BioTek Instruments, Inc., Winooski, Vt., USA). Then, the following equation was used to calculate apparent paracellular permeability (Papp) based on a standard curve and using the following formula:

$${P}_{app} = \frac{{Q}_{R} * {Q}_{D}}{SA * ( {Q}_{R} + {Q}_{D} )} * ln [ 1 - \frac{{C}_{R , 0} * ( {Q}_{R} + {Q}_{D} )}{( {Q}_{R} * {C}_{R , 0} + {Q}_{D} * {C}_{D , 0} )} ]$$

Here, SA is the surface area of sections of the channels that overlap (0.17 cm²), Q_{R} and Q_{D} are the fluid flow rates in the dosing and receiving channels respectively, in units of cm^{3/s}, C_{R,0} and C_{D,0} are the recovered concentrations in the dosing and receiving channels respectively, in any consistent units. IgG permeability was also evaluated after dosing the vascular channel and measuring the IgG content on the brain channel. Detection and quantitation of serum immunoglobulin G (IgG1; Abcam) was performed using the ELISA kit (Abcam), after 24 hrs of perfusion.

Morphological analysis. Immunocytochemistry, including immunofluorescence microscopy, was conducted as previously described (Pediaditakis et al., 2020). Brief examples of methods are described herein. Brain-Chips were fixed with 4% paraformaldehyde in PBS for 10 min and then washed with PBS. Cells were blocked on the Brain-Chip in phosphate-buffered saline (PBS) containing 10% donkey serum (Sigma) at 4° C. overnight. Immunostaining was performed after permeabilization in PBS with 0.1% Saponin and blocking for 30 min in 10% donkey serum in PBS with 0.1% Saponin. Immunostaining was performed with specific primary antibodies (anti-TH, anti-GFAP, anti-NG2, anti-TMEM119, anti-pSer129-αSyn, anti-Cleaved Caspase-3, anti-CD68; Abcam), (anti-MAP2, and anti-CD31; Thermo Fisher Scientific), (anti-Claudin-1, anti-Claudin-5, anti-Occludin, anti-ZO-1; Invitrogen), in a 1:100 dilution in 10% donkey serum in PBS with 0.1% Saponin and incubated overnight on the Brain-Chip at 4° C. Additional primary antibodies were MAP2 (Thermo Fisher Scientific; MA512826), VGLUT1 (Thermo Fisher Scientific; 48-2400), GFAP (Abcam; ab53554), NG2 (Abcam; ab83178), CD68 (Abcam; ab213363), ICAM-1 (R&D Systems; BBA3), ZO-1 (Thermo Fisher Scientific; 402200), GLUT1 (Thermo Fisher Scientific; SPM498). Fluorescently conjugated secondary antibodies with Alexa Fluor-488, Alexa Fluor-568, or Alexa Fluor-647 (Abcam) were then used when the primary antibodies are not conjugated, incubated in the dark for 2 hr at room temperature. Cells were then counterstained with DAPI nuclear stain. Images were acquired with either an Olympus fluorescence microscope (IX83) or an inverted laser-scanning confocal microscope, e.g. Zeiss confocal microscope (AxiovertZ1 LSM880).

ELISA analysis. The levels of IFNγ, IL-1β, and IL-6 were measured by M.S.D. 96-well plate Human Pro-Inflammatory V-PLEX Human Pro-Inflammatory Assay kits. The secreted levels of Glutamate were measured by Glutamate Assay Kit (Fluorometric) (Abcam; ab138883).

Visualization of transferrin receptor internalization. Human iPS-derived Brain Microvascular Endothelial cells were treated with 25 μg/ml fluorescent transferrin conjugate (Thermo Fisher Scientific) and incubated at 37° C. for 30 minutes. Cells were washed twice with LCIS and fixed with P.F.A. Cells labeled with Alexa Fluor™ Plus 647 Phalloidin and DAPI and then imaged with Zeiss LSM 880.

Mitochondrial membrane potentials assay. JC-1 probe was employed to evaluate the mitochondrial depolarization in cells seeded at the brain channel. Briefly, cells were incubated with 2 μM of JC-1 dye at 37° C. for 20 min and rinsed twice with PBS, then replaced in fresh medium. Finally, images were taken in the green and red fluorescence channel by confocal laser scanning microscopy imaging. The images were obtained at 488 nm excitation and 530 nm emission for green (JC-1 monomers) and 543 nm excitation and 590 nm emission for red fluorescence (JC-1 aggregates). Four frames per chip at 10× magnification were selected for each treatment, and fluorescence intensity was measured using Fiji/ImageJ.

Intracellular ROS Measurement. Intracellular ROS production was measured using CellROX Green Reagent (Thermo Fisher Scientific) according to the manufacturer's protocol. At day 8, CellROX reagent was added to the brain channel at a final concentration of 5 μM, and cells were incubated for 60 min at 37° C. in the dark, followed by triple washing with prewarmed PBS. Then, cells were examined with a confocal laser scanning microscope at an excitation/emission wavelength of 485/520 nm. Four frames per chip at 10× magnification were selected for each treatment, and particles were counted using Fiji/ImageJ.

Viability Assay. The cell viability was assessed using the LIVE/DEAD staining kit (Thermo Fisher Scientific). The neuronal channel of the Brain-Chips was incubated for 30 min in PBS containing 1 μM Calcein-AM and 2 μM ethidium homodimer-1 (EthD1). The channel was then washed with PBS and imaged under a motorized fluorescent microscope (Zeiss confocal microscope). Four frames per chip at 10× magnification were selected for each treatment, and particles were counted using Fiji/ImageJ. Data were expressed as the average live cells/total number of cells (sum of Calcein-AM positive and ethidium homodimer positive). In order to confirm the efficiency and reliability of this assay, a positive control (DMSO treatment) and negative control (no treatment) was used in parallel experiments with the αSyn treatment.

Cytokine Secretion. The levels of TNF-α, and IL-6 were measured by commercial ELISA kit (Abeam) according to the manufacturers' instructions. The assays were performed in duplicate in 96-well plates, and the results were presented as picograms per milliliter.

RNA isolation and sequencing. RNA was extracted using TRIzol (T.R.I. reagent, Sigma) according to manufacturer's guidelines. The collected samples were submitted to GENEWIZ South Plainfield, N.J., USA, for next-generation sequencing. After quality control and RNA-seq library preparation the samples were sequenced with Illumina HiSeq 2×150 system using sequencing depth ˜50M paired-end reads/sample.

RNA sequencing bioinformatics. Using Trimmomatic v. 0.36 the sequence reads were trimmed to filtered-out poor quality nucleotides and possible adapter sequences. The remaining trimmed reads were mapped to the homo sapience reference genome GRCh38 using the STAR aligner v 2.5.2b which generated the B.A.M. files. Using the B.A.M. files, it was calculated that for each sample the unique gene hit-counts were identified by applying the feature Counts from the subread package v. 1.5.2. Note that only unique reads that fell within the exon region were counted. Finally, the generated hit-counts were used to perform Differentially Gene Expression (DGE) analysis using the “DESeq2” R package by Bioconductor.

G.O. term enrichment analysis. The gene sets resulted from the DGE analyses were subjected to Gene Ontology (GO) enrichment analysis. The GO terms enrichment analysis was performed using the Gene Ontology knowledgebase (Gene Ontology Resource http://geneontology.org/).

GTEx human adult substantia nigra samples selection procedure. GTEx Portal provides 114 RNA-seq samples for human adult substantia nigra. Eight representative samples out of the 114 samples were selected and combined with 8 samples from a Brain-Chip and CCC samples to generate a balanced dataset. For the selection of the 8 representative samples the following criteria was used: (1) The samples belonged to donors who were reasonably healthy and they had fast and unexpected deaths from natural causes; and (2) Have the smaller transcriptomic distances⁸² from the average transcriptomic expression profile of the samples that satisfy criterion (1). Next, the “removeBatchEffect” function of the “limma” R package was used in order to remove shifts in the means between samples (Brain-Chip and CCC) and the 8 human SN samples retrieved from the GTEx portal. The dataset was used to perform DGE analyses between the different conditions. For the DGE analyses, the ‘DESeq2’ R package by bioconductor⁸³ was used.

Statistical Analysis. Experiments were performed with controls (monomers or PBS) side-by-side and in random order and they were reproduced for at least two times to confirm data reliability. Some of the experiments were replicated at least 3 times. GraphPad Prism was used to perform statistical analyses (GraphPad Software). Numeric results are shown as mean±standard error of the mean (SEM) and represent data from a minimum of two independent experiments of distinct sample measurements (n>3). Analysis of significance was performed by using two-way ANOVA with Tukey's multiple comparisons test or unpaired t-test depending on the data sets. Significant differences are depicted as follows: *P<0.05, **P<0.01, ***P<0.001, and ****P<0.0001. In some embodiments, error bars represent standard error of the mean (s.e.m); p values<0.05 and above were considered significant.

### REFERENCES, HEREIN INCORPORATED IN THEIR ENTIRETY

1. Peng, C., Gathagan, R. J. & Lee, V. M. Y. Distinct α-Synuclein strains and implications for heterogeneity among α-Synucleinopathies. Neurobiology of Disease vol. 109 209-218 (2018).
2. Peelaerts, W., Bousset, L., Baekelandt, V. & Melki, R. α-Synuclein strains and seeding in Parkinson's disease, incidental Lewy body disease, dementia with Lewy bodies and multiple system atrophy: similarities and differences. Cell and Tissue Research vol. 373 195-212 (2018).
3. Luk, K. C. et al. Pathological α-synuclein transmission initiates Parkinson-like neurodegeneration in nontransgenic mice. Science 338, 949-53 (2012).
4. Goedert, M., Jakes, R. & Spillantini, M. G. The Synucleinopathies: Twenty Years on. Journal of Parkinson's Disease vol. 7 S53-S71 (2017).
5. Ouzounoglou, E. et al. In silico modeling of the effects of alpha-synuclein oligomerization on dopaminergic neuronal homeostasis. BMC Syst. Biol. 8, (2014).
6. El-Agnaf, O. M. A. et al. Detection of oligomeric forms of α-synuclein protein in human plasma as a potential biomarker for Parkinson's disease. FASEB J. 20, 419-425 (2006).
7. Tokuda, T. et al. Detection of elevated levels of α-synuclein oligomers in CSF from patients with Parkinson disease. Neurology 75, 1766-1772 (2010).
8. Beach, T. G. et al. Multi-organ distribution of phosphorylated α-synuclein histopathology in subjects with Lewy body disorders. Acta Neuropathol. 119, 689-702 (2010).
9. Donadio, V. et al. Skin nerve α-synuclein deposits A biomarker for idiopathic Parkinson disease. Neurology 82, 1362-1369 (2014).
10. Kortekaas, R. et al. Blood-brain barrier dysfunction in Parkinsonian midbrain in vivo. Ann. Neurol. 57, 176-179 (2005).
11. Rektor, I. et al. Impairment of brain vessels may contribute to mortality in patients with Parkinson's disease. Mov. Disord. 27, 1169-1172 (2012).
12. Sui, Y.-T., Bullock, K. M., Erickson, M. A., Zhang, J. & Banks, W. A. Alpha synuclein is transported into and out of the brain by the blood-brain barrier. Peptides 62, 197-202 (2014).
13. Lee, H. & Pienaar, I. S. Disruption of the blood-brain barrier in parkinson's disease: Curse or route to a cure? Front. Biosci.—Landmark 19, 272-280 (2014).
14. Carvey, P. M. et al. 6-Hydroxydopamine-induced alterations in blood-brain barrier permeability. Eur. J. Neurosci. 22, 1158-1168 (2005).
15. Peelaerts, W. et al. α-Synuclein strains cause distinct synucleinopathies after local and systemic administration. Nature 522, 340-344 (2015).
16. Stefanis, L., Larsen, K. E., Rideout, H. J., Sulzer, D. & Greene, L. A. Expression of A53T mutant but not wild-type α-synuclein in PC12 cells induces alterations of the ubiquitin-dependent degradation system, loss of dopamine release, and autophagic cell death. J. Neurosci. 21, 9549-9560 (2001).
17. Vekrellis K., Xilouri M., Emmanouilidou E., S. L. Inducible over-expression of wild type alpha-synuclein in human neuronal cells leads to caspase-dependent non-apoptotic death. J. Neurochem. 109, 1348-1362 (2009).
18. Kuan, W.-L. et al. α-Synuclein pre-formed fibrils impair tight junction protein expression without affecting cerebral endothelial cell function. Exp. Neurol. 285, 72-81 (2016).
19. Lane, E. & Dunnett, S. Animal models of Parkinson's disease and L-dopa induced dyskinesia: How close are we to the clinic? Psychopharmacology vol. 199 303-312 (2008).
20. Banks, W. A., Kovac, A. & Morofuji, Y. Neurovascular unit crosstalk: Pericytes and astrocytes modify cytokine secretion patterns of brain endothelial cells. J. Cereb. Blood Flow Metab. 38, 1104-1118 (2018).
21. Kaisar, M. A. et al. New experimental models of the blood-brain barrier for CNS drug discovery. Expert Opinion on Drug Discovery vol. 12 89-103 (2017).
22. Bhatia, S. N. & Ingber, D. E. Microfluidic organs-on-chips. Nature Biotechnology vol. 32 760-772 (2014).
23. Haring, A. P., Sontheimer, H. & Johnson, B. N. Microphysiological Human Brain and Neural Systems-on-a-Chip: Potential Alternatives to Small Animal Models and Emerging Platforms for Drug Discovery and Personalized Medicine. Stem Cell Rev. Reports 13, 381-406 (2017).
24. Kasendra, M. et al. Duodenum Intestine-Chip for preclinical drug assessment in a human relevant model. Elife 9, (2020).
25. Huh, D. et al. A human disease model of drug toxicity-induced pulmonary edema in a lung-on-α-chip microdevice. Sci. Transl. Med. 4, (2012).
26. Jang, K. J. et al. Reproducing human and cross-species drug toxicities using a Liver-Chip. Sci. Transl. Med. 11, (2019).
27. Agarwal, A., Goss, J. A., Cho, A., McCain, M. L. & Parker, K. K. Microfluidic heart on a chip for higher throughput pharmacological studies. Lab Chip 13, 3599-3608 (2013).
28. Moreno, E. L. et al. Differentiation of neuroepithelial stem cells into functional dopaminergic neurons in 3D microfluidic cell culture. Lab Chip 15, 2419-2428 (2015).
29. Freundt, E. C. et al. Neuron-to-neuron transmission of α-synuclein fibrils through axonal transport. Ann. Neurol. 72, 517-524 (2012).
30. Vatine, G. D. et al. Human iPSC-Derived Blood-Brain Barrier Chips Enable Disease Modeling and Personalized Medicine Applications. Cell Stem Cell 24, 995-1005.e6 (2019).
31. Park, T. E. et al. Hypoxia-enhanced Blood-Brain Barrier Chip recapitulates human barrier function and shuttling of drugs and antibodies. Nat. Commun. 10, (2019).
32. Shin, Y. et al. Blood-Brain Barrier Dysfunction in a 3D In Vitro Model of Alzheimer's Disease. Adv. Sci. (Weinheim, Baden-Wurttemberg, Ger. 6, 1900962 (2019).
33. Ahn, S. I. et al. Microengineered human blood-brain barrier platform for understanding nanoparticle transport mechanisms. Nat. Commun. 11, (2020).
34. Choi, J. H., Santhosh, M. & Choi, J. W. In vitro blood-brain barrier-integrated neurological disorder models using a microfluidic device. Micromachines vol. 11 (2020).
35. Ganjam, G. K. et al. Mitochondrial damage by α-synuclein causes cell death in human dopaminergic neurons. Cell Death Dis. 10,865 (2019).
36. Hirsch, E. C., Vyas, S. & Hunot, S. Neuroinflammation in Parkinson's disease. Park. Relat. Disord. 18, (2012).
37. Obermeier, B., Daneman, R. & Ransohoff, R. M. Development, maintenance and disruption of the blood-brain barrier. Nature Medicine vol. 19 1584-1596 (2013).
38. Qian, T. et al. Directed differentiation of human pluripotent stem cells to blood-brain barrier endothelial cells. Sci. Adv. 3, e1701679 (2017).
39. Kniesel, U. & Wolburg, H. Tight junctions of the blood-brain barrier. Cell. Mol. Neurobiol. 20, 57-76 (2000).
40. Shi, L., Zeng, M., Sun, Y. & Fu, B. M. Quantification of blood-brain barrier solute permeability and brain transport by multiphoton microscopy. J. Biomech. Eng. 136, (2014).
41. Yuan, W., Lv, Y., Zeng, M. & Fu, B. M. Non-invasive measurement of solute permeability in cerebral microvessels of the rat. Microvasc. Res. 77, 166-173 (2009).
42. Battle A, Brown C D, Engelhardt B E, M. S. B. Genetic effects on gene expression across human tissues. Nature 550, 204-213 (2017).
43. Hesari, Z. et al. A hybrid microfluidic system for regulation of neural differentiation in induced pluripotent stem cells. J. Biomed. Mater. Res.—Part A 104, 1534-1543 (2016).
44. Samal, P., van Blitterswijk, C., Truckenmüller, R. & Giselbrecht, S. Grow with the Flow: When Morphogenesis Meets Microfluidics. Adv. Mater. 31, (2019).
45. Marques, O. & Outeiro, T. F. Alpha-synuclein: From secretion to dysfunction and death. Cell Death and Disease vol. 3 (2012).
46. Volpicelli-Daley, L. A. et al. Exogenous α-Synuclein Fibrils Induce Lewy Body Pathology Leading to Synaptic Dysfunction and Neuron Death. Neuron 72, 57-71 (2011).
47. Anderson, J. P. et al. Phosphorylation of Ser-129 is the dominant pathological modification of α-synuclein in familial and sporadic lewy body disease. J. Biol. Chem. 281, 29739-29752 (2006).
48. Arawaka, S., Sato, H., Sasaki, A., Koyama, S. & Kato, T. Mechanisms underlying extensive Ser129-phosphorylation in α-synuclein aggregates. Acta Neuropathol. Commun. 5, 48 (2017).
49. Moon H E, P. S. Mitochondrial Dysfunction in Parkinson's Disease. Exp. Neurobiol. 24, 103-116 (2015).
50. Sivandzade, F., Bhalerao, A. & Cucullo, L. Analysis of the Mitochondrial Membrane Potential Using the Cationic JC-1 Dye as a Sensitive Fluorescent Probe. BIO-PROTOCOL 9, (2019).
51. Gelders, G., Baekelandt, V. & Van der Perren, A. Linking Neuroinflammation and Neurodegeneration in Parkinson's Disease. J. Immunol. Res. 2018, U.S. Pat. No. 4,784,268 (2018).
52. Desai, B. S., Monahan, A. J., Carvey, P. M. & Hendey, B. Blood-brain barrier pathology in Alzheimer's and Parkinson's disease: Implications for drug therapy. in Cell Transplantation vol. 16 285-299 (Cognizant Communication Corporation, 2007).
53. Munji, R. N. et al. Profiling the mouse brain endothelial transcriptome in health and disease models reveals a core blood-brain barrier dysfunction module. Nat. Neurosci. 22, 1892-1902 (2019).
54. Gandhi, P. N., Chen, S. G. & Wilson-Delfosse, A. L. Leucine-rich repeat kinase 2 (LRRK2): A key player in the pathogenesis of Parkinson's disease. Journal of Neuroscience Research vol. 87 1283-1295 (2009).
55. Wakabayashi, K. et al. Synphilin-1 is present in lewy bodies in Parkinson's disease. Atm. Neurol. 47, 521-523 (2000).
56. Youdim, M. B. H. & Bakhle, Y. S. Monoamine oxidase: Isoforms and inhibitors in Parkinson's disease and depressive illness. British Journal of Pharmacology vol. 147 (2006).
57. Carpanini, S. M., Torvell, M. & Morgan, B. P. Therapeutic inhibition of the complement system in diseases of the central nervous system. Frontiers in Immunology vol. 10 (2019).
58. Del Giudice, R. et al. Amyloidogenic variant of apolipoprotein A-I elicits cellular stress by attenuating the protective activity of angiogenin. Cell Death Dis. 5, (2014).
59. Gosselet, F. et al. Transcriptional profiles of receptors and transporters involved in brain cholesterol homeostasis at the blood-brain barrier: Use of an in vitro model. Brain Res. 1249, 34-42 (2009).
60. Furuno, T. et al. Expression polymorphism of the blood-brain barrier component P-glycoprotein (MDR1) in relation to Parkinson's disease. Pharmacogenetics 12.529-534 (2002).
61. Jin, U., Park, S. J. & Park, S. M. Cholesterol metabolism in the brain and its association with Parkinson's disease. Experimental Neurobiology vol. 28 554-567 (2019).
62. Yoon, Y.-S. et al. Is trehalose an autophagic inducer? Unraveling the roles of non-reducing disaccharides on autophagic flux and alpha-synuclein aggregation. Cell Death Dis. 8, e3091 (2017).
63. Hoffmann, A.-C. et al. Extracellular aggregated alpha synuclein primarily triggers lysosomal dysfunction in neural cells prevented by trehalose. Sci. Rep. 9,544 (2019).
64. Larocca, T. J. et al. Translational evidence that impaired autophagy contributes to arterial ageing. J. Physiol. 590, 3305-3316 (2012).
65. Rodriguez-Navarro, J. A. et al. Trehalose ameliorates dopaminergic and tau pathology in parkin deleted/tau overexpressing mice through autophagy activation. Neurobiol. Dis. 39, 423-38 (2010).
66. Lan, D. M. et al. Effect of trehalose on PC12 cells overexpressing wild-type or A53T mutant α-synuclein. Neurochem. Res. 37, 2025-2032 (2012).
67. Lázaro, D. F., Pavlou, M. A. S. & Outeiro, T. F. Cellular models as tools for the study of the role of alpha-synuclein in Parkinson's disease. Experimental Neurology vol. 298 162-171 (2017).
68. Wood, S. J. et al. α-Synuclein fibrillogenesis is nucleation-dependent: Implications for the pathogenesis of Parkinson's disease. J. Biol. Chem. 274, 19509-19512 (1999).
69. McGeer, P. L., Itagaki, S., Boyes, B. E. & McGeer, E. G. Reactive microglia are positive for HLA-DR in the: Substantia nigra of Parkinson's and Alzheimer's disease brains. Neurology 38, 1285-1291 (1988).
70. Lofrumento, D. D. et al. MPTP-induced neuroinflammation increases the expression of pro-inflammatory cytokines and their receptors in mouse brain. Neuroimmunomodulation 18, 79-88 (2010).
71. Mogi, M. et al. Tumor necrosis factor-α (TNF-α) increases both in the brain and in the cerebrospinal fluid from parkinsonian patients. Neurosci. Lett. 165, 208-210 (1994).
72. Mogi, M., Harada, M., Kondo, T., Riederer, P., Inagaki, H., Minami, M. and Nagatsu, T. Interleukin-1β, interleukin-6, epidermal growth factor and transforming growth factor-α are elevated in the brain from parkinsonian patients. Neurosci. Lett. 180, 147-150 (1994).
73. Lee, H. J., Suk, J. E., Bae, E. J. & Lee, S. J. Clearance and deposition of extracellular α-synuclein aggregates in microglia. Biochem. Biophys. Res. Commun. 372, 423 128 (2008).
74. Man, S. et al. CXCL12-induced monocyte-endothelial interactions promote lymphocyte transmigration across an in vitro blood-brain barrier. Sci. Transl. Med. 4, (2012).
75. Zhao, C., Ling, Z., Newman, M. B., Bhatia, A. & Carvey, P. M. TNF-α knockout and minocycline treatment attenuates blood-brain barrier leakage in MPTP-treated mice. Neurobiol. Dis. 26, 36-46 (2007).
76. Jangula, A. & Murphy, E. J. Lipopolysaccharide-induced blood brain barrier permeability is enhanced by alpha-synuclein expression. Neurosci. Lett. 551, 23-27 (2013).
77. Gray, M. T. & Woulfe, J. M. Striatal blood-brain barrier permeability in Parkinson's disease. J. Cereb. Blood Flow Metab. 35, 747-750 (2015).
78. Rite, I., Machado, A., Cano, J. & Venero, J. L. Blood-brain barrier disruption induces in vivo degeneration of nigral dopaminergic neurons. J. Neurochem. 101, 1567-1582 (2007).
79. Logsdon, A. F., Erickson, M. A., Rhea, E. M., Salameh, T. S. & Banks, W. A. Gut reactions: How the blood-brain barrier connects the microbiome and the brain. Exp. Biol. Med. (Maywood). 243, 159-165 (2018).
80. Gonçalves, A., Ambrósio, A. F. & Fernandes, R. Regulation of claudins in blood-tissue barriers under physiological and pathological states. Tissue Barriers 1, e24782 (2013).
81. Maoz, B. M. et al. A linked organ-on-chip model of the human neurovascular unit reveals the metabolic coupling of endothelial and neuronal cells. Nat. Biotechnol. 36, 865-877 (2018).
82. Manatakis, D. V., VanDevender, A. & Manolakos, E. S. An information-theoretic approach for measuring the distance of organ tissue samples using their transcriptomic signatures. bioRxiv 2020.01.23.917245 (2020) doi:10.1101/2020.01.23.917245.
83. Love, M. I., Huber, W. & Anders, S. Moderated estimation of fold change and dispersion for RNA-seq data with DESeq2. Genome Biol. 15, (2014).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Various modifications and variations of the described methods and system of the invention will be apparent to those skilled in the art without departing from the scope and spirit of the invention. Although the invention was described in connection with specific preferred embodiments, it should be understood that the invention as claimed should not be unduly limited to such specific embodiments. Indeed, various modifications of the described modes for carrying out the invention that are obvious to those skilled in biochemistry, chemistry, microbiology, molecular biology, space biology, engineering and medicine, or related fields are intended to be within the scope of the following claims.

1-135. (canceled)

136. A method, comprising,
a) providing,
i) an inflammation inducing compound;
ii) a microfluidic device comprising a membrane, said membrane separating first and second microfluidic channels;
iii) a plurality of cells comprising microglial cells mixed with cells, said cells selected from the group consisting of pericytes, astrocytes, and neurons and combinations thereof; and
iv) a population of endothelial cells;
b) culturing said plurality of cells in said first channel and culturing said population of endothelial cells in said second channel;
c) contacting said cultured cells with said inflammation inducing compound under conditions such that cytokine secretion is induced; and
d) detecting said induced cytokine secretion. wherein the amount of cytokine produced is larger than the amount produced in the absence of said microglial cells.

137. The method of claim 136, wherein said inflammation inducing compound is TNF-alpha.

138. The method of claim 136, wherein said cytokine is IL-6.

139. A method, comprising,
a) providing,
i) an inflammation inducing compound;
ii) a microfluidic device comprising a membrane, said membrane separating first and second microfluidic channels;
iii) a plurality of cells comprising microglial cells mixed with cells, said cells selected from the group consisting of pericytes, astrocytes, and neurons and combinations thereof; and
iv) a population of endothelial cells;
b) culturing said plurality of cells in said first channel and culturing said population of endothelial cells in said second channel; and
c) contacting said cultured cells with said inflammation inducing compound under conditions such that inflammation is induced, wherein said contacting comprises flowing said inflammation inducing compound into said second channel; and
d) detecting transcriptomic changes in the cells.

140. The method of claim 139, wherein said detecting of transcriptomic changes comprises detecting differentially expressed genes.

141. The method of claim 140, wherein the differentially expressed genes are different from those expressed when said inflammation inducing compound is introduced into said first channel.

142. The method of claim 140, wherein increased expression in a plurality of glia-associated genes is detected only after exposure to the inflammation inducing compound through the second channel.

143. The method of claim 142, wherein said glia-associated genes are selected from the group consisting of GFAP, XYLT1, H19, RGS4, TREM2, PADI2, and PADI4.

144. The method of claim 139, wherein said inflammation inducing compound is TNF-α.

145. A method, comprising,
a) providing,
i) α-synuclein (αSyn) fibrils;
ii) a microfluidic device comprising a membrane, said membrane separating first and second microfluidic channels; and
iii) a plurality of dopaminergic neurons in said first channel, said first channel further comprising astrocytes and microglial cells;
b) contacting said dopaminergic neurons with said α-synuclein fibrils; and
c) detecting induced cytokine secretion.

146. The method of claim 145, wherein said contacting comprises flowing said α-synuclein fibrils into said first channel.

147. The method of claim 145, further comprising detecting accumulation of phosphorylated αSyn in said neurons.

148. The method of claim 145, further comprising detecting mitochondrial damage in said neurons.

149. The method of claim 145, further comprising detecting an increase in reactive oxygen species over time.

150. The method of claim 145, further comprising detecting an increase in caspase 3-positive neurons over time.

151. The method of claim 145, further comprising detecting neuroinflammation.

152. The method of claim 145, further comprising detecting apoptosis.

153. The method of claim 145, further comprising detecting neuronal death.

154. The method of claim 145, further comprising detecting microglia activation.

155. The method of claim 145, further comprising detecting astrocyte activation.

156. The method of claim 145, further comprising detecting astrogliosis.

157. A method, comprising,
a) providing,
i) α-synuclein (αSyn) fibrils;
ii) a microfluidic device comprising a membrane, said membrane separating first and second microfluidic channels;
iii) a plurality of dopaminergic neurons in said first channel; and
iv) a population of endothelial cells in said second channel;
b) introducing said α-synuclein fibrils into said device; and
c) detecting an inflammatory response of said endothelial cells.

158. The method of claim 157, wherein said αSyn fibrils are introduced into said second channel.

159. The method of claim 157, further comprising detecting accumulation of αSyn in said endothelial cells.

160. A method, comprising,
a) providing,
i) α-synuclein (αSyn) fibrils;
ii) a microfluidic device comprising a membrane, said membrane separating first and second microfluidic channels;
iii) a plurality of dopaminergic neurons in said first channel; and
iv) a population of endothelial cells in said second channel;
b) culturing said cells such that said endothelial cells form tight junctions, said tight junctions defining a barrier having a level of permeability;
c) introducing said α-synuclein fibrils into said device; and
d) detecting a change in said level of permeability.

161. The method of claim 160, further comprising c) contacting said endothelial cells with a test compound.

162. The method of claim 161, detecting the impact of said test compound on said permeability.

163. A method, comprising,
a) providing,
i) α-synuclein (αSyn) fibrils;
ii) a microfluidic device comprising a membrane, said membrane separating first and second microfluidic channels; and
iii) a plurality of dopaminergic neurons in said first channel;
b) contacting said dopaminergic neurons said α-synuclein fibrils; and
c) detecting neuroinflammation.

164. The method of claim 163, further comprising introducing a drug for testing efficacy for slowing or stopping said neuronal inflammation.

165. A method, comprising,
a) providing,
i) an inflammation inducing compound;
ii) a microfluidic device comprising a membrane, said membrane separating first and second microfluidic channels;
iii) a plurality of cells comprising microglial cells mixed with cells, said cells selected from the group consisting of pericytes, astrocytes, and neurons and combinations thereof; and
iv) a population of endothelial cells;
b) culturing said plurality of cells in said first channel and culturing said population of endothelial cells in said second channel;
c) contacting said cultured cells with said inflammation inducing compound under conditions such that inflammation is induced; and
d) introducing a test compound.

166. The method of claim 165, further comprising using biomarkers to determine the effect of said test compound.

167. The method of claim 166, wherein said using of biomarkers comprises gene expression profiling.

168. The method of claim 165, wherein said inflammation inducing compound is TNF-alpha.

169. The method of claim 165, wherein said inflammation inducing compound comprises α-synuclein (αSyn) fibrils.


SEQUENCE LISTING (1 sequence)

SEQ ID NO: 1 - protein, 140 aa, Artificial Sequence
  Synthetic
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA